(19) United States
(12) Patent Application Publication
(10) Pub. No.: US 2004/0209282 A1
(43) Pub. Date: Oct. 21, 2004

(54) Methods for producing polypeptide-tagged collections and capture systems containing the tagged polypeptides

(76) Inventors: Dana Ault-Riche, Los Gatos, CA (US); Bruce Atkinson, Burlingame, CA (US)

Correspondence Address: FISH & RICHARDSON, PC, 12390 EL CAMINO REAL, SAN DIEGO, CA, 92130-2081, US

(21) Appl. No.: 10/699,088

(22) Filed: Oct. 30, 2003

Related U.S. Application Data
(60) Provisional application No. 60/422,923, filed on Oct. 30, 2002.
(60) Provisional application No. 60/423,018, filed on Oct. 30, 2002.

(51) Int. Cl. C12Q 1/68; G01N 33/53; G01N 33/543

(52) U.S. Cl. 435/6; 435/7.1; 436/518

(57) ABSTRACT

Methods for evenly distributing tags on collections of molecules are provided. Also provided are assay methods that employ the tagged collections.

### RELATED APPLICATIONS

Benefit of priority under 35 U.S.C. §119(e) is claimed to U.S. provisional application Serial No. 60/422,923, filed Oct. 30, 2002, to Dana Ault-Riche and Bruce Atkinson, entitled “METHODS FOR PRODUCING POLYPEPTIDE-TAGGED COLLECTIONS AND CAPTURE SYSTEMS CONTAINING THE TAGGED POLYPEPTIDES” and to U.S. provisional application Serial No. 60/423,018, filed Oct. 30, 2002, to Dana Ault-Riche, Bruce Atkinson, Lynne Jesaitis, Krishnanand D. Kumble and Gizette Sperinde, entitled “SYSTEMS FOR CAPTURE AND ANALYSIS OF BIOLOGICAL PARTICLES AND METHODS USING THE SYSTEMS” is claimed.

This application is related to U.S. application Ser. No. 09/910,120, filed Jul. 18, 2001, to Dana Ault-Riche and Paul D. Kassner, entitled “COLLECTIONS OF BINDING PROTEINS AND TAGS AND USES THEREOF FOR NESTED SORTING AND HIGH THROUGHPUT SCREENING”, published as U.S. application Ser. No. 20020137053, and to U.S. provisional application Serial No. 60/219,183, filed Jul. 19, 2000, to Dana Ault-Riche entitled “COLLECTIONS OF ANTIBODIES FOR NESTED SORTING AND HIGH THROUGHPUT SCREENING”. This application is related to International PCT application No. WO 02/06834. This application also is related to U.S. provisional application Serial No. 60/352,011, filed Jan. 24, 2002, to Dana Ault-Riche and Paul D. Kassner, entitled “USE OF COLLECTIONS OF BINDING PROTEINS AND TAGS FOR SAMPLE PROFILING,” to U.S. patent application Ser. No. 10/351,011 filed Jan. 24, 2003, to Dana Ault-Riche and Paul D. Kassner, entitled “USE OF COLLECTIONS OF BINDING PROTEINS AND TAGS FOR SAMPLE PROFILING,” and to International PCT application No. WO03/062402. This application also is related to U.S. provisional application Serial No. 60/446,687, filed Feb. 10, 2003, to Dana Ault-Riche, Krishnanand D. Kumble, Rainer Schulz and Kenneth Schulz, entitled “SELF-ASSEMBLING ARRAYS AND USES THEREOF.” This application also is related to U.S. application Ser. No. attorney dkt no. 25885-1754PC, entitled “METHODS FOR PRODUCING POLYPEPTIDE-TAGGED COLLECTIONS AND CAPTURE SYSTEMS CONTAINING THE TAGGED POLYPEPTIDES,” to U.S. application Ser. No. attorney dkt. nos. 25885-1759 and 25885-1759PC, each entitled “SYSTEMS FOR CAPTURE AND ANALYSIS OF BIOLOGICAL PARTICLES AND METHODS USING THE SYSTEMS”, and to U.S. application Ser. No. attorney dkt. nos. 25885-1755 and 25885-1755PC, each entitled, “SELF-ASSEMBLING ARRAYS AND USES THEREOF”, filed the same day herewith.

The subject matter of each of the above-noted applications, international applications, published applications and provisional applications is incorporated in its entirety by reference thereto.

### FIELD OF INVENTION

Capture systems that contain collections of binding proteins, called capture agents herein, and polypeptide-tagged molecules, and, particularly to methods for preparing the systems are provided. The systems, methods and collection technology integrate robotic high throughput screening, addressable array and related products and methods.

### BACKGROUND OF THE INVENTION

There are a multitude of technologies designed to gather biological information on a faster and faster scale. Robotics and miniaturization technologies lead to advances in the rate at which information on complex samples is generated. High throughput screening technologies permit routine analysis of tens of thousands of samples; microfluidics and DNA microarray technologies permit information from a single sample to be gathered in a massively parallel manner. DNA microarray chips can simultaneously measure the quantity of more than 10,000 different RNA molecules in a sample in a single experiment.

The sequencing of the human genome has led to the identification of approximately 30,000 genes. These 30,000 genes can generate many-fold greater diversity in message RNA transcripts through alternate splicing reactions. Even more diversity is created through processing of the message RNA into proteins and further post-translational modifications. The combination of these chemical processes (alternative RNA splicing, protein processing and post-translational modifications) increase the diversity of chemical entities into the millions. New tools are therefore needed to begin to understand this molecular complexity.

The chemical environment of a cell is largely controlled by the proteins in the cell. Therefore, information about the abundance, modification state, and activity of the proteins in a cellular sample is extremely valuable in understanding cellular biology. This information is needed to develop new pharmaceuticals and better diagnostic tests for the treatment of disease. DNA microarray technologies provide tools for measuring the abundance of messenger RNA in a sample. There is little correlation between the abundance of messenger RNA for a given protein and the amount of actual protein in the sample. DNA microarrays provide no information about the abundance, modification state or activities of the proteins in a sample.

Proteomics, the large-scale parallel study of proteins, is built upon technologies that simultaneously separate and detect multiple proteins in a solution. A technology in the field of proteomics is two dimensional (2-D) gel electrophoresis. In 2-D gel methods, proteins are separated by charge in one dimension and by size in the other. Following separation, proteins are identified by excision from the gel and analyzed by mass spectrometry. Although 2-D gel methods simultaneously analyze over 1,000 different proteins, these methods are limited by large sample requirements, poor resolution, low sensitivity, inconsistencies in the results and low throughput. Because of its limitations, other methods have been developed, such as ICAT (isotope-coded affinity tags) and MALDI-TOF (matrix-assisted laser desorption ionization time of flight) coupled to chromatography and chip-based SELDI (surface enhanced laser desorption ionization) mass spectrometry methods.

Other approaches employ microarrays of antibodies. In these, antibodies of known specificity are arrayed at discrete physical locations on a solid surface and reacted with antigen-containing mixtures. Unbound material is washed off and the amount of bound antigen is detected. Detection can be effected by indirect detection methods such as reaction with a secondary antibody labeled to produce a fluorescent or chemiluminescent signal, or direct detection such as by detecting changes in the surface plasmon resonance or optical properties of the surface.

Factors, such as an aging population and a need for new pharmaceuticals create enormous pressures for new and more rapid technologies to discover new and better pharmaceutical and diagnostic products. Improved methods for the separation and detection of components of complex mixtures can provide improved diagnostic tests. Improved methods for the separation and detection of components of complex mixtures can provide improved diagnostic tests.

Hence, there remains a need for new methods to separate and detect chemical entities in complex mixtures and to assess complex intra and extracellular pathways. There is a need for new methods to separate and detect chemical entities in complex mixtures, as well as a need to develop new diagnostics and new pharmaceuticals. Therefore, among the objects herein, it is an object to provide methods and products for developing pharmaceutical and diagnostics.

### SUMMARY OF THE INVENTION

Provided herein are methods and systems for developing pharmaceuticals and diagnostics. Methods for discovering compounds, such as antibodies, that have pharmaceutical and diagnostic applications are provided. The methods and systems are tools that provide a way to discover a broad and diverse range of candidate therapeutics and to provide diagnostic tests.

Capture systems that contain addressed collections of capture agents with linked tagged molecules are provided. The tags are either linked to molecules (directly or indirectly or otherwise associated) or are linked by producing fusion proteins from nucleic acid encoding the tags linked directly or indirectly to nucleic acids encoding molecules. The capture agents at each loci to one set of tagged molecules. The diversity displayed at each locus results from the diversity of molecules that share the same tag, which is designed to specifically bind to the capture agent at a single locus. Methods for ensuring that tags are evenly distributed among a collection of molecules are provided. The diversity at each locus can be adjusted to a desired level depending upon the intended application. For an even distribution of tags and uses of the resulting capture systems, it is desirable for each tagged molecule to be unique in each resulting tagged library.

The capture systems provided herein provide an information linkage that does not rely upon a genotype/phenotype linkage. For example, in typical cell-based methods, a cell includes nucleic acid, which is manifested as a particular phenotype. Screening selects for the phenotype, whereby the genotype (gene) responsible for the phenotype is identified. In the systems provided herein, the tags provide an informational link between a phenotype identified by screening and the genotype. This system permits display and screening of increased diversity and of more molecules, by orders of magnitude. Because of the high diversity that is possible at each locus, and also because each locus can be doped or can bind by virtue of a plurality of binding events, it permits screening for weak interactions.

Methods for evenly distributing tags, such as polypeptide tags, among members of a starting (master) library of molecules are provided. The diversity of the starting library, for example, can be 10², 10³, 10⁴, 10⁵, 10⁶, 10⁷, 10⁸, 10⁹, 10¹⁰, 10¹¹, 10¹² or greater. The method includes steps of optionally, adjusting the diversity of a starting library so that the diversity is within an order of magnitude of the number of molecules in the library (generally diversity of the starting library is adjusted to about equal to the number of molecules in the library); dividing the starting library into “n” sub-libraries designated 1 to n, wherein n is equal to or less than the number of unique tags; attaching a unique tag to each sub-library to produce “n” tagged sub-libraries containing tagged members, wherein each member has the same tag and the tag is unique to each sub-library; mixing some or all of the tagged sub-libraries to produce a mixed library, wherein the number of tagged molecules added from each sub-library is the same; and splitting the mixed library into “q” array libraries, wherein q is from 1 up to a predetermined number of arrays.

When tags are evenly distributed the diversity of molecules linked to each tag is about the same, typically within 2, 1, 0.5, 0.1, 0.05 or 0.01 orders of magnitude. The tags are any molecules, such as polypeptides, that specifically bind to capture agents, the library contains any types of molecules, such as, but are not limited to, nucleic acid molecules and polypeptides and proteins. In exemplified embodiments, the libraries are nucleic acid libraries, and the tags are linked to the encoded polypeptides by linking nucleic acid molecules that encode the polypeptide tags to the members of the nucleic acid library.

The tagged molecules are contacted with one or a plurality (up to q) addressed collections of capture agents, in which the agents at each loci specifically bind to the same tag, under conditions which the tags bind to loci on the capture agents to produce capture systems. The resulting capture systems can be used in a variety of methods including methods in which the arrayed tagged molecules are assessed and identified, and methods in which the capture systems are used to bind to additional molecules and/or biological particles in order to assess interactions of the molecules with the capture systems and/or with test and or known compounds and/or conditions, such as pH, temperature, ionic strength, pressure, and other parameters.

Particular exemplary embodiments and methods that are provided include the following. In one embodiment, for example, provided are methods for evenly distributing nucleic acid molecules that encode polypeptide tags among members of a starting library, such as a nucleic acid library, by optionally, adjusting the diversity of a starting library so that the diversity is within an order of magnitude of, typically about equal to, the number of members in the library. Generally the diversity of the starting library is about within about one, or half, 0.1, 0.05, 0.05 or 0.01 of an order of magnitude of the number of members of the library. The method then includes the steps of: dividing the starting library into “n” sub-libraries designated 1 to n, wherein n is equal to or less than the number of different nucleic acid molecules having nucleic acid molecules encoding different polypeptide tags; attaching a nucleic acid molecule encoding a polypeptide tag to members of each sub-library to produce “n” tagged sub-libraries containing tagged members, wherein the encoded polypeptide tag is unique to each sub-library; mixing some or all of the tagged sub-libraries to produce a mixed library, wherein the number of tagged nucleic acid molecules added from each sub-library is the same; splitting the mixed library into “q” array libraries, where q is from 1 to a predetermined number of arrays, and producing, such as by translation and/or expression where the library is a nucleic acid library, the tagged polypeptides in each array library. Generally, the polypeptide tag encoding a portion of the tag is in reading frame with a polypeptide encoded by the nucleic acid molecule.

After distributing the tags among members of a library, the resulting tagged library or tagged array libraries are contacted with 1 up to q collections of addressed collections of capture agents under conditions in which the tags bind to the capture agents to produce 1 to q capture systems. The capture agents at each locus in the addressed collection specifically bind to the same tag. The methods can further include, contacting array libraries with addressed capture agents. The capture agents at each address bind to the same polypeptide tag, thereby sorting the tagged polypeptides according to the bound nucleic acid molecule. The methods can further include producing a capture system from each array library by contacting members of the array library with addressable collections of capture agents and/or preparing up to “q” arrays from the array libraries.

In the resulting array libraries, on the average, each tagged molecule can be unique in each array library. The diversity of the starting library is about equal to the number of molecules in the library or the diversity is within about one, 0.5, 0.1, 0.05 or 0.01 of an order of magnitude of the number of molecules in the library. In the resulting tagged collections of molecules, the diversity of each sub-library of tagged molecules is the same or within about one, 0.5, 0.1, 0.05 or 0.01 of an order of magnitude of all other tagged sub-libraries. The tagged molecules can have any diversity and typically have a diversity of at least about 10², 10³, 10⁴, 10⁵, 10⁶, 10⁷, 10⁸, 10⁹, 10¹⁰, 10¹¹ and 10¹² and greater.

Tags can be linked directly or via a linker to the molecules. For example, where the tag is introduced by linking encoding nucleic acids to a nucleic acid encoding a tag, the resulting encoded polypeptide tag is linked,:directly or via linking amino acids, in frame to polypeptides encoded by nucleic acid molecule members of the library.

In exemplary embodiments, the starting library encodes antibodies or fragments thereof, such as single chain fragments (scFvs), or is comprised of antibodies or fragments thereof. The antibodies and/or fragments specifically bind to capture agents, which can be antibodies or fragments thereof. In other exemplary embodiments, the starting library is a nucleic acid library, for example a cDNA library, or a library encoding antibodies or fragments thereof, such as scFvs.

In an exemplary embodiment, the starting library is a nucleic acid library; and the step of attaching a nucleic acid molecule encoding a polypeptide tag to members of each sub-library is effected by cloning members of the nucleic acid sub-libraries into sets of plasmids or vectors that contain nucleic acid encoding the polypeptide tags; there are up to “n” sets of plasmids; each set of plasmids comprises nucleic acid that encodes a single polypeptide tag and each set encodes a unique polypeptide tag; the members of each sub-library are cloned into a set of plasmids, whereby each member of a sub-library is tagged with the same tag-encoding nucleic acid, and each sub-library is tagged with a unique tag-encoding nucleic acid. Host cells can be transformed or transfected with the resulting plasmids and host cells are then maintained under conditions, such as by cooling or freezing them, whereby the number of plasmids does not increase.

The host cells are then titered and the compositions containing the host cells are normalized so that the titer of each library is about the same (i.e. within 1, 0.5., 0.1, 0.05, 0.01 order of magnitude of each other). Mixed libraries are produced by mixing sets of host cells. The mixed libraries can be used directly or split into from 2 to “q” equal portions, where “q” is a predetermined number. Polypeptides can be produced by expressing and purifying the tagged polypeptides encoded in the plasmids to produce from 1 to q array libraries of tagged polypeptides. Capture systems are then produced by contacting the 1 to q array libraries, with a corresponding number of addressed capture agents to produce from 1 to q capture systems.

The resulting collections of tagged polypeptides and capture systems are provided. For example, a capture system that contains resulting tagged molecules, such as polypeptide tagged polypeptides, and an addressable collection of capture agents, such as capture antibodies is provided. Each locus in the addressable collection contains capture agents that specifically bind to the same tag; and the tagged molecules are specifically bound to capture-agents. In the capture systems provided herein the tags are evenly distributed among the tagged polypeptides; and the tags are evenly distributed among the tagged molecules such that the diversity of tagged molecules at each locus in the collection is within one order of magnitude (generally 0.5, 0.1, 0.05, 0.01) between and among loci. The capture agents can be antibodies or fragments thereof, and the tagged molecules can be polypeptide tagged antibodies or fragments thereof in which the polypeptide tag specifically binds to the antibody (or fragment thereof) capture agent.

The capture systems can further contain an additional agent or plurality thereof at each locus. The amounts and/or the additional agents can vary from locus to locus. The additional agents can be compounds with known activity, and can be drugs, antibodies, nucleic acid molecules, receptors, co-receptors, adhesion molecules, drugs, receptors, enzymes and combinations thereof. They can be organic compounds, inorganic compounds, metal complexes, receptors, enzymes, protein complexes, antibodies, proteins, nucleic acids, peptide nucleic acids, DNA, RNA, polynucleotides, oligonucleotides, oligosaccharides, lipids, lipoproteins, amino acids, peptides, polypeptides, peptidomimetics, carbohydrates, cofactors, prodrugs, lectins, sugars, glycoprotein, biomolecules, macromolecules, antibody conjugates, biopolymers, hormones, growth factors, polymers and any combination, portion, salt, and derivative thereof. Exemplary of these are: adhesion molecules (e.g. ALCAM, BCAM, CADs, EpCAM, ICAMs, Cadherins, Selectins, MCAM, NCAM, PECAM and VCAM); angiogenic factors (e.g. Angiogenin, Angiopoietins, Endothelins, Flk-1, Tie-2 and VEGFs); binding proteins (e.g. IGF binding proteins); cell surface proteins (e.g. B7s, CD14, CD21, CD28, CD34, CD38, CD4, CD6, CD8a, CD64, CTLA-4, decorin, LAMP, SLAM, ST2 and TOSO), cell surface receptors; chemokines (e.g. 6Ckine, BLC/BCA-1, ENA-78, eotaxins, fractalkine, GROs, HCCs, MCPs, MDC, MIG, MIPs, MPIF-1, PARC, RANTES, TARK, TECK and SDF-1); chemokine receptors (e.g. CCRs, CX3CR-1 and CXCRs); cytokines and their receptors (e.g. Epo, Flt-3 ligand, G-CSF, GM-CSF, interferons, IGFs, IK, leptin, LIF, M-CSF, MIF, MSP, oncostatin M, osteopontin, prolactin, SARPs, PD-ECGF, PDGF A and B chains, Tpo, TIGF and PREF-1, AXL, interferon receptors, c-kit, c-met, Epo R, Flt-s/Flk-2 R, G-CSF R, GM-CSF R, etc.); ephrin and ephrin receptors; epidermal growth factors (e.g. amphiregulin, betacellulin, cripto, erbB1, erbB3, erbB4, HB-EGF and TGF-α); fibroblast growth factors (FGFs) and receptors (FGFRs); platelet-derived growth factors (PDGFs) and receptors (PDGFRs); transforming growth factors beta (TGFs-β, e.g. activins, bone morphogenic proteins (BMPs) and receptors (BMPRs), endometrial bleeding associated factor (EBAF), inhibin A and MIC-1); transforming growth factors alpha (TGFs-α); insulin-like growth factors (IGFs); integrins (alphas and betas); interleukins and interleukin receptors; neurotrophic factors (e.g. BDNF, b-NGF, CNTF, CNTF Rα, GDNF, GRFαs, midkine, MUSK, neuritin, neuropilins, NGF R, NT-3, semaphorins, TrkA, TrkB and TrkC); interferons and their receptors; orphan receptors (e.g. Bob, ChemR23, CKRLs, GRPs, RDC-1 and STRL33/Bonzo); proteases and release factors (e.g. matrix metalloproteinases (MMPs), caspases, furin, plasminogen, SPC4, TACE,; TIMPs and urokinase R); T cell receptors; MHC peptides; MHC peptide complexes; B cell receptors; intracellular adhesion molecules (ICAMs); Toll-like receptors (TLRs; recognize extracellular pathogens, such as pattern recognition receptors (PRR receptors)) and PPAR ligands (peroxisome proliferative-activated receptors); ion channel receptors; neurotransmitters and their receptors (e.g. nicotinic acetylcholine, acetylcholine, serotonin, γ-aminobutyrate (GABA), glutamate, aspartate, glycine, histamine, epinephrine, norepinephrine, dopamine, adenosine, ATP and nitric oxide); muscarinic receptors; small molecule receptors (e.g. NO and CO₂ receptors); steriod hormones and their receptors (e.g. progesterone, aldosterone, testosterone, estradiol, cortisol, retinoic acid receptors (RARs), retinoid X receptors (RXRs) and PPARs); peptide hormones and their receptors (e.g. human placental lactogen, prolactin, gonadotropins, corticotropins, calcitonin, insulin, glucagon, somatostatin, gastrin and vasopressin); tumor necrosis factors (TNFs, e.g. April, CD27, CD27L, CD30, CD30L, CD40, CD40L, DR-3, Fas, FasL, HVEM, lymphotoxin β, osteoprotegerin, RANK, TRAILs, TRANCE and TWEAK) and their receptors; nuclear factors; and G proteins and G protein coupled receptors (GPCRs). Others include drugs, such as the anti-Her-2 monoclonal antibody trastuzumab (Herceptin®) and the anti-CD20 monoclonal antibodies rituximab (Rituxan®), tositumomab (Bexxar™) and Ibritumomab (Zevalin™), the anti-CD52 monoclonal antibody Alemtuzumab (Campath™), the anti-TNFα antibodies infliximab (Remicade™) and CDP-571 (Humicade®), the monoclonal antibody edrecolomab (Panorex®), the anti-CD3 antibody muromab-CD3 (Orthoclone®), the anti-IL-2R antibody daclizumab (Zenapax®), the omalizumab antibody against IgE (Xolair®), the monoclonal antibody bevacizumab (Avatin™), and small molecules such as erlotinib-HCl (Tarceva™).

The additional agents can serve to alter the binding surface of the capture system or, for example, to permit identification of co-receptors or drugs that enhance the activity of known drugs. The additional agent can serve to anchor captured molecules and biological particles, to act as a co-stimulatory molecule, to bind to surface receptors different from the first capture agents, to exert a biological effect, to further select the molecules and/or biological particles that bind to a locus. Capture agents also can be selected from among the agents listed as additional agents.

Also provided are collections of tagged molecules, where the tags are evenly distributed among the tagged molecules such that the number of molecules having each tag is within one, 0.5, 0.1, 0.05, or 0.01 order of magnitude; and the collection has a diversity of at least 10³, 10⁴, 10⁵, 10⁶, 10⁷, 10⁸, 10⁹, 10¹⁰, 10¹¹, 10¹², 10¹³ and greater. Embodiments of such collections include nucleic acid library tagged with oligonucleotides that encode polypeptide tags, collections tagged with polypeptide tags, collections of polypeptides tagged with polypeptide tags and addressable collections where the diversity of different tagged molecules at each locus in the array is within one order of magnitude. The collections can be bound to capture agents, such as those described herein.

Methods for capturing molecules and/or biological particles using the capture systems provided herein as well as the capture systems produced as described in co-pending U.S. application Ser. No. 09/910,120, published as U.S. application Ser. No. 20020137053 and as International PCT application No. WO 02/06834, and to U.S. provisional application Serial No. 60/219,183 are provided. In the methods a capture system is contacted with molecules under conditions whereby molecules bind to the capture system. As noted the capture systems include a plurality of addressed loci, such as by positional addressing or labeling, such as by association with electronic, chemical, optically or color-coded labels; the capture systems contain an addressed collection of tagged molecules bound to addressed capture agents at each locus; the capture agents at each locus bind to the same tag; the tag to which the capture agent binds is different among the loci; each locus in the capture system contains a plurality of different molecules each with the same tag bound to the capture agents; and the tags can be evenly distributed among the tagged molecules such that the diversity of tagged molecules at each locus in the capture system is within one order of magnitude or less as described herein (i.e., within 0.5, 0.1, 0.05, 0.01 order of magnitude). The tags can be anything that binds to the capture agents, and typically are polypeptides (i.e., also referred to herein as epitope tags). The tagged molecules can have a diversity of at least 10³, 10⁴, 10⁵, 10⁶, 10⁷, 10⁸, 10⁹, 10¹⁰, 10¹¹, 10¹², 10¹³ and greater. The tagged molecules can be any molecules, including, polypeptides. For example, the tagged polypeptides can be tagged antibodies or fragments thereof, such as single-chain antibody fragments (scFvs).

The tagged molecules can be a library, such as an antibody library and can be produced from a library of nucleic acid molecules encoding an antibody library. The capture agents can be any molecules, such as polypeptides, nucleic acids, receptors, ligands, drugs, enzymes, enzymes that are modified to have reduced catalytic activity, and/or analogs and combinations of any molecules, that specifically bind to the tags. For example, the capture agents can be antibodies or fragments thereof.

The resulting capture systems are typically addressable arrays, such as a positionally addressable array. They can contain the capture agents immobilized at discrete loci on a solid support. Exemplary solid supports, include, but are not limited to, selected from the group consisting of silicon, celluloses, metal, polymeric surfaces, radiation grafted supports, such as radiation grafted polytetrafluoroethylene, gold, nitrocellulose, polyvinylidene fluoride (PVDF), polystyrene, glass and activated glass. The support can include a well or a pit or plurality thereof in or on a surface of the solid support. The capture agents are addressably tagged by linking them to electronic, chemical, optically or color-coded labels, for example labels associated with particulate supports. Particulate supports include, but are not limited to, silicon, celluloses, metal, polymeric surfaces, radiation grafted supports, gold, nitrocellulose, polyvinylidene fluoride (PVDF), radiation grafted polytetrafluoroethylene, polystyrene, glass and activated glass The methods for capturing molecules and/or biological particles can further include at each locus in the capture system an additional agent or plurality thereof at one or more loci, wherein the additional agents are common to a plurality of loci, and bind to and/or interact with the captured biological particles and/or captured molecules. Such additional agents are described herein and above. The amounts of the additional agents-can vary from locus to locus.

Methods that use the capture systems can further include the step of assessing the effects of capture on a captured molecule or plurality thereof. These methods employ the capture systems produced by the methods provided herein, and also by the methods described in co-pending U.S. application Ser. No. 09/910,120, published as U.S. application Ser. No. 20020137053 and as International PCT application No. WO 02/06834. Effects, include, for example, a change in activity, a physical change, a chemical change. These effects can be detected, for example, by visualizing the captured molecules, such as by staining or labeling captured molecules. The methods can further include detecting or identifying captured molecules and/or identifying tagged molecules that capture the molecules or labeled molecules. Molecules can be labeled prior to, during or after capture. The stain can be selected to specifically react with one or a plurality of the captured molecules. Also, a plurality of different stains can be used to visualize different molecules or events or portions of molecules. For example, one stain can be selected to react with a feature common to all molecules of a particular type, and at least one other stain reacts with a subset thereof. Patterns of staining can be identified and analyzed. Stains include, but are not limited to, fluorescent dyes, luminescent labels, enzyme labels, green fluorescent protein, red fluorescent protein, blue fluorescent protein, immunostains and semiconductor crystals.

Contacting of molecules can be performed in the presence and absence of a test compound or a condition. Results can be compared to identify test compounds that alter binding of molecules to the capture system. The test compound or exposure to a condition(s) can be performed before, during or after contacting the capture system with the molecules.

Methods of identifying modulators of interactions between capture systems and molecules by preparing capture systems and assessing and adding a test compound or exposing the capture system to a condition before, during or after contacting the capture system with the molecules or before, during or after contacting the capture agents with the tagged molecules; and identifying changes in the interactions of the molecules with the capture system or tagged molecules with the capture agents to identify test compounds that modulate interactions between the molecules and the capture system or between tagged molecules and capture agents. Changes can be assessed by detecting a change in binding pattern or a physical or chemical change in the bound molecules or a conformational change in the bound molecules and/or tagged molecules.

Methods of sorting molecules or reducing the diversity using the capture systems and profiling are provided. These methods are described in copending U.S. application Ser. No. 09/910,120, published as U.S. application Ser. No. 20020137053 and as International PCT application No. WO 02/06834, and to U.S. provisional application Serial No. 60/219,183. Briefly, for example, the methods can include contacting tagged molecules with an array of addressed capture agents, where the agents-at each addressed locus specifically bind the same tag, which differs from the tag to which agents at other loci bind; identifying from among the tagged molecules those having a predetermined activity or property; based upon the tag(s) of the identified molecules, identifying the molecules linked to the tag.

The capture systems are those as described above, and can contain any type of capture agent, and tagged molecule, such as polypeptide-tagged molecules. Capture agents for use herein, include, but are not limited to, enzymes and other catalytic polypeptides, including, but are not limited to, portions thereof to which substrates specifically bind, enzymes modified to retain binding activity lacking catalytic activity; antibodies and portions thereof that specifically bind to antigens or sequences of amino acids; nucleic acids; and cell surface receptors, opiate receptors and hormone receptors and other receptors that specifically bind to ligands, such as hormones. Exemplary capture agents include T cell receptors, MHC peptides, MHC peptide complexes, B cell receptors, ICAMs, Toll-like receptors (recognize extracellular pathogens, such as pattern recognitions receptors (PRR receptors)), PPAR ligands (peroxisome proliferative-activated receptors), ion channels, chemokine receptors, nicotinic acetylcholine receptors, dopamine receptors, muscarinic receptors, small molecule receptors (NO), ICAMs, TNF receptors, interleukin receptors, VCAMS (vascular cell adhesion molecules), interferons and any of those noted above as additional agents.

Biological particles for use with the capture systems and in the methods herein include, but are not limited to, cells, portions of cells, cell membranes, viruses, viral capsids, viral particles, bacterial cells, subcellular compartments, organelles and micelles. For example, biological particles include prokaryotic cells, eukaryotic cells, intracellular particles, nuclei, cell membranes, cell membrane fragments, nuclear membranes, nuclear membranes fragments, viral vectors or viral capsids with or without packaged nucleic acid, phage, phage vectors, phage capsids with or without encapsulated nucleic acid,,liposomes and other micellar agents. The biological particles can be cells that contain a reporter gene construct that includes a transcriptional regulatory region whose activity is modulated by interaction of a protein in or on the cell with a modulator of the activity of the protein. Exemplary biological particles, include, but are not limited to, immune cells, neurons, cancer cells, bacterial cells and infected cells, such as subcellular compartments, organelles, viral particles.

Also provided are methods for generating capture agent/binding partner pairs. In embodiment, a methods for generating such pairs is provided in which binding partner pairs are designed and then used to produce, select or generate capture agents. This method includes steps of: a) ranking amino acids based upon their frequency in a pre-selected set of antigenic polypeptides, wherein “n” amino acids are ranked; b) based upon the ranking using the top “n−1” to “n−n+1,” generating all combinations of the amino acids in a polypeptide of pre-selected length “m” residues to produce a set of polypeptides of length m residues; and c) based upon pre-determined criteria for dissimilarity, selecting a subset of set of dissimilar polypeptides.

### DESCRIPTION OF THE DRAWINGS

FIGS. 1A and 1B depict exemplary methods for isolating capture agent/tag pairs; FIG. 1A shows a panning method and FIG. 1B shows an immunization method.

FIG. 2 illustrates nested sorting using sorting by pools.

FIG. 3 also illustrates nested sorting using sorting by pools, decreasing pool diversities; this sort is identical to the sort illustrated in

FIG. 4 except that the F2 and F3 sort libraries have been arranged into arrays.

FIG. 4 further illustrates nested sorting and the reduction in diversity that is achieved by sorting by pools, screening large diversity libraries.

FIG. 5 depicts a collection of capture agents with bound tagged-agents, showing the diversity of tagged reagent on a surface. Each tag is bound to a plurality of different agents resulting in a surface with a large diversity of binding sites.

FIGS. 6A and 6B depict steps for evenly distributing tags throughout a collection of polypeptides.

FIGS. 7A and 7B depict screening for test compounds or conitions that modulate interactions and screening for test compounds or conitions that modulate the effect of interactions, respectively. The figures depict different screening methods using capture systems to capture cells in the presence and absence of test compounds and conditions.

FIG. 8 depicts the plasmid map for-the pBAD/gIII vector (Invitrogen, Carlsbad, Calif.).

FIG. 9 depicts cells that have been captured on the capture systems provided herein.

FIG. 10 depicts idiotype receptors from cell lysates that have been specifically captured by anti-idiotype antibodies on arrays.

FIG. 11 depicts an exemplary process for designing polypeptide binding partners.

For clarity of disclosure, and not by way of limitation, the detailed description is divided into the subsections that follow.

### DETAILED DESCRIPTION OF EXEMPLARY EMBODIMENTS

A. Definitions

B. Capture Agents and Polypeptide Tags

1. Capture Agents

2. Polypeptide Tags and Preparation Thereof

3. Identification of Capture Agents—Polypeptide Tag Pairs

a. Panning Phage Displayed Peptide Libraries

b. Analysis of Complementarity-determining Regions (CDRs) of the Antibody

c. Theoretical Molecular Modelling of Three-Dimensional Antibody Structure

d. Raising Antibodies from Exposure of a Subject to an Antigen

4. Preparation of Capture Agent Arrays

5. Preparation of Other Addressable Collections

6. Interactions Between Capture Agents and Polypeptide Tags

7. Design and Preparation of Oligonucleotides/Primers

8. Supports for Immobilizing Capture Agents

a. Natural Support Materials

b. Synthetic Supports

c. Immobilization and Activation

C. Preparation of the Capture Systems

1. Determining the Required Diversity of the Master Library

2. Creation of the Master library and Division into Sub-libraries

3. Adjusting the diversity of a master library so that the diversity is about equal to the number of members of the library

4. Dividing the Master Library into Sub-libraries

5. Creation of Tagged Libraries

a. Ligation to create circular plasmid vector for introduction of tags

b. Ligation of sequences resulting in linear tagged cDNA

c. Primer extension and PCR for tag incorporation

d. Insertion by Gene Shuffling

e. Recombination strategies

f. Incorporation by transposases

g. Incorporation by splicing

6. Mixing some or all of the tagged sub-libraries to produce a mixed library, where the number of tagged nucleic acid molecules added from each tagged sub-library is the same

7. Splitting the mixed library into “q” array libraries, wherein q is from 1 to a predetermined number of arrays

8. Expression of Array Libraries and Purification of Tagged Molecules to produce collections of tagged molecules with even distributions of tags

9. A plurality of polypeptide tags

D. Nested Sorting Using Addressable Arrays

E. Sample Profiling Using Collections of Capture Agents and Polypeptide Tags

F. Staining of Bound Molecules

1. Methods of Staining

2. Molecules for Staining

G. Use of capture systems for capturing and analyzing biological particles and for drug discovery and other screening applications

1. Capture of biological particles

a. Doping of Loci with Secondary Agents

b. Fixation of Cells to Capture Array

2. Methods to Detect Secondary Effects of Cell Binding to Capture Systems

a. Transcription Reporters

(1) Reporter gene constructs

(2) Reporter genes

(3) Transcriptional control elements

b. Immunostaining

(1) Enzymes and Chromagens for Immunostaining

(a) Luminescent Labels

(b) Horseradish Peroxidase (HRP)

(c) Alkaline Phosphatase (AP)

(2) Avidin-Biotin Staining Methods

(3). Chain Polymer-Conjugated Technology

c. Resonance Energy Transfer

(1) Luminescence Processes

(a) The Fluorescence Process

(b) Quenching Processes

i) Photobleaching

ii) Self-quenching, Static quenching and Collisional quenching

(2) Luminescent Resonance Energy Transfer (LRET)

(a) Förster Distance

(b) Donor/Acceptor Pairs

(3) Luminescent Labels

(a) Fluorophores and Quenchers

(b) Bioluminescent Molecules

(c) Phosphorescent Molecules

3. Identifying Test Compounds and/or Conditions that modulate Interactions among Biological Particles and Capture Systems or Secondary Effects of the Interactions

a. Perturbations and screening methods

b. Perturbations for Assessing Interactions or the Effect of the Interaction

4. Other Exemplary Applications

a. Cell Surface Profiling

b. Receptor Agonist/antagonist Discovery

c. Protein-protein Interactions Including Association-disseciation Assays and Changes in Protein Conformation

d. Biopolymer D gradation Assays Protein Trafficking Assays

f. Analysis of Modulation of Subcellular Conditions and Processes

g. Assays for Assessing Cell Growth and Proliferation

h. Assays for Assessing Apoptosis

i. Assays to Assess Changes in Cell Morphology

j. mRNA Expression Change Assays

k. Receptor Internalization Assays

l. Receptor-Mediated Cell Activation Assays

m. Receptor Activated Cell Signaling

n. Epitope Mapping

o. Sorting Through Library Diversity and Cell Type Diversity

p. Expression of Secreted Polypeptides by Tumor Cells

q. Differentiation/Dedifferentiation Assays

r. Cell-cell Interactions

s. Discover Molecules that Block Binding/Cleavage/Post-translational Modifications

t. Simultaneous Capture of Multiple Cell Types Followed by Functional Assays for Drug Interactions

u. Organ Cultures (e.g. Promotion of Hair- Growth)

v. Discovery of Antibodies to Apically-localized Cell-surface Proteins, Carbohydrates and Lipids

w. Infectious Agents on Arrays

x. Monitoring of Endocytosis, Exocytosis and Phagocytosis

y. Internalization of Libraries by Cultured Cells

z. Detection of Phosphorylation and Dephosphorylation Activities

aa. Determination and Monitoring of Chemical or Enzymatic Kinetics

H. Identification of binding partner polypeptides

1. Overview of the methods

2. Description of the methods

a. Use of non-naturally occurring amino acids for polypeptide design and generation

b. Generation of polypeptides

I. Identification of binding proteins for polypeptide binding partner pairs

1. Raising antibodies

2. Phage display

3. Generation of Binding protein-binding partner pairs

J. EXAMPLES

### A. DEFINITIONS

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, nested sorting refers to the process of decreasing diversity using the addressable collections of antibodies provided herein.

As used herein, profiling refers to detection and/or identification of a plurality of components, generally 3 or more, such as 4, 5, 6, 7, 8, 10, 50, 100, 500, 1000, 10⁴, 10⁵, 10⁶, 10⁷ or more, in a sample. A profile refers to the identified loci to which components of a sample detectably bind. The profile can be detected as a pattern on a solid surface, such as in embodiments when the addressable collection includes an array of capture agents on a solid support, in which case the profile can be presented as a visual image. In embodiments, such as those in which the capture agents and bound tagged molecules are on color-coded beads or are otherwise detectably labeled, a profile refers to the identified polypeptide tags and/or capture agents to which components is(are) detectably bound, which can be in the form of a list or database or other such compendium.

As used herein, an image refers to a collection of datapoints representative of the profile. An image can be a visual, graphical, tabular, matrix or other depiction of such data. It can be stored in a database.

As used herein, a database refers to a collection of data items.

As used herein, a relational database is a collection of data items organized as a set of formally-described tables from which data can be accessed or reassembled in many- different ways without having to reorganize the database tables. Such databases are readily available commercially, for example, from Oracle, IBM, Microsoft, Sybase, Computer Associates, SAP, or multiple other vendors. Databases can be stored on computer-readable media, such as floppy disks, compact disks, digital video disks, computer hard drives and other such media.

As used herein, an address refers to a unique identifier whereby an addressed entity can be identified. An addressed moiety is one that can be identified by virtue of its address. Addressing can be effected by position on a surface or by other identifiers, such as a tag encoded with a bar code or other symbology, a chemical tag, an electronic, such RF tag, a color-coded tag or other such identifier.

As used herein, a capture system refers to an addressable collection of capture agents and polypeptide-tagged molecules bound thereto, where each different polypeptide tag specifically binds to a different capture agent.

As used herein, a molecule, such as capture agent, that specifically binds to a polypeptide, such as a polypeptide tagged molecule provided herein, typically has a binding affinity (Kₐ) of at least about 10⁶ l/mol, 10⁷ l/mol, 10⁸ l/mol, 10⁹ l/mol, 10¹⁰ l/mol or greater (generally 10⁸ or greater) and binds generally with greater affinity (typically at least 10-fold, generally 100-fold or) than to the molecules and biological particles that are to be detected or assessed in the methods that employ the capture systems. Thus, affinity refers to the strength of interaction between a capture agent and a polypeptide tag.

As used herein, specificity (or selectively) with respect to the tags and capture agents refers to the greater affinity the tag and capture agent exhibit compared to the molecules and biological particles that are to be detected by the capture systems.

As used herein, used to “bind” to a capture system means to interact with sufficient affinity to immobilize the bound moiety (biological particle) temporarily under the conditions of a particular experiment. For purposes herein, it is an interaction that permits biological particles, such as cells, to be retained at a locus when cells are contacted with the capture systems so that they no longer move by Brownian motion or other microcurrents in a composition.

As used herein, a landscape is the information produced or presented on a canvas or array.

As used herein, an addressable collection of anti-tag capture agents (also referred to herein as an addressable collection of capture agents) is a collection of protein agents (i.e., receptors), such as antibodies, that specifically bind to pre-selected polypeptide tags that contain sequences of amino acids, such as epitopes in antigens, in which each member of the collection is labeled and/or is positionally located to permit identification of the capture agent, such as the antibody, and tag. The addressable collection is typically an array or other encoded collection in which each locus contains capture agents, such as antibodies, of a single specificity and is identifiable. The collection can be in the liquid phase if other discrete identifiers, such as chemical, electronic, colored, fluorescent or other tags are included. Capture agents, include antibodies and other anti-tag receptors. Any moiety, such as a protein, nucleic acid or other such moiety, that specifically binds to a pre-determined sequence of amino acids, such as an epitope, is contemplated for use as a capture agent.

As used herein, an addressable collection of binding sites refers to the resulting sites produced upon binding of the capture agents provided herein to polypeptide-tagged reagents. Each capture agent sorts reagents (such as molecules and biological particles) by virtue of their tags, each tag is linked to a plurality of different molecules, generally polypeptides. As a result, upon sorting, the capture agent and polypeptide tagged-reagent form a complex and the resulting complex can bind to further molecules. Since the tagged reagents-specific for each capture agent can contain a plurality of different molecules that share the same tag, when bound to a plurality of different capture agents the resulting collection presents a highly diverse collection of binding sites. The collection is addressable because the identity of the tags is known or can be ascertained.

As used herein, polypeptide tags (also referred to as epitope tags, although the polypeptide tag is not necessarily an epitope) generically refer to the tags that include a sequence of amino acids, that specifically binds to a capture agent.

As used herein, a polypeptide tag generally refers to a sequence of amino acids that includes the sequence of amino acids, herein also referred to as an epitope, to which a capture agent, such as an antibody specifically binds. The epitope can be other than a polypeptide; as long as at least a portion of it specifically binds to a capture agent. Furthermore, as described in more detail below, the tags (or encoding nucleic acid molecules) can include a plurality of domains, including, but are not -limited to, a tag-specific amplification sequence (herein referred to as an R-tag) and nucleic acid encoding a ligand-binding domain.

For polypeptide tags, the specific sequence of amino acids to which each binds is referred to herein generically as an epitope. Any sequence of amino acids that binds to a receptor (capture agent) therefor is contemplated. For purposes herein the sequence of amino acids of the tag, such as epitope portion of the polypeptide (epitope) tag, that specifically binds to a capture agent is designated “E”, and each unique epitope is an Eₘ. Depending upon the context “Eₘ” also can refer to the sequences of nucleic acids encoding the amino acids constituting the tag. The polypeptide tag, i.e., the epitope tag, also can include additional amino acids and/or the oligonucleotide or nucleic acid molecule encoding the tag can include additional sequences of nucleotides that can serve as primers or portions of primers. In particular, the polypeptide (epitope) tag is encoded by the oligonucleotides provided herein, which are used to introduce the tag. When reference is made to an epitope tag (i.e. binding pair for a particular capture agent or portion thereof) with respect to a nucleic acid, it is nucleic acid encoding the tag to which reference is made. For simplicity each polypeptide tag is referred to as Eₘ; when nucleic acids are being described the Eₘ is nucleic acid and refers to the sequence of nucleic acids that encode the epitope; when the translated proteins are described Eₘ refers to amino acids (the actual epitope). The number of Es corresponds to the number of antibodies in an addressable collection. “m” is typically at least 10, 30 or more, 50 or 100 or more, and can be as high as desired and as is practical. Generally “m” is about 100, 250, 500, 1000 or more. As discussed below, other moieties that function as binding partners for capture agents also are contemplated.

The polypeptide (epitope) tag is encoded by nucleic acid that can include a plurality of domains, including: one domain that encodes a sequence of amino acids that specifically binds to a capture agent; and a second, optional, domain that serves a primer site (or portion thereof) for specific amplification of the binding amino acids and any other amino acids fused thereto. The second domain, as a whole or in part, may or may not be translated into a protein. A second or further domain also can include other functional signals, such as stop codons, or ribosome binding sites, translation initiation sites and other such sites. The domains can be adjacent to each other or separated or overlapping. In some embodiments, the second domain, is referred to herein as an R-tag.

As used herein, Dₙ refers to each divider sequence, which are optional components of the nucleic acid molecule that encodes a polypeptide, and is not employed in the method provided herein for even distribution of tags. As with each Eₘ the Dₙ is either nucleic acid or amino acids depending upon the context. Each Dₙ is a divider sequence that is encoded by a nucleic acid that serves as a priming site to amplify a subset of nucleic acids. The resulting amplified subset of nucleic acids contains all of the collection of Eₘ sequences and the Dₙ sequences used as a priming site for the amplification. As described herein, the nucleic acids can include a portion, generally at the end, that encodes each EₘDₙ. Generally the encoding nucleic acid is 5′-Eₘ-Dₙ-3′ on the nucleic acid molecules in the library. D is an optional unique sequence of nucleotides for specific amplification to create the sub-libraries. For large libraries, the original library can be divided into sub-libraries and then the tag-encoding sequences added, rather than adding the tag-encoding sequences to the master library. The size of D is a function of the library to be sorted, since the larger the library the longer the sequence needed to specify a unique sequence in the library. Generally D, depending upon the application, is at least 14 to 16 nucleic acid bases long and it may or may not encode a sequence of amino acids, since its function in the method is to serve as a priming site for PCR amplification, D is 2 to n, where n is 0 or is any desired number and is generally 10 to 10,000, 10 to 1000, 50 to 500, and about 100 to 250. The number of D can be as high as 10⁶ or higher. The divider sequences D are used to amplify each of the “n” samples from the tagged master library, and generally is equal to the number of antibody collections, such as arrays, used in an initial sort. * The more collections (divisions) in the initial screen, the lower diversity per addressable locus. The initial division number is selected based upon the diversity of the library and the number of capture agents. As used herein, operably linked to/associated with means that a regulatory DNA sequence is “operably linked to” or “associated with” a coding DNA sequence if the two sequences are situated such that the regulatory DNA sequence affects expression of the coding DNA sequence. The coding regions of two or more genes or gene fragments are likewise “operably linked to” or “associated with” each other if the two or more sequences are situated such that the transcription and translation of the adjacent coding regions results in a fusion protein.

As used herein, a fusion protein refers to a polypeptide that contains at least two components, such as a biomolecular component of a target and a polypeptide tag, and is produced by expression of nucleic acid in a host cell.

As used herein, diversity (Div) refers to the number of unique (non-duplicated) molecules in a library, such as a nucleic acid library. Diversity is distinct from the total number of molecules in any library, which is equal to or greater than the diversity.

As used herein, an “even distribution of tags” means that the diversity of molecules to be tagged is approximately equivalent for each of the tags so that in any collection of tagged molecules on average each tagged molecule is unique. As a result, the diversity of different tagged molecules on the loci (spots in a solid phase array) in each array provided herein is approximately the same (i.e., to within, one order of magnitude, or 0.5 orders of magnitude, or 0.25 orders of magnitude or less). In addition, the diversity of different tags at each locus approaches 1, and is typically less than 100, 50, 10 or 5. The tolerance for variation in diversity in tags at each locus is a function of the application of the resulting capture systems or arrays.

Diversity of tags at a locus is not to be confused with the diversity of molecules at each locus. When tags are evenly distributed amongst molecules in a collection, then the diversity of tagged molecules at each locus is approximately (i.e., to within, one order of magnitude, or 0.5 orders of magnitude, or 0.25 orders of magnitude or less). While the diversity of tags at each locus ideally approaches 1, the diversity of tagged molecules can be any desired number and is typically at least 10², 10³, 10⁴, 10⁵, 10⁶, 10⁷, 10⁸, 10⁹, 10¹⁰, 10¹¹, 10¹² or greater. The diversity of tagged molecules is a function of the application. For example, in embodiments in which molecules present in low copy number or that have a small effect are detected, then a lower variation in diversity among the loci is advantageous. In embodiments in which an effect that is screened is readily detectable and/or the molecules that exhibit the effect are present in higher copy numbers, then a greater variation in diversity (i.e., one order of magnitude) can be tolerated. Tagged libraries produced by the method provided herein for achieving even distribution have an even distribution of tags.

An even distribution can be assessed by any suitable method, such as by taking a sample from a plurality of loci, and sequencing the tags or sequencing samples from the mixed library. Alternatively, ELISA using samples of the tagged molecules can be performed using an antibody specific for the tag. The results will show relative abundance of the tag in each sample. Alternatively, the expressed proteins can be chewed up and the resulting fragments assessed by mass spectrometry to assess diversity.

As used herein, an array refers to a collection of elements, such as antibodies, containing three or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interaction of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a canvas is a collection of arrays, such as those provided herein. The size of each-array and number in a canvas can vary and is at least two and is up to a predetermined number, such as q, which is 2 to 10, 20, 30, 50, 100, 200, 250, 300, 500, 1000, 2000, 3000, 4000, 5000, 10,000 and more, including 96 and multiples thereof (i.e., 384, 1536 and higher densities).

As used herein, a support (also referred to as a matrix support, a matrix,- an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein can be particulate or can be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which can be any shape, including random shapes, needles, fibers, and elongated. Roughly spherical “beads”, particularly microspheres that can be used in the liquid phase, also are contemplated. The “beads” can include additional components, such as magnetic or paramagnetic particles (see, e.g., Dynabeads® (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and can be on the order of cubic microns. Such particles are collectively called “beads.”

As used herein, a capture agent, which is used interchangeably with a receptor, refers to a molecule that has an affinity for a given ligand or with a defined sequence of amino acids. Capture agents can be naturally-occurring or synthetic molecules, and include any molecule, including nucleic acids, small organics, proteins and complexes that specifically bind to specific sequences of amino acids. Capture agents are receptors and also are referred to in the art as anti-ligands. As used herein, the terms, capture agent, receptor and anti-ligand are interchangeable. Capture agents can be used in their unaltered state or as aggregates with other species. They can be attached or in physical contact with, covalently or noncovalently, a binding member, either directly or indirectly via a specific binding substance or linker. Examples of capture agents, include, but are not limited to: antibodies, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, and organelles. For example, the capture agents can specifically bind to DNA binding proteins, such as zinc fingers, leucine zippers and modified restriction enzymes.

Examples of capture agents, include, but are not restricted to:

a) enzymes and other catalytic polypeptides, including, but are not limited to, portions thereof to which substrates specifically bind, enzymes modified to retain binding activity lacking catalytic activity;

b) antibodies and portions thereof that specifically bind to antigens or sequences of amino acids;

c) nucleic acids;

d) cell surface receptors, opiate receptors and hormone receptors and other receptors that specifically bind to ligands, such as hormones.

For the collections herein, the other binding partner, referred to herein as a polypeptide tag for each refers to the substrate, antigenic sequence, nucleic acid binding protein, receptor ligand, or binding portion thereof.

As noted, contemplated herein, are pairs of molecules, generally proteins that specifically bind to each other. One member of the pair is a polypeptide that is used as a tag and encoded by nucleic acids linked to the library; the other member is anything that specifically binds thereto. The collections of capture agents, include receptors, such as antibodies or enzymes or portions thereof and mixtures thereof that specifically bind to a known or knowable defined sequence of amino acids that is typically at least about 3 to 10 amino acids in length. Other examples of capture agents are set forth throughout the disclosure.

As used herein, master library refers to a collection of molecules, such as a cDNA library encoding proteins, to be analyzed or displayed or assessed. These molecules do not contain polypeptide tags nor nucleic acid molecules encoding the tags. In the methods provided herein, for evenly distributing tags in libraries the master libraries are libraries of nucleic acid molecules, such as cDNA libraries.

As used herein, sub-library refers to the initial collection of different libraries produced by subdividing a master library. The sub-libraries are created by physical separation of a master library into n number of discrete collections.

As used herein, tagged library refers to the resulting collections of molecules after the sub-libraries have been separately tagged.

As used herein, normalized tagged libraries refers to resulting collections of molecules after the number of molecules in each tagged library has been estimated and then adjusted such that each normalized tagged library contains approximately the same diversity and number of molecules.

As used herein, mixed library refers to the resulting collection of molecules after normalized tag libraries have been combined.

As used herein, array library refers to the collections of molecules created by physical separation of the mixed library into q number of discrete collections. The array libraries serve as the genetic source for the tagged molecules to be expressed and purified and contacted with arrays of capture agents. Nucleic acid molecules from these libraries also serve as the source of template DNA used in the amplification protocols to recover the desired tagged molecules once identified using the arrays.

As used herein, printing refers to immobilization of capture agents onto a solid support, such as, but not limited to, a microarray.

As used herein, self-sorting refers to separation of a library of epitope-tagged molecules based on the affinity of the epitope for a specific capture agent.

As used herein, the total display refers to the total diversity of molecules being displayed on the arrays.

As used herein, a B cell refers to a lymphocyte that develops from hematopoietic stem cells in the bone marrow of adults and the liver of fetuses and is responsible for the production of circulating antibodies.

As used herein, a T cell refers to a lymphocyte that develops in the thymus from precursor cells that migrate there from the hematopoietic tissues via the blood. T cells fall into two main classes, cytotoxic T cells and helper T cells. Cytotoxic T cells kill infected cells, whereas helper T cells help to activate macrophages, B cells and cytotoxic T cells.

As used herein, antibody refers to an immunoglobulin, whether natural or partially or wholly synthetically, such as recombinantly, produced, including any derivative thereof that retains the specific binding ability of the antibody. Hence antibody includes any protein having a binding domain that is homologous or substantially homologous to an immunoglobulin binding domain. For purposes herein, antibody includes antibody fragments, such as Fab fragments, which are composed of a light chain and the variable region of a heavy chain. Antibodies include members of any immunoglobulin class, including IgG, IgM, IgA, IgD and IgE. Also contemplated herein are receptors that specifically bind to a sequence of amino acids.

Hence for purposes herein, any set of pairs of binding members, referred to generically herein as a capture agent/polypeptide tag, can be used instead of antibodies and epitopes per se. The methods herein rely on the capture agent/polypeptide tag, such as an antibody/epitope tag, for their specific interactions, any such combination of capture agents (receptors/ligands; epitope tag) can be used. Furthermore, for purposes herein, the capture agents, such as antibodies employed, can be binding portions thereof.

As used herein, a monoclonal antibody refers to an antibody secreted by a hybridoma clone. Because each such clone is derived from a single B cell, all of the antibody molecules are identical. Monoclonal antibodies can be prepared using standard methods known to those with skill in the art (see, e.g., Köhler et al. Nature 256:495 (1975) and Köhler et al. Eur. J. Immunol. 6:511 (1976)). For example, an animal is immunized by standard methods to produce antibody-secreting somatic cells. These cells are then removed from the immunized animal for fusion to myeloma cells.

Somatic cells with the potential to produce antibodies, particularly B cells, are suitable for fusion with a myeloma cell line. These somatic cells may be derived from the lymph nodes, spleens and peripheral blood of primed animals. Specialized myeloma cell lines have been developed from lymphocytic tumors for use in hybridoma-producing fusion procedures (Köhler and Milstein, Eur. J. Immunol. 6:511 (1976); Shulman et al. Nature 276: 269 (1978); Volk et al. J. Virol. 42: 220 (1982)). These cell lines have been developed for at least three reasons. The first is to facilitate the selection of fused hybridomas from unfused and similarly indefinitely self-propagating myeloma cells. Usually, this is accomplished by using myelomas with enzyme deficiencies that render them incapable of growing in certain selective media that support the growth of hybridomas. The second reason arises from the inherent ability of lymphocytic tumor cells to produce their own antibodies. The purpose of using monoclonal techniques is to obtain fused hybrid cell lines with unlimited life spans that produce the desired single antibody under the genetic control of the somatic cell component of the hybridoma. To eliminate the production of tumor cell antibodies by the hybridomas, myeloma cell lines incapable of producing endogenous light or heavy immunoglobulin chains are used. A third reason for selection of these cell lines is for their suitability and efficiency for fusion. Other methods for producing hybridomas and monoclonal antibodies are well known to those of skill in the art.

As used herein, antibody fragment refers to any derivative of an antibody that is less than full length, retaining at least a portion of the full-length antibody's specific binding ability. Examples of antibody fragments include, but are not limited to, Fab, Fab′, F(ab)₂, single-chain Fvs (scFv), Fv, dsFv, diabody and Fd fragments. The fragment can include multiple chains linked together, such as by disulfide bridges. An antibody fragment generally contains at least about 50 amino acids and typically at least 200 amino acids.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stabilizes the V_{H}-V_{L} pair.

As used herein, an F(ab)₂ fragment is an antibody fragment that results from digestion of an immunoglobulin with pepsin at pH 4.0-4.5; it can be recombinantly produced.

As used herein, an Fab fragment is an antibody fragment that results from digestion of an immunoglobulin with papain; it can be recombinantly produced.

As used herein, scFvs refers to antibody fragments that contain a variable light chain (V_{L}) and variable heavy chain (V_{H}) covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, hsFv refers to antibody fragments in which the constant domains normally present in an Fab fragment have been substituted with a heterodimeric coiled-coil domain (see, e.g., Arndt et al. (2001) J Mol Biol. 7:312:221-228).

As used herein, diabodies are dimeric scFv; diabodies typically have shorter peptide linkers than scFvs, and they preferentially dimerize.

As used herein, humanized antibodies refer to antibodies that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, idiotype refers to a set of one or more antigenic determinants specific to the variable region of an immunoglobulin molecule.

As used herein, anti-idiotype antibody refers to an antibody directed against the antigen-specific part of the sequence of an antibody or T cell receptor. In principle an anti-idiotype antibody inhibits a specific immune response.

As used herein, phage display refers to the expression of proteins or peptides on the surface of filamentous bacteriophage.

As used herein, panning refers to an affinity-based selection procedure for the isolation of phage displaying a molecule with a specificity for a desired capture molecule or epitope.

As used herein, transformation efficiency refers to the number of bacterial colonies produced per mass of plasmid DNA transformed (colony forming units (cfu) per mass of transformed plasmid DNA).

As used herein, titer with reference to phage refers to the number of colony forming units (cfu) per ml of transformed cells.

As used herein, normalization refers to the equilibration of the titer or concentration of all members of a tag library so that the number of tagged members in two samples or portions are about the same.

As used herein, staining refers to the visualization of molecules bound to the capture system. Staining can be non-specific, semi-specific or specific depending on what is labelled in a sample and when it is detected. Non-specific staining refers to the labelling of non-fractionated or all components in a particular sample generally, although not necessarily, prior to exposure to the capture system. Semi-specific staining as used herein refers to labelling of a portion of a sample, such as, but not limited to, the proteins located on the cell surface or on cellular membranes, either before, during or after exposure to the capture system. Specific staining as used herein refers to the labelling of a specific component of a sample, typically after the exposure of the sample to the capture system. The stain can be any molecule that associates with and that permits visualization or detection of bound molecules.

As used herein, non-radioactive energy transfer reactions, such as FET (fluorescent energy transfer) assays, FRET (fluorescent resonance energy transfer) assays, fluorescence polarization (FP) assays and HTRF (homogeneous time-resolved fluorescence), are homogeneous luminescence assays based on energy transfer and are carried out between a donor luminescent label and an acceptor label (see, e.g., Cardullo et al. (1988) Proc. Natl. Acad. Sci. U.S.A. 85:8790-8794; Peerce et al. (1986) Proc. Natl. Acad. Sci. U.S.A. 83:8092-8096; U.S. Pat. No. 4,777,128; U.S. Pat. No. 5,162,508; U.S. Pat. No. 4,927,923; U.S. Pat. No. 5,279,943; and International PCT Application No. WO 92/01225).

As used herein, Fluorescence Resonance Energy Transfer (FRET) refers to non-radiative energy transfer between chemical and/or proteinfluors. Fluorescent resonance energy transfer (FRET) is an art-recognized process in which one fluorophore (the acceptor) can be promoted to an excited electronic state through quantum mechanical coupling with and receipt of energy from an electronically excited second fluorophore (the donor). This transfer of energy results in a decrease in visible fluorescence emission by the donor and an increase in fluorescent energy emission by the acceptor.

For FRET to occur efficiently, the absorption and emission spectra between the donor and acceptor have to overlap. Dye pairs are characterized by their spectral overlap properties. Emission spectrum of donors must overlap acceptor absorption spectrum. Extent of overlap determines the efficiency of energy transfer. Extent of overlap also determines the optimal distance for which the assay is sensitive. Where the overlap of spectra is large, the transfer is efficient, so it is only sensitive to long distances. The selection of donor/acceptor depends upon the distances considered.

Significant energy transfer can only occur when the donor and acceptor are sufficiently closely positioned since the efficiency of energy transfer is highly dependent upon the distance between donor and acceptor fluorophores. The fluorophores can be chemical fluors and protein fluors. For example, energy transfer between two fluorescent proteins (FRET) as a physiological reporter has been reported (Miyawaki et al. (1997) Nature 388:882-887),-in which two different GFPs were fused to the carboxyl and amino termini of calmodulin. Changes in calcium ion concentration caused a sufficient conformational change in calmodulin to alter the level of energy transfer between the GFP moieties.

As used herein, fluorescence polarization (FP) or anisotropy (see, e.g., Jameson et al. (1995) Methods Enzymol. 246:283-300) refers to procedures in which fluorescently labeled molecules are illuminated in solution with plane-polarized light. When fluorescently labeled molecules in solution are so-illuminated, the emitted fluorescence is in the same plane provided that the molecules remain stationary. Since all molecules tumble as a result of collisional motion, depolarization phenomenon is proportional to the rotational relaxation time (μ) of the molecule, which is defined by the expression 3ηV/RT. At constant viscosity (η) and temperature (T) of the solution, polarization is directly proportional to the molecular volume (V) (R is the universal gas constant). Hence changes in molecular volume or molecular weight due to binding interactions can be detected as a change in polarization. For example, the binding of a fluorescently labeled ligand to its receptor results in significant changes in measured fluorescence polarization values for the ligand. Measurements can be made in a “mix and measure” mode without physical separation of the bound and free ligands. The polarization measurements are relatively insensitive to fluctuations in fluorescence intensity when working in solutions with moderate optical intensity.

As used herein, a fluorescent protein refers to a protein that possesses the ability to fluoresce (i.e., to absorb energy at one wavelength and emit it at another wavelength). These proteins can be used as a fluorescent label or marker and in any applications in which such labels are used, such as immunoassays, CRET, FRET, and FET assays. For example, a green fluorescent protein (GFP) refers to a polypeptide that has a peak in the emission spectrum at about 510 nm. Green, blue and red fluorescent proteins are well known and readily available (Stratagene, see, U.S. Pat. Nos. 6,247,995 and 6,232,107).

As used herein, fluorophore refers to a fluorescent compound. Fluorescence is a physical process in which light is emitted from the compound following absorption of radiation. Generally, the emitted light is of lower energy and longer wavelength than that absorbed. Preferred fluorophores herein are those whose fluorescence can be detected using standard techniques.

As used herein, a donor molecule is a chemical or biological compound that is capable of transferring energy from itself to another molecule. The energy that is transferred can include, but is not limited to, fluorescence resonance energy.

As used herein, an acceptor molecule is a chemical or biological compound that is capable of accepting energy from another molecule. The energy that is transferred can include, but is not limited to, fluorescence resonance energy.

As used herein, attachment refers to the attachment of a label to a biomolecule. The attachment can include, but is not limited to, covalent attachment, an affinity interaction, hybridization, electrostatic interaction and an operably linked macromolecule, such as a fusion protein.

As used herein, a label is a detectable marker that can be attached or linked directly or indirectly to a molecule or associated therewith. The detection method can be any method known in the art.

As used herein, a modulator is any molecule or condition that alters an interaction or reaction between or among molecules.

As used herein, an inhibitor is any molecule or condition that inhibits an interaction or reaction, between or among molecules.

As used herein, an enhancer is any molecule or condition that enhances an interaction or reaction between or among molecules.

As used herein, a subcellular compartment or an organelle is a membrane-enclosed compartment in a eukaryotic cell that has a distinct structure, macromolecular composition, and function. Organelles include, but are not limited to, the nucleus, mitochondrion,, chloroplast, and Golgi apparatus.

As used herein, screening refers to the process of analyzing molecules, such as sets of molecules and library compounds, by methods that include, but are not limited to, ultraviolet-visible (UV-VIS) spectroscopy, infra-Red (IR) spectroscopy, fluorescence spectroscopy, fluorescence resonance energy transfer (FRET), NMR spectroscopy, circular dichroism (CD), mass spectrometry, other analytical methods, high throughput screening, combinatorial screening, enzymatic assays, antibody assays and other biological and/or chemical screening methods or any- combination thereof.

As used herein, in silico refers to research and experiments performed using a computer. In silico methods include, but are not limited to, molecular modelling studies, biomolecular docking experiments, and virtual representations of molecular structures and/or processes, such as molecular interactions.

As used herein, cell capture refers to the immobilization of a cell by a capture system provided herein.

As used herein, biological sample refers to any sample obtained from a living or viral source and includes any cell type or tissue of a subject from which nucleic acid or protein or other macromolecule can be obtained. Biological samples include, but are not limited to, body fluids, such as blood, plasma, serum, cerebrospinal fluid, synovial fluid, urine and sweat, tissue and organ samples from animals and plants. Also included are soil and water samples and other environmental samples, viruses, bacteria, fungi, algae, protozoa and components thereof. Hence bacterial and viral and other contamination of food products and environments can be assessed. The methods herein are practiced using biological samples and in some embodiments, such as for profiling, also can be used for testing any sample.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically, or using recombinant molecular biology methods.

As used herein, the term “biopolymer” is a biological molecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein, respectively. Biopolymers include, but are not limited to, nucleic acids, proteins, polysaccharides, lipids and other macromolecules. Nucleic acids include DNA, RNA, and fragments thereof. Nucleic acids can be derived from genomic DNA, RNA, mitochondrial nucleic acid, chloroplast nucleic acid and other organelles with separate genetic material.

As used herein, a biomolecule is any compound found in nature, or derivatives thereof. Biomolecules include, but are not limited to: oligonucleotides, oligonucleosides, proteins, peptides, amino acids, peptide nucleic acids (PNAs), oligosaccharides and monosaccharides.

As used herein, a biological particle refers to a virus, such as a viral vector or viral capsid with or without packaged nucleic acid, phage, including a phage vector or phage capsid, with or without encapsulated nucleic acid, a single cell, including eukaryotic and prokaryotic cells or fragments thereof, a liposome or micellar agent or other packaging particle, and other such biological materials.

As used herein, a molecule refers to any molecule that is linked to the solid support. Typically such molecules are compounds or components or precursors thereof, such as peptides, amino acids, small organics, oligonucleotides or monomeric units thereof. A monomeric unit refers to one of the constituents from which the resulting compound is built. Thus, monomeric units include, nucleotides, amino acids, and pharmacophores from which small organic molecules are synthesized.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof.

As used herein “nucleic acid” refers to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about-fifty to one hundred nucleotides in length.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well known methods (see, for example, Weiler et al., Nucleic acids Res. 25:2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, production by recombinant means by using recombinant DNA methods means the use of the well known methods of molecular biology for expressing proteins encoded by cloned DNA.

As used herein, substantially identical to a product means sufficiently similar so that the property of interest is sufficiently unchanged so that the substantially identical product can be used in place of the product.

As used herein, equivalent, when referring to two sequences of nucleic acids, means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 1, below) that do not substantially alter the activity or function of the protein or peptide. When “equivalent” refers to a property, the property does not need to be present to the same extent, but the activities are generally substantially the same. “Complementary,” when referring to two nucleotide sequences, means that the two sequences of nucleotides are capable of hybridizing, generally with less than%25%, with less than 15%, and even with less than 5% or with no mismatches between opposed nucleotides. Generally to be considered complementary herein the two molecules hybridize under conditions of high stringency.

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1×SSPE or SSC, 0.1% SDS, 65° C.

2) medium stringency: 0.2×SSPE or SSC, 0.1% SDS, 50° C.

3) low stringency: 1.0×SSPE or SSC, 0.1% SDS, 50° C.

Equivalent conditions refer to conditions that select for substantially the same percentage of mismatch in the resulting hybrids. Additions of ingredients, such as formamide, Ficoll, and Denhardt's solution affect parameters such as the temperature under which the hybridization is conducted and the rate of the reaction. Thus, hybridization in 5×SSC, in 20% formamide at 42° C. is substantially the same as the conditions recited above as hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring:Harbor Laboratory Press, Chapter 8; see, Sambrook et al., vol. 3, p. B.13, see, also, numerous catalogs that describe commonly used laboratory solutions). It is understood that equivalent stringencies can be achieved using alternative buffers, salts and temperatures.

The term “substantially” identical or homologous or similar varies with the context as understood by those skilled in the relevant art and generally means at least 70%, preferably means at least 80%, more preferably at least 90%, and most preferably at least 95% identity.

As used herein, a reporter gene construct is a nucleic .acid molecule that includes a nucleic acid encoding a reporter operatively linked to a transcriptional control sequences. Transcription of the reporter gene is controlled by these sequences. The activity of at least one or more of these control sequences is directly or indirectly regulated by a cell surface protein or other protein that interacts with tagged molecules or other molecules in the capture system. The transcriptional control sequences include the promoter and other regulatory regions, such as enhancer sequences, that modulate the activity of the promoter, or control sequences that modulate the activity or efficiency of the RNA polymerase that recognizes the promoter, or control sequences are recognized by effector molecules, including those that are specifically induced by interaction of an extracellular signal with a cell surface protein. For example, modulation of the activity of the promoter may be effected by altering the RNA polymerase binding to the promoter region, or, alternatively, by interfering with initiation of transcription or elongation of the mRNA. Such sequences are herein collectively referred to as transcriptional control elements or sequences. In addition, the construct may include sequences of nucleotides that alter translation of the resulting mRNA, thereby altering the amount of reporter gene product.

As used herein, staining or labeling refers to moieties used to visualize or detect biological particles or molecules.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety can be expressed as a fusion protein with a protein of interest or under the control of a promoter of interest. As used herein, the phrase “operatively linked” generally means the sequences or segments have been covalently joined into one piece of DNA, whether in single- or double-stranded form, whereby control or regulatory sequences on one segment control or permit expression or replication or other such control of other segments. The two segments are not necessarily contiguous. It means a juxtaposition between two or more components so that the components are in a relationship permitting them to function in their intended manner. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression a sequence of nucleotides and a regulatory sequence(s) are connected in such a way as to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be ciszacting or can be responsive to trans acting factors. Promoters, depending upon the nature of the regulation, can be constitutive or regulated.

As used herein, the term “regulatory region” means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present, or at increased concentration, gene expression increases. Regulatory regions also include sequences that confer repression of gene expression (i.e., a substance or stimulus decreases transcription). When a repressor is present or at increased concentration, gene expression decreases. Regulatory regions are known to influence, modulate or control many in vivo biological activities including cell proliferation, cell growth and death, cell differentiation and immune-modulation. Regulatory regions typically bind one or more trans-acting proteins which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome entry sites (IRES) for the creation of multigene, or polycistronic, messages, polyadenylation signals to provide proper polyadenylation of the transcript of a gene of interest and stop codons and can be optionally included in an expression vector.

As used herein, regulatory molecule refers to a polymer of deoxyribonucleic acid (DNA) or ribonucleic acid (RNA), or an oligonucleotide mimetic, or a polypeptide or other molecule that is capable of enhancing or inhibiting expression of a gene.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof.

As used herein, kit refers to a packaged combination, optionally including instructions and/or reagents for their use.

As used herein, fluid refers to any composition that can flow. Fluids thus encompass compositions that are in the form of semi-solids, pastes, solutions, aqueous mixtures, gels, lotions, creams and other such compositions.

As used herein, antigenic means that a polypeptide induce an immune response. Highly antigenic polypeptides are those that reproducibly and predictably induce an immune response.

As used herein, antigenic ranking refers to a statistical probability that an amino acid or set thereof occurs in an antigenic polypeptide, including epitopes in naturally occurring polypeptides.

As used herein, highly antigenic, highly specific polypeptides (HAHS) mean polypeptides that specifically bind to a capture agent and that are antigenic such that specifically binding capture agents are readily designed or prepared. For example, the polypeptides that result from application of the methods raise or produce high titer antiserum in rodents, such as mice. Hence methods readily produce pairs of polypeptides (the highly antigenic highly specific polypeptides) and capture agents.

As used herein, a similarity ranking refers to a comparison among amino acids and is represented or determined as a probability or fraction that two amino acids are structurally and/or functionally similar. For example, two identical amino acids have a similarity ranking of 100; two very dissimilar amino acids, such as proline and tyrosine have a ranking of 0.

As used herein, a subset of a set contains at least one less member than the set.

As used herein, a critical residue or amino acid in an HAHS polypeptide is one that influences the affinity or specificity of binding to the binding protein (capture agent). Critical residues taken from the set of naturally occurring amino acids can only be replaced by a subset of amino acids (usually 1 or 2 amino acids).or in some cases, can not be replaced by any other amino acid from this set.

As used herein, a non-critical residue or amino acid in an HAHS polypeptide is one that does not influence the affinity or specificity of binding to the binding protein (capture agent). Noncritical residues can be replaced by a larger subset of amino acids (for example, when taken from the set of naturally occurring amino acids, they can be replaced usually 10 or more amino acids or in some cases,;,by any other amino acid from this set) without affecting the affinity or specificity of binding. In some cases, non-critical residues are used to confer additional functionalities or properties on polypeptides. In this case, they can typically only be replaced by a limited number of amino acids to retain the functionality or property.

As used herein, suitable conservative substitutions of amino acids are known to those of skill in this, art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions can be made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with known conservative substitutions.

As used herein, an amino acid is an organic compound containing an amino group and a carboxylic acid group. A polypeptide comprises two or more amino acids. For purposes herein, amino acids include the twenty naturally-occurring amino acids non-natural amino acids, and amino acid analogs. These include amino acids wherein α-carbon has a side chain.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, naturally occurring amino acids refers to the 20 L-amino acids that occur in polypeptides.

As used herein, the term “non-natural amino acid” refers to an organic compound that has a structure similar to a natural amino acid but has been modified structurally to mimic the structure and reactivity of a natural amino acid. Non-naturally occurring amino acids thus include amino acids or analogs of amino acids other than the 20 naturally occurring amino acids and include, but are not limited to, the D-isostereomers of amino acids. Exemplary non-natural amino acids are described herein and are known to those of skill in the art.

As used herein, the abbreviations for any protective groups, amino acids and other compounds, are, unless-indicated otherwise, in accord with their common usage, recognized abbreviations, or the IUPAC-IUB Commission on Biochemical Nomenclature (see, (1972) Biochem. 11:1726). Each naturally occurring L-amino acid is identified by the standard three letter code (or single letter code) or the standard three letter code (or single letter code) with the prefix “L-”; the prefix “D-” indicates that the stereoisomeric form of the amino acid is D.

The methods and collections herein are described and exemplified with particular reference to antibody capture agents, and polypeptide tags that include epitopes to which the~antibodies bind, but is it to be understood that the methods herein can be practiced with any capture agent and any polypeptide tag therefor. It also is to be understood that combinations of collections of any capture agents and polypeptide tags therefor are contemplated for use in any of the embodiments described herein. It also is to be understood that reference to an array is intended to encompass any addressable collection, whether it is in the form of a physical array or labeled collection, such as capture agents bound to colored beads.

### B. Capture Agents and Polypeptide Tags

Provided herein are capture systems that include addressable collections of capture agents and polypeptide-tagged molecules. The polypeptide tags specifically bind to capture agents to produce the capture systems.

### 1. Capture Agents

As noted, a capture agent is a molecule that has an affinity for a defined sequence of amino acids or other site on another molecule, such as a ligand, or for purposes herein a polypeptide tag. For purposes herein, the term capture agent, receptor and anti-ligand are interchangeable. Capture agents include any agent that specifically binds with sufficient affinity (for further use of the resulting capture systems) to polypeptide tags in a tagged library. Any molecule that specifically binds to another is a capture agent. Capture agents can be naturally-occurring or synthetic molecules, and include any molecule, including nucleic acids, small organics, proteins and complexes that specifically bind to specific sequences of amino acids. Capture agents are receptors and also are referred to as anti-ligands in the art. Capture agents can be used in their unaltered state or as aggregates with other species. They can be attached or in physical contact with, covalently or noncovalently, a binding member, either directly or indirectly via a specific binding substance or linker. As noted, as contemplated herein, capture agents are one of a pair of molecules that specifically bind to each other. One member of the pair is a polypeptide that is used as a tag and encoded by nucleic acids that can be linked to a nucleic acid library; the other member, the capture agent, is anything that specifically binds thereto. Examples of capture agents, include, but are not limited to: antibodies and binding fragments thereof, cell membrane receptors, surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, and organelles.

The methods provided herein rely upon the ability of the capture agents, such as antibodies, to specifically bind to the polypeptide tags, which are linked to libraries (or collections) of molecules, particularly proteins. The specificity of each capture (or other receptor in the collection) for a particular tag is known or can be readily ascertained, such as by arraying the capture agent so that all of the agents at a locus have the same specificity. Agents to which each locus binds can be identified.

Capture agents can be positionally addressed. Alternatively, each can be addressed by associating them with unique identifiers, such as by linkage to optically encoded tags, including colored beads or bar coded beads or supports, or linked to electronic tags, such as by providing microreactors with electronic tags or bar coded supports (see, e.g., U.S. Pat. No. 6,025,129; U.S. Pat. No. 6,017,496; U.S. Pat. No. 5,972,639; U.S. Pat. No. 5,961,923; U.S. Pat. No. 5,925,562; U.S. Pat. No. 5,874,214; U.S. Pat. No. 5,751,629; U.S. Pat. No. 5,741,462), or chemical tags (see, U.S. Pat. No. 5,432,018; U.S. Pat. No. 5,547,839) or colored tags or other such addressing methods that can be used in place of physically addressable arrays. For example, each antibody type can be bound to a support matrix associated with a color-coded tag (i.e. a colored sortable bead) or with an electronic tag, such as a radio-frequency tag (RF), such as IRORI MICROKANS® and MICROTUBES® microreactors (see, U.S. Pat. No. 6,025,129; U.S. Pat. No. 6,017,496; U.S. Pat. No. 5,972,639; U.S. Pat. No. 5,961,923; U.S. Pat. No. 5,925,562; U.S. Pat. No. 5,874,214; U.S. Pat. No. 5,751,629; U.S. Pat. No. 5,741,462; International PCT application No. WO98/31732; International PCT application No. WO98/15825; and, see, also U.S. Pat. No. 6,087,186 ). For the methods and collections provided herein, the antibodies of each type can be bound to the MICROKAN or MICROTUBE microreactor support matrix and the associate RF tag, bar code, color, colored bead or other identifier serves to identify the capture agents, such as antibodies, and hence the polypeptide tag to which the capture agent, such as an antibody, binds.

Examples of capture agents, include, but are not limited to:

a) enzymes and other catalytic polypeptides, including, but are not limited to, portions thereof to which substrates specifically bind, enzymes modified to retain binding activity lack catalytic activity;

b) antibodies and portions thereof that specifically bind to antigens or sequences of amino acids;

c) nucleic acids;

d) cell surface receptors, opiate receptors and hormone receptors and other receptors that specifically bind to ligands, such as hormones.

For the collections herein, the other binding partner, referred to herein as a polypeptide tag for each refers to the substrate, antigenic sequence, nucleic acid binding protein, receptor ligand, or binding portion thereof. The collections of capture agents, include receptors, such as antibodies or enzymes or portions thereof and mixtures thereof that specifically bind to a known or knowable defined sequence of amino acids that is typically at least about 3 to 10 amino acids in length. These agents include, but are not limited to, immunoglobulins of any subtype (IgG, IgM, IgA, IgE, IgE) or those of any species, such as, for example, IgY of avian species (Romito et al. (2001) Biotechniques 31:670, 672, 674-670, 672, 675.; Lemamy et al. (1999) Int. J. Cancer 80:896-902; Gassmann et al. (1990) FASEB J. 4:2528-2532), or the camelid antibodies lacking a light chain (Sheriff et al. (1996) Nat. Struct. Biol. 3:733-736; Hamers-Casterman et al. (1993) Nature 363:446-448) can be raised against virtually limitless entities Polyclonal and monoclonal immunoglobulins can be used as capture agents. Additionally, fragments of immunoglobulins derived by enzymatic digestion (Fv, Fab) or produced by recombinant means (scFv, diabody, Fab, dsFv, single domain Ig) (Arbabi et al. (1997) FEBS Lett. 414:521-526; Martin et al. (1997) Protein Eng 10:607-614; Holt et al. (2000) Curr. Opin. Biotechnol. 11:445-449) are suitable capture agents. Additionally, entirely new synthetic proteins and peptide mimetics and analogs can be designed for use as capture agents (Pessi et al. (1993) Nature 362:367-369).

Many different protein domains have been engineered to introduce variable regions to mimic the diversity seen in antibody molecules. Lipocalin (Skerra (2000) Biochim. Biophys. Acta 1482:337-350), fibronectin type III domains (Koide et al. (1998) J. Mol. Biol. 284:1141-1151), protein A domains (Nord et al. (2001) Eur. J. Biochem. 268:4269-4277; Braisted et al. (1996) Proc. Natl. Acad. Sci. U.S.A. 93:5688-5692), protease inhibitors (Kunitz domains, cysteine-knots (Skerra (2000) J. Mol. Recognit. 13:167-187; Christmann et al. (1999) Protein Eng 12:797-806), thioredoxin (Xu et al. (2001) Biochemistry 40:4512-4520; Westerlund-Wikstrom, B (2000) Int. J. Med. Microbiol. 290:223-230), and GFP (Peelle et al. (2001) Chem. Biol. 8:521-534; Abedi et al. (1998) Nucleic Acids Res. 26:623-630) have been modified to function as binding agents. Many domains in proteins have been implicated in direct protein-protein interactions. With modifications, these interactions can be manipulated and: controlled. For example, it is known that src homology-2 (SH2) domains are known to bind proteins containing a phosphorylated tyrosine (Ward et al. (1996) J. Biol. Chem. 271:5603-5609). The phosphotyrosine alone does not determine specificity, but amino acids surrounding it contribute to the binding affinity and specificity (Songyang et al. (1993) Cell 72:767-778). The SH2 domain can function as a capture agent. For example, altering amino acids in the binding pocket where new specificities result. Similarly, src homology 3 domains (SH3) bind a ten-residue consensus sequence, XPXXPPPFXP (where X is any amino acid residue, F is phenylalanine and P is proline; SEQ ID No. 102) (Sparks et al. (1998) Methods Mol. Biol. 84:87-103) can function as capture agents. Mutant SH3 domains can be selected to bind to polypeptide tags with the above consensus sequence. The epidermal growth factor (EGF) domain has a two-stranded beta-sheet followed by a loop to a C-terminal short two-stranded sheet. This domain has been implicated in many protein-protein interactions, it can form the basis for a family of capture agents following manipulation of the loop between the two beta sheets. Long alpha-helical coils are known to interact with other alpha-helical segments to cause proteins to dimerize and trimerize. These coiled-coil interactions can be of very high affinity and specificity (Arndt et al. (2000) J. Mol. Biol. 295:627-639), and therefore can be used as capture agents when paired-with complementary polypeptide tags. Nearly any protein domain can be modified such that the variability introduced into one or more exposed regions of the molecule can constitute a potential binding site. Mutant enzymes, designated substrate trapping enzymes, that do not exhibit catalytic activity but retain substrate binding activity can be used (see, e.g., International PCT application No. WO 01/02600).

While most of the reagents used for affinity interactions with proteins are proteins, there are many other protein-binding agents. Nucleic acids constitute a family of molecules that have inherent diversity of structure. Although there are only five naturally occurring subunits (ATP, CTP, TTP, GTP and UTP) compared to the twenty naturally occurring amino acids that make up proteins, they have the potential to fold into an immense variety of different structures capable of binding to a huge number of protein elements. Selection strategies for single-stranded RNA (Sun (2000) Curr. Opin. Mol. Ther. 2:100-105; Hermann et al. (2000) Science 287:820-825; Cox et al. (2001) Bioorg. Med. Chem. 9:2525-2531) and single-stranded DNA (or RNA) aptamers (Ellington et al. (1992) Nature 355:850-852) have been developed. These methods have proven successful for discovery of high affinity binders to small molecules as well as proteins. Using these methods, aptamers that bind with high specificity and affinity to polypeptide tags can be selected and then used as capture agents.

Single-stranded DNA or RNA can fold into diverse structures. Double-stranded nucleic acids, while more restricted in overall structure, can be used as capture agents with the correct polypeptide tags. DNA binding proteins such as proteins containing zinc finger domains (Kim et al. (1998) Proc. Natl. Acad. Sci. U.S.A. 95:2812-2817) and leucine zipper (Alber (1992) Curr. Opin. Genet. Dev. 2:205-210) domains bind with high specificity to double stranded DNA molecules of defined sequence. Zinc finger domains bind to dsDNA in an arrayed format (see, e.g., Bulyk et al. (2001) Proc. Natl. Acad. Sci. U.S.A. 98:7158-7163). Additionally, DNA modifying enzymes can be modified for use as polypeptide tags to bind to DNA used as an affinity capture agent. For example, the DNA restriction endonuclease BamHI has specific target sequence of GGATCC, but with mutation of the active site, a new enzyme is created that recognizes the sequence GCATGC. It also has been demonstrated that basepairs outside the specific target sequence play an important role in the binding affinity, and that the catalytic event can be eliminated in the absence of the cofactor Mg²⁺ (Engler et al. (2001) J. Mol. Biol. 307:619-636). Mutations in some restriction enzymes abolish the cleavage event and leave the DNA binding domain bound to the dsDNA target (Topal et al. (1993) Nucleic Acids Res. 21:2599-2603;.Mucke et al. (2000) J. Biol. Chem. 275:30631-30637). Thus, panels of double-stranded nucleic acids can serve as capture agents.

Small chemical entities also can be designed to be capture agents. The highest affinity non-covalent interaction involving a protein is between proteins such as egg-white avidin or the bacterial streptavidin and the small, naturally-occurring chemical entity biotin. Biotin-like molecules can be used as capture agents if the polypeptide tags are avidin-like proteins. Panels of chemically synthesized biotin analogs, and a corresponding panel of avidin mutants each capable of specific, high affinity binding to those biotin analogs can be employed. Other chemical entities have specific affinity for protein sequences. For example, immobilized metal affinity chromatography has been widely used for purification of proteins containing a hexa-histidine tag. Iminodiacetic acid, NTA or other metal chelators are used. The metal used determines the strength of interaction and possibly the specificity. Similarly, proteins that bind to other metals (Patwardhan et al. (1997). J. Chromatogr. A 787:91-100) can be selected.

Similarly, digoxin and a panel of digoxin analogs can be used as capture agents if the polypeptide tags are designed to bind to those analogs. Antibodies and scFvs have been created that bind with high specificity to these analogs (Krykbaev et al. (2001) J. Biol. Chem. 276:8149-8158) and the recombinant scFvs can be used as polypeptide tags. Carbohydrates, lipids, gangliosides can be used as capture agents for polypeptide tags in the form of lectins (Yamamoto et al. (2000) J. Biochem. (Tokyo) 127:137-142; Swimmer et al. (1 992) Proc. Natl. Acad. Sci. U.S.A. 89:3756-3760), fatty acid binding proteins (Serrero et al. (2000) Biochim. Biophys. Acta 1488, 245-254.) and peptides (Matsubara et al. (1999) FEBS Lett. 456:253-256). Hence, any member of a pair of molecules that specifically bind is contemplated.

For exemplary purposes herein, reference is made to antibodies and tags that encode epitopes to which the antibody specifically binds. It is understood that any pair of molecules that specifically bind are contemplated; for purposes herein the molecules, such as antibodies, are designated receptors, and the polypeptides that specifically bind thereto are polypeptide tags.

Also, for exemplary purposes herein, reference is made to positional arrays. It is understood, however, that such other identifying methods can be readily adapted for use with the methods herein. It is only necessary that the identity (i.e., polypeptide-tag specificity) of the capture agent, such as an antibody, is known. The resulting collections of addressable capture (i.e., antibodies) can be linked to identifiers, such as optically encoded beads or colored supports or RF tags or other bar-coded identifiers can be employed in the capture systems.

### 2. Polypeptide Tags and Preparation Thereof

As described above, any moiety, generally a protein that specifically binds to a capture agent is contemplated as a polypeptide tag, also referred to as an epitope tag. The term “epitope” is not to be construed as limited to an antibody-binding polypeptide, but as any specifically binding moiety. A polypeptide (or epitope) tag refers to a sequence of amino acids that includes the sequence of amino acids, herein referred to as an epitope, to which a capture agent, such as an antibody and any agent described above, specifically binds. For polypeptide (epitope) tags, the specific sequence of amino acids or region of a molecule to which each binds is referred to herein generically as an epitope (but is not an epitope in the immunological sense). Any sequence of amino acids that binds to a receptor therefor is contemplated for use as a polypeptide tag. For purposes herein, the sequence of amino acids of the tag, such as epitope portion of the polypeptide tag, that specifically binds to the capture agent is designated “E”, and each unique epitope is an Eₘ. Depending upon the context, “Eₘ” also can refer to the sequences of nucleic acids encoding the amino acids constituting the epitope.

In particular, the polypeptide tag can be encoded by an oligonucleotide, which are used to introduce the tag. When reference is made to a polypeptide or-epitope tag (i.e. binding pair for a particular receptor or portion thereof) with respect to a nucleic acid, it is nucleic acid encoding the tag to which reference is made. Each polypeptide tag is referred to as Eₘ (again E is not intended to limit the tags to “epitopes”, but includes any sequence of amino acids that specifically binds to a capture agent); when nucleic acids are being described, the Eₘ is nucleic acid and refers to the sequence of nucleic acids that encode the binding portion of the polypeptide; when the translated proteins are described, Eₘ refers to amino acids (the actual binding polypeptide or epitope). The number of Es corresponds to the number of unique capture agents, such as antibodies, in an addressable collection. “m” is typically at least 10, 30 or more, 50 or 100, 250 or more, and can be as high as desired and as is practical. Generally “m” is about 100, 250, 500, 1000 or more.

Any of the proteins or polypeptides described as possible capture agents also can be used as polypeptide tags as long as the capture agents are addressable, such as by arraying, labeling with nanobarcodes or other such codes, encoded with colored beads and other such addressing products. The polypeptide tags are not necessarily small peptide sequences.

In some cases, it can be necessary or desirable to have the oligonucleotides used for subdivision of a library or recovery of a sub-library distinct from the polypeptide tag portion of the nucleic acid encoding the tags. In addition, the linked molecule can have a plurality of tags that serve different purposes.

Nucleic acid encoding a polypeptide tag (epitope tag) also can include sequences of nucleotides that can aid in unique or convenient priming, or can encode amino acids that confer desired properties, such as trafficking signals, detection, solubility alteration, facilitation of purification or conjugation or other functions or provide other functions. For example, tags such as, but not limited to, green fluorescent protein (GFP), red fluorescent protein (RFP), blue fluorescent protein (BFP) or other commercially available tags can be used for the detection of expressed polypeptide tags in culture or as in purified fusion molecule. Tags that result in the secretion of the polypeptide tagged molecule include, but are not limited to, RsaA, CBP, MBP, OmpT, OmpA, PelB or other commercially available tags. Tags that facilitate purification such as, but not limited to, polyhistidine and polylysine tags, FLAG, calmodulin binding peptide (CBP), biotin carboxycarrier protein (BCCP), Strep, maltose-binding protein (MBP) intein/chitin-binding domain, cellulose-binding domain (CBP), myc tags or other commercially available tags are known and can be appended to the polypeptide tagged molecule by any method known to those skilled in the art. In addition, a capture can be used as an affinity ligand for the purification of a polypeptide tagged molecule. Further, a plurality of tags, both in number and function, can be used within a single tagged molecule. Selection of the tags, including, but not limited to, those listed above, for placement in a particular library can be determined by those skilled in the art.

Furthermore, particularly for certain applications, such as profiling, the polypeptide tag does not have to be fused to the library of interest such that a single protein is synthesized. It is possible to prepare tags that are encoded as separate polypeptides that are physically or otherwise associated or linked with the library member. For example, dimerizing domains can be used to couple two separate proteins expressed in the same cell (Chao et al. (1998) J. Chromatogr. B Biomed. Sci. Appl. 715:307-329; Hodges (1996) Biochem. Cell Biol. 74, 133-154; Alber (1992) Curr. Opin. Genet. Dev. 2:205-210). One of the dimerizing-domains is fused to the library protein, and its partner dimerizing-domain is fused to the polypeptide tagged molecule. The dimerizing domains cause association of the library protein and tag. These tags serve the same purpose of subdivision of the library on the addressable array. Also, the DNA encoding such tag is still associated with one specific subset of the total DNA library (since it is in the same plasmid or linear expression construct), and therefore indicates which subset to recover.

Another example is a two-domain polypeptide tag, in which DNA sequences used for subdivision of a library or recovery of a sub-library are distinct from the protein-encoding portion, the polypeptide tags, which are larger proteins. For example, a larger protein such as a series of zinc finger (ZF) domains can be used as a polypeptide tag capable of binding to double-stranded DNA (dsDNA, used as a capture agent). Specific fingers can be selected that bind to dsDNA sequences (Wu et al. (1995) Proc. Natl. Acad. Sci. U.S.A. 92:344-348; Jamieson et al. (1994) Biochemistry 33:5689-5695; and Rebar (1 99) Science 263:671-673). These zinc fingers are modular and can be combined to give increased specificity and affinity for the dsDNA target (Isalan et al. (2001) Nat. Biotechnol. 19:656-660; Kim (1998) Proc. Natl. Acad. Sci. U.S.A. 95:2812-2817).

Due to the modular nature of these domains. (see, Bulyk et al. (2001) Proc. Natl. Acad. Sci. U.S.A. 98:7158-7163 and modified), the conserved sequences in each module and the overall size, it can be difficult to design oligonucleotide primers that correspond to the protein-encoding region and specifically amplify only a single class of tags. Each polypeptide tag is a DNA binding protein composed of three zinc finger -domains that are arranged in a different order. The order as well as the composition of each domain will determine the specificity for the dsDNA capture agent. Oligonucleotide primers specific for a single domain can still amplify multiple different polypeptide tags.

Nucleic acid encoding a polypeptide tag can include a tag-specific amplification sequence (recovery or R-tag ) that can be associated with a specific tag in a predetermined manner. This R-tag can encode protein, but does not need to be part of the binding portion of the encoded polypeptide tag. An R-tag does not necessarily encode protein, and can be located prior to the translational start site, or following the translational termination site or elsewhere. For example, a different recovery tag is associated with each polypeptide tag. By separating the amplification portion from the epitope-encoding portion, it is possible to optimize each for the desired function, i.e., the R-tag portion can be an optimal amplification sequence, and the capture-agent-binding portion can be optimized for binding to a selected capture agent.

Therefore, while no oligonucleotide- corresponding to a single domain in the polypeptide tag can be used to specifically amplify a given sub-library each of the R-tags can be used to specifically amplify its corresponding sub-library. Because the R-tags do not need to encode protein, there is considerable flexibility in designing sequences that allow the specific hybridization (and, thus amplification) of only the correct corresponding sequences. Many available DNA sequence analysis software packages (Lasergene's DNAStar®, Informax's VectorNTi®, etc.) allow the analysis of oligonucleotides for melting temperature, primer-dimer formation, hairpin formation: as well as cross-reactivity and mis-priming.

To increase specificity further, two specific R-tags can be associated with each particular tag such that one is prior to the translation initiation site, and the other follows the translation termination signal. Therefore, neither R-tag is encoded into the protein, but the inclusion of a second R-tag increases the stringency to ensure recovery of only the correct corresponding encoded polypeptides. Instead of flanking the cDNA library and tag encoding regions, the two recovery tags associated with:each tag can be nested primers on only one side of the protein-encoding region. These nested primers are used in succession in two sequential reactions.

Furthermore, tags are not necessarily polypeptides. It is possible that the ligand for the capture agent is a protein modification such as a phosphorylated amino acid. Capture agents can distinguish combinations of phosphorylated and non-phosphorylated residues contained in a peptide. For example, mutated SH2 domains are arrayed as capture agents such that one binds the sequence His-PO₄Tyr-Ser-Thr-Leu-Met, a second binds His-Tyr-PO₄Ser-Thr-Leu-Met and a third binds His-Tyr-Ser-PO₄Thr-Leu-Met and a fourth binds PO₄His-Tyr-Ser-Thr-Leu-Met. Each of these peptide sequences is the same, but the position of the phosphate group determines specificity. In each of these cases, the peptide is fused to the library member, but an additional encoded protein (Serine, Histidine, Threonine, or Tyrosine kinases) directs the phosphorylation event separately.

In this case the polypeptide tag has two separate determinants, the peptide portion that binds to a capture agent, and the kinase responsible for the phosphorylation event. Recovery entails two sequential amplification steps. As above, these tags serve the same purpose of subdivision of the library in an addressable collection. Also, the nucleic acid encoding this tag (the peptide and the kinase) are associated with one specific subset of a total DNA library, since they are in the same plasmid or linear expression construct, and therefore indicate which subset to recover. Other protein modifying enzymes include, but are not limited to, those that are involved in fatty acid acylation, glycosylation, and methylation.

While the above descriptions exemplify methods for designing primers, it also can be desirable to use a non-encoding associated R-tag. R-tags in some instances can be designed for the PCR amplification steps, since they are not constrained by the amino acids used in the tag. The R-tag is associated with its corresponding capture agent-binding portion during the library creation process. For example, in embodiments in which cDNA is subcloned into a panel of vectors each containing a polypeptide tag, the R-tag also is included in the vector.

In addition, modifications of the use of an enzyme modification of the tags before binding the capture agent can alter binding specificity. In such embodiments, the enzyme is not required to be physically linked to the polypeptide tag. The enzyme-catalyzed modification is used to alter specificity of the tag for the capture agent or of a capture agent for a tag.

### 3. Identification of Capture Agents—Polypeptide Tag Pairs

For preparation of the capture systems herein, pairs of capture agents and tags are required. These can be identified and/or designed or otherwise selected. The tags are immobilized by the capture agents by any interaction that is specific and of high affinity, generally equal to or greater affinity than moieties, such as molecules, cells and other biological particles, that bind to immobilized tagged molecules in the capture system. Any interaction, including, but are not limited to, covalent, ionic, hydrophobic, van der Waals and other such interactions, that result in the immobilization of a tagged molecule by a capture agent. As noted, capture agents and tags can be any molecule or compound known in the art. Hence, selection of binding pairs can be empirically determined by those with skill in the art or can include pairs with known high specificity and affinity. Such methods are exemplified herein with respect to antibody capture agents and polypeptide tags, but it is understood that any capture agent/tag pairs obtained or made by any method are contemplated.

Antibodies or fragments thereof and their cognate antigens can serve as capture agents and tags, respectively. An antibody binds to a small portion of its cognate antigen, known as its epitope, which contains as few as 3-6 amino acid residues (Pellequer et al. (1991) Methods in Enzymology 208:176). The amino acid residues can be contiguous, or they can be discontinuous within the antigen sequence. When the amino acid residues of the antigen sequence are discontinuous, they are presented in close proximity for recognition by the cognate antibody through three-dimensional folding of the antigen.

Candidate capture agent—polypeptide binding pairs can be identified by any method known to the art, including, but are not limited to, one or several of the following methods, such as, for example:

a) phage display of a random peptide library followed by biopanning with the antibody of interest;

b) analysis of complementarity-determining regions (CDRs) of the antibody of interest;

c) theoretical molecular modeling of three-dimensional antibody structure;

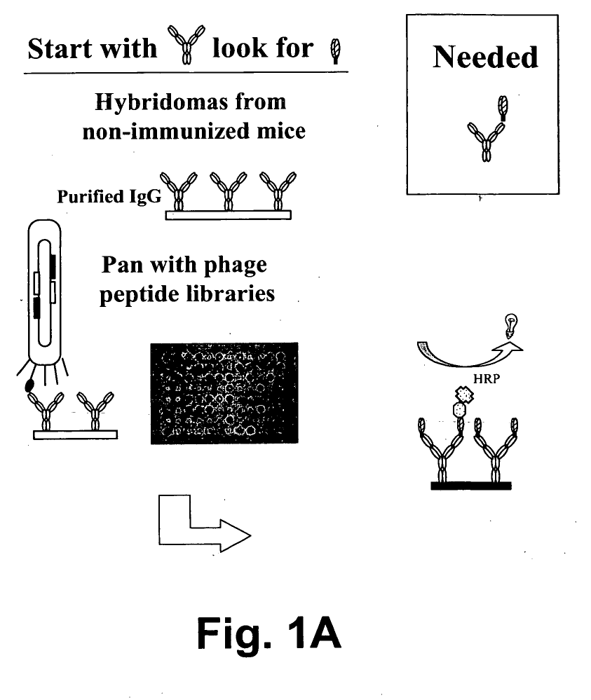

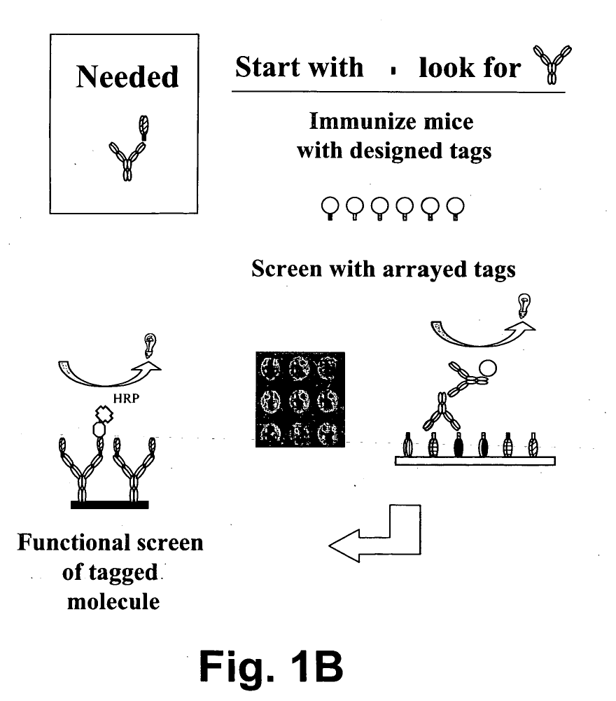

d) raising antibodies from exposure of a subject to an antigen and any method known to those of skill in the art for identifying pairs of molecules that bind with high affinity and specificity. The following discussion provides exemplary methods; others can be employed. Exemplary methods are depicted in FIGS. 1A-1B.

### a. Panning Phage Displayed Peptide Libraries

One method for identifying pairs employs phage displayed peptide libraries, such as random peptide libraries. Hybridoma cells are created either from non-immunized mice or mice immunized with a protein expressing a library of random epitopes or other random peptide libraries (see, e.g., FIG. 1A). Stable hybridoma cells are initially screened for high Ig production and epitope binding. Ig production is measured in culture supernatants by ELISA using a goat anti-mouse IgG antibody. Epitope binding also is measured by ELISA in which the mixture of haptens (epitope tagged proteins) used for immunization are immobilized to the ELISA plate and bound IgG from the culture supernatants is measured using a goat anti-mouse IgG antibody. Both assays are done in 96-well formats or other suitable formats. For example, approximately 10,000 hybridomas are selected from these screens (see, e.g., Example 1).

Next, the Ig are separately purified using 96-well or higher density purification plates containing filters with immobilized Ig-binding proteins (proteins A, G or L). The quantity of purified Ig is measured using a standard protein assay formatted for 96-well or higher density plates. Low microgram quantities of Ig from each culture are expected using this purification method.

The purified Ig are spotted separately onto a nitrocellulose filter using, for example, a standard pin-style arraying system. The purified Ig also are combined to produce a mixture with equal quantities of each Ig. The mixed Ig are bound to paramagnetic beads which are used as a solid-phase support to pan a library of bacteriophage expressing the random disulfide-constrained heptameric epitopes. The batch panning enriches the phage display library for phage expressing epitopes to the purified Ig. This enrichment dramatically reduces the diversity in the phage library.

The enriched phage display library is then bound to the array of purified Ig and stringently washed. Ig-binding phage are detected by staining with an anti-phage antibody-HRP conjugate to produce a chemiluminescent signal detectable with a charge coupled device (CCD)-based imaging system. Loci in the array producing the strongest signals are cut out and the phage eluted and propagated. Epitopes expressed by the recovered phage are identified by DNA sequencing and further evaluated for affinity and specificity. This method generates a collection of high-affinity, high-specificity antibodies that recognize the cognate epitopes. Continued screening produces larger collections of antibodies of improved quality.

Example 1 outlines-a high throughput screen for discovering immunoglobulin (Ig) produced from hybridoma cells for use in generating antibodies for use in the collections. Hybridoma cells are created either from non-immunized mice or mice immunized with a protein expressing a library of random disulfide-constrained heptameric epitopes or other random peptide libraries. Stable hybridoma cells are initially screened for high Ig production and epitope binding. Ig production is measured in culture supernatants by ELISA using a goat anti-mouse IgG antibody. Epitope binding also is measured by ELISA in which the mixture of haptens (epitope tagged proteins) used for immunization are immobilized to the ELISA plate and bound IgG from the culture supernatants is measured using a goat anti-mouse IgG antibody. Both assays are done in 96-well formats or other suitable formats. For example, approximately 10,000 hybridomas are selected from these screens.

Next, the Ig are separately purified using 96-well or higher density purification plates containing filters with immobilized Ig-binding proteins (proteins A, G or L). The quantity of purified Ig is measured using a standard protein assay formatted for 96-well or higher density plates. Low microgram quantities of Ig from each culture are expected using this purification method.

The purified Ig are spotted separately onto a nitrocellulose filter using a standard pin-style arraying system. The purified Ig also are combined to produce a mixture with equal quantities of each Ig. The mixed Ig are bound to paramagnetic beads which are used as a solid-phase support to pan a library of bacteriophage expressing the random disulfide-constrained heptameric epitopes. The batch panning enriches the phage display library for phage expressing epitopes to the purified Ig. This enrichment dramatically reduces the diversity in the phage library.

The enriched phage display library is then bound to the array of purified Ig and stringently washed. Ig-binding phage are detected by staining with an anti-phage antibody-HRP conjugate to produce a chemiluminescent signal detectable with a charge coupled device (CCD)-based imaging system. Loci in the array producing the strongest signals are cut out and the phage eluted and propagated. Epitopes expressed by the recovered phage are identified by DNA sequencing and further evaluated for affinity and specificity. This method generates a collection of high-affinity, high-specificity antibodies that recognize the cognate epitopes. Continued screening produces larger collections of antibodies of improved quality.

### b. Analysis of Complementarity-Determining Regions (CDRs) of an Antibody

Capture agent-polypeptide pairs can be identified by analyzing complementarity-determining regions (CDRs) in the antibody of interest. Translation of available cDNA sequences of the variable light and variable heavy chains of a particular antibody permit the delineation of the CDRs by comparison to the database of protein sequences compiled in “Sequences of Proteins of Immunological Interest”, Fifth Edition, Volume 1, Editors: Kabat et al. (1991) (see, e.g., table on page xvi). In some cases, CDR peptides can mimic the activity of an antibody molecule (Williams et al. Proc. Natl. Acad. Sci. U.S.A. 86: 5537 (1989)). CDR peptides may bind their cognate antibody, thus effecting displacement of the antibody from the antigen. To increase the efficiency of the above procedures in identifying candidate releasing peptides, biospecific interaction analysis using surface plasmon resonance detection through the use of the Pharmacia BIAcore™ system can be used. This technology provides the ability to determine binding constants and dissociation constants of antibody-antigen interactions. Analysis of multiple antibodies and the number of biopanning steps (at set antibody concentrations) required to identify a tight-binding consensus peptide sequence will provide a database on which to compare kinetic binding parameters with the ability to identify tight binding polypeptide tags. The use of the BIAcore™ system requires purified antibody and a source of soluble antigen. Phage display-selected clones can be used as a source of peptide antigen and directly analyzed for antibody binding.

### c. Theoretical Molecular Modelling of Three-Dimensional Antibody Structure

In silico methods can be used to determine capture agent—polypeptide tag pairs. Structural information (NMR and X-ray) is known for numerous immunoglobulins and is accessible, for example, at the Protein Databank (online at rcsb.org/pdb/) and ImMunoGeneTics (online at imgt.cnusc.fr:8104/home.html). Using one of a number of available molecular modeling programs such as HyperChem (Hypercube, Inc.), InsightII (Molecular Simulations, Inc.); SpartanPro (Schrodinger, Inc.) Sybyl (Tripos, Inc.) and XtalView (Tripos, Inc.) the structural data can be manipulated in silico to identify potential molecules that can interact with the variable region of the antibody. The energy of interaction between the antibody and potential epitope can be determined using a molecular docking program such as DOCK, which is commercially available; see, also, e.g., (online at cmpharm.ucsf.edu/kuntz/dock.html), AutoDock (online at scripps.edu/pub/olson-web/doc/autodock/), IDock (online at archive.ncsa.uiuc.edu/Vis/Projects/Docker/) or SPIDeR (online at simbiosys.ca/sprout/eccc/spider.html). Once identified and the binding energy is determined in silico, polypeptides that constitute the tags can be synthesized or purchased commercially and tested in vitro for their specificity and affinity for the antibody in question.

### d. Raising Antibodies from Exposure of a Subject to an Antigen

Antibodies have traditionally been obtained by repeatedly injecting a suitable animal (e.g., rodents, rabbits and goats) with an antigen or antigen with adjuvant (see, e.g., FIG. 1B). If the animal's immune system has responded, specific antibodies are secreted into the serum. The antibody-rich serum (antiserum) that is collected contains a heterogeneous mixture of antibodies, each produced by a different B lymphocyte. The different antibodies recognize different parts of the antigen, and are thus a heterogeneous mixture of antibodies. A homogeneous preparation of antibodies can be prepared by propagating an immortal cell line wherein antibody producing B cells are fused with cells derived from an immortal B-cell tumor. Those hybrids (hybridoma cells) that are producing the desired antibody and have the ability to multiply indefinitely are selected. Such hybridomas are propagated as individual clones, each of which can provide a permanent and stable source of a single antibody (a monoclonal antibody) which is specific for the antigen of interest. The antibodies can be purified from the propagating hybridomas by any method known to those skilled in the art. Fragments thereof can be synthesized or produced and modified forms thereof produced.

### 4. Preparation of Capture Agent Arrays

By reacting a collection of capture agents with libraries of polypeptide tag-labeled molecules so that-the tags bind to their cognate capture agent, capture systems are prepared. The resulting capture systems can be used in a variety of methods (see, e.g., U.S. application Ser. No. 09/910,120, published as U.S. application Ser. No. 20020137053; published International PCT application No. WO 02/06834; and U.S. provisional application Serial No. 60/352,011), including, for example, a reduction in the diversity of a library encoding the tagged molecules is achieved by identifying the members of the collection of the capture agents to which polypeptide-tagged molecules of a desired property have bound. Each collection of capture agents serves as a sorting device for effecting this reduction in diversity. Repeating the process a plurality of times can effect a rapid and substantial reduction in diversity. The collections of capture agents, and also the capture systems provide surfaces with diverse binding properties. Methods that exploit these surface properties, binding specificity and addressable loci of the capture systems are contemplated.

Each locus of a collection of capture agents contains a multiplicity of capture agents, such as antibodies with a single specificity. In solid phase embodiments, in which the capture agents are displayed as loci, each locus is of a size suitable for detection. Loci can be on the order of 1 to 360 microns, typically 1 to 100, 1 to 50, and 1 to 10 microns, depending upon the size of the array, target molecules and other parameters. Generally the loci are 50 to 300 microns. In preparing the arrays, a sufficient amount is delivered to the surface to functionally cover it for detection of proteins having the desired properties. Generally the volume of antibody-containing mixture delivered for preparation of the arrays is a nanoliter volume (1 up to about 99 nanoliters) and is generally about a nanoliter or less, typically between about 50 and about 200 picoliters. This is very roughly about 10 million to 100,000 molecules per locus, where each locus has capture agents, such as antibodies, that recognize a single epitope. For example, if there are 10 million molecules and 1000 different ones in the protein mixture reacting with the locus, there are 10⁴ of each type of molecule per locus. The size of the array and each locus is such that positive reactions in the screening step can be imaged, generally by imaging the entire array or a plurality thereof, such as 24, 96, or more arrays, at the same time.

A support (see below for exemplary supports), such as KODAK paper plus gelatin, plastic or other suitable matrix can be used, and then ink jet and stamping technology or other suitable dispensing methods and apparatus, are used to reproducibly print the arrays. The arrays are printed with, for example, a piezo or inkjet printer or other such nanoliter or smaller volume dispensing device. For example, arrays with 1000 loci can be printed. A plurality of replicate arrays, such as 24 or 48, 96 or more can be placed on a sheet the size of a conventional 96-well plate.

Among the embodiments contemplated herein, are sheets of arrays each with replicates of the antibody array. These are prepared using, for example, a piezo or inkjet dispensing system. A large number, for example, 1000 can be printed at a time using, for example, a print head with 1000 different holes (like a stamp with 500 μM holes). It can be fabricated from, for example, molded plastic with many holes, such as 1000 holes each filled with 1000 different capture agents, such as antibodies. Each hole can be linked to reservoirs that are linked to conduits of decreasing size, which ultimately dispense the capture agents, such as antibodies into the print head. Each array on the sheet can be spatially separated, and/or separated by a physical barrier, such as a plastic ridge, or a chemical barrier, such a hydrophobic barrier (i.e., hydrogels separated by hydrophobic barriers). The sheets with the arrays can be conveniently the size of a 96-well plate or higher density. Each array contains a plurality of addressable anti-tag antibodies specific for the pre-selected set of polypeptide tags. For example, 33×33 arrays contain roughly 1000 antibodies, each locus on each array containing antibodies that specifically bind to a single pre-selected epitope. A plurality of arrays separated by barriers can be employed.

For dispensing the antibodies onto the surface, the goal is functional surface coverage, such that a screened desired protein is detectable. To achieve this, for example, about 1 to 2 mg/ml from the starting collection are used and about 500 picoliters per antibody are deposited per locus on the array. The exact amount(s) can be empirically determined and depend upon several variables, such as the surface and the sensitivity of the detection methods. The antibodies are generally covalently linked, such as by free sulfhydryl linkages to maleimides or free amine linkage to NHS-esters on the surface.

Other exemplary dispensing and immobilizing systems include, but are not limited to, for example, systems available from Genometrix, which has a system for printing on glass; from Illumina, which employs the tips of fiber optic cables as supports; from Texas Instruments, which has chip surface plasmon resonance (i.e., protein derivatized gold); inkjet systems, such as those from Microfab Technologies, Piano Tex.; Incyte, Palo Alto, Calif., Protogene, Mountain View, Calif., Packard BioSciences, Meriden Conn., and other such systems for dispensing and immobilizing proteins to suitable support surfaces. Other systems such as blunt and quill pins, solenoid and piezo nanoliter dispensers and others also are contemplated.

### 5. Preparation of Other Addressable Collections

Also provided herein are capture agents that are linked to beads or other particulate supports that are associated with an identifier. For example, the capture agents are linked to optically encoded microspheres, such as those available from Luminex, Austin Tex., that contain fluorescent dyes encapsulated therein. The microsphere, which encapsulate dyes, are prepared from any suitable material (see, e.g., International PCT application Nos. WO 01/13119 and WO 99/19515; see description below), including styrene-ethylene-butylene-styrene block co-polymers, homopolymers, gelatin, polystyrene, polycarbonate, polyethylene, polypropylene, resins, glass, and any other suitable support (matrix material), and are of a size of about a nanometer to about 10 millimeters in diameter. By virtue of the combination of, for example, two different dyes at ten different concentrations, a plurality microspheres (100 in this instance), each identifiable by a unique fluorescence, are produced.

Alternatively, combinations of chromophores or colored dyes or other colored substances are encapsulated to produce a variety of different colors encapsulated in microspheres or other particles, which are then used as supports for the capture agents, such as antibodies. Each capture agent, such as an antibody, is linked to a particular colored bead, and, is thereby identifiable. After producing the beads with linked capture agents, such as antibodies, reaction with the epitope-tagged molecules can be performed in liquid phase. The beads that react with the epitopes are identified, and as a result of the color of the bead the particular epitope and is then known. The sub-library from which the linked molecule is derived is then identified.

### 6. Interactions between Capture Agents and Polypeptide Tags

As noted, the interactions between the capture agents and polypeptide tags are designed or selected to be of relatively high affinity and specificity. Any interaction, including, but are not limited to, hydrophobic, ionic, covalent and van der Waals and combinations thereof is contemplated, as long as it meets the criteria of affinity and specificity.

Generally the interaction between the capture agent and tag is reversible, such as the interaction between an antibody and an epitope, and has an association constant sufficient for detection of subsequent binding events, between the resulting capture system and other moieties.

Capture agents can be modified following the specific affinity interaction, such as by cross-linking between the tag/binding protein and the capture agent. For example, covalent cross-linking reagent (through chemical, electrical, or photoactivatable means) can be used to fix or stabilize interactions between proteins (Besemer et al. (1993) Cytokine 5:512-519; Meh et al. (1996) J. Biol. Chem. 271:23121-23125; Behar et al. (2000) J. Biol. Chem. 275:9-17; Huber et al. (1993) Eur. J. Biochem. 218, 1031-1039). A cross-link ensures that the interaction between the capture agent and polypeptide tag is long-lasting and stable. The initial interaction between the capture agent and the polypeptide tag determine the specificity while the cross-linking agent provides infinite affinity (Chmura et al. (2001) Proc. Natl. Acad. Sci. U.S.A. 98:8480-8484). This can be an added synthetic bi-functional cross-linking agent (Besemer et al. (1993) Cytokine 5:512-519; Meh et al. (1996) J. Biol. Chem. 5 271:23121-23125; Behar et al. (2000) J. Biol. Chem. 275:9-17; Huber et al. (1993) Eur. J. Biochem. 218, 1031-1039), or through a reactive group incorporated into the capture agent and the corresponding polypeptide tag (Chmura et al. (2002) J. Control Release 78:249-258; Kiick et al. (2002) Proc. Natl. Acad. Sci. U.S.A. 99:19-24; Saxon et al. (2000) Org. Lett. 10 2:2141-2143; Lemieux et al. (1998) Trends Biotechnol. 16:506-513).

The covalent cross-link can result from the enzymatic function of the polypeptide tag or capture agent. For example, self-splicing proteins known as inteins have been used for the ligation of peptides to a larger protein (Ayers et al. (2000) J. Biol. Chem. 275:9-17), and for the ligation of two subunits of a split-intein protein (Wu et al. (1998) Biochim. Biophys. Acta 1387:422-432; Southworth et al. (1998) EMBO J. 17:918-926). Alternately, several DNA modifying enzymes use a mechanism that involves an intermediate in which the enzyme is covalently bound to its DNA substrate (Chen et al. (1995) Nucleic Acids Res. 23:1177-1183; Topal et al. (1993) Nucleic Acids Res. 21:2599-2603; Thomas et al. (1990) J. Biol. Chem. 265:5519-5530). It is likely that mutation of these enzymes can result in the stabilization of that intermediate, and thus the covalent linkage is retained. These modifying enzymes are highly sequence specific, and presumably can be mutated to create enzymes with distinct specificities. Thus, dsDNA can be used as an effective capture agent with a restriction enzyme or topoisomerase (or binding domain thereof as a polypeptide tag.

### 7. Design and Preparation of Oligonucleotides/Primers

The polypeptide tag of known sequence is an advantage of the capture systems provided herein. Because the tag sequence and the loci to which each tag binds are known, it is possible to then identify molecules or specifically amplify nucleic acid molecules encoding linked polypeptides.

Thus, sorting large diversity libraries onto arrays and amplifying specific pools containing clones with the desired properties is dependent on the ability to uniquely tag a library with specific polypeptide tags and to then specifically amplify oligonucleotides encoding the tags. Oligonucleotide sets can be chemically synthesized, randomly combined by overlapping sequences, and ligated together to produce a template for enzymatic synthesis of the collection of primers or linkers.

The oligonucleotides are either single-stranded or double-stranded depending upon the manner in which they are to be incorporated into a tagged library. For example, they can be incorporated, by ligation of the double-stranded version, such as through a convenient restriction site, followed by amplification with a common region, or they can be incorporated by PCR amplification, in which case the oligonucleotides are single-stranded. In the methods herein, they are incorporated by introducing nucleic acid molecules into plasmids that also include the oligonucleotides encoding tags.

The primers, which are employed in some of the embodiments of the methods for tagging molecules, are central to the practice of some of the sorting methods. The primers and double-stranded oligonucleotides can include restriction site(s) and sequences to aid in unique or convenient priming, or can encode amino acids that confer desired properties, such as increased solubility, trafficking signals, and other properties. These primers can be forward or reverse primers, where the forward primer is that used for the first round in an amplification. Any suitable method for constructing double-stranded or single-stranded oligonucleotides may be employed. Methods for preparing large numbers of such oligomers have been described (see, e.g., International PCT application No. WO 02/06834 and published U.S. application Serial No. 20020137053).

### 8. Supports for Immobilizing Capture Agents

Supports for immobilizing capture agents include any of the insoluble materials known for immobilization of ligands and other molecules, used in many chemical syntheses and separations, such as in affinity chromatography, in the immobilization of biologically active materials, and during chemical syntheses of biomolecules, including proteins, amino acids and other organic molecules and polymers. Suitable supports include any material, including biocompatible polymers, that can act as a support matrix for attachment of the antibody material. The support material is selected so that it does not interfere with the chemistry or biological screening reaction.

Supports that also are contemplated for use herein include fluorophore-containing or fluorophore-impregnated supports, such as microplates and beads (commercially available, for example, from Amersham, Arlington Heights, Ill.; plastic scintillation beads from Nuclear Technology, Inc., San Carlos, Calif. and Packard, Meriden, Conn., and colored bead-based supports (fluorescent particles. encapsulated in microspheres) from Luminex Corporation, Austin, Tex. (see, International PCT application No. WO/0114589, which is based on U.S. application Ser. No. 09/147,710; see International PCT application No. WO/0113119, which is U.S. application Ser. No. 09/022,537). The microspheres from Luminex, for example, are internally color-coded by virtue of the encapsulation of fluorescent particles and can be provided as a liquid array. The capture agents, such as antibodies (epitopes) are linked directly or indirectly by any suitable method and linkage or interaction to the surface of the bead and bound proteins can be identified by virtue of the color of the bead to which they are linked. Detection can be effected by any method, and can be combined with chromogenic or fluorescent detectors or reporters that result in a detectable change in the color of the microsphere (bead) by virtue of the colored reaction and color of the bead. Detection methods include, but are not limited to, methods including, ultraviolet-visible (UV-VIS) spectroscopy, infra-red (IR) spectroscopy, fluorescence spectroscopy, fluorescence resonance energy transfer (FRET), NMR spectroscopy, circular dichroism (CD), mass spectrometry, other analytical methods, enzymatic assays for detection, antibody assays and other biological and/or chemical detection methods or any combination thereof.

For the bead-based arrays, the anti-tag capture agents are attached to the color-coded beads in separate reactions. The code of the bead identifies the capture agent, such as antibody, attached to it. The beads then can be mixed and subsequent binding steps performed in solution. They then can be arrayed, for example, by packing them into a microfabricated flow chamber, with a transparent lid, that permits only a single layer of beads to form resulting in a two-dimensional array. The beads on which a protein is bound are identified, thereby identifying the capture agent and the tag. The beads are imaged, for example, with a CCD camera to identify beads that have reacted. The codes of such beads are identified, thereby identifying the capture agent, which in turn identifies the polypeptide tag and, ultimately, the protein of interest.

The support also can be a relatively inert polymer, which can be grafted by ionizing radiation to permit attachment of a coating of polystyrene or other such polymer that can be derivatized and used as a support. Radiation grafting of monomers. allows a diversity of surface characteristics to be generated on supports (see, e.g., Maeji et al. (1994) Reactive Polymers 22:203-212; and Berg et al. (1989) J. Am. Chem. Soc. 111:8024-8026). For example, radiolytic grafting of monomers, such as vinyl monomers, or mixtures of monomers, to polymers, such as polyethylene and polypropylene, produce composites that have a wide variety of surface characteristics. These methods have been used to graft polymers to insoluble supports for synthesis of peptides and other molecules.

The supports are typically insoluble substrates that are solid, porous, deformable, or hard, and have any required structure and geometry, including, but not limited to: beads, pellets, disks, capillaries, hollow fibers, needles, solid fibers, random shapes, thin films and membranes, and most generally, form solid surfaces with addressable loci. The supports also can include an inert strip, such as a TEFLON® (polytetrafluoroethylene) strip or other material to which the capture agents, antibodies and other molecules do not adhere, to aid in handling the supports, and can include an identifying symbology.

The preparation of and use of such supports are well known to those of skill in this art; there are many such materials and preparations thereof known. For example, naturally-occurring materials, such as agarose and cellulose, can be isolated from their respective sources, and processed according to known protocols, and synthetic materials can be prepared in accord with known protocols. These materials include, but are not limited to, inorganics, natural polymers, and synthetic polymers, including, but are not limited to: cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (see; Merrifield (1 964) Biochemistry 3:1385-1390), polyacrylamides, latex gels, polystyrene, dextran, polyacrylamides, rubber, silicon, plastics, nitrocellulose, celluloses, natural sponges, polystyrene, radiation grafted polymers, polyvinylidene fluoride (PVDF), and many others. Selection of the supports is governed, at least in part, by their physical and chemical properties, such as solubility, functional groups, mechanical stability, surface area swelling propensity, hydrophobic or hydrophilic properties and intended use.

### a. Natural Support Materials

Naturally-occurring supports include, but are not limited to, agarose, other polysaccharides, collagen, celluloses and derivatives thereof, glass, silica, and alumina. Methods for isolation, modification and treatment to render them suitable for use as supports is well known to those of skill in this art (see, e.g., Hermanson et al. (1992) Immobilized Affinity Ligand Techniques, Academic Press, Inc., San Diego). Gels, such as agarose, can be readily adapted for use herein. Natural polymers such as polypeptides, proteins and carbohydrates; met alloids, such as silicon and germanium, that have semiconductive properties, also can be adapted for use herein. Also, metals such as platinum, gold, nickel, copper, zinc, tin, palladium, silver can be adapted for use herein. Other supports of interest include oxides of the met al and met alloids such as Pt—PtO, Si—SiO, Au—AuO, TiO2, Cu—CuO, and the like. Also compound semiconductors, such as lithium niobate, gallium arsenide and indium-phosphide, and nickel-coated mica surfaces, as used in preparation of molecules for observation in an atomic force microscope (see, e.g., III et al. (1993) Biophys J. 64:919) can be used as supports. Methods for preparation of such matrix materials are well known.

For example, U.S. Pat. No. 4,175,183 describes a water insoluble hydroxyalkylated cross-linked regenerated cellulose and a method for its preparation. A method of preparing the product using near stoichiometric proportions of reagents is described. Use of the product directly in gel chromatography and as an intermediate in the preparation of ion exchangers also is described.

### b. Synthetic Supports

There are innumerable synthetic supports and methods for their preparation known to those of skill in this art. Synthetic supports typically produced by polymerization of functional matrices, or copolymerization from two or more monomers from a synthetic monomer and naturally occurring matrix monomer or polymer, such as agarose.

Synthetic matrices include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield (1964) Biochemistry 3:1385-1390; Berg et al. (1990) in Innovation Perspect. Solid Phase Synth. Collect. Pap., Int. Symp., 1st, Epton, Roger (Ed), pp. 453-459; Berg et al. (1989) in Pept., Proc. Eur. Pept. Symp., 20th, Jung, G. et al. (Eds), pp. 196-198; Berg et al. (1989) J. Am. Chem. Soc. 111:8024-8026; Kent et al. (1979) Isr. J. Chem. 17:243-247; Kent et al. (1978) J. Org. Chem. 43:2845-2852; Mitchell et al. (1976) Tetrahedron Lett. 42:3795-3798; U.S. Pat. No. 4,507,230; U.S. Pat. No. 4,006,117; and U.S. Pat. No. 5,389,449). Methods for preparation of such support matrices are well-known to those of skill in this art.

Synthetic support matrices include those made from polymers and co-polymers such as polyvinylalcohols, acrylates and acrylic acids such as polyethylene-co-acrylic acid, polyethylene-co-methacrylic acid, polyethylene-co-ethylacrylate, polyethylene-co-methyl acrylate, polypropylene-co-acrylic acid, polypropylene-co-methyl-acrylic acid, polypropylene-co-ethylacrylate, polypropylene-co-methyl acrylate, polyethylene-co-vinyl acetate, polypropylene-co-vinyl acetate, and those containing acid anhydride groups such as polyethylene-co-maleic anhydride, polypropylene-co-maleic anhydride and the like. Liposomes also have been used as solid supports for affinity purifications (Powell et al. (1989) Biotechnol. Bioeng. 33:173).

For example, U.S. Pat. No. 5,403,750, describes the preparation of polyurethane-based polymers. U.S. Pat. No. 4,241,537 describes. a plant growth medium containing a hydrophilic polyurethane gel composition prepared from chain-extended polyols; random copolymerization can be performed with up to 50% propylene oxide units so that the prepolymer is a liquid at room temperature. U.S. Pat. No. 3,939,123 describes lightly cross-linked polyurethane polymers of isocyanate terminated prepolymers containing poly(ethyleneoxy) glycols with up to 35% of a poly(propyleneoxy) glycol or a poly(butyleneoxy) glycol. In producing these polymers, an organic polyamine is used as a cross-linking agent. Other supports and preparations thereof are described in U.S. Pat. Nos. 4,177,038, 4,175,183, 4,439,585, 4,485,227, 4,569,981, 5,092,992, 5,334,640, 5,328,603.

U.S. Pat. No. 4,162,355 describes a polymer suitable for use in affinity chromatography, which is a polymer of an aminimide and a vinyl compound having at least one pendant halo-methyl group. An amine ligand, which affords sites for binding in affinity chromatography is coupled to the polymer by reaction with a portion of the pendant halo-methyl groups and the remainder of the pendant halo-methyl groups are reacted with an amine containing a pendant hydrophilic group. A method of coating a substrate with this polymer also is described. An exemplary aminimide is 1,1-dimethyl-1-(2-hydroxyoctyl)amine methacrylimide and vinyl compound is a chloromethyl styrene.

U.S. Pat. No. 4,171,412 describes specific supports based on hydrophilic polymeric gels, generally of a macroporous character, which carry covalently bonded D-amino acids or peptides that contain D-amino acid units. The basic support is prepared by co-polymerization of hydroxyalkyl esters or hydroxyalkylamides of acrylic and methacrylic acid with cross-linking acrylate or methacrylate co-monomers are modified by the reaction with diamines, amino acids or dicarboxylic acids and the resulting carboxy terminal or amino terminal groups are condensed with D-analogs of amino acids or peptides. The peptide containing D-amino acids also can be synthesized step-wise on the surface of the carrier.

U.S. Pat. No. 4,178,439 describes a cationic ion exchanger and a method for preparation thereof. U.S. Pat. No. 4,180,524 describes chemical syntheses on a silica support.

Immobilized artificial membranes (IAMs; see, e.g., U.S. Pat. Nos. 4,931,498 and 4,927,879) also can be used. IAMs mimic cell membrane environments and can be used to bind molecules that preferentially associate with cell membranes (see, e.g., Pidgeon et al. (1990) Enzyme Microb. Technol. 12:149).

Among the supports contemplated herein are those described in International PCT application Nos WO 00/04389, WO 00/04382 and WO 00/04390; KODAK film supports coated with a matrix material; see also, U.S. Pat. Nos. 5,744,305 and 5,556,752 for other supports of interest. Also of interest are colored “beads”, such as those from Luminex (Austin, Tex.).

### C. Immobilization and Activation

Numerous methods have been developed for the immobilization of proteins and other biomolecules onto solid or liquid supports (see, e.g., Mosbach (1976) Methods in Enzymology 44; Weet all (1975) Immobilized Enzymes, Antigens, Antibodies, and Peptides; and Kennedy et al. (1983) Solid Phase Biochemistry, Analytical and Synthetic Aspects, Scouten, ed., pp. 253-391; see, generally, Affinity Techniques. Enzyme Purification: Part B. Methods in Enzymology, Vol. 34, ed. W. B. Jakoby, M. Wilchek, Acad. Press, N.Y. (1974); Immobilized Biochemicals and Affinity Chromatography, Advances in Experimental Medicine and Biology, vol. 42, ed. R. Dunlap, Plenum Press, N.Y. (1974)).

Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, ImmunoTechnology Catalog & Handbook, 1992-1993, which describes the preparation of and use of such reagents and provides a commercial source for such reagents; and Wong (1993) Chemistry of Protein Conjugation and Cross Linking, CRC Press; see, also DeWitt et al. (1993) Proc. Natl. Acad. Sci. U.S.A. 90:6909; Zuckermann et al. (1992) J. Am. Chem. Soc. 114:10646; Kurth et al. (1994) J. Am. Chem. Soc. 116:2661; Ellman et al. (1994) Proc. Natl. Acad. Sci. U.S.A. 91:4708; Sucholeiki (1994) Tetrahedron Lttrs. 35:7307; and Su-Sun Wang (1976) J. Org. Chem. 41:3258; Padwa et al. (1971) J. Org. Chem. 41:3550 and Vedejs et al. (1984) J. Org. Chem. 49:575, which describe photosensitive linkers).

To effect immobilization, a solution of the protein or other biomolecule is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO/86 03840)

A large variety of methods are known for attaching biological molecules, including proteins and nucleic acids, molecules to solid supports (see. e.g., U.S. Pat. No. 5451683). For example, U.S. Pat. No. 4,681,870 describes a method for introducing free amino or carboxyl groups onto a silica support. These groups can subsequently be covalently linked to other groups, such as a protein or other anti-ligand, in the presence of a carbodiimide. Alternatively, a silica matrix can be activated by treatment with a cyanogen halide under alkaline conditions. The anti-ligand is covalently attached to the surface upon addition to the activated surface. Another method involves modification of a polymer surface through the successive application of multiple layers of biotin, avidin and extenders (see, e.g., U.S. Pat. No. 4,282,287); other methods involve photoactivation in which a polypeptide chain is attached to a solid substrate by incorporating a light-sensitive unnatural amino acid group into the polypeptide chain and exposing the product to low-energy ultraviolet light (see, e.g., U.S. Pat. No. 4,762,881). Oligonucleotides also have been attached using photochemically active reagents, such as a psoralen compound, and a coupling agent, which attaches the photoreagent to the substrate (see, e.g., U.S. Pat. No. 4,542,102 and U.S. Pat. No. 4,562,157). Photoactivation of the photoreagent binds a nucleic acid molecule to the substrate to give a surface-bound probe.

Covalent binding of the protein or other biomolecule or organic molecule or biological particle to chemically-activated solid matrix supports such as glass, synthetic polymers, and cross-linked polysaccharides is a more frequently used immobilization technique. The molecule or biological particle can be directly linked to the matrix support or linked via a linker, such as a met al (see, e.g., U.S. Pat. No. 4,179,402; and Smith et al. (1992) Methods: A Companion to Methods in Enz. 4:73-78). An example of this method is the cyanogen bromide activation of polysaccharide supports, such as agarose. The use of perfluorocarbon polymer-based supports for enzyme immobilization and affinity chromatography is described in U.S. Pat. No. 4,885,250. In this method the biomolecule is first modified by reaction with a perfluoroalkylating agent such as perfluorooctylpropylisocyanate described in U.S. Pat. No. 4,954,444. Then, the modified protein is adsorbed onto the fluorocarbon support to effect immobilization.

The activation and use of supports are well known and can be effected by any such known methods (see, e.g., Hermanson et al. (1992) Immobilized Affinity Ligand Techniques, Academic Press, Inc., San Diego). For example, the coupling of the amino acids can be accomplished by techniques familiar to those in the art and provided, for example, in Stewart and Young, 1984, Solid Phase Synthesis, Second Edition, Pierce Chemical Co., Rockford.

Molecules also can be attached to supports through kinetically inert met al ion linkages, such as Co(III), using, for example, native met al binding sites on the molecules, such as IgG binding sequences, or genetically modified proteins that bind met al ions (see, e.g., Smith et al. (1992) Methods: A Companion to Methods in Enzymology 4, 73 (1992); III et al. (1993) Biophys J. 64:919; Loetscher et al. (1992) J. Chromatography 595:113-199; U.S. Pat. No. 5,443,816; Hale (1995) Analytical Biochem. 231:46-49).

Other suitable methods for linking molecules and biological particles to solid supports are well known to those of skill in this art (see, e.g., U.S. Pat. No. 5,416,193). These linkers include linkers that are suitable for chemically linking molecules, such as proteins and nucleic acid, to supports including, but are not limited to, disulfide bonds, thioether bonds, hindered disulfide bonds, and covalent bonds between free reactive groups, such as amine and thiol groups. These bonds can be produced using heterobifunctional reagents to produce reactive thiol groups on one or both of the moieties and then reacting the thiol groups on one moiety with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid dihydrazide, that are cleaved in more acidic intracellular compartments; cross-linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30:379-386).

Exemplary linkages include direct linkages effected by adsorbing the molecule or biological particle to the surface of the support. Other exemplary linkages are photocleavable linkages that can be activated by exposure to light (see, e.g., Baldwin et al. (1995) J. Am. Chem. Soc. 117:5588; Goldmacher et al. (1992) Bioconj. Chem. 3:104-107, which linkers are herein incorporated by reference). The photocleavable linker is selected such that the cleaving wavelength that does not damage linked moieties. Photocleavable linkers are linkers that are cleaved upon exposure to light (see, e.g., Hazum et al. (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl. chloride cross-linking reagents that produce photocleavable linkages). Other linkers include fluoride labile linkers (see, e.g., Rodolph et al. (1995) J. Am. Chem. Soc. 117:5712), and acid labile linkers (see, e.g., Kick et al. (1995) J. Med. Chem. 38:1427)). The selected linker depends upon the particular application and, if needed, can be empirically selected.

### C. Preparation of the Capture Systems

Capture systems provided herein can be used in a variety of methods, such as those described herein (see, also, published International PCT application No. WO 02/06834; published U.S. application Serial No. US20020137053; U.S. provisional application Serial No. 60/352,011). Important to many methods that employ these systems is the distribution of tags on polypeptide-tagged molecules.

In many applications even distribution of tags is advantageous. For example, an even distribution of the tags among tagged molecules allows for the control of the diversity of, the tags among the loci of an addressable array. Ideally, the diversity of tags of a locus is about 1, but on the average can be more than 1, up to,about 100, 50, 25, 10, 5, 1.5 or 1.1.

An even distribution of tags permits a higher diversity of tagged molecules at each locus. The diversity of tagged molecules at each locus can be 10², 10³, 10⁴, 10⁵, 10⁶, 10⁷, 10⁸, 10⁹, 10¹⁰, 10¹¹, 10¹² or greater. If there is an even distribution of tags, then the diversity of molecules at each locus is substantially the same, generally within 1, 0.5, 0.1 order of magnitude. If the tags, however, are not evenly distributed, then the same tagged molecules will be at a plurality of loci in a capture system. Once the tags are evenly distributed, the diversity of tagged molecules at each locus can be selected or adjusted as desired and depends upon the application.

In many applications, high diversity of tagged molecules at each locus is advantageous; in others it may be disadvantageous. For example, if a locus has too high a diversity of tags, then the variety of molecules displayed by the interaction between the capture agent and the polypeptide tag will be less than at a locus where the diversity of tagged molecules is less. A high diversity of displayed tagged molecules, however, can result in missed binders because of concentration effects. If a locus has too low a diversity of tagged molecules, then the concentration of the variety of displayed molecules can result in falsely positive signals due to the inclusion of molecules which interact weakly with the displayed molecules. Thus, the level of diversity at a locus is a function of the purpose for which the capture system is employed, and can be empirically selected.

In some experimental situations, it may be desirable to skew the diversity of tagged molecules on the loci in one direction or the other. For example, the use of the capture system to immobilize whole cells can require a lower diversity of tagged molecules on a locus as fixation of the cell can require multiple surface-array interactions rather than a one-to-one interaction. One of skill in the art can assess the level of diversity of tag molecules among the loci required for a particular experimental situation and determine this value empirically.

For most applications, however, the tags should be distributed on molecules from the master library, such that, on the average each different tagged molecule is uniquely tagged so that the same molecule is not captured at a plurality of loci. It is understood that some molecules, by virtue of the operation of probability, will be tagged with more than one tag. In addition, for some applications, having the same molecule with different tags so that they are captured on a plurality of loci, is acceptable. In most instances, even distribution of tags is desirable so that a molecule will only be captured at one loci (or rarely two) in a collection of capture agents.

Methods for effecting even distribution sufficient for use of the capture systems have been described (see, e.g., published International PCT application No. WO 02/06834; published U.S. application Serial No. US20020137053; U.S. provisional application Serial No. 60/352,011). In these methods, the tags were linked to molecules in the master library, prior to subdivision.

Provided herein is another method for effecting even distribution. This method, which can be practiced to distribute any type of tag on any collection of molecules, is particularly adaptable for instances in which the master library is a nucleic acid library and the tags that bind to the capture agents are polypeptide tags. In this method, described with reference to nucleic acid, such as DNA libraries, the nucleic acid library is subdivided, tags are added to produce tagged sub-libraries, in which the nucleic acid encodes the same tag for all members of the sub-library, the tagged sub-libraries are pooled to form a mixed tag library such that the same number of tagged molecules is added from each sub-library. This can be achieved by adjusting the concentration of each tagged sub-library or an aliquot thereof or determining the concentration of tagged molecules of each sub-library and pooling equivalent-numbers of tagged molecules. The mixed tag library is contacted with addressed collection of capture agents in which the capture agents at or of each loci bind to the same tag, which generally differs from the tag to which the agents at other loci bind. Alternatively, the mixed library is divided or aliquots are removed and contacted with a predetermined number “q”, where q is from 2 or more, generally, 2 to 10, 20, 30, 50, 100, 200, 250, 300, 500, 1000, 2000, 3000, 4000, 5000, 10,000 and more, of addressable arrays, generally, although not necessarily, replicate arrays, of capture agents. As noted, generally, in the addressed collection of capture agents, the capture agents at or of each loci bind to the same tag, which generally differs from the tag to which the agents at other loci bind.

The method for evenly distributing tags on tagged-molecules that is provided herein includes some or all of the following steps:

a) determining the diversity of molecules required;

b) producing or obtaining a master library;

c) optionally, adjusting the diversity of a master library so that the diversity is substantially equal to, typically within an order of magnitude (i.e., within one order of magnitude, typically within 0.5 orders of magnitude or 0.1 orders of magnitude), the number of members of the library;

d) dividing the master library into “n” sub-libraries designated 1−n, where n is equal to or less than the number of different tags, i.e., nucleic acid molecules having different sequences encoding different polypeptide tags in the exemplified embodiment;

e) attaching a nucleic acid molecule encoding a polypeptide tag (or attaching a tag) to members of each sub-library to produce “n” tagged sub-libraries containing encoded tagged members, whereby the polypeptide tag encoding portion is in reading frame with a polypeptide encoded by the nucleic acid molecule, and such that the encoded polypeptide tag is unique to each sub-library;

f) mixing some or all of the tagged sub-libraries to produce a mixed library, where the number of tagged molecules added from each sub-library is about the same (i.e., within one order of magnitude, typically within 0.5 orders of magnitude or 0.1 orders of magnitude);

g) optionally normalizing the mixed library such that the relative number of molecules from each sub-library represented in the mixed library is within 0.5 orders of magnitude, typically 0.2, 0.1 or 0.05 orders of magnitude.

h) splitting the mixed library into “q” array libraries, where q is from 1 to a predetermined number of arrays;

i) if the libraries are nucleic acid libraries, producing the tagged polypeptides in each array library.

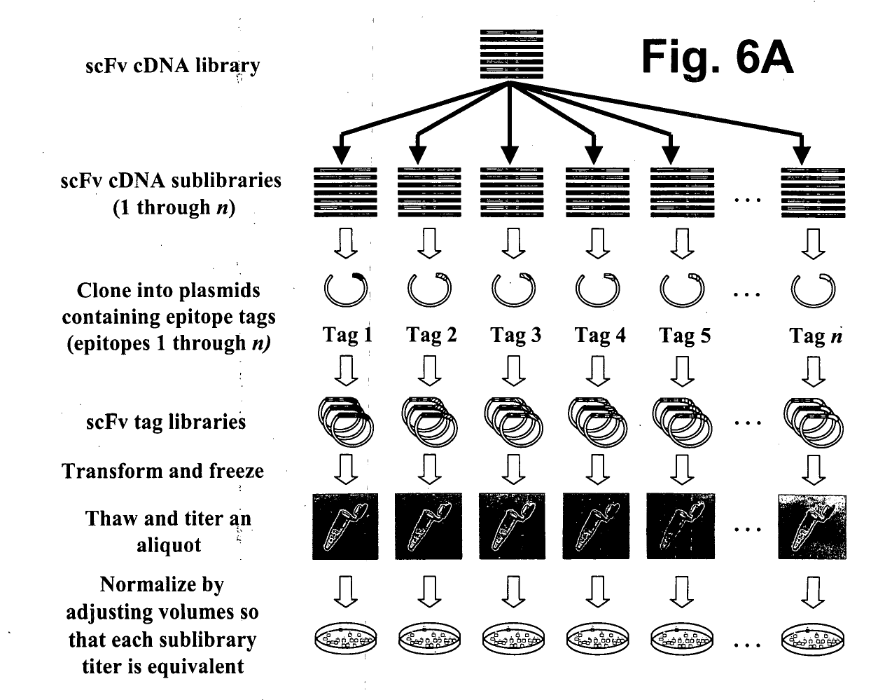

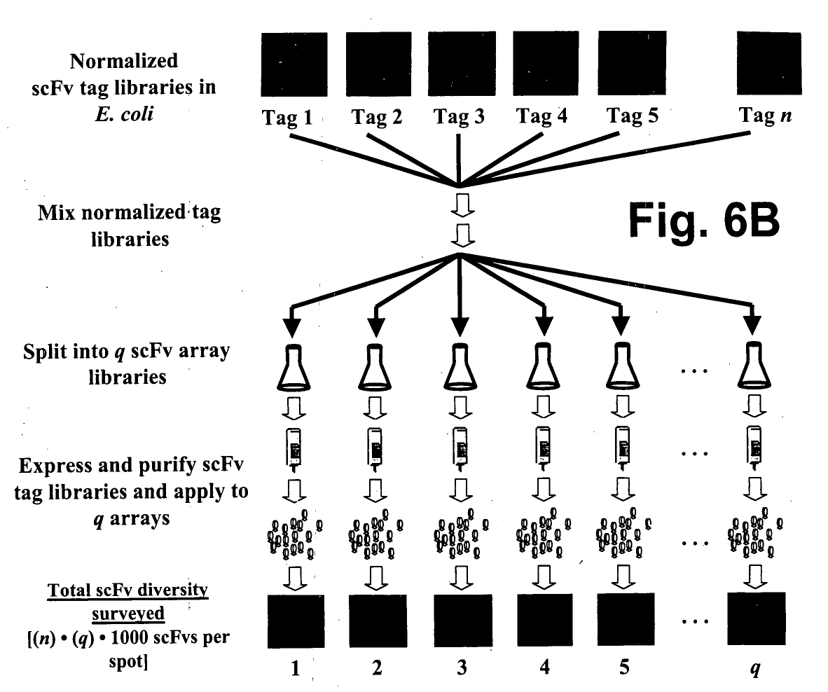

An exemplary embodiment of the process is outlined in FIGS. 6A and 6B. Application of the method for evenly distributing polypeptide tags on proteins encoded by a master library is described. It is noted that practice of this method is not limited to polypeptide tagged proteins, but can be adapted for distribution of any tags on any collection of molecules. In all instances, the methods include steps in which molecules in the library are separated into a predetermined number of sub-libraries less than or equal to the number of different tags, and then, after attaching a tag members of each sub-library, equal numbers of tagged molecules are mixed to produce a mixed tagged collection of molecules.

As noted the following sections describe the process with reference for exemplification purposes to evenly distributing polypeptide tags on collections of polypeptides that are encoded by a master library.

### 1. Determining the Required Diversity of the Master Library

Prior to preparing or obtaining the Master library for tag incorporation, the diversity of molecules required for a particular intended application can be determined. This value either is predetermined or calculated based on one or more parameters, which include, for example, the total display desired for the arrayed capture system, the number of arrays to be screened, the number of loci per array and the diversity of. molecules to be displayed on each locus. These factors are interrelated and can be defined before preparing the capture system using the equations set forth below.

The total display of the arrayed capture system is dependent on the number of arrays of capture systems, the number of loci per array and the diversity per locus:

Total Display=(Arrays)(Loci)(Diversity per Locus)

The number of arrays and the number of loci can be decided and the array meeting the specifications can be prepared or can be a function of materials available for production of the arrays. For example, if an experimental setup includes 500 arrays with 10 loci per array and a diversity of 1000 per spot, then the total diversity displayed is equal to (500)(10)(1000) or 5×10⁶. As stated above, the diversity per locus is a function of the information required from the arrayed capture systems. If the system is being used to immobilize a specific molecule followed for purposes of monitoring a secondary reaction at the surface, then the diversity per locus required may be reduced. If the system is being used for high throughput screening of a particular pharmacological compound, then a higher diversity of potential reactants and, thus, the molecules displayed on the arrays may be desired. When determining the diversity to be displayed per spot, dilution of the signal or falsely positive signals can be considered.

Number of Loci=Number of Tags EQ 2

The number of loci per array is constrained by the number of unique capture agent-tag pairs available and the mechanical ability to localize loci within an array. For example, if there are 1000 known capture agent-tag pairs, then each array can have a maximum of 1000 loci. The array can have less than 1000 loci. More than 1000 loci will reduce the sorting capabilities of the tagged molecules as some loci within the array will share common immobilized capture agents, resulting in two addresses for the complementary tagged molecules.

An array library is formed from a splitting of the mixed library into q subsets of tagged molecules wherein q is the number of arrays. The diversity of an array library is therefore dependent only on the parameters present within an individual array, the number of loci and the diversity of displayed molecules on each spot.

Diversity of Array libraries=(Loci)(Diversity per Spot) EQ 3

For example, if an array has 10 loci and each locus has a diversity of 1000 then the array library has a diversity, of 10⁴.

The mixed library results from the pooling of an equal number of molecules from each tagged library, which is, in turn, formed from the insertion of nucleic acid molecules encoding a polypeptide tag into individual sub-libraries of the master library. Thus, the diversity of the mixed library is equal to the diversity of the total display (EQ 4), which is equal to the sum of the diversities of each array library (EQ 5):

Diversity of Mixed library−Total Display EQ 4

Total Display=(Arrays)(Loci)(Diversity per spot) EQ 5

For example, if an experimental setup has 500 arrays with 10 loci per array and each locus has a diversity of 1000 then the total diversity displayed and the diversity of the mixed libraries equals (500)(10)(1000) or 5×10⁶. The tagged libraries are formed directly from the incorporation of unique tags into the individual sub-libraries.

Div of Tagged libraries=(Arrays)(Div per Spot)

Div of Tagged Libraries=(Total Display)/(Loci)

Div of Tagged Libraries=((Div of Array libraries)(Arrays))/Loci

Incorporation of the polypeptide tags into the members of the sub-libraries is governed by a Gaussian distribution. In addition, cloning efficiency and the efficiency of other steps in the methods are 100%. Correction factors, which if necessary can be empirically determined, and included in the calculation of the diversity of the molecules within the sub-libraries. For the exemplified embodiment, it is recognized by those of skill in the art that cloning efficiency is about 10%. For different systems, efficiency can be empirically determined if needed. It is understood, since in general very large numbers of molecules are involved and the methods do not require a precise determination of diversity, precise determination of such numbers and correction factors is not necessary to achieve the desired result. Thus, the diversity of the sub-libraries is determined by the diversity of the tagged libraries with a correction for inefficiencies, such as inefficiencies in ligation or transfection or other processes, which for purposes herein in the exemplified embodiment and other embodiments where it has not been empirically determined, can be assumed to be about 10%.

Div of Sub-libraries=(Div of Tagged libraries)(1.0/Cloning efficiency)

For example, if the diversity of the tagged libraries is 5×10⁵ and the cloning efficiency is assumed to be about 0.1, then the diversity of the sub-libraries is 5×10⁶. This decrease in diversity from the sub-libraries to the tagged libraries results from known-and recognized inefficiencies in the ligation and transformation process. The diversity of the sub-libraries also can be determined from the diversity of the source of the sub-libraries, the master library, divided by the number of loci in the array.

Div of the Sub-libraries=(Div of Master library/Loci) EQ 6

The master library is subdivided into sub-libraries. The number of sub-libraries is dependent on the number of unique tags and ultimately the number of capture agent/tag pairs. The number of loci in an array is determined by the number of different capture agents, which depends on the number of different tags. Therefore, as stated above, the number of loci is equal to the number of tags and the diversity of the sub-libraries is indirectly proportional to the number of loci. If the number of loci per array increases, the number of sub-libraries also increases resulting in a decrease in the diversity of each sub-library. For example, if the diversity of the master library is 5×10⁷ and there are 10 loci per array then the diversity of the sub-libraries is (5×10⁷)/(10) or 5×10⁶. If the diversity of the master library is 5×10⁷ and the number of loci per array is increased to 250, then there are 250 sub-libraries each with a diversity of 2×10⁵.

Using the inverse of the equation above, the diversity of the master library can be calculated from the number of loci (or the number of sub-libraries) and the diversity of each sub-library.

Div of Master Library=(Div of Sub-libraries)(Loci) EQ 7

For example, if there are 50 sub-libraries or loci and each sub-library has a diversity of 1×10⁵, then the master library has to have a diversity of (50)(1×10⁵) or 5×10⁵.

If the diversity is known, then the number of arrays required, the number of loci per array, the diversity per locus or the total display of the arrayed capture systems can be calculated. Alternatively, any of the other parameters mentioned 4000 arrays with 100 loci and each locus is required to have a diversity of 500, then a master library has to be prepared or commercially obtained that has a diversity of 2×10⁸. If a master library is obtained that has a diversity of 2×10⁸, a diversity of 1000 per locus is required and the slide has space for 1000 arrays, then 250 loci need to be placed in each array. Table 2 below shows other examples of the relationships among the parameters defining the arrayed capture system. One of skill in the art can recognize that diversity of the master library, the number of arrays and loci per array and the diversity per locus can all be defined adjusted to suit any experimental situation.

TABLE 2

| | | | | | | | |
|---|---|---|---|---|---|---|---|
| Total Display | 5 × 10⁶ | 10⁷ | 2.5 × 10⁸ | 10⁹ | 2 × 10⁸ | 10⁹ | 10⁹ |
| Arrays | 500 | 1000 | 1000 | 4000 | 4000 | 2000 | 4000 |
| Loci | 10 | 10 | 250 | 250 | 100 | 500 | 500 |
| Div per Locus | 1000 | 1000 | 1000 | 1000 | 500 | 1000 | 500 |
| Master Library | 5 × 10⁷ | 10⁸ | 2.5 × 10⁹ | 10¹⁰ | 2 × 10⁹ | 10¹⁰ | 10¹⁰ |
| Sub-libraries | 5 × 10⁶ | 10⁷ | 10⁷ | 4 × 10⁷ | 2 × 10⁷ | 2 × 10⁷7 | 2 × 10⁷7 |
| Tag libraries | 5 × 10⁵ | 10⁶ | 10⁶ | 4 × 10⁶ | 2 × 10⁶ | 2 × 10⁶ | 2 × 10⁶7 |
| Mixed Libraries | 5 × 10⁶ | 10⁷ | 2.5 × 10⁸ | 10⁹ | 2 × 10⁸ | 10⁹ | 10⁹ |
| Array Libraries | 10⁴ | 10⁴ | 2.5 × 10⁵ | 2.5 × 10⁵ | 5 × 10⁴ | 5 × 10⁵ | 2.5 × 10⁵7 |

### 2. Creation of the Master Library and Division into Sub-libraries

A master library is a collection of molecules such as, but not limited to, organic compounds, inorganic compounds, polypeptides and nucleic acids. Examples of master libraries for use with the methods provided herein include, but are not limited to, cDNA libraries, combinatorial small molecule and peptide libraries and BAC and PAC libraries. These master libraries can be produced synthetically using any method known to those skilled in the art (see, e.g., EXAMPLE 4), or can be purchased commercially from companies such as Invitrogen (online at resgen.com/intro/libraries.php3) and Jerini Peptide Technology (online at jerini.de/base.htm). For exemplification of the methods herein, the master library is a collection of nucleic acid molecules that encode polypeptides. The diversity of the master library is equal to the number of unique members within the collection. The diversity of the master library can be determined by empirical methods or is known when the library is constructed or obtained. The master library is then diluted such that the diversity of the library is equal to or nearly equal to the number of molecules within the library so that each molecule is represented once.

The diluted master library is then divided into sub-libraries numbered 1 to n, wherein n is equal to the total number of sub-libraries. Each of the sub-libraries can then be contacted with a tag such that each sub-library is covalently attached to a unique tag, yielding a set of tagged libraries.

A master library can contain typically from 10⁴ to 10¹², generally 10⁶ to 10¹² different (i.e., unique) members. The particular manner in which the libraries are prepared for the methods described herein is a function of the library. For example, for cloning into a selected vector, such as a plasmid for bacterial expression, suitable restriction sites can be included as needed. Other modifications are routine and known to those of skill in the art.

In some embodiments, the libraries have fewer than the selected diversity. In such instances, different libraries can be obtained or generated and then combined, or, as described herein, separately used to produce the sub-libraries. This permits generation of tagged libraries, and ultimately arrays and canvases, of high diversity.

Nucleic acid libraries are contacted with nucleic acid molecules encoding the polypeptide tag sequences such that, when translated, encoded members of each sub-library are attached to the same polypeptide tag. Due to inefficiencies in ligation and transformation during cloning in the methods for evenly distributing tags, the diversity of tagged libraries is lower, estimated for purposes herein to about 10%, of the diversity of each sub-library. Although 10% generally serves as a good estimate, if needed the precise numbers can be empirically determined for a particular sub-library and tagged library.

### 3. Adjusting the Diversity of a Master Library so that the Diversity is About Equal to the Number of Members of the Library

If necessary, the diversity of a master library is adjusted so that its diversity is approximately equal to the number of members of the library. Typically, approximately equal is within one order of magnitude or less, such as 0.5 orders of magnitude and generally; 0.1 orders of magnitude. This adjustment can be accomplished, for example, by estimating the diversity of the library and estimating the total number of molecules in the library. It is understood that determination of diversity and numbers of members in a library are estimates, not exact determinations. A composition is prepared such that the number of estimated molecules and the estimated diversity is about the same (i.e., within about one order of magnitude, 0.5 orders of magnitude or generally 0.1 orders of magnitude). For example, if the diversity of the library is estimated to be 2.5×10¹⁰, then a sample containing 2.5×10¹⁰ molecules is prepared.

Diversity can be estimated by any method known to those of skill in the art and is a function of the type of library. For example, for a single chain antibody encoding library, the diversity is estimated to be the number of transformants produced upon introduction of the library into a bacterial host. It is assumed by those of skill in the art that each transformant is unique.

### 4. Dividing the Master Library into Sub-libraries

The master library is divided into up to “n” sub-libraries designated 1−n, where n is equal to or less than the number of different nucleic acid molecules that encode different tags. Where the diversity of the master library is equal to the number of molecules within the collection, the sub-libraries are all of equal volume, number of molecules and diversity. If the diversity does not equal the number of molecules in the collection, then appropriate adjustment of the volume of the sub-libraries may be required.

Separation of a master library can be accomplished, for example, by initially estimating the diversity of molecules in a master library and then preparing a solution in which the number of molecules is equal to, or nearly equal to, the diversity of molecules in the master library. For example, if the diversity of molecules in the master library is estimated to be 2.5×10¹⁰, then a composition of 2.5×10¹⁰ molecules is prepared. The resulting composition is then physically divided into n number of aliquots, each of equal volume such that each aliquot contains approximately the same number of molecules. The molecules contained in these aliquoted solutions are the sub-libraries.

As stated above, the number of different tag-encoding nucleic acid molecules can be predetermined, and constrains the number of sub-libraries prepared from the master library. The number of sub-libraries is typically equal to, but can be less than, the number of unique tag-encoding nucleic acid molecules.

### 5. Creation of Tagged Libraries

Tagged libraries are produced by attaching, directly or indirectly, a a nucleic acid molecule encoding a tag to members of each sub-library to produce “n” tagged sub-libraries containing tagged members, whereby the polypeptide (epitope) tag encoding portion of the tag is in frame with a polypeptide encoded by the nucleic acid molecule. The encoded polypeptide tag is unique to each sub-library

As noted, division of the master library into sub-libraries is based on the number of unique tag encoding nucleic acid molecules available. Preparation of the tagged library results from the incorporation of a sequence of nucleotides that encodes a unique tag into the molecules of each sub-library. Any methods known to those of skill in the art to add and incorporate a double-stranded DNA fragment into nucleic acid may be used. In the method provided herein, the tag-containing fragments are ligated directly or via linkers to the molecular members of the sub-libraries (exemplified herein). The amplified or ligated product, if needed, can be further amplified or manipulated such as by the ligation of additional tags or insertion of other properties using methods that can be readily devised by those of skill in the art in light of the description herein.

In the initial tagging step, when adding the tag-encoding set of oligonucleotides on the constituent members of the nucleic acid sub-library, a goal is to get an even distribution of all nucleic acid molecules encoding the tags, so that on the average each different molecule has a unique nucleic acid tag. To effect this, the master library is divided into sub-libraries, identified as S₁-Sₙ, wherein n is equal to or less than the number of unique encoded tags. Each sub-library is then contacted labeled with a unique polypeptide tag, yielding a collection of sub-libraries each tagged with a unique tag.

Any method known to one of skill in the art to link a tag, such as a nucleic acid molecule encoding a polypeptide tag or a polypeptide epitope tag, to another molecule, such as a nucleic acid or a polypeptide is contemplated. For example, a variety of such methods are described. As noted, they are described with particular reference to antibody capture agents, and polypeptide tags that include epitopes to which the antibodies bind, but it is to be understood that the methods herein can be practiced with any capture agent and polypeptide tag therefor.

### a. Ligation to Create Circular Plasmid Vector for Introduction of Tags

As noted above, in addition to use of amplification protocols for introducing the primers into the library members, the primers may be introduced by direct ligation, such as by introduction into plasmid vectors that contain the nucleic acid that encode the tags and other desired sequences. Subcloning of a nucleic acid molecule, such as a cDNA molecule, into double-stranded plasmid vectors is well known to those skilled in the art, and is exemplified herein in Example 4 below. Any suitable vector for such subcloning can be used, and includes any that infect bacteria or that can be propagated in eukaryotic cells. Plasmids (designed 1−n, wherein n is the number of unique polypeptide tags to be distributed among members of the library) with nucleic acid encoding each of the tags are prepared kept separate. Nucleic acid from the master library is introduced into the 1−n plasmids such that encoded polypeptides are in reading frame, although not necessarily adjacent, with the polypeptide tag, such that upon expression of the nucleic acid molecule a polypeptide with the tag, typically at one end is produced.

As exemplified, digesting purified double-stranded plasmid with a site-specific restriction endonuclease creates 5′ or 3′ overhangs also known as sticky ends. Double-stranded members of a DNA library are digested with the same restriction endonuclease to generate complementary sticky ends. Alternately, blunt ends in the vector DNA and DNA in the library are created and used for ligation. The digested DNA and plasmid DNA are mixed with a DNA ligase in an appropriate buffer (commonly, T4 DNA ligase and buffer obtained from New England Biolabs are used) and incubated (typically at 16° C.) to allow ligation to proceed. A portion of the ligation reaction is transformed into a suitable host, such as E. coli, that has been rendered competent for uptake of DNA by any of a variety of methods, such as, but not limited to, electroporation, calcium phosphate uptake, lipid-mediated transfection and heat shock of chemically competent cells are common methods. Aliquots of the transformation mixture can be plated onto semi-solid selective medium, such as medium containing the antibiotic appropriate for the plasmid used. Only those bacteria receiving a circular plasmid gives rise to a colony on this selective medium. For each set of plasmids that encode a tag, samples of the DNA library are inserted (see, e.g., FIGS. 6A and 6B).

For directional cloning of cDNA clones, which is desirable for the creation of a library used for expression of proteins from the cDNA library in reading frame with a tag, two different restriction endonuclease, which generate different sticky ends can be used for digestion of the plasmid. The cDNA library members are created such that they contain these two restriction endonuclease recognition sites at opposite ends of the cDNA. Alternatively, for example, different restriction endonuclease that generate complementary overhangs are used (for example digestion of the plasmid with NgoMIV and the cDNA with BspEl leave a 5′CCGG overhang and are thus compatible for ligation). Furthermore, directional insertion of the cDNA into the plasmid vector brings the cDNA under the control of regulatory sequences contained in the vector. Regulatory sequences can include promoter, transcriptional initiation and termination sites, translational initiation and termination sequences and RNA stabilization sequences. If desired,, insertion of the cDNA also places the cDNA in the same translational reading frame with sequences coding for additional protein elements including those used for the purification of the expressed protein, those used for detection of the protein with affinity reagents, those used to direct the protein to subcellular compartments, those that signal the post-translational processing of the protein.

For example, as described in Example 4, the pBAD/gIII vector (Invitrogen, Carlsbad Calif.) was used as an expression vector for the scFv cDNA library obtained from mouse spleens (see Examples). This vector contains cloning sites that are useful for insertion of cDNA clones. When ligating a nucleic acid library into an expression vector, the cloning sites can be designed and/or chosen such that the inserted cDNA clones are not internally digested with the enzymes used and such that the cDNA is in the same reading frame as the desired coding regions contained in the vector. For example, it is common to use SfiI and NotI sites for insertion of single chain antibodies (scFv) into expression vectors. Therefore, to modify the pBAD/gIII vector for expression of scFvs, oligonucleotides containing these restriction sites were hybridized and inserted into restriction sites already present in the vector. The resultant vector permits insertion of scFvs (created with standard methods such as the “Mouse scFv Module” from Amersham-Pharmacia) in the same reading frame as the gene III leader sequence and the polypeptide tag.

As exemplified herein, a library of expressed proteins is subdivided using a plurality of polypeptide tags and the antibodies that recognize them. To create the library for expressing proteins with a plurality of polypeptide tags, slight modifications of the subcloning techniques described above are used. A plurality of cDNA clones are divided into sub-libraries and each sub-library is inserted into a distinct plasmid vector containing a unique polypeptide tag encoding nucleic acid sequence (instead of a single type of plasmid vector) such that the resulting library contains cDNA clones tagged with the different polypeptide tags, and each polypeptide tag is represented equally. Multiple plasmid vectors are created such that they differ in the polypeptide tag that is translated in frame with the inserted cDNA member. For example, if there are 1000 polypeptide tag sequences, 1000 different vectors are constructed; if there are 250 polypeptide tag sequences, 250 different vectors are constructed.

There are a variety of methods for construction of these vectors known to those of skill in the art. For illustration purposes, the myc epitope encoding region of the pBAD/gIII plasmid is removed by digestion with XbaI and SalI restriction enzymes, and the large 4.1 kb fragment is isolated. The hybridization of oligonucleotides HAFor (SEQ ID No. 8) and HARev2 (SEQ ID No. 74) creates overhangs compatible with XbaI and SalI, such that the product is inserted directionally, and encodes the epitope for the HA11 antibody (see Tables 3 and 4 below). Insertion of the hybridization product of M2For (SEQ ID No. 10) and M2Rev2 (SEQ ID No. 11) results in a vector with the FLAG M2 epitope (see Tables 3 and 4 below) in frame with the inserted cDNA. Insertion of the hybridization product of V5For (SEQ ID No. 75) and V5Rev (SEQ ID No. 76) results in a vector with the V5 epitope (see table below) in frame with the inserted cDNA. Hybridization and insertion of pairs of oligos listed below result in the creation of the epitopes in frame with the cDNA.

TABLE 3

| | | |
|---|---|---|
| | | SEQ |
| oligo name | Sequence 5′ to 3′ | ID No. |

| | | |
|---|---|---|
| SfilNotlFor | catggcggcccagccggcctaatgagcggccgca | 6 |
| SfilNotlRev | agcttgcggccgctcattaggccggctgggccgc | 7 |
| HAFor | ctagaatatccgtatgatgtgccggattatgcgaatagcgccg | 8 |
| HARev | tcgacggcgctattcgcataatccggcacatcatacggataaa | 9 |
| HARev2 | tcgacggcgctattcgcataatccggcacatcatacggatatt | 74 |
| M2For | ctagaagattataaagatgacgacgataaaaatagcgccg | 10 |
| M2Rev2 | tcgacggcgctatttttatcgtcgtcatctttataatctt | 11 |
| V5for | CTAGAAggtaagcctatccctaaccctctcctcggtctcgattctacgAATAGCGCCG | 75 |
| V5rev | TCGACGGCGCTATTcgtagaatcgagaccgaggagagggttagggataggcttaccTT | 76 |
| StagFor | CTAGAAaaagaaaccgctgctgctaaattcgaacgccagcacatggacagcAGCGCCG | 77 |
| StagRev | TCGACGGCGCTgctgtccatgtgctggcgttcgaatttagcagcagcggtttctttTT | 78 |
| HSVtagFor | CTAGAAcagccggaactggcgccggaagatccggaagatAATAGCGCCG | 79 |
| HSVtagRev | TCGACGGCGCTATTatcttccggatcttccggcgccagttccggctgTT | 80 |
| T7tagFor | CTAGAAatggctagcatgactggtggacagcaaatgggtAATAGCGCCG | 81 |
| T7tagRev | TCGACGGCGCTATTacccatttgctgtccaccagtcatgctagccatTT | 82 |
| GluGluFor | CTAGAAgaagaggaggaatatatgccgatggaaAATAGCGCCG | 83 |
| GluGluRev | TCGACGGCGCTATTttccatcggcatatattcctcctcttcTT | 84 |
| KT3For | CTAGAAaaaccgccgaccccgccgccggaaccggaaaccAATAGCGCCG | 85 |
| KT3Rev | TCGACGGCGCTATTggtttccggttccggcggcggggtcggcggtttTT | 86 |
| EtagFor | CTAGAAggtgcgccggtgccgtatccggatccgctggaaccgcgtAATAGCGCCG | 87 |
| EtagRev | TCGACGGCGCTATTacgcggttccagcggatccggatacggcaccggcgcaccTT | 88 |
| VSVGfor | CTAGAAtacaccgacatcgaaatgaaccgtctgggtaaaAATAGCGCCG | 89 |
| VSVGrev | TCGACGGCGCTATTtttacccagacggttcatttcgatgtcggtgtaTT | 90 |
| Ab2For | ctagaaTTGACTCCTCCTATGGGTCCTGTTATTGATCAGCGGc | 168 |
| Ab2Rev | tcgagCCGCTGATCAATAACAGGACCCATAGGAGGAGTCAAtt | 169 |
| Ab4For | ctagaaTATAATATGGAATCGTATCTGTGGTATTTGGCGCCGc | 170 |
| Ab4Rev | tcgagCGGCGCCAAATACCACAGATACGATTCCATATTATAtt | 171 |
| B34For | ctagaaGATCTTCATGATGAGCGTACTCTTCAGTTTAAGCTTc | 172 |
| B34Rev | tcgagAAGCTTAAACTGAAGAGTACGCTCATCATGAAGATCtt | 173 |
| P5D4aFor | ctagaaCATCCGAATTTGCCTGAGACTCGTCGTTATGCGCTGc | 174 |
| P5D4aRev | tcgagCAGCGCATAACGACGAGTCTCAGGCAAATTCGGATGtt | 175 |
| P5D4bFor | ctagaaTCTTATACTGGGATTGAGTTTGATCGTTTGTCGAATc | 176 |
| P5D4bRev | tcgagATTCGACAAACGATCAAACTCAATCCCAGTATAAGAtt | 177 |
| 4C1OFor | ctagaaATGGTGGATCCTGAGGCGCAGGATGTGCCGAAGTGGc | 178 |
| 4C1ORev | tcgagCCACTTCGGCACATCCTGCGCCTCAGGATCCACCATtt | 179 |

TABLE 4

Antibody Epitopes

| | | | |
|---|---|---|---|
| | Epitope | | |
| Antibody | name | Sequence | SEQ ID |

| | | | |
|---|---|---|---|
| 9E10 | myc | EQKLISEEDL | 91 |
| HA.11, HA.7, or | HA | YPYDVPDYA | 92 |
| 12CA5 | | | |
| M1, M2, M5 | FLAG | DYKDDDDK | 93 |
| GluGlu | GluGlu | EEEEYMPME | 94 |
| V5-tag | V5 | GKPIPNPLLGLDST | 95 |
| T7-tag | T7 | MASMTGGQQMG | 96 |
| HSV-tag | HSV | QPELAPEDPED | 97 |
| S protein | S-tag | KETAAAKFERQHMDS | 98 |
| (not an antibody) | | | |
| KT3 | KT3 | KPPTPPPEPET | 99 |
| E-tag | E-tag | GAPVPYPDPLEPR | 100 |
| P5D4 | VSV-g | YTDIEMNRLGK | 101 |
| B34 | B34 | DLHDERTLQFKL | 180 |
| P5D4 | VSV-1 | HPNLPETRRYAL | 181 |
| P5D4 | VSV-2 | SYTGIEFDRLSN | 182 |
| 4C10 | 4C10 | MVDPEAQDVPKW | 183 |

Each of these vectors still shares the SfiI and NotI restriction endonuclease sites to allow subcloning of cDNA clones into the vectors. Similarly, additional oligonucleotides can be designed to encode a wide variety of polypeptide tags that can be inserted in the same position to create a collection of different vectors.

Plasmid DNA corresponding to the vectors containing different polypeptide tags is prepared using methods known to those in the art (QIAGEN columns, CsCl density gradient purification, etc). Purified double-stranded DNA from each of the plasmids is quantified by OD260 and ethidium bromide staining on an agarose gel confirms quantification. Other methods known to those skilled in the art can be used for quantification of plasmid DNA.

In order to evenly distribute the polypeptide tags among the cDNA clones, a series of plasmid vectors encoding the polypeptide tag sequences is created such that each vector in the series contains a unique polypeptide tag-encoding sequence. Each of these vectors shares restriction endonuclease sites to allow subcloning (generally directional) of cDNA clones into the vectors. Double stranded cDNA representing the library of interest also is digested with restriction endonuclease to create ends that are compatible for ligation to the ends created by vector digestion. This is accomplished by using the same enzymes for vector and cDNA digestion or by using those that generate complementary overhangs (for example NgoMIV and BspEl both leave a 5′CCGG overhang and are thus compatible for ligation). Alternatively, blunt ends in both vector DNA and cDNA are created and used for ligation. Digested cDNA clones and digested vector DNAs are ligated using a DNA ligase such as T4 DNA ligase, E. coli DNA ligase, Taq DNA ligase or other comparable enzyme in an appropriate reaction buffer. The resultant DNA is transformed into bacteria, yeast, or used directly as template for in vitro transcription of RNA. The design of the vectors is such that insertion of the cDNA at the restriction endonuclease sites places the cDNA under control of promoter sequences to allow expression of the cDNA. Additionally, the cDNA are in the same reading frame as the nucleic acid sequence encoding the polypeptide tag such that upon protein expression from this vector, a fusion protein containing the cDNA-encoded polypeptide fused to the polypeptide tag is produced. The E sequence is positioned in the vector such that the encoded polypeptide tag is fused to either the N- or the C-terminus of the resultant protein (for restriction enzyme digestion, DNA ligation, and transformation, see, e.g., see, Sambrook et al. (1989) Molecular Cloning: A Laboratory Manual, 2nd Edition, Cold Spring Harbor Laboratory Press, Chapter 1).

### b. Ligation of Sequences Resulting in Linear Tagged cDNA

Following creation of the cDNA library, the library is divided into a number of sub-libraries, and sequences are appended to cDNA clones via ligation. Linear, double-stranded DNA containing each of the sequences encoding the polypeptide tags is created via various methods (synthesis, digestion out of plasmid containing the sequences, assembly of shorter oligonucleotides, etc.). These linear dsDNAs containing the different polypeptide tag sequences are individually combined with the members of a double-stranded cDNA sub-library and ligated using a nucleic acid ligase in an appropriate buffer. This is generally a DNA ligase, but an RNA ligase is used if the nucleic acid encoding the tags is composed of RNA or are RNA/DNA hybrid molecules and the library also is in the form of an RNA or RNA/DNA hybrid. In one embodiment, the tag-encoding molecule is blunt-ended on both ends yet only one end is phosphorylated such that ligation occurs in a directional manner (with respect to the tag sequence) and the tag-encoding molecule is brought into the same reading frame as the cDNA (at either the N- or C-terminus of the resulting protein). In another embodiment, the tag-encoding molecule is blunt-ended at one end and has an overhang on the other end such that ligation occurs in a directional manner (see, Sambrook et al. (1989) Molecular Cloning: A Laboratory Manual, 2nd Edition, Cold Spring Harbor Laboratory Press Chapter 8). The tag-encoding molecule can be continuously double-stranded, or partially double-stranded with a single-stranded central portion.

In another embodiment, the cDNA library is created to contain a restriction endonuclease site and the same restriction site is included in the tag-encoding molecule such that upon digestion of each with the appropriate enzyme, compatible ends are created. The cDNA library is divided into sub-libraries and each sub-library is digested. Each digested sub-library is then ligated to a unique digested tag-encoding molecule using a DNA ligase in an appropriate buffer. In another embodiment, the cDNA library is created to contain a restriction endonuclease site and the tag-encoding molecules are designed to contain a restriction site that leaves an overhang compatible to the overhang generated on the cDNA. Upon ligation of these two compatible sites, a sequence is generated that is not susceptible to cleavage with either of the enzymes used to generate the overhangs. In this case, the products of the ligation reaction are digested with the enzymes used to generate the overhangs. Alternately, the ligation reaction occurs in the presence of the enzymes used to generate the overhangs (Biotechniques (1999) August 27(2): 328-30, 332-4, Biotechniques (1992) January 12(7): 28, 30).

This method reduces and/or eliminates the step of ligation of cDNA to cDNA or tag-encoding sequence to tag-encoding molecule, and thus enriches for the cDNA-polypeptide tag-encoding product. Pairs of enzymes capable of generating such compatible overhangs include AgeI/XmaI, AscI/MluI, BspEI/NgoMIV, NcoI/PciI and others (New England Biolabs 2000-2001 catalog pgs. 218-231 for partial list). The polypeptide tag sequences and the cDNA are designed such that they are in the same reading frame following ligation. Therefore, upon protein expression from this construct, a fusion protein containing the cDNA-encoded polypeptide fused to the tag is produced. The tag is positioned, in the final construct such that the encoded tag is fused either directly or indirectly to the N- or the C-terminus of the resulting polypeptide.

In another embodiment, the cDNA, the tag-encoding molecule or both are created such that they contain a region with RNA hybridized to DNA. The RNA can be removed by digestion with the appropriate RNAse (including type 2 RNAse H) such that a single-stranded DNA overhang results. This overhang can be ligated to compatible overhangs generated either by the above method or by restriction endonuclease digestion. Additionally, overhangs and flanking sequences are designed in such a way that if a tag-encoding molecule is ligated to another polypeptide tag-encoding molecule, the resulting molecule is susceptible to digestion with a particular restriction enzyme. Likewise, if a cDNA is ligated to another cDNA, the resulting sequence is susceptible to cleavage by another restriction enzyme. Ligation reactions occur in the presence of those restriction enzymes, or are subsequently treated with those enzymes to reduce the incidence of cDNA-cDNA or tag-encoding molecule-polypeptide tag-encoding molecule ligation events (see enzymes pairs and references above). The polypeptide tag encoding sequences and the cDNA are designed such that they are in the same reading frame following ligation. Therefore, upon protein expression from this construct, a fusion protein containing the cDNA-encoded polypeptide fused directly or via a polypeptide linker to the tag is produced. The tag-encoding portion is positioned in the final construct such that the encoded tag is fused directly or indirectly to either the N- or the C-terminus of the resulting protein.

In another embodiment, amplification is used to generate the cDNA and the various tag-encoding molecules using primers that contain regions of RNA sequences that cannot be copied by certain thermostable DNA polymerases. Therefore RNA overhangs remain that can be ligated to complementary overhangs generated by the same method or by restriction enzyme digestion. RNA or DNA overhang cloning is described by Coijee et al. (Nat Biotechnol 2000 Jul. 18(7): 789-91).

In another embodiment, a tag-encoding nucleic acid molecule is brought into close apposition to a cDNA sequence by hybridization to a splint oligonucleotide that is complementary to the 3′ region of the cDNA and also the 5′ region of the tag-encoding molecule (Landegen et al. Science 241: 487 (1988)). Joining of the cDNA and polypeptide tag sequence is accomplished by a nucleic acid ligase under appropriate reaction conditions. In another embodiment, the splint oligonucleotide is complementary to the 5′ region of the cDNA and the 3′ region of the tag-encoding molecule. In both cases, the different members of the cDNA library share a common sequence (at the 3′ or 5′ end), and the different polypeptide tag sequences also share a common sequence (at the 5′ or 3′ end), such that a single splint oligonucleotide sequence can hybridize to any member of the cDNA library and also to any individual of the series of tag-encoding sequences. In each of these embodiments, the splint oligonucleotide, the cDNA and the tag-encoding sequences can be single or double-stranded DNA, or combinations of DNA and RNA. Mixtures of the members of a sub-library of cDNA, a unique polypeptide tag sequence and splint oligonucleotides are denatured at elevated temperatures to eliminate secondary structure and existing hybridization. The reaction is then cooled to allow hybridization to occur. In cases where the splint oligonucleotide is present in molar excess, a hybridization product containing the three desired components (cDNA, polypeptide tag sequence and splint oligonucleotide) is obtained. A nucleic acid ligase is added and the reaction is incubated under appropriate conditions.

In another embodiment, the splint oligonucleotide, cDNA library and tag-encoding sequences are designed as in the above example. The ligase chain reaction (see, e.g., LCR, F. Barany (1991) The Ligase Chain Reaction in a PCR World, PCR Methods and Applications, vol. 1 pp. 5-16; see, also, U.S. Pat. No. 5,494,810) is then performed using multiple cycles of denaturation, hybridization, and ligation with a thermostable ligase. For geometric amplification of cDNA-tag-encoding sequence product, double-stranded cDNA and double-stranded polypeptide tag sequences are needed.

### C. Primer Extension and PCR for Tag Incorporation

In another embodiment, a unique polypeptide tag sequence is appended to members of each sub-library of a mRNA master library. In this case, the tag-encoding molecule is designed such that it can hybridize to a desired population of mRNA. This tag sequence serves as a primer and the RNA serves as a template for synthesis of DNA using reverse transcriptase (AMV-RT, M-MuLV-RT or other enzyme that synthesizes DNA complementary to RNA as template). The newly synthesized cDNA is complementary to the RNA and has a tag-encoding sequence at the 5′end. Second strand synthesis using a DNA polymerase results in double-stranded DNA with the polypeptide tag sequence at the end corresponding to the 3′ end of the RNA. In this embodiment, all members in the series of tag-encoding sequences share a common 3′ end for hybridization to the RNA (e.g., in the case of a library of similar members of a gene family). Alternatively, tag-encoding sequences have a sequence of random nucleotides at the 3′ end for random priming of RNA (Molecular cloning: a laboratory manual 2^{nd} edition, Sambrook et al, Chapter 8).

In another embodiment, the polymerase chain reaction (PCR) is used to append unique tag-encoding sequences to members of sub-libraries of cDNA clones. A cDNA master library is created in such a way that all members share a common sequence at the 3′ end (e.g., prime first strand cDNA synthesis with an oligonucleotide containing this common sequence, or ligation of linker sequences to double-stranded cDNA clones). Additionally, each member of the cDNA master library shares a different common sequence (“C”) at the 5′ end. Each unique member in the series of polypeptide tag sequence has a common 3′ end that is complementary to one of the common regions in the cDNA. The polypeptide tag sequences serve as one of the amplification primers in a polymerase chain reaction. An oligonucleotide complementary to the common region at the opposite end of the cDNA serve as the second amplification primer. The cDNA library is subdivided after the addition of the common sequences, and aliquots are combined with individual polypeptide tag sequences, the second primer and a thermostable polymerase (Taq, Vent, Pfu, etc) in the appropriate buffer conditions and multiple cycles of denaturation, hybridization, and DNA polymerization are executed.

### d. Insertion by Gene Shuffling

In another embodiment, polypeptide tag sequences are appended to cDNA clones via “DNA shuffling” or molecular breeding (see, e.g., Gene (1995) October 16 164(1): 49-53; Proc Natl Acad Sci USA (1994) October 25 91(22): 10747-51; U.S. Pat. No. 6,117,679). Each member in the series of polypeptide tag sequences have a common 3′ end that is complementary to one of the common regions in the cDNA library members. During mutagenesis of the individual sub-libraries of the cDNA library, different polypeptide tag sequences are included in the PCR reaction to allow the polypeptide tag sequences to be assembled along with the fragments of the cDNA clones.

### e. Recombination Strategies

Recombination strategies also can be used for introduction of tags into cDNA clones. For example, triple-helix induced recombination is used to append polypeptide tag sequences to cDNA clones. A cDNA library is created in such a way that all members share a common sequence at one end. The series of polypeptide tag sequences is designed to include a region with considerable homology to the common sequence in the cDNA library. An individual tag-encoding sequence and a sub-library of the cDNA library are combined in a cell-free recombination system (J Biol Chem (2001) May 25 276(21): 18018-23) with a third homologous oligonucleotide and recombination is allowed to occur.

In another embodiment, site-specific recombination is used to append tag-encoding sequences to cDNA clones. Site-specific recombination systems include loxP/cre (U.S. Pat. No. 6,171,861; U.S. Pat. No. 6,143,557; ), FLP/FRT (Broach et al. Cell 29: 227-234 (1982)), the Lambda integrase with attB and attP sites (U.S. Pat. No. 5,888,732), and a multitude of others. The series of polypeptide tag sequences as well as the members of the cDNA library are designed to include a common sequence recognized by the recombinase protein (e.g. loxP sites). To insure an even distribution of the polypeptide tags among the cDNA library members, an individual polypeptide tag sequence and a sub-library of the cDNA library are combined in a cell-free recombination system (Protein Expr Purif (2001) June 22(1):135-40) including the site-specific recombinase (e.g. cre recombinase) under appropriate conditions to allow recombination to take place. Alternatively, the recombination events take place inside cells such as bacteria, fungus, or higher eukaryotic cells expressing the desired recombinase (see U.S. Pat. Nos. 5,916,804, 6,174,708 and 6,140,129 as examples).

In another embodiment, homologous recombination in cells is used to append polypeptide tag sequences to cDNA clones. E. coli (Nat Genet (1998) October 20(2): 123-8), yeast (Biotechniques (2001) March 30(3): 520-3), and mammalian cells (Cold Spring Harb Symp Quant Biol. (1984) 49: 191-7) are used for recombination of DNA segments. The polypeptide tag sequences are designed to contain both 5′ and 3′ regions with homology to two separate regions in a plasmid vector containing the cDNA. The lengths of homologous regions are dependent on the cell type being used. Members of a sub-library of the cDNA master library and a unique polypeptide tag sequence are co-transformed into the cells and homologous recombination is carried out by recombination/repair enzymes expressed in the cell (see, e.g., U.S. Pat. No. 6,238,923).

### f. Incorporation by Transposases

In another embodiment, transposases are used to transfer polypeptide tag sequences to cDNA clones. Integration of transposons can be random or highly specific. Transposons such as Tn7 are highly site-specific and are used to move segments of DNA (Lucklow et al. J. Virol. 67: 4566-4579 (1993)). The polypeptide tag sequences are contained between inverted repeat sequences (specific to the transposase used). The members of the cDNA library (or the plasmid vectors they are in) contain the target sequence recognized by the transposase (e.g., attTn7). In vitro or in vivo transposition reactions insert the polypeptide tag sequences into this site.

### 9. Incorporation by Splicing

In another embodiment, polypeptide tag sequences flanked by RNA splice acceptor and donor sequences are inserted into the genome of various cell lines in such a way as to incorporate them into the mRNA being transcribed and translated (See U.S. Pat. No. 6,096,717 and U.S. Pat. No. 5,948,677). Proteins isolated from these organisms, or cell lines therefore contain the polypeptide tags and are amenable to separation by our collection of antibodies.

In another embodiment, polypeptide tag sequences are appended to library members via trans-splicing of RNA. The RNA form of a unique polypeptide tag sequence, and preceded by RNA splice acceptor sequences, or followed by splice donor sequences is expressed in cells that then receive an individual sub-library of the master library of cDNA clones. Trans-splicing of RNA (Nat Biotechnol (1999) March 17 (3)4: 246-52, and U.S. Pat. No. 6,013,487) appends the polypeptide tag sequence to the sub-library member.

### 6. Mixing Some or All of the Tagged Sub-libraries to Produce a Mixed Library, Where the Number of Tagged Nucleic Acid Molecules Added From Each Tagged Sub-library is the Same

Tagged libraries are combined to produce a mixed library such that each tagged molecule is approximately equally represented. As a result, tags are evenly distributed among the member tagged molecules of the mixed library. The determination of the number of tagged members within each tagged library and the mixing of the tagged libraries to give a mixed library can be accomplished by any suitable method. For example, the concentration of tagged molecules in sub-libraries to be mixed is determined and equal numbers are mixed. Concentration is determined by any suitable method such as by titering the number of transformants or colony forming units produced upon introduction of the tagged molecule into an appropriate host. Other methods of concentration determination include spectrometric and physical assay, such as the Bradford assay. Spectrometric methods monitor the increase or decrease in absorbance of light at a particular wavelength. According to Beer's Law, the absorbance of a molecule at a particular wavelength is proportional to its extinction coefficient, the pathlength of the light and the concentration of the absorbing species. Therefore, determination of ultraviolet or visible light at a predetermined wavelength can be used to calculate the concentration of the absorbing species within a known volume. Fluorescent molecules, such as GFP, emit light at a particular wavelength.

Prior to determining the concentration of the tagged libraries, separation of the fused molecule-tag product from the non-combined molecule and tag reactants may be required. Any means of separation known to those skilled in the art can be used. For example, electrophoretic methods can be used to identify and separate the fused nucleic acid molecules that encode the molecule and tag from the individual components. Other means, such as, but not limited to, transformation of the complex into a suitable host followed by antibiotic or other selection method, affinity chromatography, and co-expression of a detectable molecule such as GFP, are also contemplated. As stated above, the polypeptide tag itself may contain secondary tags that can be used for selection of fused molecule—polypeptide tag molecules.

Once the concentration of tagged molecules in each tagged library is known, an aliquot from each tagged sub-library which contains the same number of tagged members can be pooled to give the mixed library.

Optionally, the tagged libraries can be normalized prior to mixing such that the tagged libraries all contain an equivalent number of tagged members. An aliquot of equal volume from each of the normalized tagged sub-libraries can then be combined to give a mixed library. Optionally, the tagged libraries can be normalized subsequent to mixing by taking an aliquot of the mixed library and determining the representation of each tag within the aliquot. The number of tagged molecules from each of the sub-libraries can then be adjusted such that the relative number (proportion) of molecules from each sub-library represented in the mixed library is even, for example generally within 1 or 0.5 orders of magnitude, typically 0.2, 0.1 or 0.05 orders of magnitude.

In one embodiment, an aliquot from each tagged sub-library which contains approximately the same number of tagged members is pooled to give a mixed library. The concentration of each tag within the mixed library is then assessed and an adjustment factor is determined for each tag. The adjustment factor is used to adjust the number of molecules from each corresponding tagged sub-library. A new mixed library is then generated from the sub-libraries using the adjustment factors for each sub-library and a mixed library with equal representation of each tag is produced.

Adjustment factors for adjusting each sub-library can be obtained by determining the representation of each tag in a mixed library. The concentration or representation of each tag can be determined by any suitable method such as by transforming an aliquot of the mixed library into an appropriate host and determining the number of colony forming units with each tag as a percentage of the total. Other methods for determining the concentration of tagged molecules in the mixed library include assessing the concentration of tagged polypeptides from the mixed library by methods such as mass spectrometry, ELISA or by contacting some or all of the mixed library with a capture agent collection and assessing the number or percentage of tagged molecules of each type within the mixed library.

An adjustment factor is determined for each sub-library by determining the representation of each tag in the mixed library and calculating the adjustment needed such that the number of molecules added after adjusting yields an equivalent number of each tag represented in the mixed library. For example, if in the initial mixed library aliquot of tagged sub-libraries, it is determined that one tag (e.g. tag A) is represented as 20% of the total, instead of the expected 10%, then the number of molecules in the sub-library with tag A is adjusted to add half as much and a new mixed library is constructed by mixing the sub-libraries as adjusted by this adjustment factor. Similarly, if in the initial mixed library aliquot of 10 tagged sub-libraries, it is determined that two tags (e.g. tag A and B) are represented as 15% and 20% of the total, normalization factors for sub-libraries with tag A and tag B are adjusted with the calculated adjustment factors to produce a mixed library with equivalent numbers of tagged molecules from each sub-library.

The number of tagged molecules from each of the sub-libraries represented in the mixed library is even, for example, generally within 1 or 0.5 orders of magnitude, typically 0.2, 0.1 or 0.05 orders of magnitude. The proportion of tagged molecules from each sub-library can be influenced by the number of tags available and thus the number of different tagged sub-libraries that are constructed and mixed. For example, with 100 tags, each tagged sub-library is theoretically represented as 1% of the mixed library. Variations, for example from sample handling and pipetting error, can contribute to representations greater or less than 1% in the mixed library. As the number of tags is increased, the range of variation from the theoretical representation decreases since the errors have less effect in the representation. For example, in a mixed library constructed from 10,000 sub-libraries each tagged sub-library is theoretically represented at 0.01% of the mixed library. The range of variation in sub-library representation should be smaller than in mixed libraries constructed from fewer tags, for example, in a mixed library from 100 sub-libraries.

### 7. Splitting the Mixed Library into “q” Array Libraries, Wherein q is From 1 to a Predetermined Number of Arrays

The mixed library is split into q array libraries wherein q is equal to the number of arrays to be developed. As stated above, the number of arrays present is predetermined based on the number of loci per array, the desired diversity per locus and the diversity of the master library. Once this value has been determined, the pooled mixed library is split into aliquots of equal volume wherein the number of aliquots is equal to or less than the number of arrays.

### 8. Expression of Array Libraries and Purification of Tagged Molecules to Produce Collections of Tagged Molecules With Even Distributions of Tags

The tagged members of the array libraries are translated and the resulting polypeptides are purified yielding a collection of tagged molecules wherein the distribution of polypeptide tags is even throughout the collection of molecules. The purification of the molecules can be performed by any method known to those: skilled in the art, such as, for example affinity purification.

### 9. A Plurality of Polypeptide Tags

A plurality of tags can be added to each library member. This can be accomplished by the above methods, except that additional tag-encoding nucleic acid is attached to the library member, generally when the first tag is added. A second or additional tags can be the same among all members in the library, such as tags that facilitate purification, such as His tags, or can be different from the first tag and different in each sub-library or different among members in a tagged sub-library. Further tags can be added adjacent to the first tag, at the other terminus of the tagged molecules, linked via spacers or linkers or in other arrangements.

### D. Nested Sorting Using Addressable Arrays

Prior methods for identifying and selecting proteins of interest are hampered by selection biases that are created during successive rounds of enrichment. Selection biases can be avoided with the use of identification methods based on sorting rather than selection (see, e.g., U.S. application Ser. No. 09/910,120, published International PCT application No. WO 02/06834; published U.S. application Serial No. US20020137053 and U.S. provisional application Serial No. 60/352,011). Briefly, these methods rely upon the use of collections of capture agents, such as a plurality of substantially identical, generally replicate, collections of agents, such as antibodies, that specifically bind to preselected sequences of amino acids (generally at least about 5 to 10, typically at least 7 or 8 amino acids, such as epitopes), that are linked to proteins in a target library or encoded by a target nucleic acid library. Combinations of the capture agents and polypeptide tags that contain the sequence of amino acids to which the capture agent or a binding portion thereof specifically binds are provided. The nucleic acid molecule encoding the tags can be linked to members of a nucleic acid library or other library of molecules to be sorted.

The addressable anti-tag capture agent collections, such as a positionally addressable array, contains a collection of different capture agents, such as antibodies that bind to pre-selected-and/or pre-designed polypeptide tags, such as polypeptide tags, with high affinity and specificity. A typical collection contains at least about 30, 100, 500, and generally at least 1000 capture agents, such as antibodies, that are addressable, such as by occupying a unique locus on an array or by virtue of being bound to bar-coded support, color-coded, or RF-tag labeled support or other such addressable formats. Each locus or address contains a single type of capture agent, such as an antibody, that binds to a single specific tag. Tagged proteins are contacted with the collection of receptors, such as antibodies in an array, under conditions suitable for complexation with the receptor, such as an antibody, via the polypeptide tag. As a result, proteins are sorted according to the tag each possesses.

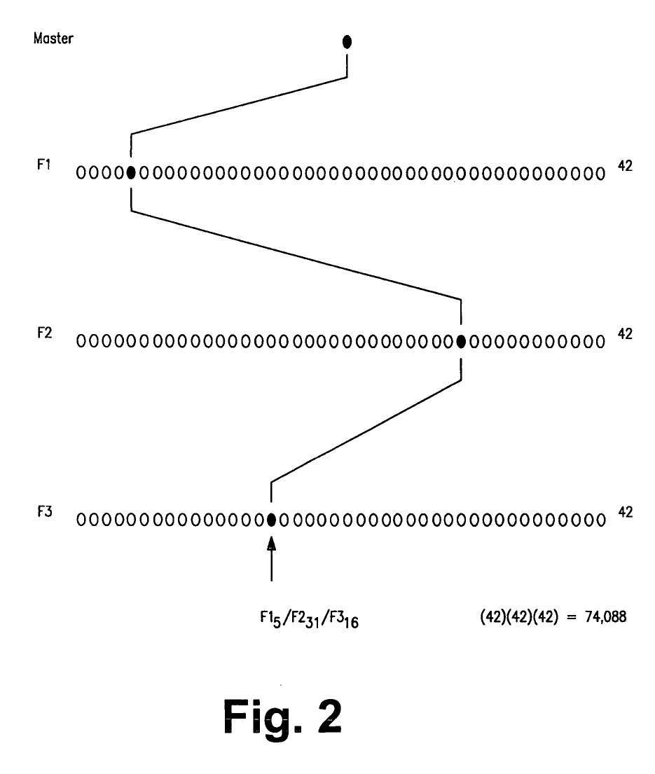

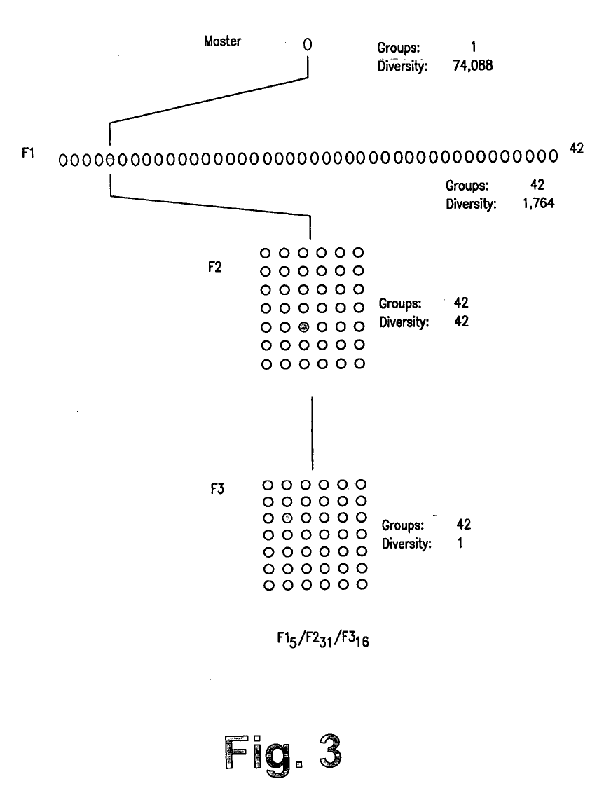

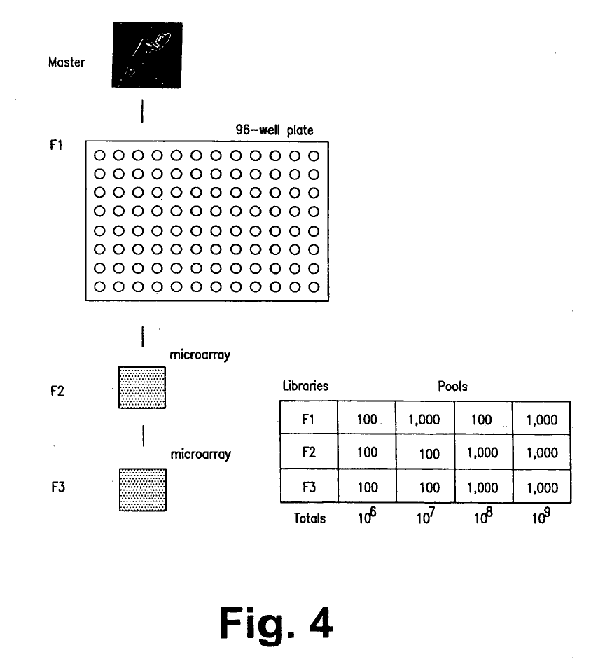

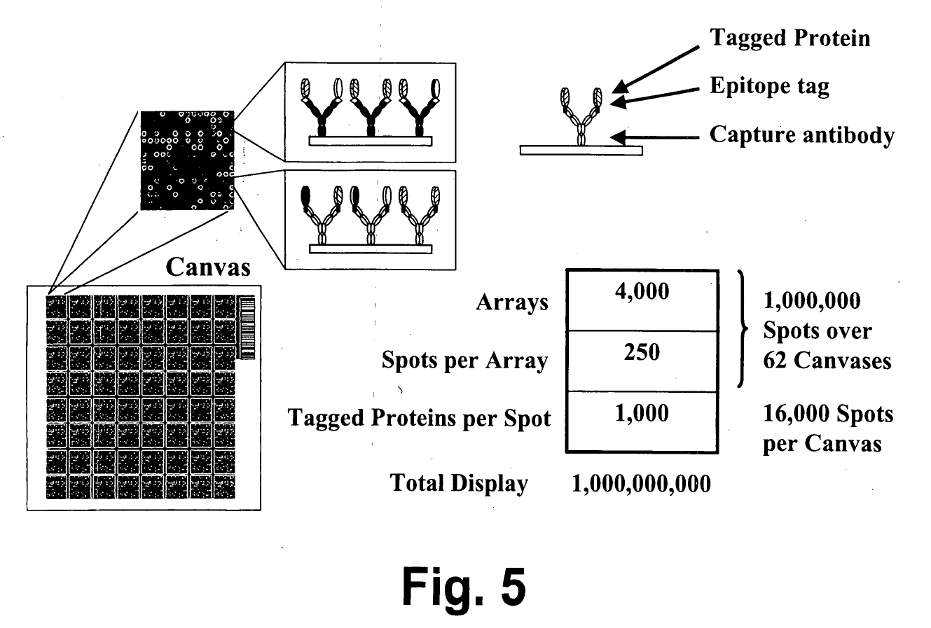

These addressable anti-tag antibody collections have a variety of applications including, but not limited to, rapid identification of antibodies; for therapeutics, diagnostics, reagents, and proteomics affinity matrices; in enzyme engineering applications such as, but not limited to, gene shuffling methodologies; for identification of improved catalysts, for antibody affinity maturation; for identification of small molecule capture proteins, sequence-specific DNA binding proteins, for single chain T-cell receptor binding proteins, and for high affinity molecules that recognize MHC; and for protein interaction mapping. Exemplary protocols are depicted in FIGS. 2-4.

The first sorting step substantially reduces diversity. If desired, further sorts are performed or the resulting library is screened by any method known to those of skill in the art. The optional second sort, which is started from the nucleic acid reaction mixture that contains the nucleic acid from which the protein of interest was translated, is performed. In this step, a new set of nucleic acid molecules encoding the polypeptide tags is added to the nucleic acid by amplification or ligation followed by amplification. Prior to, or simultaneously with this, the nucleic acid encoding the prior polypeptide tag is removed either by cleavage, such as with a restriction enzyme or by amplification with a primer that destroys part or all of the epitope-encoding nucleic acid. The new tags are added, the resulting nucleic acids are translated and then reacted with a single addressable collection of capture agents, such as, antibodies. The proteins sort according to their polypeptide tag, and a screen is run to identify the protein of interest.

At this point, the diversity of the molecules at the addressable locus of the antibody collection is 1 (or on the order of 1 to 100, typically 1 to 10). The nucleic acids that contain the protein of interest are then amplified with a primer that amplifies nucleic acid molecules that contain the nucleic acids encoding the identified polypeptide tag, to thereby produce nucleic acid encoding a protein of interest. The primer for amplification includes all or only a sufficient portion of the tag to serve as a primer to thereby remove the epitope from the encoded protein. Hence the methods, provided herein permit sorting (i.e., reduction of diversity) of diverse collections. A sort that involves one step will substantially reduce diversity. The use of optional sorting steps generally reduces diversity to less than 10, generally one.

### E. Sample Profiling Using Collections of Capture Agents and Polypeptide Tags

The capture agent collections and capture agent collections with bound molecules containing polypeptide tags can serve as devices for profiling samples, particularly biological samples, and are described in U.S. provisional application Serial No. No. 60/219,183. Briefly, any sample can be contacted with a capture agent collection or capture system and whatever binds can be detected by any suitable method, such as by enzyme or fluorescent labeling. Each sample produces a characteristic profile, such as a pattern when solid support arrays are used, which can serve as an identifier of the source of a sample or components thereof. Alternatively, the loci in the collection that react with a particular sample can be identified, such as by virtue of the bound polypeptide tag and used to produce sub-collections specific for a particular sample.

As in the embodiments for sorting, the addressable collection of capture agents is a collection of such agents, such that each loci is identifiable. A loci can be an addressable position on an array or a detectable label, such as a colored bead or nanobarcode or RF tag, linked or associated with a capture agent. For isolation and/or identification of molecules bound to the tagged-agents and other aspects of making and using, the addressable collection all of the methods described throughout the disclosure can be employed as needed in these embodiments.

For profiling, the collections are used either by themselves or with other reagents bound via their polypeptide tags. In the latter embodiment, the reagents bound via the polypeptide tags are not all the same, so that each loci represents a collection of such reactions, such as scFvs, bound via their polypeptide tags. As described herein, the polypeptide tags are distributed such that the linked agents are different. The resulting collection provides a highly diverse collection of capture agent-polypeptide tag-linked reagents for binding to any sample, such as a cell lysate, cells, blood samples, body fluid samples, tissue samples. Any method for sample preparation known to those of skill may be employed.

In some embodiments, a sample that has been subjected to a particular condition or treated with a particular agent is contacted with the collection, generally a collection of capture agents with epitope-tagged reagents, such as scFvs, bound thereto, and labeled components of the sample are permitted to react with the collection. After reacting and washing away or otherwise removing unbound material, a profile is produced, which is characteristic of the sample and particular collection. The profile can be imaged and, if needed, compared to the profile that results from a control for such condition or in the absence of the agent. For example, the same reaction can be performed with a duplicate or replicate collection, except that the sample may not be treated with the same condition. The resulting profile serves as a control. The difference between the two arrays represents a profile for the particular condition or sample.

In addition, upon identifying particular capture agent/polypeptide tag linked agent/sample component complexes specific for the test condition, the epitope-tagged reagents can be used to produce a sub-collection specific for the test condition. Such sub-collections can be repackaged as a collection, such as an array with a collection of binding agents, that when contacted with a sample provides a specific profile that is specific for a particular disorder or other test condition of interest. Also, since the polypeptide tags are known and can be used to design primers to amplify and identify nucleic acids encoding the linked polypeptides, specific binding proteins can be identified and used in the repackaged product and/or new binding agents can be identified.

### F. Staining of Bound Molecules

Bound polypeptide-tagged molecules and molecules bound thereto can be stained by any suitable method known to those of skill in the art and is a function of the target molecules. Exemplary stains include the use of chemiluminescence and bioluminescence generating reagents, such as horseradish peroxidase (HRP) systems, luciferin/luciferase systems, alkaline phosphatase (AP), labeled antibodies, fluorophores and isotopes. These molecules can be detected using film, photon collection, scanning lasers, waveguides, ellipsometry, CCDs and other imaging devices and methods.

As noted, uses of the capture systems include, but are not limited to: searching a recombinant antibody scFv library to identify scFv includes, but is not limited to, finding single antigen or multiple antigens; searching mutation libraries, including tagging mutant libraries; mutation by error prone PCR; mutation by gene shuffling for searching for small molecule binders, searching for increased antibody-affinity, searching for enhanced enzymatic properties (alkaline phosphatase (AP), horse radish peroxidase (HRP), luciferase and photoproteins, fluorescent proteins, such as green, blue or red fluorescent proteins (GFP, BFP, RFP); searching for sequence-specific DNA binding proteins; searching a cDNA library for protein-protein interactions; and any other such application. The type of stain used and the portion of the sample to be stained can be determined by the purpose of the experiment and will be known to those skilled in the art.

### 1. Methods of Staining

The staining of the sample can be non-specific, semi-specific or specific depending on when the sample is stained and what is stained. The staining of the sample, such as molecules or biological particles, can occur prior to, subsequent or during contacting the capture systems. Samples can be non-differentially or differentially stained. In each instance, the level of specificity of the molecules assessed varies.

For example, a cellular culture can be disrupted and the resulting lysate can be non-selectively stained, such as by biotinylation. The stained solution or lysate can then be contacted with the capture system, and the stained components are visualized by exposure to a horseradish peroxidase (HRP) conjugated anti-biotin antibody. Alternatively, the biological particles themselves are stained, such as by biotinylation, and then cells are lysed and, optionally, receptors are liberated from the membrane. In this instance, not all the sample components applied to the capture system are stained, so only stained particles that resided on the surface of the biological particle are detected. Therefore, subfractions can be semi-specifically stained and analyzed. For example, proteins and other molecules present on the cell surface can be identified. In other applications, organelles can be prepared and molecules on the surfaces of the organelle can be identified.

In other embodiments, the sample is contacted with the capture system and then stained, such as by visualization with a specific stain. Specific staining results in the visualization of a specific molecule or class of molecules to which a stain can bind specifically. The stain for a specific molecule can be any molecule or compound which interacts exclusively with the molecule or class of molecules of interest. To stain for a class of molecules, such as the immunoglobulins, the class of molecules contains a constant domain to which the stain can bind specifically and a variable domain which can interact with the capture system. Once the sample is overlayed on the array, the arrays are stained with a label, such as, but not limited to, an antibody, specific for a particular molecule or class of molecules. Thus, only the specific molecule or class of molecules stained is visualized on the array.

Specific staining can be used to assess and monitor changes in the levels of a specific molecule or class of molecules within a sample as the result of, for example, time, exposure to a condition or perturbation and the propagation of a diseased state. For example, when B cells initially develop, an IgM immunoglobulin is displayed on the surface of the cell. IgM is a member of the immunoglobulin superfamily, where all members possess similar structure by virtue of a constant domain and a variable domain. Different classes of immunoglobulins (IgG, IgA, IgE, IgD and IgM) vary in the amino acid sequence of their respective constant domains. Also, each immunoglobulin generally has different isotypic constant domains. For example, IgG has multiple isoforms including IgG1, IgG4 and IgG3. T cells and MHC molecules, which also belong to the immunoglobulin superfamily, have variable regions attached to a constant region but these regions do not have homology with each other or the members of other classes of the immunoglobulin superfamily. These differences in the constant regions of the various members of this diverse family allow for the specific staining of a particular class of immunoglobulins of interest.

For example, to monitor alterations in the idiotype of a subject, the B cells of a subject can be harvested, combined and lysed to obtain a lysate containing all of the IgM molecules present on the surface of the B cells. The lysate can then be overlayed on arrays displaying a library of scFv molecules such that the variable regions of the various IgM molecules interact with their complementary scFvs on the arrays. The immobilized IgM molecules can then be specifically stained with an anti-Ig-Fc antibody which recognizes the constant region (Fc) of all the IgM molecules attached to the arrays. The stain is specific for the IgM molecules because the constant region of the various immunoglobulins such as IgG, IgA, IgE and IgD are different from one another. The resulting pattern visualized on the arrays presents an image of the variable regions present in the IgM molecules within the sample due to their interaction with the scFvs displayed on the arrays. This pattern can then be used as a baseline for monitoring changes in the idiotypic landscape of the subject, for example, over time, following the administration of a drug molecule or during the course of a disease. Further, this pattern can be compared to similar samples from other subjects to assess the effect of varied environments on the display of IgM molecules by the B cells. Once IgM molecules are identified as being of interest, the arrays can be tailored to allow for the monitoring of the levels of IgM produced as a result of a change in the environment of the subject.

In a similar manner, the interaction between T cell receptors (TCR) and the scFv library can be monitored by specific staining. T cell receptors contain a constant domain and a variable domain which can be exploited for specific staining using an anti-TCR constant domain antibody. TCRs are responsible for the recognition of fragments of protein antigens on the surfaces of antigen presenting cells, which results in the activation of the T cell. The patterns discerned from arrays overlayed with a sample containing T cells can be used to assess and monitor the immune state and response of a subject at a particular time or over an extended time period. Variations in the pattern also can be used to monitor the effect of various drug molecules on a disease state or the progression or regression of a disease on the immune system response. Identification and monitoring of a particular TCR or group of TRCs of interest also can be performed utilizing the capture system and specific staining.

Presentation of peptide fragments of antigens by an antigen-presenting cell (APC) is performed by the major histocompatibility complex (MHC) during an immune response. Similar to immunoglobulins and TCRs, MHC has a variable region that interacts with the antigen fragment and a constant region. This constant region can be exploited for specific staining using the capture systems provided herein resulting in the high resolution mapping of antigen presentation during an immune response. The mapping of antigen presentation is an invaluable tool in the early diagnosis of disease, bacterial or viral infection. If levels of a particular MHC increase, then a particular disease state may be present. Similarly, the effect of drug molecules or an alteration in the cellular conditions can be monitored by assessing the pattern of antigen presentation.

Specific staining also can be used to monitor changes in receptor landscapes. For example, a library of molecules, such as scFvs, which interact with cell surface receptors can be displayed on the arrays. The arrays are then exposed to a cellular sample. The interaction between the cell surface receptors and the scFvs displayed on the arrays can result in the transduction of a signal from the surface to the interior of the cell, resulting in a response. The response can be monitored in a specific or semi-specific manner. For example, a cytotoxic T cell activates a death-inducing caspase cascade in the target cell by interacting with transmembrane receptor proteins, Fas. Binding of the Fas ligand on the T cell to the Fas proteins on the target cell alters the Fas proteins so that their clustered cytosolic tails recruit procaspase-8 in the complex via an adaptor protein. The recruited procaspase-8 molecules cross-cleave and activate one another to begin the caspase cascade that leads to apoptosis. The death of the cell can be monitored by specific dyes that are released upon cell death, however, the cause of death is unknown due to the non-specific nature of the apoptosis visualization. Instead, scFv molecules can be displayed on arrays and exposed to cellular samples. The cells can then be fixed and permeabilized such that a stain specific for caspase, such as the anti-Zap70 antibody, can enter the interior of the cell and be visualized. The presence of activated caspase, as indicated by the staining, highlights those cells where the caspase cascade has been activated by the interaction between the scFv library and the cell surface receptors of the proteins.

Similarly, but less specifically, the initiation of classes of enzymes, such as the kinases, can be monitored by specific staining. For example, a capture system containing an scFv library can be contacted to a cellular sample. The cells can then be fixed and permeabilized. Upon permeabilization, the arrays are stained with an anti-Phos Tyr antibody which is specific for peptides containing phosphorylated tyrosines. Cells which are visualized indicate a cellular system where the interaction of the scFv on the array resulting in a cellular signal that initiated kinase activity.

Another example demonstrates the use of specific stain, such as an anti-SH2/SH3 antibody,.that is used to stain cells where a signaling pathway incorporating peptides with SH2 or SH3 domains has been initiated by interaction between the cell surface receptors and the scFv library.

### 2. Molecules for Staining

There are many staining methods used to localize molecules that are known to those skilled in the art, and any can be used in the methods herein. Selection of the stain can be made by those of skill in the art and depends upon the particular application. For example, factors that affect the method chosen, include, for example, the type of sample, the degree of sensitivity needed and the processing time and cost requirements. Staining of molecules can be performed directly or indirectly. Direct staining involves the staining and detection of a specific molecule or class of molecules of interest. Indirect staining involves the staining and detection of a molecule resulting from a secondary reaction of the molecule or class of molecules of interest, such as a signal transduction product or the product of an enzymatic reaction. Molecules used for staining can be any compound that is detectable or produces a detectable signal. Molecules that can be used for staining include, but are not limited to, an organic compound, inorganic compound, metal complex, receptor, enzyme, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, biopolymer, polymer, sub-cellular structure, sub-cellular compartment or any combination, portion, salt, or derivative thereof. These molecules can be detected directly or labelled with a detectable label, such as a luminescent molecule.

Molecules, such as antibodies, are commercially available conjugated to a detectable label or are synthetically producible for use in specific staining depending on the particular molecule or class of molecules of interest. Proteins which can be used as a detectable label include, but are not limited to, GFP, RFP and BFP. A wide variety of luminescent molecules are commercially available, and include, but are not limited to, FITC, fluorescein, rhodamine, Cascade Blue, Marina Blue, Alexa Fluor® 350, red-fluorescent Alexa Fluor® 594, Texas Red, Texas Red-X and the red- to infrared-fluorescent Alexa Fluor® 633, Alexa Fluor® 647, Alexa Fluor® 660, Alexa Fluor® 680, Alexa Fluor® 700 and Alexa Fluor® 750 dyes (Molecular Probes). Attachment of the luminescent molecule can be performed by any means known to those skilled in the art, such as with the Zenon One Mouse IgG₁ labeling kit from Molecular Probes. Conjugated antibodies also can be commercially purchased with the luminescent label already attached from companies such as Molecular Probes (online at probes.com), Invitrogen (www.invitrogen.com), Amersham Biosciences (online at amershambiosciences.com) and Pierce Biotechnologies (online at piercenet.com).

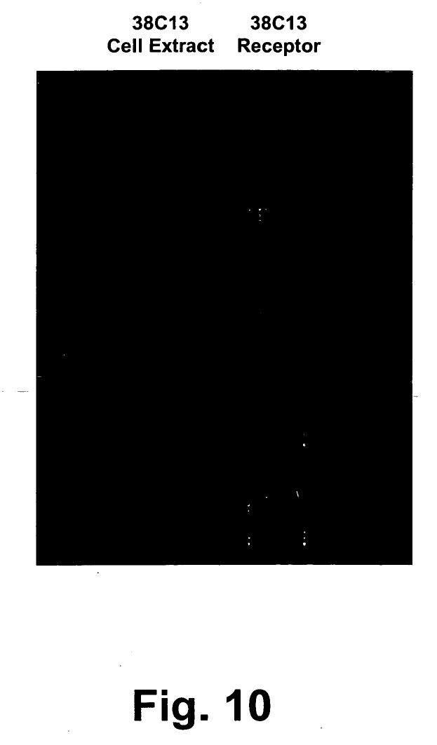

A particular embodiment of specific staining is exemplified in Example 6. Briefly, idiotype receptors can be used to identify lymphoma cells. These receptors are IgM molecules that reside on the surface of lymphoma cells. In order to identify a scFv that interacts with an idiotype receptor from a particular lymphoma cell, a sample lysate from a lymphoma culture is exposed to a capture system displaying a master library of tagged scFv molecules. Once lysate components are bound to the capture system, IgM molecules are specifically stained with a detection antibody, such as an anti-Ig-Fc antibody, that is specific for the constant domain of IgM molecules. The secondary antibody is then visualized by any method known to those skilled in the art, indicating which loci within the arrays contain IgM molecules from the lymphoma cells of the sample that are interacting with a scFv through the IgM receptor (FIG. 10).

### G. Use of Capture Systems For Capturing and Analyzing Biological Particles and For Drug Discovery and Other Screening Applications

The capture systems described herein can be used to capture and analyze biological particles, including, but not limited to, whole cells, eukaryotic and prokaryotic cells and fragments or organelles thereof or protein complexes; viruses, such as a viral vector or viral capsids with or without packaged nucleic acid; phage, including a phage vector or phage capsid, with or without encapsulated nucleotide acid; liposomes, other micellar agents or other packaging particles; and other such biological materials.

The capture systems with captured biological particles provided herein serve as an “artificial synapse” or point of synapse between the cells (or other biological particles) and the capture system surface which is mimicking a biological particle, such as a cell surface. The capture systems herein provide the ability to sort and/or to assess functional effects of test conditions and/or compounds, such as drug compounds, on biological particles. The biological particles, such as cells, can be seeded on the capture systems either by washing them over the system and allowing them to settle to the surface or by applying them under conditions in which they are washed to promote specific interactions. The cells or other biological particles then can be assessed by functional assays or staining. Optionally, the biological particles can be fixed to the capture system and then stained or otherwise detected. The capture agents on the surface can serve to anchor the cells and/or to provide signals via cell surface receptors.

The following sections and subsections describe the preparation of and use of capture systems with arrayed biological particles. It is understood that these are exemplary only and other applications are intended to be included.

### 1. Capture of Biological Particles

Biological particles can be exposed to the capture system using any method known to those skilled in the art. For example, the biological particles can be bathed over the capture system or seeded within the system, with and without washing. Once exposed to the capture system, the biological particles can be monitored by any method known to those skilled in the art, such as visually by microscopic methods or with spectroscopic methods. The monitoring of the biological particles can take place in real time or at designated time intervals by fixing the biological particles to the capture system then staining or other variations thereof. The biological particles can optionally be made permeable to exogenous molecules by any method known to those skilled in the art such as, but not limited to, electroporation and calcium chloride exposure.

In addition to profiling the surface of a biological particle and identifying compounds and/or conditions that modulate secondary mechanisms within a biological particle bound to a capture system, conditions and compounds that affect the life cycle of a particular biological particle also can be assessed. For example, biological particles can be exposed to a capture system prior to, simultaneously with or after the addition of a test compound and/or condition. The ability of the captured biological particle to propagate can be assessed and, thus, the effect of the test compound and/or condition on the biological particle life cycle can be determined. With this type of application, test conditions and/or compounds that facilitate cell growth, that inhibit cell growth and facilitate apoptosis and that reverse either the aging or the propagation process can be identified.

In a particular embodiment, as shown in Example 7, a capture system was prepared wherein the anti-IgM antibody (S1C5: anti-idiotype monoclonal antibody from B cells), its equivalent scFv (S1C5 scFv), the anti-T cell receptor antibody (C6VL) and the scFv for Human fibronectin (HFN) were printed onto loci within two arrays. One array in the capture system then was exposed to B cells that recognize the S1C5 antibody and scFv and the other array was exposed to T cells that recognize the C6VL antibody. The captured cells were immediately imaged. The B cells bound only to those loci containing the S1C5 antibody or scFv, while the T cells bound only to those loci containing the C6VL antibody.

### a. Doping of Loci with Secondary Agents

In addition to the displayed libraries of tagged molecules attached to the capture agents, one or a plurality of identical or varied secondary agents can be present within one or a plurality of loci within the capture system. The doping of a locus in the capture system results in secondary agents with a known effect or function being displayed in addition to tagged molecules with an unknown effect or function within an individual locus. The secondary agents can serve one or a plurality of functions within the capture system, including, but not limited to, co-stimulatory functions, binding to surface receptors different from the tagged molecules, exertion of a biological effect, exertion of an anchoring function to increase the stability of the interaction between the biological particle and the capture system and further selection of the biological particles that bind to a locus. The secondary agent can be addressably arrayed with the capture agents of the capture system or can be added exogenously prior to, simultaneously with or after the exposure of the biological particle to the capture system.

Secondary agents include, but are not limited to, an organic compound, inorganic compound, met al complex, receptor, enzyme, protein complex, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, an antibody or fragment thereof, antibody conjugate, biopolymer, polymer or any combination, portion, salt, or derivative thereof. Some exemplary molecules that can serve as secondary agents include, but are not limited to, adhesion molecules (e.g. ALCAM, BCAM, CADs, EpCAM, ICAMs, Cadherins, Selectins, MCAM, NCAM, PECAM and VCAM); angiogenic factors (e.g. Angiogenin, Angiopoietins, Endothelins, Flk-1, Tie-2 and VEGFs); binding proteins (e.g. IGF binding proteins); cell surface proteins (e.g. B7s, CD14, CD21, CD28, CD34, CD38, CD4, CD6, CD8a, CD64, CTLA-4, decorin, LAMP, SLAM, ST2 and TOSO); chemokines (e.g. 6Ckine, BLC/BCA-1, ENA-78, eotaxins, fractalkine, GROs, HCCs, MCPs, MDC, MIG, MIPs, MPIF-1, PARC, RANTES, TARK, TECK and SDF-1); chemokine receptors (e.g. CCRs, CX3CR-1 and CXCRs); cytokines and their receptors (e.g. Epo, Flt-3 ligand, G-CSF, GM-CSF, interferons, IGFs, IK, leptin, LIF, M-CSF, MIF, MSP, oncostatin M, osteopontin, prolactin, SARPs, PD-ECGF, PDGF A and B chains, Tpo, TIGF and PREF-1, AXL, interferon receptors, c-kit, c-met, Epo R, Flt-s/Flk-2 R, G-CSF R, GM-CSF.R, etc.); ephrin and ephrin receptors; epidermal growth factors (e.g. amphiregulin, betacellulin, cripto, erbB1, erbB3, erbB4, HB-EGF and TGF-α); fibroblast growth factors (FGFs) and receptors (FGFRs); platelet-derived growth factors (PDGFs) and receptors (PDGFRs); transforming growth factors beta (TGFs-β, e.g. activins, bone morphogenic proteins (BMPs) and receptors (BMPRs), endometrial bleeding associated factor (EBAF), inhibin A and MIC-1); transforming growth factors alpha (TGFs-α); insulin-like growth factors (IGFs); integrins (alphas and betas); interleukins and interleukin receptors; neurotrophic factors (e.g. BDNF, b-NGF, CNTF, CNTF Rα, GDNF, GRFαs, midkine, MUSK, neuritin, neuropilins, NGF R, NT-3, semaphorins, TrkA, TrkB and TrkC); interferons and their receptors; orphan receptors (e.g. Bob, ChemR23, CKRLs, GRPs, RDC-1 and STRL33/Bonzo); proteases and release factors (e.g. matrix metalloproteinases (MMPs), caspases, furin, plasminogen, SPC4, TACE, TIMPs and urokinase R); T cell receptors; MHC peptides; MHC peptide complexes; B cell receptors; intracellular adhesion molecules (ICAMs); Toll-like receptors (TLRs; recognize extracellular pathogens, such as pattern recognition receptors (PRR receptors) and PPAR ligands (peroxisome proliferative-activated receptors); ion channel receptors; neurotransmitters and their receptors (e.g. nicotinic acetylcholine, acetylcholine, serotonin, γ-aminobutyrate (GABA), glutamate, aspartate, glycine, histamine, epinephrine, norepinephrine, dopamine, adenosine, ATP and nitric oxide); muscarinic receptors; small molecule receptors (e.g. NO and CO₂ receptors); steroid hormones and their receptors (e.g. progesterone, aldosterone, testosterone, estradiol, cortisol, retinoic acid receptors (RARs), retinoid X receptors (RXRs) and PPARs); peptide hormones and their receptors (e.g. human placental lactogen, prolactin, gonadotropins, corticotropins, calcitonin, insulin, glucagon, somatostatin, gastrin and vasopressin); tumor necrosis factors (TNFs, e.g. April, CD27,. CD27L, CD30, CD30L, CD40, CD40L, DR-3, Fas, FasL, HVEM, lymphotoxin β, osteoprotegerin, RANK, TRAILs, TRANCE and TWEAK) and their receptors; nuclear factors; and G proteins and G protein coupled receptors (GPCRs). Other compounds for doping include drugs, such as the anti-Her-2 monoclonal antibody trastuzumab (Herceptin®) and the anti-CD20 monoclonal antibodies rituximab (Rituxan®), tositumomab (Bexxar™) and Ibritumomab (Zevalin™), the anti-CD52 monoclonal antibody Alemtuzumab (Campath™), the anti-TNFα antibodies infliximab (Remicade™) and CDP-571 (Humicade®), the monoclonal antibody edrecolomab (Panorex®), the anti-CD3 antibody muromab-CD3 (Orthoclone®), the anti-IL-2R antibody daclizumab (Zenapax®), the omalizumab antibody against IgE (Xolair®), the monoclonal antibody bevacizumab (Avatin™), small molecules such as erlotinib-HCI (Tarceva™) and others that bind to receptors or cell surface proteins.

Many cellular processes require the binding events, molecular interactions or reactions to yield the end result of the process. For example, activation of a T cell to proliferate and differentiate into an effector cell requires two signals from an antigen presenting cell, such as a dendritic cell. The two signals are co-stimulatory in that in the absence of the second signal, the first signal results in inactivation or apoptosis of the T cell. In order to investigate molecular and cellular systems which have multiple interactions occurring simultaneously or sequentially, the loci of the capture system can be doped with one or more of the molecules required for a particular signal and then used to identify the second signal within a library of tagged molecules randomly displayed among the loci resulting in a particular function within the biological particle. For example, the loci of a capture system can be doped with co-stimulatory B7 proteins from an APC, which interact with co-receptor CD28 proteins from a T cell, yielding a signal required, in addition to the interaction of the MHC peptide of the APC and TCR of the T cell, for proliferation of a T cell following exposure to an APC. The capture system is then prepared such that a library of tagged MHC peptides is randomly displayed among the loci by interactions with the capture agents. The completed capture system is then exposed to a sample containing T cells. Those T cells that proliferate possess the required T cell receptor for the MHC displayed as well as the CD28 protein required for interaction with the B7 protein. This doped capture system can be expanded to contain one or a plurality of secondary agents required for a particular interaction, thus serving as a type of artificial environment for mimicking cellular interactions.

In addition, probing with the libraries of tagged molecules in the presence of a secondary agent can identify molecules that can modulate the interaction between the secondary agent and the biological particle or can assess a separate interaction and/or secondary reactions. Further, the effects of test conditions and compounds with unknown effects also can be assessed. For example, test compounds such as, co-stimulants (in the case of the drugs) or compounds and conditions that stimulate activity of known drugs can be added either prior to, simultaneously with or after the exposure of the biological particles to the doped capture system. The effect of these compounds and/or conditions can be assessed as discussed above.

### b. Fixation of Cells to Capture Array

For methods where the preservation of the biological particles on the array is desired, the biological particles can be fixed in place on the capture system. A fixative is employed to prevent autolysis by inactivating lysosomal enzymes and inhibiting the growth of bacteria and molds, that produce putrefactive changes. Furthermore, fixatives stabilize the biological particles to protect them from the rigors of subsequent processing and staining.

In performing their protective role, fixatives can denature proteins by coagulation, by forming additive compounds or by a combination of the two. Conformational changes in the structure of proteins can occur causing inactivation of enzymes. Fixatives can also cause physical changes to cellular and extracellular constituents.

Viable cells are encased in an impermeable membrane. Fixation breaks down this barrier and allows relatively large molecules to penetrate and escape. In addition, the cytoplasm undergoes a sol-gel transformation with the formation of a proteinaceous network sufficiently porous to allow further penetration of large molecules. Different fixatives result in different degrees of porosity. Coagulant fixatives, such as B5 and formal sublimate, result in a larger pore size than do non-coagulant fixatives, such as formalin. Most fixative solutions contain chemicals, which stabilize proteins, since this is how protection of the cellular structure is effectively accomplished.

As shown in the methods provided herein, formaldehyde-based fixatives can be used to fix biological particles to a capture system. Formaldehyde-based fixatives contain formalin (40% w/v formaldehyde in water), usually in a neutral salt to maintain tonicity and often a buffering system to maintain pH. Formaldehyde fixes not by coagulation but by reacting with basic amino acids to form cross-linking methylene bridges. Thus, there is a relatively low permeability to macromolecules and the structures of the intracytoplasmic proteins are not significantly altered. Other fixatives include, but are not limited to, mercuric chloride-based fixatives, such as B5 and Zenker's solution, periodate-lysine paraformaldehyde (PLP), ethanol and acetone. As stated above, the fixatives vary in their coagulative and additive properties and one skilled in the art can empirically determined the most effective fixative for a particular use.

### 2. Methods to Detect Secondary Effects of Cell Binding to Capture Systems

Interaction of a biological particle with a capture system can cause secondary interactions within or on the exterior of the biological particle. The interactions resulting from the interaction among the biological particles and the capture systems can include any interaction that molecules and biological particles exhibit. Such interactions include, but are not limited to, protein:protein, protein:nucleic acid, nucleic acid:nucleic acid, protein:lipid, lipid-lipid, protein:small molecule, receptor:signal, antibody:antigen, peptide nucleic acid:nucleic acid, and small molecule:nucleic acid. These interactions, and therefore, the targets, are involved in a variety of chemical and biological processes, including, but not limited to, conformational changes; binding interactions; complexation; hybridization; transfection; hydrophobic interactions; signal transduction; membrane translocation; electron transfer; conversion of a reactant to a product via a catalytic mechanism; chaperoning of compounds inter- and intracellularly; fusion of liposomes to membranes; infection of a foreign pathogen into a host cell or organism, such as a virus (HIV, influenza virus, polio virus, adenovirus, etc.) or bacteria (Escherichia coli, Pseudomonas aeruginosa, Salmonella enteritidis, etc.); initiation of a regulatory cascade, detoxification of cells and organisms; and cell replication and division.

The methods to detect these secondary interactions include, but are not limited to, transcription reporters, immunostaining, spectroscopic product detection and resonance energy transfer techniques. Some techniques, such as transcription reporters, require that the target interaction be identified prior to exposure of the biological particles to the capture system. For example, using transcription reporters to identify interactions between the biological particle and the capture system that result in the initiation of caspase synthesis requires insertion of the transcription reporter construct into the gene encoding the caspase prior to exposure of the biological particle to the capture system. Other techniques, such as immunostaining and spectroscopic methods, have a less stringent requirement regarding the knowledge of the interaction prior to the exposure of the biological particles. For example, interactions between the biological particle and the capture system that result in the formation of a product detectable by spectroscopy or immunostaining or another method can be identified without altering the biological particle prior to exposure to the capture system. One skilled in the art can recognize the level of knowledge needed for a particular detection technique and select a method of detection appropriately.

### a. Transcription Reporters

Transcription reporters are nucleic acid molecules that contain reporter genes that encode easily assayed proteins. These reporter genes are used to replace or assist in the detection of other coding regions whose protein products are more difficult to assay. As used with the capture systems provided herein, these transcription reporters can be used to identify and assess a secondary reaction resulting from the interaction of the biological particle with the capture system. The reporter gene can be used to replace a gene encoding a suspected transcription product or can be placed in frame with the transcription product, yielding a detectable fused transcription product.

Reporter genes are generally joined to a regulatory DNA sequence in an expression vector that is usually propagated in the appropriate bacterial host before transfection into the cell type of interest. A control reporter driven by a strong, constitutive promoter is cotransfected with the experimental reporter plasmid to normalize for transfection efficiency and to account for the fact that expression of the experimental reporter may vary in different cell types. After allowing time for gene expression, the cells are assayed for reporter mRNA, the reporter protein itself, or for the activity of the reporter protein. Detection of the reporter gene product usually requires cell lysis, although some products are amenable to in situ analysis.

### (1) Reporter Gene Constructs

Reporter gene constructs are prepared by operatively linking a reporter gene with at least one transcriptional regulatory element. If only one transcriptional regulatory element is included, it can be a regulatable promoter. At least one of the selected transcriptional regulatory elements can be indirectly or directly regulated by the activity of the selected cell surface receptor whereby activity-of the receptor can be monitored via transcription of the reporter genes.

The construct may contain additional transcriptional regulatory elements, such as a FIRE sequence, or other sequence, that is not necessarily regulated by the cell surface protein, but is selected for its ability to reduce background level transcription or to amplify the transduced signal and to thereby increase the sensitivity and reliability of the assay. Many reporter genes and transcriptional regulatory elements are known to those of skill in the art and others may be identified or synthesized by methods known to those of skill in the art.

### (2) Reporter Genes

A reporter gene includes any gene that expresses a detectable gene product, including, but not limited to, RNA or polypeptide. Among the reporter genes contemplated for the methods provided herein are those that encode readily detectable transcription products. The reporter gene can replace an identified target transcription gene or can be included in the construct in the form of a fusion gene with a gene that includes desired transcriptional regulatory sequences or exhibits other desirable properties. Ideally, a reporter gene encodes for a protein whose activity can be detected with high sensitivity above any endogenous activity and that displays a wide dynamic range of response (over several orders of magnitude). Choosing the best reporter gene depends on the type of study (regulation of gene expression or determination of transfection efficiency), organism and cell type, type of information sought (temporal versus spatial), and preferred detection method (e.g., liquid scintillation, spectrophotometry, or luminometry). Many reporters have been adapted for a broad range of assays, including calorimetric, fluorescent, bioluminescent, chemiluminescent, ELISA, and/or in situ staining.

Examples of reporter genes include, but are not limited to, chloramphenicol acetyltransferase (CAT) (Alton and Vapnek (1979) Nature 282: 864-869) luciferase, and other enzyme detection systems, such as beta-galactosidase; firefly luciferase (deWet et al. (1987) Mol. Cell. Biol. 7: 725-737); bacterial luciferase (Engebrecht and Silverman (1984), PNAS 1: 4154-4158; Baldwin et al. (1984) Biochemistry 23: 3663-3667); alkaline phosphatase (Toh et al. (1989) Eur. J. Biochem. 182: 231-238, Hall et al. (1983) J. Mol. Appl. Gen. 2: 101); secreted alkaline phosphatase (SEAP) (Yang et al. (1994) CLONTECHniques IX(3): 1-5; Berger et al. (1988) Gene 66: 1-10; and Cullen & Malim (1992) Methods Enzymol. 216: 362-368); β-galactosidase (β-GAL) (MacGregor et al. (1987) Somat. Cell Mol. Genet. 13: 253-265); β-glucuronidase (β-GUS); and fluorescent proteins such as GFP, RFP and BFP. These reporter genes are commercially available at companies such as Invitrogen (online at invitrogen.com), Novagen (online at novagen.com), Applied Biosystems (online at appliedbiosystems.com) and Molecular Probes (online at probes.com).

### (3) Transcriptional Control Elements

Transcriptional control elements include, but are not limited to, promoters, enhancers, and repressor and activator binding sites, Suitable transcriptional regulatory elements can be derived from the transcriptional regulatory regions of genes whose expression is rapidly induced, generally within minutes, of contact between the biological particle and the capture system that modulates the activity of the biological particle. Examples of such genes include, but are not limited to, the immediate early genes (see, Sheng et al. (1990) Neuron 4: 477-485), such as c-fos and jun. Immediate early genes are genes that are rapidly induced upon binding of a ligand to a cell surface protein. Exemplary transcriptional control elements for use in the gene constructs include transcriptional control elements from immediate early genes, elements derived from other genes that exhibit some or all of the characteristics of the immediate early genes, or synthetic elements that are constructed such that genes in operative linkage therewith exhibit such characteristics. Attributes of exemplary genes from which the transcriptional control elements are derived include, but are not limited to, low or undetectable expression in quiescent cells, rapid induction at the transcriptional level within minutes of extracellular stimulation, induction that is transient and independent of new protein synthesis, subsequent shut-off of transcription requires new protein synthesis, and mRNAs transcribed from these genes have a short half-life. It is not necessary for all of these properties to be present.

Other promoters and transcriptional control elements, in addition to those described above, include the vasoactive intestinal peptide (VIP) gene promoter (cAMP responsive; Fink et al. (1988), Proc. Natl. Acad. Sci. 85: 6662-6666); the somatostatin gene promoter (cAMP responsive; Montminy et a. (1986), Proc. Natl. Acad. Sci. 83: 6682-6686); the proenkephalin promoter (responsive to cAMP, nicotinic agonists, and phorbol esters; Comb et al. (1986) Nature 323: 353-356); the phosphoenolpyruvate carboxy-kinase gene promoter (cAMP responsive; Short et al. (1986) J. Biol. Chem. 261: 9721-9726); the NGFI-A gene promoter (responsive to NGF, cAMP, and serum; Changelian et al. (1989). Proc. Natl. Acad. Sci. 86: 377-381); and others that may be known to or prepared by those of skill in the art.

### b. Immunostaining

There are many immunostaining methods used to localize antigens known to those skilled in the art. Many factors affect the method of choice including the type of sample, the degree of sensitivity needed and the processing time and cost requirements. Immunostaining of antigens can be performed directly or indirectly. Direct staining is a method in which an enzyme-linked primary antibody reacts with the antigen in the sample. Subsequent use of substrate-chromagen concludes the reaction sequence and results in a detectable product. Indirect staining is a method in which an unconjugated primary antibody binds to an antigen. An enzyme-labelled secondary antibody directed against the primary antibody is then applied, followed by substrate-chromagen solution that results in a detectable product. The secondary antibody generally is prepared in a subject different from the subject in which the primary antibody was prepared. For example, if the primary antibody is made in rabbit or mouse, the secondary antibody should be directed against rabbit or mouse immunoglobulins. Additional layers of secondary antibodies are also contemplated. The enzyme or enzymes can be attached to the antibody by any method known to those skilled in the art (Wild The Immunoassay Handbook, Nature Publishing Group (2001) and Van der Loos Immunoenzyme Multiple Staining Methods, Bios Scientific Pub Ltd (2000)) or can be purchased commercially as an enzyme-antibody conjugate. The reaction product can be detected by any method known to those skilled in the art including, but not limited to, calorimetric, spectroscopic and electrochemical (Kulis et al. J. Electroanal. Chem. 382: 129 (1995); Bauer et al Anal. Chem. 68: 2453 (1996); and Bagel et al. Anal. Chem. 69: 4688).

### (1) Enzymes and Chromagens for Immunostaining

Most immunoenzymatic staining methods utilize enzyme-substrate reactions to convert colorless chromagens into colored end products. Any enzyme that can react with a chromagen directly or a substrate to yield a product that can then react with a chromagen to yield a detectable signal and can be attached to an antibody that interacts either directly or indirectly with an antigenic species can be used. Some exemplary enzymes include, but are not limited to, horseradish peroxidase (HRP) and calf intestine alkaline phosphatase (AP), galactosidase and glucose oxidase. Additionally, luminescent proteins such as green fluorescent protein (GFP), red fluorescent protein (RFP) and blue fluorescent protein (BFP) or other luminescent molecules, such as, FITC, rhodamine, fluorescein and Alexa Fluor® dyes (Molecular Probes), can be attached to the antibody being used and visualized directly.

### (a) Luminescent Labels

In immunostaining techniques, a luminescent label is a molecule that can be attached to either a primary or secondary antibody and visualized without the addition of a substrate or a chromagen. Proteins which can be used include, but are not limited to, GFP, RFP and BFP. A wide variety of luminescent molecules are commercially available, and include, but are not limited to, FITC, fluorescein, rhodamine, Cascade Blue, Marina Blue, Alexa Fluor® 350, red-fluorescent Alexa Fluor® 594, Texas Red, Texas Red-X and the red- to infrared-fluorescent Alexa Fluor® 633, Alexa Fluor® 647, Alexa Fluor® 660, Alexa Fluor® 680, Alexa Fluor® 700 and Alexa Fluor® 750 dyes (Molecular Probes). Attachment of the luminescent molecule can be performed by any means known to those skilled in the art, such as with the Zenon One Mouse IgG₁ labeling kit from Molecular Probes. Conjugated antibodies also can be commercially purchased with the luminescent label already attached from companies such as Molecular Probes (online at probes.com), Invitrogen (online at invitrogen.com), Amersham Biosciences (online at amershambiosciences.com) and Pierce Biotechriologies (online at piercenet.com).

### (b) Horseradish Peroxidase (HRP)

HRP is a heme-containing enzyme isolated from the root of the horseradish plant. The heme substituent of HRP forms a complex with hydrogen peroxide, which then decomposes resulting in water and atomic oxygen. HRP oxidizes several substances, such as polyphenols and nitrates. HRP can be covalently or non-covalently attached to other proteins, such as antibodies, using any method known to those skilled in the art (see, e.g., Sternberger Immunocytochemistry (2nd Ed.) New York: Wiley, 1979) or can be purchased as part of a conjugated antibody-enzyme complex from commercial sources such as Invitrogen, Pierce Biotechnologies and Arnersham Biosciences.

HRP activity in the presence of an electron donor, such as hydrogen peroxide, first results in the formation of an enzyme-substrate complex, and then in the oxidation of the electron donor. The electron donor provides the driving force in the continuing catalysis of hydrogen peroxide, while its absence effectively stops the reaction. Electron donors, called chromagens, become colored products when oxidized and include, but are not limited to, 3,3′-Diaminobenzidine (DAB), 3-Amino-9-ethylcarbazole (AEC), 4-Chloro-1-naphthol (CN), p-Phenylenediamine dihydrochloride/pyrocatechol (Hanker-Yates reagent), chloro-1-naphthol, luminol, ECF substrate and 3,3′,5,5′-tetramethylbenzidine (TMB). These compounds can be synthetically prepared by any method known to those skilled in the art or can be purchased from commercial sources.

### (c) Alkaline Phosphatase (AP)

Calf intestine alkaline phosphatase removes and transfers phosphate groups from organic esters by breaking the phosphate-oxygen bond. The chief metal activators are divalent magnesium, manganese and calcium. Alkaline phosphatase can be covalently or non-covalently attached to other proteins, such as antibodies, synthetically using any method known to those skilled in the art, or can be purchased as an antibody-enzyme complex.

In the immunoalkaline phosphatase staining method, the enzyme hydrolyzes naphthol phosphate esters (substrate) to phenolic compounds and phosphates. The phenols couple to colorless diazonium salts (chromagen) to produce insoluble, colored azo dyes. Substrates used in conjunction with alkaline phosphatase include, but are not limited to, Naphthol AS-MX phosphate, naphthol AS-BI phosphate, naphthol AS-TR phosphate and 5-bromo-4-chloro-3-indoxyl phosphate (BCIP). Chromagens used include, but are not limited to Fast Red TR, Fast Blue BB, new fuchsin, Fast Red LB, Fast Garnet GBC, Nitro Blue Tetrazolium (NBT) and iodonitrotetrazolium violet (INT). These compounds can be synthetically prepared by any method known to those skilled in the art or can be purchased from commercial sources.

### (2) Avidin-Biotin Staining Methods

As described above, immunostaining can be accomplished either directly or indirectly using enzymatic reaction for visualization of the antigenic site. In an extension of these methods, the interaction between avidin and biotin has been exploited to develop an immunostaining method that has an inherent amplification of sensitivity when compared with other methods. Avidin (chicken egg) is a tetramer containing four identical subunits. Each subunit contains a high affinity binding site fore biotin, an egg white protein, with a dissociation constant of approximately 10⁻¹⁵ M. The binding is undisturbed by extremes of pH, buffer salts or chaotropic agents such as guanidine hydrochloride. Streptavidin, from Streptomyces avidinii, can be exchanged for avidin in the interaction with biotin.

This strong interaction is the focus of three immunostaining methods. The labelled avidin-biotin (LAB) method (Guesdon et al. J. Histochem. Cytochem. 27: 1131 (1983)) utilizes a biotinylated antibody which is reacted either with an antigen or a primary antibody, followed by a second layer of enzyme-labelled avidin. After the avidin-enzyme conjugate binds to the biotinylated antibody, chromagen-is added to detect the antigen. The bridged avidin-biotin method (BRAB) (Guesdon et al. J. Histochem. Cytochem. 27: 1131 (1983)) is essentially the same as the LAB method, except that the avidin is not conjugated to an enzyme. The BRAB method utilizes avidin as a bridge between the biotinylated antibody and a biotinylated enzyme. Due to the multiple binding sites on avidin, more biotinylated enzymes can be complexed to increase the intensity of the chromagen color development. The avidin-biotin complex (ABC) method (Hsu et al. Am. J. Clin. Path. 75: 734-738 (1981); Hsu et al. Am. J. Clin. Path. 75: 816 (1981); and Hsu et al. J. Histochem. Cytochem. 29: 577-580 (1981)) utilizes the initial complex as in the LAB or BRAB system, but requires that the biotinylated enzyme be preincubated with the avidin, forming large complexes to be incubated with the biotinylated antibody. The avidin and biotinylated enzyme are mixed together in a specified ratio for about 15 minutes at room temperature to form these complexes. An aliquot of this solution is then added to the sample, and any remaining biotin-binding sites will bind to the biotinylated antibody. The result is a greater concentration of enzyme at the antigenic site in the sample and an increase in sensitivity.

### (3) Chain Polymer-Conjugated Technology

To achieve high sensitivity, the most commonly used staining methods in immunohistochemistry to date have been based on a multi-layer technique. Conjugates used in multi-layer techniques normally consist of one or two enzyme molecules per antibody or avidin-streptavidin molecules. A biotinylated secondary antibody and an avidin-streptavidin conjugate are used to exploit the high affinity of avidin-streptavidin for biotin. Sensitivity is enhanced by increasing the number of enzyme molecules bound to the antigen through the detecting antibody. A technology recently developed by DAKO (online at dako.com) enables the coupling of a high number of molecules to a dextran backbone. This chemistry permits binding of a large number of enzyme molecules (e.g., horseradish peroxidase or alkaline phosphatase) to a secondary antibody via the dextran backbone. The resulting polymeric conjugate can consist of up to 100 enzyme molecules and up to 20 antibody molecules per backbone and is kept water-soluble by using hydrophilic, non-charged dextran as the backbone. The increase in the number of enzymes per antigen results in an increase in sensitivity, a minimization of non-specific background staining and a reduction in the total number of assay steps as compared to conventional technologies. Staining kits and reagents, such as the Enhanced Polymer One-Step Method (EPOS™) and EnVision® systems, that utilize this technology can be purchased commercially from DAKO.

### c. Resonance Energy Transfer

Molecular interactions and biological and/or chemical reactions can be detected by any methods that analyze, assay, or observe the molecules that participate in these interactions and/or reactions. As a non-limiting example, interactions and reactions can be analyzed by detecting the emission of light from molecules involved in the interactions and reactions. Such emission of light can stem from luminescence phenomena, such as, but not limited to, fluorescence, phosphorescence, chemiluminescence, and bioluminescence.

Luminescence signals, such as fluorescence signals, can be measured as single or multiple parameters corresponding to different laser excitation and fluorescence emission wavelengths. Multiple and/or different luminescers, such as fluorophores and bioluminescers and quenchers, also can be used in the same reaction. Certain combinations of fluorochromes, phospholuminescers, bioluminescers and quenchers cannot be used simultaneously; those of skill in the art can identify such combinations.

Molecular interactions can be detected by energy transfer experiments in which one molecule (i.e. the donor molecule) absorbs radiation at an appropriate wavelength (excitation) and transfers energy to another molecule (i.e. the acceptor molecule) which can emit light at a detectable wavelength or merely quench the radiation. Donor-acceptor combinations that can be used in energy transfer analyses include, but are not limited to, fluorescent donors with fluorescent or phosphorescent acceptors, or phosphorescent donors with phosphorescent or fluorescent acceptors. In an exemplary embodiment, the energy that is transferred from donor to acceptor molecules is fluorescence energy (i.e. FRET).

The molecular and/or biological particle components of the targets identified herein can be labeled with at least two labels on a single component or on multiple components. Other combinations, including, but not limited to, three or more labelled components, one component with three or more labels and one component with one or more labels and a second component with one or more labels, will be apparent to those with skill in the art based upon the disclosure herein.

### (1) Luminescence Processes

Any luminescent label can be selected. For purposes herein the processes are exemplified with reference to fluorescence. It is understood that any label, particularly those for use in energy transfer protocols, is contemplated.

### (a) The Fluorescence Process

Fluorescence is the result of a three-stage process that occurs can be described as three phases, excitation, excited-state lifetime, and emission. During excitation, a photon of energy hv_{EX} is supplied by an external source such as an incandescent lamp or a laser and absorbed by the fluorophore, creating an excited electronic singlet state (S₁′). This process distinguishes fluorescence from chemiluminescence, in which the excited state is populated by a chemical reaction.

The excited state exists for a finite time (typically 1-10 nanoseconds), and is termed the excited-state lifetime. During this time, the fluorophore undergoes conformational changes and is also subject to a multitude of possible interactions with its molecular environment. These processes have two important consequences. First, the energy of S₁′ is partially dissipated, yielding a relaxed singlet excited state (S₁) from which fluorescence emission originates. Second, not all the molecules initially excited by absorption (excitation stage) return to the ground state (S₀) by fluorescence emission. Other processes such as collisional quenching, Fluorescence Resonance Energy Transfer (FRET) and intersystem crossing may also depopulate S₁. The fluorescence quantum yield, which is the ratio of the number of fluorescence photons emitted to the number of photons absorbed, is a measure of the relative extent to which these processes occur.

A photon of energy hv_{EM} is emitted, returning the fluorophore to its ground state S₀. Due to energy dissipation during the excited-state lifetime, the energy of this photon is lower, and therefore of longer wavelength, than the excitation photon hv_{EX}. The difference in energy or wavelength represented by (hv_{EX}−hv_{EM}) is called the Stokes shift. The Stokes shift is fundamental to the sensitivity of fluorescence techniques because it allows emission photons to be detected against a low background, isolated from excitation photons. In contrast, absorption spectrophotometry requires measurement of transmitted light relative to high incident light levels at the same wavelength.

### (b) Quenching Processes

### i) Photobleaching

The fluorescence process is a cyclical one, where the fluorophore is repeatedly raised to an excited state and relaxes back to the ground state with emission of a fluorescent photon. This process can occur many times. One of the consequences of this repeated excitation and emission is the loss of fluorescence from the molecule. This process is often referred to as photobleaching, photofading or photodestruction. Some dyes are much more sensitive than others to photobleaching, for example fluorescein photobleaches very easily. Often the rate of decomposition is proportional to the intensity of illumination. So a simple practical way to overcome this is to reduce the incident radiation.

Photobleaching can occur when the excited state is more chemically reactive than the ground state. A few of the dye molecules in the excited state will take part in chemical reactions leading to the loss of fluorescence. Frequently the reactions leading to photobleaching involve the singlet oxygen species. Singlet oxygen is extremely reactive and can react with dyes to quench their fluorescence. The singlet oxygen can be generated by the interaction of excited state dyes with triplet state oxygen leading to singlet state dyes and singlet state oxygen. It is sometimes possible to introduce antioxidants such as phenylalanine or azide, or to use anoxic conditions.

### ii) Self-Quenching, Static Quenching and Collisional Quenching

Multiple labelling of a molecule with a bright fluorophore does not always lead to an increase in fluorescent intensity. For a biological molecule that is labeled with N dye molecules, the overall brightness can described as,

Brightness=ε×F×N

where ε is the extinction coefficient of the fluorophore, F is Farraday's constant and N is the number of dye molecules. In many cases as N increases, the overall brightness decreases due the phenomenon of “self quenching”. Different dyes quench variably under certain conditions. Many dyes exhibit self-quenching where the presence of large concentrations of dyes will significantly impact on the quantum yield and it is clear that the dyes differ in their ability to self quench. The more hydrophobic the dye the lower the ratio of dye:protein where quenching will occur.

Static quenching is due to the formation of a ground state complex between the fluorescent molecule and the quencher with formation constant K_{c}, described by:

Iₒ/I=I+K_{c}

where Iₒ is the fluorescence intensity in the absence of quencher, I is the intensity in the presence of quencher at concentration [Q]. The observed lifetime does not appear in this equation and is independent of quencher concentration in static quenching.

Collisional quenching is described by the Stern-Volmer Equation

Iₒ/I=I+k_{q}[Q]t

where Iₒ is the fluorescence intensity in the absence of quencher, I is the intensity in the presence of quencher at concentration [Q], k_{q} is the rate of collisional quenching, and t is the observed lifetime. Collisional quenching is clearly observed when there is a linear decrease in the observed luminescence lifetime with increasing quencher concentration. Collisional quenching involves collisions with other molecules that results in the loss of excitation energy as heat instead of as emitted light. This process is always present to some extent in solution samples; species that are particularly efficient in inducing the process are referred to as collisional quenchers (e.g. iodide ions, molecular oxygen, nitroxide radical).

Static quenching processes involve the interaction of the fluorophore with the quencher, thus forming a stable non-fluorescent complex. Since this complex typically has a different absorption spectrum from the fluorophore, presence of an absorption change is diagnostic of this type of quenching (by comparison, collisional quenching is a transient excited state interaction and so does not affect the absorption spectrum). A special case of static quenching is self-quenching, where the fluorophore and the quencher are the same species. Self-quenching is particularly evident in concentrated solutions of tracer dyes.

Nonfluorescent acceptors such as dabcyl and QSY dyes (Molecular Probes) have the particular advantage of eliminating the potential problem of background fluorescence resulting from direct (i.e., nonsensitized) acceptor excitation. Probes incorporating fluorescent donor/non-fluorescent acceptor combinations have been developed primarily for detecting proteolysis and nucleic acid hybridization.

### (2) Luminescent Resonance Energy Transfer (LRET)

As noted above, LRET refers to non-radiative energy transfer between chemical and/or biological luminescent molecules, such as, but not limited to fluorophores, bioluminescers and phosphorescers (Heim et al. Curr. Biol. 6:178-182 (1996); Mitra et al. Gene 173:13-17 (1996); Selvin et al. Meth. Enzymol. 246:300-345 (1995); Matyus J. Photochem. Photobiol. B: Biol. 12: 323-337 (1992); Wu et al. Anal. Biochem. 218:1 -13 (1994)). The type of LRET observed is dependent on the luminescent molecules present in the sample. LRET among fluorophores gives fluorescent resonance energy transfer (FRET), among bioluminescent molecules gives bioluminescent resonance energy transfer (BRET) and among phosphorescent molecules gives LRET. The efficiency of LRET is dependent on the inverse sixth power of the intermolecular separation making it useful over distances comparable with the dimensions of biological macromolecules (Stryer and Haugland Proc Natl Acad Sci USA 58: 719-726 (1967)). Thus, LRET is an important technique for investigating a variety of biological phenomena that produce changes in molecular proximity (dos Remedios et al. J Struct Biol 1715: 1 75-185 (1995); Selvin Methods Enzymol 246: 300-334 (1995); Boyde et al. Scanning 17: 72-85 (1995); Wu et al. Anal Biochem 218: 1-13 (1994); Van der Meer et al. Resonance Energy Transfer Theory and Data pp. 133-168 (1994); dos Remedios et al. J Muscle Res Cell Motil 8: 97-117 (1987); Kawski Photochem Photobiol 38: 487 (1983); Stryer Annu Rev Biochem 47: 819-846 (1978); Fairclough et al. Methods Enzymol 48: 347-379 (1978)). When LRET is used as a contrast mechanism, co-localization of proteins and other molecules can be imaged with spatial resolution beyond the limits of conventional optical microscopy (Kenworthy Methods 24: 289-296 (2001); Gordon et al. Biophys J 74: 2702-2713 (1998)).

### (a) Förster Distance

The rate of energy transfer is inversely proportional to the sixth power of the distance between the donor and acceptor, thus, the energy transfer efficiency is extremely sensitive to distance changes. Energy transfer is said to occur with detectable efficiency in the 1-10 nm distance range. The distance at which energy transfer is 50% efficient (i.e., 50% of excited donors are deactivated by LRET) is defined by the Förster radius (Rₒ). The magnitude of Rₒ is dependent on the spectral properties of the donor and acceptor molecules and can be calculated from the spectral overlap integrals by using the equation:

Rₒ=[8.8×10²³∘κ²∘n⁻⁴QY_{D}∘J(λ)]^{1/6} Å

where κ²=dipole orientation factor (range 0 to 4; κ²=⅔ for randomly oriented donors and acceptors)

QY_{D}=luminescent quantum yield of the donor in the absence of the acceptor

n=refractive index

J(λ)=spectral overlap integral (see below)

=∫ε_{A}(λ)∘F_{D}(λ)∘λ⁴dλcm³M⁻¹

where ε_{A}=extinction coefficient of acceptor

F_{D}=luminescent molecule emission intensity of donor as a fraction of the total integrated intensity.

This distance is considered in selecting the locus for attachment of the luminescent labels. The loci are selected so that changes in distance between the loci are detectable as a change in the energy transfer. These distances can be empirically determined or can be calculated.

### (b) Donor/Acceptor Pairs

In most applications wherein energy transfer is detected, the donor and acceptor dyes are different, and energy transfer, such as FRET, is detected by the appearance of sensitized fluorescence of the acceptor or by quenching of donor fluorescence.

When the donor and acceptor are the same, FRET can be detected by the resulting fluorescence depolarization (Runnels et al. Biophys. J. 69: 1569-1583 (1995)). Extensive compilations of Rₒ values can be found in the art (Wu et al. Anal. Biochem. 218: 1-13 (1994); dos Remedios et al. J. Muscle Res. Cell Motil. 8: 97-117 (1987); Fairclough et al. Methods Enzymol. 48: 347-379 (1978)). Note that because the component factors of Rₒ (see above) are dependent on the environment, the actual value observed in a specific experimental situation is somewhat variable.

Again luminescent labels are selected so that the spectra overlap, and such that changes in distance between labeled loci can be detected as a change in energy transfer.

### (3) Luminescent Labels

Any luminescent labels, such as fluorophore donor and acceptor reagents can be selected by one of skill in the art. Exemplary labels include commercially available labels, and otherwise known labels, such as for example, those described in “Molecular Probes: Handbook of Fluorescent Probes and Research Chemicals”, Richard P. Haughlan, Molecular Probes Inc. If a desired reagent is not commercially available, the luminescent label or quencher can be prepared by laboratory methods, such as, for example synthesis, isolation, expression, and purification using methods well known in the art (see, e.g., Haugland, 1996 Handbook of Fluorescent Probes and Research Chemicals-Sixth Ed., Molecular Probes, Eugene, Oreg.; U.S. Pat. Nos. 5,800,996; 5,863,727; 5,625,048; 4,351,760 and 5,998,204; Miyawaki et al., Nature 388:882-887 (1997); Delagrave et al., Biotechnology 13:151-154 (1995); Pollok et al., Trends in Cell Biol. 9:57-60 (1999); Berlman, Handbook of Fluorescence Spectra of Aromatic Molecules, 2nd Edition (Academic Press, N.Y., 1971); Griffiths, Colour and Constitution of Organic Molecules (Academic Press, N.Y., 1976); Bishop, Ed., Indicators (Pergamon Press, Oxford, 1972); U.S. Pat. No. 3,996,345; Griffin et al., Science 281:269-272, 1998), Kendall et al., Trends in Biotechnology 16:216-224, 1998).

Luminescent molecules including, but not limited to, fluorophores and quenchers, include synthetically constructed organic compounds as well as naturally fluorescent polypeptide compounds such as, for example, Green Fluorescent Protein (GFP) and luciferase. As described herein, luminescent molecules, such as, for example, fluorophores and quenchers, can be used to label molecular and/or biological particle components of a target interaction, and, optionally, test compounds to detect target interactions and biological and/or chemical activity. For example, in the methods provided herein, more than one fluorophore can be used to label the molecular and/or biological particle components of the target, and candidate compounds described herein. Alternatively, at least two labels, such as two fluorophores, can be used to label one of the molecular and/or biological particle components of the target, at least 1 fluorophore can be used to label a second molecular and/or biological particle components of the target, and, optionally, at least 1 fluorophore can be used to label the candidate compound.

### (a) Fluorophores and Quenchers

Fluorophores include, but are not limited to, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM(tetramethylrhodamine, methyl ester), TMRE(tetramethylrhodamine, ethyl ester ), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.TM. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, and fluorescent europium and terbium complexes.

In the methods provided herein, an intercalator can be used as the luminescent molecule. Suitable intercalator binding ligands include, but are not limited to, furocoumarins and phenanthridines. For binding to DNA, aminomethyl psoralen, aminomethyl angelicin and aminoalkyl ethidium or methidium azides are useful. Although these compounds preferentially bind to double-stranded DNA, conditions can be employed to denature the DNA to avoid simultaneous interaction of these compounds with two strands. Exemplary binding ligands are “mono-adduct” forming compounds such as isopsoralen or other angelicin derivatives, such as 4′-aminomethyl, 4,5′-dimethyl angelicin, 4′-aminomethyl 4,5′,8-trimethyl psoralen, 3-carboxy-5- or 8-amino- or hydroxy-psoralen, as well as mono- or bis-azido aminoalkyl methidium or ethidium compounds. For examples of other photoreactive intercalators, see e.g., U.S. Pat. No. 4,734,454.

Quenchers that can be used in the methods provided herein include, but are not limited to, diarylrhodamine derivatives, such as the QSY 7, QSY 9, and QSY 21 dyes available from Molecular Probes; dabcyl and dabcyl succinimidyl ester; dabsyl and dabsyl succinimidyl ester; QSY 35 acetic acid succinimidyl ester; QSY 35 iodoacetamide and aliphatic methylamine; Black Hole Quencher dyes from Biosearch Technologies; napthalate; and Cy5Q and Cy7Q from Amersham Biosciences.

### (b) Bioluminescent Molecules

Naturally occurring bioluminescent generating reagents also can be used with the methods provided herein. Bioluminescent groups for use herein include luciferase/luciferin couples, including firefly (Photinus pyralis) luciferase, the Aequorin system (i.e., the purified jellyfish photoprotein, aequorin). Many luciferases and substrates have been studied and well-characterized and are commercially available (e.g., firefly luciferase is available from Sigma, St. Louis, Mo., and Boehringer Mannheim Biochemicals, Indianapolis, Ind.; recombinantly-produced firefly luciferase and other reagents based on this gene or for use with this protein are available from Promega Corporation, Madison, Wis.; the aequorin photoprotein luciferase from jellyfish and luciferase from Renilla, are commercially available from Sealite Sciences, Bogart, Ga.; coelenterazine, the naturally-occurring substrate for these luciferases, is available from Molecular Probes, Eugene, Oreg. Other bioluminescent systems include crustacean, such as Cyrpidina (Vargula) systems; insect bioluminescence-generating systems including fireflies, click beetles, and other insect systems; bacterial systems; dinoflagellate bioluminescence generating systems; systems from mollusks, such as Latia and Pholas; earthworms and other annelids; glow worms; marine polycheate worm systems; South American railway beetle; fish (i.e., those found in species of Aristostomias, such as A. scintillans (see, e.g., O'Day et al. (1974) Vision Res. 14:545-550), Pachystomias, and Malacosteus, such as M. niger; blue/green emitters include cyclothone, myctophids, hatchet fish (agyropelecus), vinciguerria, howella, florenciella, and Chauliodus); and fluorescent proteins, including green (i.e., GFPs, including those from Renilla and from Ptilosarcus), red and blue (i.e., BFPs, including those from Vibrio fischeri, Vibrio harveyi or Photobacterium phosphoreum) fluorescent proteins (including Renilla mulleri luciferase, Gaussia species luciferase and Pleuromamma species luciferase) and phycobiliproteins.

These groups can be attached to the molecular and/or biological particle components of the target as a portion of a fusion protein or via a linker. Formation of a fusion protein involves the placement of two separate genes, one encoding the protein of interest and the second encoding the luminescent protein, in sequential order in an appropriate cloning vector, with the stop codon of the first gene removed so that the polymerase continues through the first gene on to the second without disengaging from the template. Several commercial kits are available for the formation of fusion proteins which contain the protein of interest fused to a luminescent protein, including, but not limited to, Green Fluorescent Protein. For example, the GFP Fusion TOPO™ cloning vector and the pcDNA-DEST47 Gateway™ vector are available from Invitrogen (Carlsbad, Calif.) for the expression of a protein of interest fused to GFP. Further, custom designed and assembled genes, including those for fusion protein production, can be commercially ordered and prepared, such as by Sigma Genosys (The Woodlands, Tex.). Linkers can include affinity interactions, including, but not limited to, multimeric histidine tags and metal complexes, and biotin-avidin interactions.

### (c) Phosphorescent Molecules

Phosphorescent molecules also can be used with the methods provided herein. These groups can be purchased commercially, such as from Molecular Probes (Eugene, Oreg.) or synthetically produced as described above. Phosphorescent molecules include, but are not limited to, eosins and erythrosins, metal complexes containing a heavy metal (as a center metal) having a large spin-orbit interaction (e.g., Ru, Rh, Pd, Os, Ir Pt, Au, etc.), iridium complexes having a ligand, such as phenylpyridine or thienyl-pyridine; and platinum porphyrin derivatives.

### 3. Identifying Test Compounds and/or Conditions that Modulate Interactions among Biological Particles and Capture Systems or Secondary Effects of the Interactions

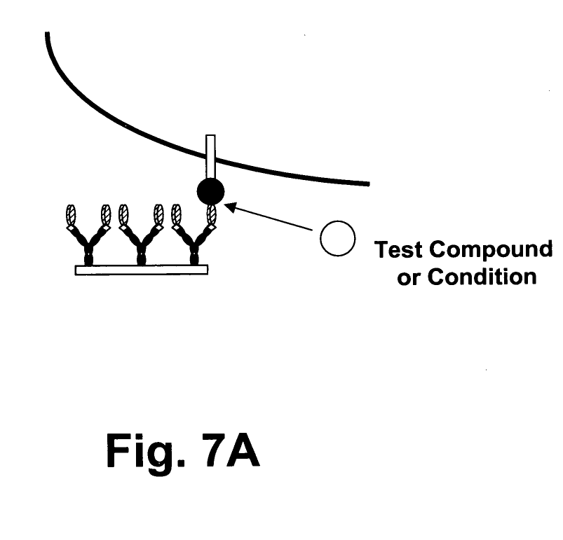

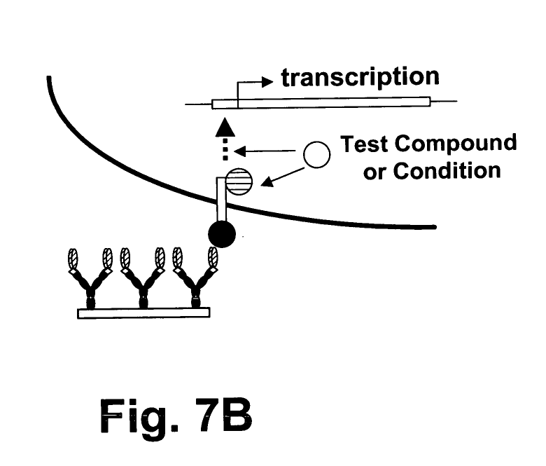

Methods using capture systems to immobilize biological particles are provided. In some embodiments, the biological particles, such as cells, are captured and a readout, i.e. stimulation of a particular pathway, expression of a reporter or other detectable event, is assessed. Alternatively, perturbations, such as test compounds or conditions, can be added or the cells exposed thereto and their effect on the interaction of the biological particle and the capture system or the effect of the interaction can be determined (FIGS. 7A and 7B). Perturbations include conditions and compounds that modulate interactions of molecules and/or biological particles. The perturbations can be conditions and test compounds that are known to modulate interactions; such perturbations are employed in methods in which the interaction is studied. Perturbations also can be conditions and test compounds whose effect is unknown. Such perturbations are identified using known interactions and effects of such interactions.

Conditions include environmental parameters which can be varied to determine the alteration of an interaction or the secondary effect resulting from an interaction, and include, but are not limited to, pH, ionic strength, aerobic versus anaerobic environment, temperature, pressure, time, concentration of components, agitation, and organic versus aqueous interaction medium. The alteration of environmental conditions can include varying one experimental parameter or multiple parameters simultaneously or sequentially.

Test compounds used in the methods provided herein include, but are not limited to, an organic compound, inorganic compound, metal complex, receptor, enzyme, antibody, protein, nucleic acid, peptide nucleic acid, DNA, RNA, polynucleotide, oligonucleotide, oligosaccharide, lipid, lipoprotein, amino acid, peptide, polypeptide, peptidomimetic, carbohydrate, cofactor, drug, prodrug, lectin, sugar, glycoprotein, biomolecule, macromolecule, biopolymer, polymer, sub-cellular structure, sub-cellular compartment or any combination, portion, salt, or derivative thereof.

The test compounds can be obtained from any source, including commercial sources (e.g. Maybridge Chemical Co. (Trevillet, Cornwall, UK), Comgenex (Princeton, N.J.), Brandon Associates (Merrimack, N.H.), and Microsource (New Milford, Conn.), Aldrich (Milwaukee, Wis.), Pan Laboratories (Bothell, Wash.) or MycoSearch (N.C.), synthetic production, collaborative exchange, compound libraries, expression, isolation, or purification techniques, or any other source known to those skilled in the art. Additionally, test compounds can be obtained from natural and synthetically-produced libraries that are readily modified through conventional chemical, physical, and biochemical methods and products. Test compounds can optionally be labelled, such as with a luminescent molecule, to facilitate detection of the interaction or the effect of the interaction using any methods known to those skilled in the art.

Test compounds and/or conditions identified or utilized by the methods described herein are molecules and/or biological particles that are screened against an interaction, to modulate and/or alter molecular interactions and chemical and/or biological activity. Test compounds and/or conditions can affect the interaction between the molecular and/or biological components of an interaction in a negative or positive fashion. As a non-limiting example, a test compound and/or condition can enhance an interaction between the molecular and/or biological components of a target by facilitating the interaction of the molecular and/or biological components of a target with one another. In contrast, a test compound and/or condition can reduce or inhibit a target interaction by preventing the molecular and/or biological components of a target from interacting with one another. Thus, test compounds and/or conditions can serve as, for example, activators, inhibitors, competitive inhibitors, agonists, partial antagonists, partial agonists, inverse agonists, antagonists, cytotoxic agents, and drugs for target interactions and chemical and/or biological activity that are studied.

If a particular interaction is implicated in diseases and/or disorders, a test compound and/or condition can have remedial, therapeutic, palliative, rehabilitative, preventative, prophylactic or disease-impeditive effects on patients suffering from, or potentially predisposed to developing, such diseases and disorders. Alternatively, screening test compounds or conditions against a target interaction can aid in the diagnosis and prognosis of patients suffering from such diseases and disorders. If a particular interaction is part of a biological mechanism or reaction, then a test compound or condition can serve as a modulator of that mechanism or activity. As a non-limiting example, screening test compounds or conditions with an interaction can aid in understanding a biological and/or chemical mechanism and/or activity.

### a. Perturbations and Screening Methods

Also provided are methods for screening for test compounds or conditions for modulatory effects on an interaction (FIG. 7A) or the secondary effect of an interaction (FIG. 7B). Test compounds and/or conditions are identified by contacting a test compound and/or condition with a capture system either prior to, simultaneously with or after exposure of a sample containing biological particles to the capture system and detecting a modulation of the interaction between the capture system and the biological particle or a secondary effect of the interaction. A change in the interaction or the secondary effect of the interaction in the presence of a test compound and/or condition compared to that in the absence of a test compound and/or condition indicates that the test compound or condition modulates the target interaction. Such test compounds and/or conditions are selected for further analyses or for use to modulate the interaction or the effect of the interaction, including, but not limited -to, as activators, inhibitors, competitive inhibitors, agonists, partial antagonists, partial agonists, inverse agonists, antagonists, cytotoxic agents, and drugs.

Optionally, the methods provided herein for screening test compounds and/or conditions as described above can be used to identify combinations of test compounds and/or conditions that, when exposed to the sample and capture system simultaneously or sequentially, result in an alteration in the interaction between the capture system and the biological particles or an alteration in particular effect of the interaction between the capture system and the biological particles, such as detection of an altered phenotype. Samples containing biological particles can be exposed to test compounds and/or conditions multiple times, such as before and after contacting a sample containing biological particles with a capture system. Multiple exposures can include the same test compounds and/or conditions or can vary, such as, for example, multiple varied test compounds, a combination of test compounds and conditions or multiple varied conditions. For example, a sample containing biological particles can be exposed to a test compound, such as an effector molecule. The exposed sample can then be contacted to a capture system; resulting in the interaction of biological particles within the exposed sample with the capture system. The capture system displaying the biological particles can then be contacted with a second identical or varied test compound, such as an additional effector molecule or a drug compound.

### b. Perturbations for Assessing Interactions or the Effect of the Interaction

Also provided are methods for assessing interactions between a capture system and biological particles by contacting a test compound and/or condition that has a known effect on a particular interaction (FIG. 7A) or on a particular effect of an interaction (FIG. 7B) prior to, simultaneously with or after exposing a sample containing biological particles to a capture system. Also provided are methods for assessing interactions between a capture system and biological particles by contacting single or combinations of test compounds and/or conditions that have a known effect on a particular interaction (FIG. 7A) or on a particular effect of an interaction (FIG. 7B) simultaneously or sequentially before and/or after exposing a sample containing biological particles to a capture system. A change in the interaction of the capture system and the biological particle or the effect of the interaction in the presence of the test compound(s) and/or condition(s) compared to that in the absence of the test compound(s) and/or condition(s) can indicate the type of interaction or the effect of the interaction within the system. In this type of screening, many targets can be screened against individual or combinations of known test compounds or conditions in order to pinpoint specific interactions. Optionally, once a particular target interaction or the effect of an interaction is identified, the interaction or effect of the interaction can then be screened as stated above for individual or combinations of test compounds or conditions that modulate the interaction or effect of the interaction.

### 4. Other Exemplary Applications

### a. Cell Surface Profiling

The cell membrane in eukaryotic and prokaryotic cells is a fluid phospholipid bilayer embedded with proteins and glycoproteins. The phospholipid bilayer is arranged so that the polar ends of the molecules form the outermost and innermost surface of.the membrane while the non-polar ends form the center of the membrane. In addition, it contains glycolipids as well as complex lipids called sterols, such as the cholesterol molecules found in animal cell membranes, that are not found in prokaryotic membranes. The sterols make the membrane less permeable to most biological molecules, help to stabilize the membrane, and probably add rigidity to the membranes aiding in the ability of eukaryotic cells lacking a cell wall to resist osmotic lysis. The proteins and glycoproteins in the cytoplasmic membrane are quite diverse and include, but are not limited to, channel proteins to form pores for the free transport of small molecules and ions across the membrane; carrier proteins for facilitated diffusion and active transport of molecules and ions across the membrane; cell recognition proteins that identify a particular cell; receptor proteins that bind specific molecules such as hormones, cytokines, and antibodies; and enzymatic proteins that catalyze specific chemical reactions.

Various cell types differ in the types and number of biomolecules present on the surface of the cell. This variation can be correlated to their function within the larger organism. For example, B cells function as antigen detectors and as a source of antibodies for the immune response within a system. The surface of a B cell typically displays over 100,000 identical molecules of a unique antibody that can function as B-cell receptors capable of binding specific epitopes of a corresponding shape. T cells help to eliminate pathogens that reside inside host cells. For this function, T cells display surface molecules such as CD4 and epitope receptors called T-cell receptors (TCRs). These receptors, in conjunction with the CD4 molecules have a shape capable of recognizing peptides from exogenous antigens bound to MHC-II molecules on the surface of antigen presenting cells and B cells.

The methods provided herein can be used to profile the surface of a cell. This profile can be used to identify the cell type and, possibly its function. For example, a sample containing B cells can be exposed to a library of tagged scFv molecules in a capture system. The interaction of the biological particles with the capture system can be used to identify the scFv molecules bound to the cells, and thus, the type of antibody present on the cell surface. Similarly, a sample containing antigen presenting cells can be exposed to a library of T cell receptors (TCRs) in a capture system and allowed to bind. The interaction of the APCs and the capture system can identify the antigenic species being displayed by the APC. In addition, test compounds and/or conditions can be identified which modulate the interaction between the biological particle and the capture system.

### b. Receptor Agonist/Antagonist Discovery

All hydrophilic molecules and the hydrophobic prostaglandins effect cellular responses via specific cell membrane receptors on the target cell. These protein receptors bind the signalling molecule with great affinity and transduce the signal into intracellular signals that affect cellular behavior. Cell surface receptors do not regulate gene expression directly, rather they relay a signal across the cell membrane and the response of the target cell depends on intracellular second messenger molecules such as cAMP, inositol phosphate, or calcium.

There are several families of cell surface receptors based on signal transduction mechanism. Channel-linked receptors are transmitter gated ion channels involved in rapid synaptic signalling as in nervous tissue or the neuromuscular junction. A specific transmitter can rapidly open or close ion channels upon binding to its receptor thus changing the ion permeability of the cell membrane. All of these receptors belong to a family of similar multipass transmembrane proteins. Catalytic receptors behave as enzymes when activated by a specific ligand. Most of these have a cytoplasmic catalytic region that behaves as a tyrosine kinase. Target proteins are phosphorylated at specific tyrosine residues thus changing their activation state. When bound to a specific ligand, G-protein linked receptors indirectly activate or inactivate a separate plasma membrane bound enzyme or ion channel. The interaction between the receptor and the affected enzyme or ion channel is mediated by a GTP binding protein. G-protein linked receptors initiate a cascade of chemical events within the target cell that usually alter the concentration of small intracellular messengers such as cAMP or inositol triphosphate. These intracellular messengers in turn alter the behavior of other intracellular proteins. The effects of all these second messengers are rapidly reversible when the extracellular signal is removed. The response of cells to external signals initiates signalling cascades that can greatly amplify and regulate various inputs.

The methods provided herein can be used to identify molecules that interact with a cell surface receptor. The interaction between the molecule and the receptor can be monitored either directly or indirectly by observing a secondary response. For example, a sample containing cells with G protein-linked receptors can be exposed to a library of tagged molecules in a capture system and allowed to interact. The interaction between the capture system and the G-protein cell surface receptor can be monitored directly through any method known to those skilled in the art or a secondary response to the interaction, such as, but not limited to, transcription of a gene, immunostaining of secondary messenger such as cAMP and detection of the stimulation of a secondary enzyme, such as a protein kinase. In addition, exogenous test compounds and/or conditions can be added to the capture system prior to, simultaneously with or after exposure of the biological particle to the capture system. Alteration in the interaction between the biological particle and the capture system and/or secondary effect of the interaction can be detected. This detection can result in the identification of test compounds and/or conditions that modulate the interaction between the biological particle and the capture system or the secondary effect of the interaction.

### c. Protein-Protein Interactions Including Association-Dissociation Assays and Changes in Protein Conformation

Interaction among proteins is responsible for many of the enzymatic reactions found in nature. Interactions include, but are not limited to electron transport from an electron source by a shuttle protein to an enzymatic protein for the conversion of reactants to products at the active site; chemical cleavage reactions, such as the formation of a mature protein from its zymogen; hetero- or homo-multimer formation for catalytic activity or complex stability; protective shuttling of toxic compounds from the source within the cell to the enzyme responsible for detoxification; chaperoning of metal or other cofactors within the cell for incorporation into an apoprotein; the post-translational modification, such as glycosylation or the hydroxylation of specific residues, of nascent polypeptides; and the more efficient folding of proteins following translation.

For example, the methods provided herein can be used to discover scFvs that bind to cell-surface receptors, whose activity in turn induces changes in protein conformation or in protein-protein interactions. Target cells can be any cell type which contains or possesses a naturally-occurring or engineered protein or proteins for which a conformation-specific readout exists (e.g., myosins) or for which an interaction-specific readout exists (e.g., BRET-based NF-κB/IkB interactions). Target cells are specifically bound to the capture system through interactions between cell-surface receptors and scFvs. By using a detection method, such as resonance energy transfer techniques, receptor-induced changes in protein conformation or protein-protein interactions can be assessed.

Renilla luciferase (Rluc) can be used as the donor protein and GFP can be used as the acceptor protein. In the presence of DeepBlueC, a cell permeable dye, Rluc emits light at 400 nm. If GFP is brought into close proximity to Rluc, the GFP will absorb the light energy and re-emit light at 510 nm. This system is used by Packard Biosystems and is referred to as BRET (Bioluminescence Resonance Energy Transfer). Other fluorescent protein pairs can be used. Fusion proteins can be made with a protein of interest using Rluc. Binding partners can be detected by making fusion proteins with GFP. GFP can be incorporated into a cDNA library to discover binding partners. Cells are then transfected with these constructs and exposed to the scFv library and binding /unbinding events can be detected using fluorescence as a read out.

### d. Biopolymer Degradation Assays

Biopolymers and small molecules often undergo chemical cleavage reactions as part of their respective synthesis and/or reaction mechanism. Most proteins undergo some means of proteolytic cleavage during post-translational modification. For example, many proteins, for example, proteolytic enzymes, are biosynthesized as larger, inactive precursors known as zymogens or proenzymes. An exemplary group, the serine proteases, are synthesized and stored in the pancreas as inactive precursors. Storage of these enzymes in their zymogenic form prevents damage to proteins in the pancreatic cells. After secretion from the pancreas into the small intestine, the zymogens are activated by selective proteolysis of one or a few select peptide bonds, resulting in the formation of the active form of the proteolytic enzymes. Similarly, many trans-membrane proteins or proteins that are destined to be secreted are synthesized with an N-terminal signal peptide. A signal recognition particle (SRP) binds a ribosome synthesizing a signal peptide to a receptor on the membrane and conducts the signal peptide and the following nascent polypeptide through it. Once the signal peptide has passed through the membrane, it is specifically cleaved from the nascent polypeptide by a signal peptidase.

For oligonucleotides, an example of chemical cleavage can be found in the processing of messenger RNA (mRNA). In eukaryotic systems, the formation of mRNA begins with the transcription of an entire structural gene, including its introns, to form pre-mRNA. Following capping and polyadenylation, the introns are excised and their flanking exons spliced together to yield the mature mRNA. A spliceosome, a large assembly of RNA and protein molecules, performs the pre-mRNA splicing. The spliceosome is a dynamic machine, which is assembled on the pre-mRNA from separate components and parts enter and leave it as the splicing reaction proceeds.

The methods provided herein can be used for monitoring chemical cleavage reactions of biopolymers. For example, RET-based systems can be used by tagging a single protein with two fluorescent probes. Cells can be transfected with this construct. When the protein is intact, the two fluorophores are in close proximity and a signal can be detected. When the protein is degraded, there is no signal. Once cells are transfected with this construct and exposed to the tagged library, molecules can be found which lead to the degradation of a specific protein of interest.

### e. Protein Trafficking Assays

The interior of the cell is organized into an array of membrane-bound compartments, each of which is composed of a specific set of resident proteins. The localization of integral membrane proteins to these compartments is, in many cases, mediated by short linear sequences of amino acids that function as specific sorting signals. The signals are recognized by receptor-like molecules that connect the signals to the sorting machinery. The methods provided herein can be used to define the molecular basis for protein biogenesis at specific sub-cellular locations, to elucidate the mechanisms responsible for intracellular protein transport and membrane fusion and to monitor the movement of proteins within a biological particle.

For example, to monitor movement (trafficking) of polypeptides within a biological particle, fusion proteins can be made with fluorescent tags such as GFP. Once cells are transfected, they can be exposed to a displayed library of molecules, such as signalling peptides and other extracellular signals, and molecules can be identified that lead to alternate localization of the protein of interest. In addition, proteins of unknown function can be tagged and tracked in a similar manner to determine their sub-cellular localization to gather some information leading towards a function determination.

### f. Analysis of Modulation of Subcelular Conditions and Processes

The cell is the basic unit of life and comprises a variety of subcellular compartments including, for example, the organelles. An organelle is a structural component of a cell that is physically separated, typically by one or more membranes, from other cellular components, and which carries out specialized cellular functions. Organelles and other subcellular compartments vary in terms of, inter alia, their composition and number in cells derived from different tissues, among normal and abnormal cells, and in cells derived from different species. Accordingly, organelles and other subcellular compartments, and macromolecules specifically associated therewith, represent targets for the development of agents that specifically impact, respectively, a particular tissue within an animal, abnormal (diseased) but not normal (healthy) cells, or cells from an undesired species but not cells from a desirable species. For example, members of the Bcl-2 family of proteins associate with the outer membranes of mitochondria and with other cellular membranes. Translocation of Bcl-2 proteins from one intracellular position to another occurs during apoptosis, a process by which some abnormal (e.g., pre-cancerous) cells are directed to undergo programmed cell death (PCD), thus eliminating their threat to their host organism. Methods for monitoring modulations in the accumulation of Bcl-2 proteins in various subcellular compartments, or their translocation from one intracellular location to another, can allow identification of agents designed to impact apoptosis, and to assay the effects of such agents in cells.

Provided herein are methods that can be used to monitor the modulation of the intracellular movement of the target as well as any simultaneous structural or chemical transformations that occur within the target as a result of or resulting in its translocation. For example, by selecting an appropriate set of luminescent labels, such as fluorophores, a subcellular compartment such as the mitochondria or a biomolecule such as Bcl-2 protein can labeled. The cells containing the labelled components are exposed to a capture system displaying tagged molecule that can interact with the biological particles. Modulations in the location of interaction on the membrane as well as the conformational adjustment on the protein or the membrane surface due to the interaction between the biological particle and the capture system can be assessed by detecting and monitoring FRET among the labels. Similarly, labeling a protein such as Bcl-2, which is transported intracellularly, the suspected source of the protein and the suspected final destination of the protein with luminescent labels, then monitoring changes in FRET among the labels on the three components in a time dependent manner can visualize any alterations in the location of the binding interactions and any conformational changes that occur as a result as well as give a timeline for the movement of the protein from its source to its destination.

### g. Assays for Assessing Cell Growth and Proliferation

Cells reproduce by duplicating their contents and dividing into two, separate entities. Coordinating cell proliferation, growth and differentiation is crucial for the development and survival of an organism. Cells divide only when they receive the proper signals from growth factors that circulate in the bloodstream or from a cell they directly contact. When a cell receives the message to divide, it goes through the cell cycle, which includes several phases for the division to be completed. To be affected by a growth factor, the target cell must have a receptor molecule, a membrane bound protein, for the growth factor. When the growth factor binds to its receptor, a series of enzymes inside the cell are activated, which in turn activates proteins called transcription factors inside the cell's nucleus. The activated transcription factors turn on genes required for cell growth and proliferation.

In some instances, a cell, such as a cancer cell, will grow out of control. Unlike normal cells, cancer cells ignore signals to stop dividing, to specialize, or to die and be shed. Growing in an uncontrollable manner and unable to recognize its own natural boundary, the cancer cells may spread to other areas of the body. In a cancerous cell, several genes mutate causing the cell to become defective. Abnormal cell division can occur either when active oncogenes, mutated normal genes, are turned on, or tumor suppressor genes are lost.

The methods provided herein can be used to identify molecules that modulate cell growth and proliferation. For example, a library of growth factors can be displayed by a capture system. A sample of cells can then be exposed to the capture system and the proliferation of the cells monitored, allowing identification of molecules that are involved in the regulation of cell growth. In addition, test compounds or conditions can be added to the capture system prior to, simultaneously with or after the sample is exposed to the capture system and alteration in cell proliferation can be monitored. Test compounds or conditions that increase or decrease cell proliferation can be identified.

### h. Assays for Assessing Apoptosis

Apoptosis, or programmed cell death, is a normal component of the development and health of multicellular organisms. Cells die in response to a variety of stimuli and during apoptosis they do so in a controlled, regulated fashion. This makes apoptosis distinct from another form of cell death called necrosis in which uncontrolled cell death leads to lysis of cells, inflammatory responses and, potentially, to serious health problems. Apoptosis, by contrast, is a process in which cells play an active role in their own death (which is why apoptosis is often referred to as cell suicide).

There are a number of mechanisms through which apoptosis can be induced in cells. The sensitivity of cells to any of these stimuli can vary depending on a number of factors such as the expression of pro- and anti-apoptotic proteins (e.g. the Bcl-2 proteins or the Inhibitor of Apoptosis Proteins), the severity of the stimulus and the stage of the cell cycle. In some cases the apoptotic stimuli comprise extrinsic signals such as the binding of death inducing ligands, such as CD95 (or Fas), TNFR1 (TNF receptor-1) and the TRAIL (TNF-related apoptosis inducing ligand) receptors DR4 and DR5, to cell surface receptors or the induction of apoptosis by cytotoxic T-lymphocytes by granzyme. The latter occurs when T-cells recognize damaged or virus infected cells and initiate apoptosis in order to prevent damaged cells from becoming neoplastic (cancerous) or virus-infected cells from spreading the infection. In other cases apoptosis is initiated following intrinsic signals that are produced following cellular stress. Cellular stress may occur from exposure to radiation or chemicals or to viral infection. It might also be a consequence of growth factor deprivation or oxidative stress. In general intrinsic signals initiate apoptosis via the involvement of the mitochondria. The relative ratios of the various bcl-2 proteins can often determine how much cellular stress is necessary to induce apoptosis.

Upon receiving specific signals instructing the cells to undergo apoptosis a number of distinctive biochemical and morphological changes occur in the cell. A family of proteins known as caspases are typically activated in the early stages of apoptosis. These proteins breakdown or cleave key cellular substrates that are required for normal cellular function including structural proteins in the cytoskeleton and nuclear proteins such as DNA repair enzymes. The caspases can also activate other degradative enzymes such as DNAses, which begin to cleave the DNA in the nucleus. The result of these biochemical changes is appearance of morphological changes in the cell.

The methods provided herein allow for detection of the modulation of cellular apoptosis resulting from the interaction of a biological particle with a capture system. Staining with stains specific for cell viability such as trypan blue or propidium iodide, can be used to determine cell viability after exposure to tagged molecules displayed by the capture system. Necrotic cells are detected by intense propidium iodide staining of the cytoplasm, due to the complete disruption of the plasma membrane. ApopNexin™ Kits (Serological Corp.) are also used to discriminate apoptotic from necrotic cells, and to label the progression of a cell through the various stages of apoptosis. As apoptosis progresses into the late-stage, the plasma membrane becomes permeable to DNA dyes such as propidium iodide, which enter the cell and stain yellow/orange.

In addition, other biomolecules involved in apoptosis, such as caspases, can be detected by using biomolecule specific substrates. Caspases are a family of proteins that are one of the main effectors of apoptosis. The caspases are a group of cysteine proteases that exist within the cell as inactive pro-forms or zymogens. These zymogens can be cleaved to form active enzymes following the induction of apoptosis. The production of these proteins from their zymogenic form is indicative of the advent of apoptosis and is therefore a target for detection.

For example, cell permeant caspase substrates such as PhiPhiLux^{R} (Oncolmmunin, Inc.); cell permeant caspase 3 and caspase 7 fluorogenic substrates from Molecular Probes; CaspSCREEN Apoptosis Detection Substrate (Chemicon); and CaspaTag™ Fluorescein Caspase Activity Kits (Serologicals Inc.) can all be used to monitor production and activity of the caspases. In addition, immunostains, such as anti-active caspase 3 monoclonal antibodies (BD Pharmingen), are also available for detection of apoptosis via the caspases.

In normal cells, most of the phosphatidylserine (PS) contained in the plasma membrane is oriented towards the cytoplasmic side of the cell membrane. In early stage apoptosis, the cell undergoes surface membrane blebbing, cytoplasmic shrinkage, nuclear DNA fragmentation, chromatin condensation and PS translocation across the plasma membrane to the exposed outer surface of the cell. It is thought that, the PS on the membrane surface identifies the cell as a target for destruction by the immune system. ApopNexin™ Apoptosis Detection Kits (Serological Corp.) exploit this biochemical event using the annexin V protein labeled with either FITC or biotin. Annexin V is a calcium-dependent phospholipid binding protein with a high affinity for PS. In the presence of calcium, annexin V binds rapidly and specifically to PS and is visualized by flow cytometry or microscopy.

Mitochondria have the ability to promote apoptosis through release of cytochrome C, which together with Apaf-1 and ATP forms a complex with pro-caspase 9, leading to activation of caspase 9 and the caspase cascade. Bax, and other Bcl-2 proteins, show structural similarities with pore-forming proteins. It has therefore been suggested that Bax can form a transmembrane pore across the outer mitochondrial membrane, leading to loss of membrane potential and efflux of cytochrome C and AIF (apoptosis inducing factor). Fluorescent probes of mitochondrial membrane potential, which drops in apoptotic cells, are available and include, MitoTracker Red, Rhodamine 123, and JC-1 (Molecular Probes); MitoLight (Chemicon); and the MitoTag™ JC-1 Assay Kit (Serologicals Corp.). Anti-cytochrome C monoclonal antibodies with a conjugated enzyme or fluorophore also can be used to detect apoptosis. Additional assays for apoptosis stages such as chromatin condensation and fragmentation, are readily available for microscopic detection of DNA fragmentation.

### i. Assays to Assess Changes in Cell Morphology

The methods provided herein can be used to sort biological particles, such as cells, onto capture systems and molecules can be identified that lead to alteration of the morphology of the cells. The biological particles can be contacted with a capture system and the captured biological particles, such as cells, can be observed, such as by light microscopy to identify changes in their physical characteristics, such as morphology. Alternatively, the biological particles, such as cells, can be labeled, such as with a luminescent label, and changes detected or identified by monitoring changes in luminescence.

To serve as an effective tracer of cell morphology, a fluorescent probe or other detectable molecule can have the capacity for localized introduction into a biological particle, as well as long-term retention within that structure. If used with live cells and tissues, the tracer can be biologically inert and nontoxic. When these conditions are satisfied, the fluorescence or other detectable properties of the tracer can be used to track the position of the tracer over time. A diverse selection of fluorescent tracers, as well as biotinylated, spin-labeled and other tracers are available commercially from Molecular Probes, and include, but are not limited to, cell-permeant cytoplasmic labels (CellTracker Blue CMAC, CellTracker Green CMFDA or CellTracker Orange CMTMR); microinjectable cytoplasmic labels (lucifer yellow CH, Cascade Blue hydrazide, the Alexa Fluor® hydrazides, sulforhodamine 101 and biocytin); membrane tracers (Dil, DiO, DiD, DiR, DiA, R18, FM 1-43, FM 4-64 and their analogs); fluorescent and biotinylated dextran conjugates, fluorescent microspheres (FluoSpheres and TransFluoSpheres fluorescent microspheres); and proteins and protein conjugates (Albumin Conjugates, Casein Conjugates, Peroxidase Conjugates, Phycobiliproteins, Fluorescent Histones, and Alexa Fluor 488 Soybean Trypsin Inhibitor). These tracers can be introduced into the biological particle using any method known to those skilled in the art including, but not limited to, microinjection, hypo-osmotic shock, scrape loading, sonication, high-velocity microprojectiles, glass beads, and electroporation (McNeil, P L Methods Cell Biol 29: 153-173 (1989)).

### j. mRNA Expression Change Assays

The methods provided herein can be used to monitor modulations in mRNA expression or real time PCR in biological particles cultured on the capture system for extended periods of time as a means to determine transcript profiling.

### k. Receptor Internalization Assays

The methods provided herein can be utilized to monitor the internalization of cell-surface receptors of biological particles exposed to the capture systems. For example, a receptor of interest is tagged with a marker that is either chemically conjugated (fluorochrome conjugated to the extracellular region) or genetically fused (GFP-receptor) and the cells expressing the receptor incubated with the tagged molecular library displayed on the capture system. After incubation, cells are fixed and the tag is visualized with a detection device to localize the receptor in intracellular compartments (Ghosh et al. (2000) Biotechniques 29(1): 170-175).

Many of fluorescent ligands available first bind to cell surface receptors, then are internalized and, in'some cases, recycled to the cell's surface. Consequently, it can be difficult to assess whether the fluorescent signal is emanating from the cell surface, the cell interior or, as is more typical, a combination of the two sites. Furthermore, the fluorophore's sensitivity to environmental factors, principally intracellular pH, can affect the signal of the fluorescent ligand. Molecular Probes has commercially available products by which these signals can be separated and, in some cases, quantitated. For example, antibodies directed to the Alexa Fluor® 488, BODIPY FL, fluorescein/Oregon Green, tetramethylrhodamine, Texas Red and Cascade Blue dyes to quench most of the fluorescence of surface-bound or exocytosed probes.

### l. Receptor-Mediated Cell Activation Assays

The methods provided herein can be used to monitor receptor-mediated cell activation resulting from the interaction of the biological particles with the capture system. For example, cells expressing a receptor of interest are incubated with the tagged molecular library displayed by the capture system and activation of cells assayed by staining cells for activation markers including but not limited to cytokines, receptors, cell adhesion molecules and transcription factors. Staining can be done using specific antibodies using standard methods.

### m. Receptor Activated Cell Signaling

The methods provided herein can be utilized to monitor or identify receptor activated cell signalling. For example, cells expressing a receptor of interest are transfected with reporter constructs that read out activation of transcription factors following a signal transduction cascade transmitting signal via intracellular proteins upon activation of receptor at cell surface. Exposure of this cell to the capture system following by monitoring of the transcription of the reporter gene identify molecules causing activation of surface receptors upon incubation of cells with a tagged molecular library.

### n. Epitope Mapping

The methods provided herein can be used to map epitopes for receptors displayed on the surface of cells. For example, a library of tagged T cell receptors (TCRs) are displayed by the capture system. The capture system is then exposed to T cells and the interaction among the cells and the capture system determined. The resulting interactions can be used to map T cell epitope specificity of naturally occurring peptides, or libraries of synthetic peptides, when presented in the context of major histocompatibility complex (MHC, class I or class II) on the surface of antigen presenting cells (APCs).

TCR libraries are tagged and expressed as recombinant proteins, in a manner similar to tagged scFv libraries exemplified herein, and arrayed as such. APCs are “pulsed” or otherwise induced to express peptide epitopes in the context of MHC, then sorted onto the array. Specific TCR-peptide MHC (pMHC) interactions bring APCs into contact with cognate, arrayed TCRs. The interactions between the APCs and the capture system allows for visualization of components within the system including, but not limited to, specifically bound APCs; various fluorescently labeled secondary stains; and various fluorescently labeled, engineered cell-specific proteins.

### o. Sorting Through Library Diversity and Cell Type Diversity

The methods provided herein can be used for sorting through molecular library and cell type diversity. For example, scFv libraries in solution are exposed to mixtures of cell types for the purpose of reducing unbound from bound scFvs, and to reduce cell-type diversity.

Cell mixtures can be produced from mixed-cell cultures, or from multiple tissues. Magnetic beads can be used as a first-pass physical separation. First, capture Ab-coated magnetic bead sets are generated. Target cells are pre-incubated with tagged scFv sub-libraries. Capture Ab-coated beads are then incubated with the scFv-coated target cells. The only cells which bind to the beads are those cells which were specifically bound by a tagged scFv. Next, magnetically separate the beads with bound cells from all unbound cells and unbound scFvs. Any of the beads with cells specifically bound will come down with the bound cells. Everything else will stay in suspension. Separation of tagged scFv-bound cells from the capture Ab-coated beads can be performed by competition with free Tag peptide in a small volume, followed by dilution into a large volume. The resulting cell fraction can be loaded onto capture systems than contain polypeptide-tagged capture Abs. The tagged scFv-bound cells sort to the correct capture Abs. Sorting of the cells in this manner allows for monitoring of, for example, changes in cellular morphology; cell type-specific secondary stains; and various fluorescently labeled, engineered cell-specific proteins. Optionally, optically coded beads (such as those available from Kodak) can be substituted for the magnetic beads. After a wash step, the beads are contacted with the captured cells on the surface, and the resulting system is visualized as above.

### p. Expression of Secreted Polypeptides by Tumor Cells

The methods provided herein can be utilized to discover or identify tumor or other cell-surface receptors which trigger expression of secreted proteins, e.g., B7-H1, which in turn induce apoptosis or other forms of cell death in secondary target cells (Nat Med 8(8): 793-800 (2002)). Primary target cells are tumor cells, of any relevant type, specifically bound to the capture system through interactions between cell-surface receptors and the tagged molecular library. Secondary target cells are HLA-matched T cells (cytotoxic CD8+T cells, CTLs) with TCR specificity for tumor cell-surface pMHC. Specific pMHC-TCR interactions will bring CTL into contact with array-bound tumor cells. CTLs will then lyse and kill bound tumor cells unless tumor cells have been activated to express molecules, e.g., B7-H1, which interact with one or more CTL-surface receptors, in turn inducing apoptosis. The methods provided herein can be used to initially monitor specific interaction of the CTLs to the capture system bound tumor cells. The methods also can be used to detect apoptotic death of CTLs as measured by, for example, biochemical dye staining for mitochondrial membrane changes and DNA fragmentation.

### q. Differentiation/Dedifferentiation Assays

The methods provided herein can be used to discover or identify cell-surface receptors which, when bound to a specific ligand on-array, induce differentiation or de-differentiation. Target cell sources are relevant cell types of choice, such as those that possess a specific, differentiation-stage-specific morphology and/or cell-surface marker which is either up-regulated or down-regulated in a stage-specific manner. Target cells are specifically bound to the capture systems through interactions between cell-surface receptors and the tagged molecular library. Once bound to the capture system, changes, such as, in differentiation state-specific morphology; an increase/decrease or loss/gain of cell-surface-expressed, differentiation stage-specific marker (revealed via binding of fluorescently labeled secondary Ab or other ligand) can be monitored.

### r. Cell-Cell Interactions

The methods provided herein can be utilized to identify antibodies which alter interactions between cells, including, but not limited to, immune cells, neutrophils, endothelial cells, and epithelial cells. The first cell type is captured on the capture system, following by addition of the second cell type and determination if binding occurs between the two cell types. In addition, altered function as a result of contact between the cells also can be followed using any of the detection methods known to those skilled in the art and described herein.

Further, using the methods provided herein, molecules can be discovered, which bind to cell-surface receptors, whose activity in turn induces or inhibits interaction of primary, array-bound target cells with secondary target cells. Primary target cells can be any cell type which is known to interact with a secondary target cell type (e.g., APCs and T cells) or which are previously not known to interact with a secondary target cell type. Target cells are specifically bound to the capture system through interactions between cell-surface receptors and a tagged molecular library. Secondary target cells are then exposed to the primary target cells captured on the capture system and allowed to specifically bind. The readout of the system can visualize, for example, specifically bound primary and secondary target cell binary complexes; various fluorescently labeled secondary stains which confirm and differentiate between bound primary and secondary target cells; and various fluorescently labeled, engineered secondary target cell-specific proteins.

### s. Discover Molecules that Block Binding/Cleavage/Post-Translational Modifications

The interaction of an exogenous molecule with a molecule on the surface of a biological particle can result in numerous functions including, but not limited to, the blockage of binding either on the surface or intracellularly, the generation of a signal for the cleavage of a second surface molecule, the generation of a signal for the post-translational modification of a second molecule, binding to a known molecule, such as, but not limited to, a protein, polypeptide, DNA, lipid, carbohydrate, and organic molecule; and enzymatic activity such as proteolysis, phosphorylation, methylation, acylation and phenylation. Detection methods, such as immunostaining, detection of the transcription of reporter genes and resonance energy transfer, can be used to monitor these functions.

For example, cleavage of surface proteins, termed protein shedding, is the proteolytic release of a cell surface protein. This shedding can serve a regulatory role by liberating soluble molecules into circulation while decreasing their concentration on the cell surface (Hooper et al. Biochem. J. 321: 265-279 (1997)). Proteins that are shed from the cell surface include, but are not limited to, growth factors, cytokine receptors, cell adhesion molecules and leukocyte receptors. Shedding of cell surface molecules is initiated by interaction between a ligand and cell-surface receptor, which results in the recruitment of a soluble proteinase that cleaves the surface protein. For example, L-selectin, a member of a family of adhesion molecules, is constitutively expressed on the surface of circulating leukocytes. The soluble, active form is released from the surface by proteolytic cleavage following cell activation.

Post-translational modification of molecules can, for example, result in the activation of a proenzyme or the formation of the final molecular product, such as conversion of a molecule from its precursor form to its mature form or a secondary form. For example, the amyloid beta (Aβ) peptide, a 40 or 42 amino acid residue peptide, has been implicated in the pathology of Alzheimer's disease. This peptide is generated from the post-translational processing of the amyloid-β precursor protein (APP) through initial cleavage by β-secretase followed by cleavage by γ-secretase. Alternatively, APP can be processed by α-secretase, which cleaves at a varied site from the β-secretase yielding a final 23 amino acid residue peptide fragment following cleavage by the γ-secretase. This smaller peptide is not believed to contribute to the Alzheimer's Disease pathology (Selkoe D. J. in The Molecular and Genetic Basis of Neurological Disease (Rosenberg et al., Eds.) pp. 601-612, Butterworth-Heinemann, Boston). The regulation of these two post-translational processing pathways can provide potential drug candidates for the regulation of amyloid-β production and Alzheimer's Disease.

The methods provided herein can be used to identify molecules and conditions that modulate the blockage of binding either on the surface or intracellularly, the generation of a signal for the cleavage of a second surface molecule or the generation of a signal for the post-translational modification of a second molecule. For example, a library of molecules can be displayed on a capture system. Biological particles containing the amyloid-β precursor protein can be exposed to the capture system. The formation of the 23 amino acid post-translational product can be monitored, such as by resonance energy transfer. Biological particles showing the formation of the 23 amino acid post-translational product can be identified and the molecule interacting with the biological particle selected for further study in its effect on the regulation of the formation of the 23 amino acid post-translational product of the amyloid-β precursor protein.

In another embodiment, a library of molecules can be displayed by a capture system. Biological particles can then be exposed to the capture system and allowed to bind in the presence of a specific proteinase, such as a metalloproteinase. The capture system can then be specifically stained for a soluble surface protein thought to be cleaved by the proteinase in the presence of a transduced signal. Those loci that show a positive reaction with the stain indicate those biological particles where a signal due to the interaction of the biological particle with the capture system has been transduced, thereby allowing identification of molecules that modulate the cleavage of molecules on the surface of the biological particles.

### t. Simultaneous Capture of Multiple Cell Types Followed by Functional Assays for Drug Interactions

The methods provided herein can be used to identify cell type specific antibodies. Once identified, these antibodies can be displayed in the capture system in order to sort different cell types from a mixture to specific addresses on a capture system. Once captured by the capture system, the different cells can be simultaneously screened for a drug response.

### i. Organ Cultures (e.g. Promotion of Hair Growth)

The methods provided herein can be used to identify molecules such as functional antibodies and cell type specific antibodies, for cells within a multicellular context. For example hair follicles and sweat glands can be teased out of skin and cultured, then exposed to a capture system displaying a library of scFv molecules. Early-stage embryos are another target for the capture systems.

The methods provided herein also can be used to culture high-precision organ slices on the capture systems. These slices are used for screening of drugs in pharmacology and for studying the potential toxicity of test compounds. These methods are similar to those above except that this method is directed to exposing cells to a capture system in the context of a tissue sample rather than a cellular sample for identification of functional antibodies.

### v. Discovery of Antibodies to Apically-Localized Cell-Surface Proteins, Carbohydrates and Lipids

The methods provided herein can be used to identify antibodies to apically-localized cell-surface proteins, carbohydrates and lipids. For example, epithelial mono-layers can be grown in culture. The tagged molecular libraries described herein can be sorted and stuck to the surface of beads that were coated with a single capture antibody/bead. These coated beads can then be applied to the apical cell surface. After washing, those beads that still stick to the cell surface indicate which tagged molecules should be further investigated. This procedure, optionally, can be carried out in a 96 well format, with only one species of beads (containing only one specific tag) used per well. This option eliminates a need for bead encoding.

### w. Infectious Agents on Arrays

The methods provided herein can be used to identify molecules, such as antibodies, that bind specifically to the surfaces of infectious agents including, but not limited to bacteria, yeast, fungi, protozoans and other microscopic parasites, viruses and prions. The identified molecules are then screened for functional consequences (e.g., cytotoxicity, mammalian cell binding) on the organism/particle of interest.

### x. Monitoring of Endocytosis, Exocytosis and Phagocytosis

The plasma membrane defines the inside and outside of the cell. It not only encloses the cytosol to maintain the intracellular environment but also serves as a formidable barrier to the extracellular environment. Because cells require input from their surroundings—in the form of hydrated ions, small polar molecules, large biomolecules and even other cells—they have developed strategies for overcoming this barrier. Many of these mechanisms involve initial formation of receptor-ligand complexes, often followed by transport of the ligand across the cell's membrane.

Provided herein are methods for the detection and monitoring of the interactions among lipids. For example, by selecting the appropriate set of labels, such as luminescent labels, two lipid molecules can be labeled in such a manner that in their native state, energy transfer, such as FRET, is observed. An enzyme, such as a flippase, can similarly be labeled, such as with a luminescent label, and contact the labelled lipid molecules. Binding of the enzyme in proximity of the labelled lipids can allow the monitoring of both binding interactions as well as the movement of the lipid molecules as the result of the flippase activity. In another example, the three label FRET assay can be used to monitor movement of polypeptides and small molecules through lipid bilayers.

### y. Internalization of Libraries by Cultured Cells

In addition, our libraries, displayed on fluorescent beads, can be tested for internalization by cultured cells.

### z. Detection of Phosphorylation and Dephosphorylation Activities

Eukaryotes employ phosphorylation and dephosphorylation of specific proteins to regulate many cellular processes (Hunter Cell 80:225-236 (1995); Karin Curr. Opin. Cell Biol. 3: 467-473 (1991)). These processes include signal transduction, cell division, and initiation of gene transcription. Thus, significant events in an organism's maintenance, adaptation, and susceptibility to disease are controlled by protein phosphorylation and dephosphorylation. These phenomena are so extensive that it has been estimated that humans have around 2,000 protein kinase genes and 1,000 protein phosphatase genes (Hunter Cell 80: 225-236 (1995)), some of these likely coding for disease susceptibility. For these reasons, protein kinases and phosphatases are prospective targets for the development of drug therapies.

Provided herein are methods for the detection and monitoring of alterations in the dephosphorylation and phosphorylation reactions within a biological particle. For example, the appropriate set of luminescent labels, such as fluorophores, can be attached to the molecule being phosphorylated (or dephosphorylated) and/or the enzyme responsible for the activity. These molecules can be transfected into the biological particles. The biological particles can then be exposed to a capture system displaying tagged molecules. Monitoring of FRET among labels can yield information about the effect of the interaction between the biological particle and the tagged molecule on the interaction between the enzyme and its substrate, and the rate of the phosphorylation (or dephosphorylation) reaction. Additionally, the additional effect that any added test compounds or conditions have on the native reaction can be monitored.

### aa. Determination and Monitoring of Chemical or Enzymatic Kinetics

Chemical reactions proceed at a certain rate dependent on the components of the reaction and the environment in which the reaction occurs. Measurement of these rates often yields valuable information regarding the mechanism of the reaction, and the resulting formation of products. Kinetic rates can be determined for catalytic reactions between an enzyme and its substrate including, but not limited to, for conversion of a protein from one conformational state to another, for formation of multimers from individual components and for the translocation of an electron.

Provided herein are methods for the determination and monitoring alterations of kinetic rates of chemical reactions. For example, the target reaction can comprise an-enzyme, whose activity is regulated by cell-surface signalling. Attachment of the appropriate set of luminescent labels, such as fluorophores, to the enzyme as well as its substrate in optimal positions permits study of the interaction between the molecules while simultaneously determining the rate of product formation by monitoring resonance energy transfer among the labels. The transfection of these molecules into the cell followed by exposure of the cell to a capture system displaying tagged molecules can yield information about the effect of the interaction between the cell and the tagged molecule of the capture system on the target reaction. Additionally, these methods can be used to monitor changes in the rate of the formation and decomposition of reactive intermediates, either chemical or conformational, which are difficult to isolate using standard spectroscopic or isolation techniques. Further, these methods can be used to monitor alterations in the binding of an electron transfer protein to its enzymatic binding partner and the resulting enzymatic reaction that converts substrate to products. The rate at which the electron is transferred from the transport protein to the active site of the enzyme can be measured by placing fluorophores at the distant sites and monitoring changes in the FRET as a result of conformational or chemical changes as electron transfer and catalysis occurs.

### H. Identification of Binding Partner Polypeptides

Any method for identifying or selecting binding partner polypeptides specific for particular capture agents can be employed. A variety are described herein and are known to those of skill in the art. Also provided herein is a method for designing polypeptide binding partners that are highly antigenic and that induce, upon administration to a host, antibodies that are specific for the polypeptides or other for screening antibody and single chain antibody or other libraries. Monoclonal antibodies and fragments thereof can be produced from the antibodies or the selected single chains or other binding agents identified from libraries are used as capture agents that are paired with the designed or generated polypeptide.

### 1. Overview of the Methods

The methods provided herein start with a set of amino acids, which typically includes some or all of the naturally-occurring amino acids and also can include selected non-naturally occurring amino acids. For exemplification, the naturally occurring 20 amino acids are included. In addition, the polypeptide that is to be designed can be any length, typically is short, at least two amino acids up to 50, but generally is 4, 5, 6, 7, 8, 9, 10, 12, 16, 20 or more. For exemplification, the polypeptides are 6 amino acids in length and contain 4 critical residues. The exemplary initial analysis is performed for 4-mers that contain any of the 20 naturally-occurring amino acids. The host for which antigenicity is targeted is mice. Accordingly, there are 20⁴ combinations possible. The methods herein provide a way to select highly antigenic specific binding polypeptides from among these combinations of amino acids. The members of the set of possible polypeptides are selected by imposing criteria based upon empirical data regarding antigenicity in a particular host and also upon properties of particular amino acids. The method for selecting polypeptides can be performed manually or by using or developing a program to impose the criteria. An exemplary process is described herein. A polypeptide of 6 amino acids in length and 4 critical residues is selected for exemplification herein.

Step 1: Select length of polypeptide and critical residue number. For exemplification a length of 6 is selected with 4 critical residues.

Step 2: Generate all combinations of 4 residues using 10 amino acids such that there are no duplications of amino acids in any polypeptide. The ten amino acids were selected based upon antigenicity ranking (see table herein and cited references for the amino acids that occur most often in antigenic polypeptides) that had been empirically determined. The resulting set contained 5040 members.

Step 3: Using the similarity table (described herein), arbitrarily select one polypeptide. Using the selected polypeptide, pick a set of predetermined number of members. These polypeptides are selected to contain a sequence of amino acids that is as dissimilar as possible from the other members in the final selected set. This is done using the similarity table to create an indexing number, a similarity score, representative of the dissimilarity. This is done by combining the numbers from the table for each amino acid in a particular polypeptide compared to the reference polypeptide to create a score for each of the 30,240 polypeptides and the selecting a predetermined number by setting a threshold similarity index.

Step 4: Since 4 residues are selected from the total selected length of 6 (step 3), the remaining 2 residues, designated “non-critical” are assigned. For exemplary purposes, the 2 non-critical residues are assigned adjacent positions and only critical residues occupy the N-terminal and C-terminal positions, thereby generating the possible 6-mers into which non-critical residues are placed. For naturally occurring amino acids, non-critical residues are those that can be replaced with more than 10 amino acids and retain the specific binding properties of resulting polypeptide. These non-critical residues are known (see, description here and publications cited) and can be empirically determined. For exemplification two possible combinations of non-critical residues were selected. These were Tyr-Gly, and Ser-Gly. These were chosen herein since they confer solubility and permit hairpin folding which is advantageous for generating capture agents/binding partners for the methods and products herein.

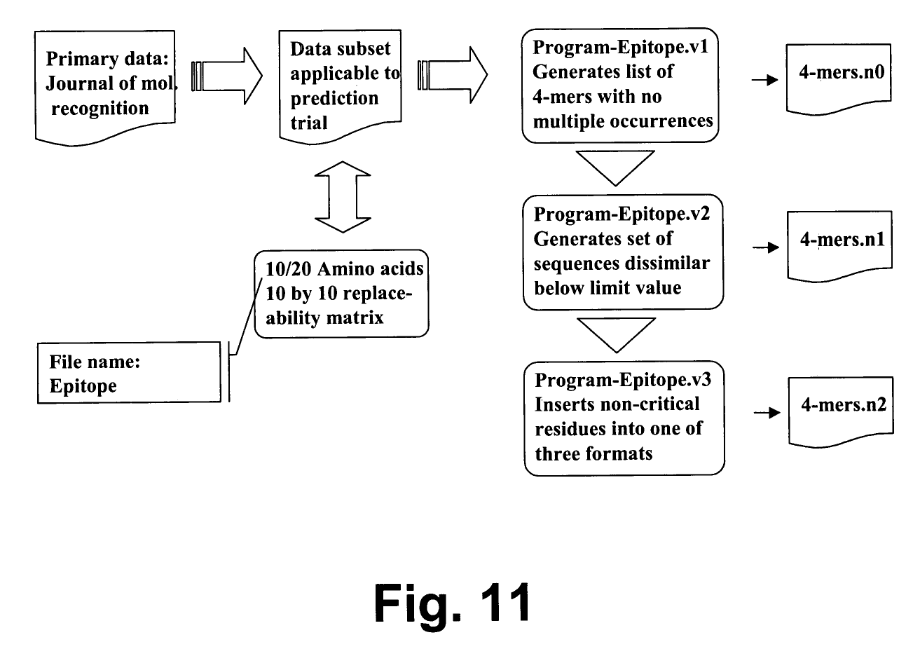

An exemplary process to carry out the steps as described is shown in FIG. 11. The final exemplary set chosen is provided herein (see discussion and Sequence Listing). As shown in the Examples, all tested polypeptides resulted in antibodies useful as capture agents specific for the 6-mer polypeptides. Thus, this method permits design of polypeptides that predictably induce production of specific antibodies upon administration, thereby providing highly specific capture agent/tag (binding polypeptides) pairs for use in the methods and products provided herein.

### 2. Description of the Methods

Provided herein are methods for obtaining highly specific, highly antigenic (HAHS) polypeptides for use as partners with capture agents (binding proteins) such as antibodies. The polypeptides contain any number of amino acids against which a specific capture agent (binding protein) can be generated or synthesized to bind. Typically such polypeptides are at least 2, 3, 4, 5, 6 to about 100 amino acids in length, usually between 2-50, 2-40, 2-30, 2-20, 4-20, 5-20, 2-50, 4-50, 5-50, and 6-20 amino acids in length. Also provided are methods for generating the binding proteins (capture agents), such as antibodies, which bind to HAHS polypeptides. Thus, methods generate pairs of HAHS polypeptides and capture agents. There is no detectable cross-reactivity, such as by ELISA assay, between or among different pairs of HAHS polypeptides and capture agents.

The method of designing highly antigenic, highly specific polypeptides constructs or designs polypeptides that contain sequences of amino acids that are antigenic (i.e., they are more likely to be antigenic than a randomly selected or generated polypeptide of the same or similar size). These polypeptides are more likely to raise an immune response in a subject and/or bind antibodies or a portion thereof with a high affinity and specificity than a randomly selected polypeptide.

The methods provided herein, which are described in detail below, use statistical probabilities that a particular amino acid appears in an antigenic polypeptide. These statistical probabilities can be generated empirically or calculated. Statistical probabilities for naturally occurring amino acids are exemplified herein. The same or similar methods can be applied to any sets of amino acids including non-naturally occurring amino acids and analogs thereof.

For example, sequences of antigenic polypeptides can be obtained by empirical methods, such as by injecting mice with polypeptides representing all the possibilities of a set length of polypeptides. The polypeptides are injected into mice and antisera is collected. The antisera then is tested on collections of polypeptides and the antigenic polypeptides are identified based on their reactivity with the antisera. Non-antigenic polypeptides are identified by their lack of reactivity with the antisera. The frequency of an amino acid appearing in a polypeptide that is antigenic is used to determine which amino acids are more likely to be found in an antigenic polypeptide.

The number of polypeptides possible for all sequence combinations is high. For example, a 4 mer has 20×20×20×20 possibilities (160,000 total). It is time consuming, costly and undesirable to test each and every polypeptide to determine its antigenicity. The methods described herein obviate the need for such tedious testings. The methods use a statistical prediction based on the frequency of an amino acid appearing in a polypeptide that is antigenic. The likelihood that an amino acid appears in a polypeptide that is antigenic can be determined based on a representative set of data, for example, based on immunizing animals with a representative subset of all the possibilities of that polypeptide length. Based on the subset of polypeptides injected which are antigenic and non-antigenic, amino acids are identified that either are more likely to be present in antigenic polypeptides or are more likely to be present on non-antigenic polypeptides. The likelihood of a amino acid's presence in an antigenic polypeptide gives an observed antigenic ranking. Using polypeptides of the 20 naturally occurring amino acids, a ranking of antigenicity for each amino acid can be obtained. Similarly, an antigenic ranking of amino acids also can be obtained by mapping epitopes in known proteins. Antibodies to known proteins are used to determine the sequence of amino acids to which they bind, for example by deletion or replacement mutagenesis or by synthesizing subsets of amino acid sequence found within the protein sequence. The antibodies are tested for reactivity with the mutants or with subsets of peptide sequences from the protein. The shortest sequence of amino acids from the protein which retains binding to the antibody defines the epitope. Epitope mapping can be performed with a representative number of proteins and antibodies and the statistical occurrence of each of the 20 amino acids found in the epitopes is determined to generate the antigenic ranking of the amino acids (see, e.g., Geysen et al., (1988). J. Molecular Recognition 1:32-41; Getzoff et al., (1988). The Chemistry and Mechanism of Antibody Binding to Protein Antigens. Academic Press. Advances in Immunology. Vol 43:1-98). Epitope mapping and antigenic ranking such as with known proteins or by injecting collections of random polypeptides can be done-in any species of interest that raises an immune response, for ; example mice, rabbit, rat, human, monkey, dog, chicken, and goat. -For example, using data obtained from epitope mapping (Geysen et al., (1988). J. Molecular Recognition 1:32-41), the amino acids were assigned the following antigenic rankings, with 1 being the highest and 20 the lowest probability (Table 5).

TABLE 5

| Ranking | amino acid |
|---|---|
| 1 | E |
| 2 | P |
| 3 | Q |
| 4 | N |
| 5 | F |
| 6 | H |
| 7 | T |
| 8 | K |
| 9 | L |
| 10 | D |
| 11 | V |
| 12 | I |
| 13 | G |
| 14 | Y |
| 15 | S |
| 16 | C |
| 17 | A |
| 18 | M |
| 19 | R |
| 20 | W |

Epitope mapping and antigenic ranking can also be performed using recombinant means, by screening libraries of antibodies or antibody fragments with polypeptides containing sequences of epitopes, such as collections of sequences of critical amino acids. The polypeptides which are bound by the antibodies can be sequenced and the frequency of the amino acids appearing in polypeptides bound by the antibodies can be determined. Experimental conditions such as washing conditions in a phage library panning assay can be used to control the affinity of the interaction between the antibodies and the peptides.

For a given length of polypeptides, amino acids are selected from the antigenic ranking list. Polypeptides can be any length sufficient for an antibody epitope, generally less than 20 amino acids. For example, the polypeptides length is between 2 and 20 amino acids, such as 2, 3, 4, 5, 6, 7, 8, 9, 10, 11, 12, 13, 14, 15, 16, 17, 18, 19 and 20 amino acids in length. In one exemplary embodiment, 4mers are selected using the antigenic ranking list of amino acids.

A threshold ranking of antigenicity can be chosen to limit the possible number of polypeptides in the subset (subset A) and to bias the subset to more antigenic sequences. For example, if the polypeptide length is 20 amino acids, each of the 20 positions can be selected from the top 19 antigenic ranking amino acids, limiting the subset from the total possibilities of all 20 amino acids at each position. The threshold can be set according to the number of polypeptides desired in the subset and the level of dissimilarity chosen for the subset. In one embodiment, the amino acids are chosen from the top n-1 antigenic ranking amino acids, where n is the total amino acids in the polypeptide length. In one aspect of the embodiment, the top 19, 18, 17, 16, 15, 14, 13, 12, 11, 10, 9, 8, 7, 6, or 5 antigenic ranking amino acids are used to design and construct the polypeptide sequences. In one exemplary embodiment, the top 10 antigenic ranking amino acids are used to design and construct polypeptide sequences. In another exemplary embodiment, the amino acids E, P, Q, N, F, H, T, K, L, and D are used to design and construct polypeptide sequences.

In a given length of polypeptides, to further bias the specificity of the polypeptides and reduce potential cross reactivity between binding proteins and polypeptides outside the partner pairs, each amino acid in the length can be unique. This further reduces the number of polypeptides in the subset (subset B). For example, if the polypeptide is a 4 mer and 10 amino acids are chosen from the antigenic ranking list, the number of possibilities in 10×9×8×7, where each amino acid is unique within a 4-mer (i.e., there is no duplication or any multiples of a chosen amino acid within the polypeptide length). Thus, for a 4 mer there are 5040 possibilities in this subset B.

Subset B represents the list of antigenic polypeptide possibilities for the chosen length of polypeptide. Optionally, these polypeptides can be incorporated in larger polypeptides, such that the polypeptides derived from subset B are designated the critical residues in the polypeptide, composed of antigenic amino acids and the remaining positions in the polypeptide length are noncritical positions (subset C). The length of such polypeptides can be generally less than 50 amino acids, typically less than 20 amino acids. For example, the polypeptides length can be between 2 and 20 amino acids, such as 2, 3, 4, 5, 6, 7, 8, 9, 10, 11, 12, 13, 14, 15, 16, 17, 18, 19 and 20 amino acids in length. The number of critical residues is larger than the number of non-critical residues. Generally, for peptides of 9 or less amino acids, the number of critical residues is approximately 55%, 60%, 70%, 80%, 85%, 90% or 95% of the total number of amino acids in the polypeptide.

The non-critical positions can be any amino acid. The non-critical positions can also be utilized to introduce added functionalities into the polypeptide, such an solubility and folding. In one exemplary embodiment, amino acids which increase solubility and permit flexibility and folding are used at the non-critical positions. For example, the amino acids S, G and Y are utilized at the non-critical positions.

The non-critical positions can be designated at specific sites within the polypeptide length to construct subset D. For example, it can be designated that the N and C terminal residues of the polypeptide are critical residues. In another example, it can be designated that the non-critical residues are found in pairs. In one exemplary embodiment 6 mer polypeptides are designed whereby the first and last (N and C terminal) positions are critical residues and 2 additional positions of the remaining 4 residues of the 6-mer are also critical residues chosen from a set of antigenic amino acids. The remaining 2 positions are non-critical residues and are designated to be in adjacent positions in the 6 mer.

In the above example, with 6 mers, 5040×3 (15120) possible polypeptides are generated for subset D as follows:

XNNXXX

XXNNXX

XXXNNX

where X's are critical residues and N's are non-critical residues and the 3 polypeptides show the possible arrangement to generate adjacent non-critical residues and polypeptides with critical residues at the ends.

Subset D can then be further restricted to generate a set of polypeptides that are dissimilar from each other, subset E. To extract a subset E, a single polypeptide is chosen at random from subset D as the first, reference polypeptide. A similarity ranking is calculated for all of the polypeptides in subset D using a replaceability matrix which compares the similarity of the amino acids at the critical positions to each other. An example of a similarity matrix is given in Table 6:

TABLE 6
Similarity Matrix

| | E | P | Q | N | F | H | T | K | L | D | G | S | Y |
|---|---|---|---|---|---|---|---|---|---|---|---|---|---|
| E | 100 | 13 | 33 | 13 | 2 | 8 | 10 | 6 | 8 | 42 | 13 | 15 | 6 |
| P | 5 | 100 | 16 | 11 | 8 | 11 | 11 | 16 | 3 | 3 | 14 | 14 | 0 |
| Q | 15 | 10 | 100 | 25 | 5 | 10 | 10 | 5 | 5 | 5 | 20 | 15 | 10 |
| N | 4 | 0 | 13 | 100 | 4 | 9 | 4 | 9 | 4 | 4 | 4 | 9 | 0 |
| F | 11 | 11 | 11 | 11 | 100 | 5 | 26 | 5 | 37 | 16 | 0 | 32 | 21 |
| H | 8 | 23 | 23 | 15 | 0 | 100 | 15 | 15 | 0 | 0 | 23 | 8 | 8 |
| T | 15 | 6 | 12 | 12 | 6 | 9 | 100 | 12 | 9 | 6 | 3 | 44 | 6 |
| K | 0 | 3 | 26 | 23 | 10 | 26 | 23 | 100 | 10 | 10 | 10 | 29 | 0 |
| L | 2 | 4 | 12 | 6 | 22 | 8 | 4 | 18 | 100 | 8 | 2 | 4 | 10 |
| D | 50 | 4 | 12 | 42 | 4 | 23 | 15 | 0 | 4 | 100 | 0 | 27 | 0 |
| G | 3 | 0 | 9 | 3 | 6 | 12 | 3 | 12 | 6 | 6 | 100 | 24 | 3 |
| S | 17 | 6 | 0 | 0 | 11 | 39 | 22 | 11 | 6 | 0 | 6 | 100 | 6 |
| Y | 0 | 0 | 0 | 0 | 29 | 0 | 0 | 14 | 14 | 0 | 0 | 0 | 100 |

A similarity score is determined for each polypeptide in subset D as compared with the first reference polypeptide chosen for subset E. The similarity score can be determined for example, by combining the similarity probabilities (represented in Table 6 above as 0-100%) to determine an overall score for the polypeptide. For example, if subset D is a collection of 6-mer polypeptides and the first polypeptide chosen is EPNGYF, each polypeptide in subset D is compared with the reference first polypeptide, EPNGYF, using the similarity matrix to calculate a similarity score by combining the similarity value at each of the 4 critical positions to the corresponding positions in the reference polypeptide. The maximum score is 100% (identical polypeptide) and the minimum score is zero.

A size for subset E is set at the desired number of polypeptides, for example 10, 20, 30, 40, 50, 100, 200 or 1000 polypeptides. A threshold value is determined which will generate the desired number of polypeptides for subset E. For example, if the threshold is set very low, and therefore the degree of similarity is very low and a smaller subset E of polypeptides will be generated. Conversely, if the threshold of similarity is set high, the subset E will be a larger number of polypeptides. The number of polypeptides can be determined by one skilled in the art based on the intended subsequent use of the polypeptides. For example, if a library of polypeptides of several thousand polypeptides is desired, the threshold can be set higher. If only 10 polypeptides are desired which are dissimilar from each other, the threshold can be set lower.

### a. Use of Non-Naturally Occurring Amino Acids for Polypeptide Design and Generation

The use of non-naturally occurring amino acids increases the diversity and thus uniqueness of the polypeptides that can be generated. For example, there are several hundred non-naturally occurring amino acids that are commercially available and a even larger number that can be synthesized by standard chemistry methods known in the art. The ability to incorporate non-naturally occurring amino acids also permits linear, cyclic and branched polypeptide structures to be designed and constructed.

Non-natural amino acids include, but are not limited to, non-natural β-amino acids; amino acids having alkyl, cycloalkyl, heterocyclyl, aromatic, heteroaromatic, electroactive, conjugated, azido, carbonyl and unsaturated side chain functionalities; isomeric N-substituted glycine, wherein the side chain of an α-amino acid is attached to the amino nitrogen instead of to the α-carbon of that molecule. The following are representative examples of non-natural amino acids:

Non-natural amino acids that are modifications of natural amino acids such that the amino group is attached to β-carbon atom of the natural amino acid (e.g., β-tyrosine). Non-natural amino acids that are modifications of natural amino acids in the side chain functionality, such that the imino groups or divalent non-carbon atoms such as oxygen or sulfur of the side chain of the natural amino acids have been substituted by methylene groups, or, alternatively, amino groups, hydroxyl groups or thiol groups have been substituted by methyl groups, olefin, or azido groups, so as to eliminate their ability to form hydrogen bonds, or to enhance their hydrophobic, properties (e.g. methionine to norleucine).

Non-natural amino acids that are modifications of natural amino acids in the side chain functionality, such that the methylene groups of the side chain of the natural amino acids have been substituted by imino groups or divalent non-carbon atoms or, alternatively, methyl groups have been substituted by amino groups, hydroxyl groups or thiol groups, so as to add ability to form hydrogen bonds or to reduce their hydrophobic properties (e.g. leucine to 2-aminoethylcysteine, or isolecine to o-methylthreonine).

Non-natural aimino acids that are modifications of natural amino acids in the side chain functionality, such that a methylene group or methyl groups have been added to the side chain of the natural amino acids to enhance their hydrophobic properties (e.g. Leucine to gamma-Methylleucine, Valine to beta-Methylvaline (t-Leucine)).

Non-natural amino acids that are modifications of natural amino acids in the side chain functionality, such that a methylene groups or methyl groups of the side chain of the natural amino acids have been removed to reduce their hydrophobic properties (e.g. Isoleucine to Norvaline).

Non-natural amino acids that are modifications of natural amino acids in the side chain functionality, such that the amino groups, hydroxyl groups or thiol groups of the side chain of the natural amino acids have been removed or methylated to eliminate their ability to form hydrogen bonds (e.g. Threonine to o-methylthreonine or Lysine to Norleucine). Non-natural amino acids that are optical isomers of the side chains of natural amino acids (e.g. Isoleucine to Alloisoleucine).

Non-natural amino acids that are modifications of natural amino acids in the side chain functionality, such that the substituent groups have been introduced as side chains to the natural amino acids (e.g. Asparagine to beta-fluoroasparagine). Non-natural amino acids that are modifications of natural amino acids where the atoms of aromatic side chains of the natural amino acids have been replaced to change the hydrophobic properties, electrical charge, fluorescent spectrum or reactivity (e.g. Phenylalanine to Pyridylalanine, Tyrosine to p-Aminophenylalanine).

Non-natural amino acids that are modifications of natural amino acids where the rings of aromatic side chains of the natural amino acids have been expanded or opened so as to change hydrophobic properties, electrical charge, fluorescent spectrum or reactivity (e.g. Phenylalanine to Naphthylalanine, Phenylalanine to Pyrenylalanine). Non-natural amino acids that are modifications of the natural amino acids in which the side chains of the natural amino acids have been oxidized or reduced so as to add or remove double bonds (e.g. Alanine to Dehydroalanine, Isoleucine to Beta-methylenenorvaline).

Non-natural amino acids that are modifications of proline in which the five-membered ring of proline has been opened or, additionally, substituent groups have been introduced (e.g. Proline to N-methylalanine). Non-natural amino acids that are modifications of natural amino acids in the side chain functionality, in which the second substituent group has been introduced at the alpha-position (e.g. Lysine to alpha-difluoromethyllysine).

Non-natural amino acids that are combinations of one or more alterations, as described supra (e.g. Tyrosine to p-Methoxy-m-hydroxyphenylalanine). Non-natural amino acids that are isomeric N-substituted glycines, wherein the side chain of an α-amino acid is attached to the amino nitrogen instead of to the α-carbon of that molecule (e.g. N-methyl glycine, N-isopropyl glycine). Non-natural amino acids which differ in chemical structures from natural amino acids but are compatible, in protected or unprotected form, with a hybrid synthesis of peptide chemistry. Non-natural amino acids are readily available and widely known. Exemplary non-natural amino acids (with their abbreviations) include, but are not limited to, for example: Aib for 2-amino-2-methylpropionic acid, β-Ala for β-alanine, α-Aba for L-α-aminobutanoic acid; D-α-Aba for D-α-aminobutanoic acid; Ac₃c for 1-aminocyclopropane-carboxylic acid; Ac₄c for 1-amino-cyclobutanecarboxylic acid; Ac₅c for 1-aminocyclopentanecarboxylic acid; Ac₆c for 1-aminocyclohexanecar-boxylic acid; Ac₇c for 1-amino-cycloheptanecarboxylic acid; D-Asp(ONa) for sodium D-aspartate; D-Bta for D-3-(3-benzo[b]thienyl)alanine; C₃al for L-3-cyclopropylalanine; C₄al for L-3-cyclobutylalanine; C₅al for L-3-cyclopentylalanine; C₆al for L-3-cyclohexylalanine; D-Chg for D-2-cyclohexylglycine; CmGly for N-(carboxymethyl)glycine; D-Cpg for D-2-cyclopentylglycine; CpGly for N-cyclopentylglycine; Cys(O₃Na) for sodium L-cysteate; D-Cys(O₃H) for D-cysteic acid; D-Cys(O₃Na) for sodium D-cysteate; D-Cys(O₃Bu₄N) for tetrabutylammonium D-cysteate; D-Dpg for D-2-(1,4-cyclohexadienyl)-glycine; D-Etg for (2S)-2-ethyl-2-(2-thienyl)glycine; D-Fug for D-2-(2-furyl)glycine; Hyp for 4-hydroxy-L-proline; IeGly for -[2-(4-imida-zolyl)ethyl]glycine; alle for L-L-alloisoleucine; D-alle for D-alloisoleucine; D-Itg for D-2-(isothiazolyl)glycine; D-tertLeu for D-2-amino-3,3-dimethyl-butanoic acid; Lys(CHO) for N⁶-formyl-L-lysine; MeAla for N-methyl-L-ala-nine; MeLeu for N-methyl-L-leucine; MeMet for N-methyl-L-methionine; Met(O) for L-methionine sulfoxide; Met(O₂) for L-methionine sulfone; D-Nal for D-3-(1-naphthyl)alanine; Nle for L-norleucine; D-Nle for D-nor-leucine; Nva for L-norvaline; D-Nva for D-norvaline; Orn for L-ornithine; Orn(CHO) for N⁵-formyl-L-ornithine; D-Pen for D-penicillamine; D-Phg for D-phenylglycine; Pip for L-pipecolinic acid; ⁱPrGly for N-isopropylglycine; Sar for sarcosine; Tha for L-3-(2-thienyl)alanine; D-Tha for D-3(2-thienyl)-alanine; D-Thg for D-2-(2-thienyl)glycine; Thz for L-thiazolidine-4-carboxylic acid; D-Trp(CHO) for Nⁱⁿ-formyl-D-tryptophan; D-trp(O) for D-3-(2,3-dihydro-2-oxoindol-3-yl)alanine; D-trp((CH₂)ₘCOR¹) for D-tryptophan substituted by a —(CH₂)ₘCOR¹ group at the 1-position of the indole ring; Tza for L-3-(2-thiazolyl)alanine; D-Tza for D-3-(2-thiazolyl)alanine; D-Tzg for D-2-(thiazolyl)glycine.

Non-naturally occurring amino acids can be ranked for antigenicity using methods applied to.the naturally occurring amino acids, for example by testing sequences against antisera or libraries of antibodies (described herein) and can be ranked along-side naturally occurringvamino acids. For example, a representive set of polypeptides composed of non-naturally occurring amino acids and/or a combination of non-naturally occurring and naturally occurring amino acids of a chosen polypeptide length can be used to immunize animals. Based on the subset of polypeptides injected which are antigenic and non-antigenic, amino acids are identified which either are more likely to be present in antigenic polypeptides or are more likely to be present on non-antigenic polypeptides. The likelihood of a amino acid's presence in antigenic polypeptide gives an observed antigenic ranking. Some non-ntural amino acids are very structurally similar to naturally occurring amino acids and to other non-naturally occurring amino acids. This similarity can be factored in to provide antigenicity rankings based on these similarities. Non-naturally occurring amino acids can also be assigned a similarity ranking for use with the methods as described, based on their structural and functional similarity to each other and to naturally occurring amino acids.

### b. Generation of Polypeptides

Once the polypeptides are designed, any of the subsets of polypeptides desrcibed herein can be generated by standard methods known in the art. The petides can be chemically synthesized by standard and/or combinatorial chemistry polypeptides can also be synthesized using recombinant means such as by expression of nucleic acids encoding the polypeptide sequences. For recombinant expression, the polypeptides are limited to the 20 naturally occurring amino acids and additionally non-naturally occurring amino acids where the expression organism of choice has been genetically engineered to generate such modifications.

### I. Identification of Binding Proteins for Polypeptide Binding Partner Pairs

Binding proteins are generated and/or selected that specifically bind the binding partners. The pairs of binding proteins and binding partners can then be used in applications such as addressable collections and capture systems. As noted, the polypeptide binding partners provided herein and the methods for generating such polypeptide binding partners provide polypeptides that are designed to be antigenic and thus antibodies or antibody fragments can be isolated which specifically bind to the polypeptides.

Candidate binding protein—polypeptide binding partner pairs can be identified by any method known to the art, including, but are not limited to, one or several of the following methods, such as, for example raising antibodies from exposure of a subject to the binding partner polypeptides and phage display of an antibody library followed by biopanning with the polypeptide binding partner of interest and any method known to those of skill in the art for identifying pairs of molecules that bind with high affinity and specificity. The following discussion provides exemplary methods; others can be employed.

### 1. Raising Antibodies

Antibodies contemplated herein include polyclonal antibodies, monoclonal antibodies and binding fragments thereof. Polyclonal antibodies are employed where high affinity (avidity) is desired. Polyclonal antibodies are typically obtained by immunizing an animal and isolating the polyclonal antibodies produced by the animal.

For example, antibodies have traditionally been obtained by repeatedly injecting a suitable animal (e.g., rodents, rabbits and goats) with an antigen or antigen with adjuvant (see, e.g., FIG. 2B). If the animal's immune system has responded, specific antibodies are secreted into the serum. The antibody-rich serum (antiserum) that is collected contains a heterogeneous mixture of antibodies, each produced by a different B lymphocyte. The different antibodies recognize different parts of the antigen, and are thus a heterogeneous mixture of antibodies. A homogeneous preparation of antibodies can be prepared by propagating an immortal cell line wherein antibody producing B-cells are fused with cells derived from an immortal B-cell tumor. Those hybrids (hybridoma cells) that are producing the desired antibody and have the ability to multiply indefinitely are selected. Such hybridomas are propagated as individual clones, each of which can provide a permanent and stable source of a single antibody (a monoclonal antibody) which is specific for the antigen of interest. The antibodies can be purified from the propagating hybridomas by any method known to those skilled in the art. Fragments of antibodies can be synthesized or produced and modified forms thereof produced.

In one exemplary embodiment, mice are immunized with a collection of polypeptide binding partners generated by the methods provided herein, for example as diphtheria toxin-6 mer polypeptide conjugates. The 6-mer has 2 non critical positions and 4 critical positions. The 2 non-critical positions of the 6-mer are adjacent to each other. The non-critical positions are not found at the ends of the polypeptide and thus are represented at two positions of positions 2, 3, 4 and 5. The 2 non-critical positions are chosen from S, G and Y. The remaining 4 critical residues are selected from the top 10 antigenic amino acids in table X: E, P, Q, N, F, H, T, K, L, and D.

Antibodies are raised against the collection of polypeptides. A library of hybridoma cells is then generated and clones are screened for their reactivity with individual polypeptides. Positive clones identify monoclonal antibodies which bind a selected polypeptide binding partner. The antibodies can be isolated by standard immunopurification techniques or by cloning methods such as by PCR with primers for conserved regions of the antibody structure.

Once the antibody is isolated, the polypeptide responsible for the identification of the antibody can be conjugated to a molecule and/or biological particle, as described below, and screened against the antibodies isolated above to determine whether the antibodies retain the ability to specifically bind the polypeptide, thereby identifying a binding protein—binding partner pair.

### 2. Phage Display

Antibodies can also be selected, for example by screening an antibody library, for example a single chain antibody library for antibodies which bind to each polypeptide. Phage display, protein expression library screening and antibody arrays as well as other screening methods well known in the art can be used to screen antibodies and antibody libraries for binding the polypeptides.

Polypeptides that interact with a specific binding protein, such as an antibody or antibody fragment, can be identified by displaying random libraries of binding proteins on the surface of a phage molecule and monitoring their interactions with the polypeptides. The bacteriophage that display binding proteins that interact with the polypeptides can be isolated through washing and then enriched through multiple panning steps, resulting in a high population of phage displaying a binding partner that can be used as a binding protein—binding partner pair.

For example, in order to identify binding proteins using panning and phage display, hybridoma cells are first created either from non-immunized mice or mice immunized with a library of random epitopes or immunized with groups or libraries of binding partners polypeptides. The mice (or other immunized animals) are initially screened for high immunoglobulin (Ig) production and epitope/peptide binding. Ig production can be measured in culture supernatants by ELISA assay using a goat anti-mouse IgG antibody. Epitope/peptide binding can also be measured by ELISA assay in which the mixture of haptens used for immunization are immobilized to the ELISA plate and bound IgG from the culture supernatants is measured using a goat anti-mouse IgG antibody. Both assays can be performed in 96-well formats or other suitable formats.

To produce an antibody library, recombinant antibody genes from mRNA isolated from spleenocytes or peripheral blood lymphocytes (PBLs). Functional antibody fragments can be created by genetic cloning and recombination of the variable heavy (V_{H}) chain and variable light (V_{L}) chain genes. The V_{H} and V_{L} chain genes are cloned by first reverse transcribing mRNA isolated from spleen cells or PBLs into cDNA. Specific amplification of the V_{H} and V_{L} chain genes is accomplished with sets of PCR primers that correspond to consensus sequences flanking these genes. The V_{H} and V_{L} chain genes are joined with a linker DNA sequence. A typical linker sequence for a single-chain antibody fragment (scFv) encodes the amino acid sequence (Gly₄Ser)₃. After the V_{H}-linker-V_{L} genes have been assembled and amplified by PCR, the products can be transcribed and translated directly or cloned into an expression plasmid such as for phage display and then expressed to produce functional recombinant antibody fragments displayed on the phage.

The phage library of binding proteins such as antibodies, is panned against the polypeptide binding partners and those which specifically bind are isolated.

### 3. Generation of Binding Protein-Binding Partner Pairs

As described herein, binding proteins can be used as capture agents in the collections of capture agents and binding partners, addressable collections and capture systems described herein. Once antibodies and/or antibody fragments are identified which bind to the HAHS polypeptides, they can be used as capture agents. The antibodies can optionally be purified such as by hybridoma selection and affinity purification. The antibodies or fragments thereof can be cloned, such as described herein and known in the art and expressed by recombinant means for use as capture agents.

The HAHS polypeptides can be used as binding partners in capture agent-binding partner pairs in the collections of capture agents and binding partners, addressable collections and capture systems described herein. The HAHS peptides are conjugated to molecules and/or biological particles as tags that specifically bind capture agents. The HAHS polypeptides can be conjugated to molecules and/or biological particles by any means known in the art such as those described herein, including, but not limited to, recombinant means and chemical linkages. The conjugation can be direct or indirectly via a linker. The HAHS polypeptides can be encoded by nucleic acid molecules which can be joined with nucleic acid molecules encoding another polypeptide to create tagged-polypeptides such as described herein. For example, a collection of nucleic acid molecules encoding HAHS polypeptides can be used to create a tagged library of molecules.

### J. EXAMPLES

The following examples are included for illustrative purposes only and are not intended to limit the scope of the invention.

### Example 1

Preparation of Anti-Tag Antibody Collections

### A. Generating a Collection of Antibody—Tag Pairs

A collection of antibodies that bind peptide tags is used to sort molecules linked to the tags. The collection of antibodies that specifically bind to the polypeptide tags can be generated by a variety of methods. One example is described below.

### 1. Hybridoma Screening

High affinity and high specificity antibodies for the array were identified by screening a randomly selected collection of individual hybridoma cells against a phage display library expressing a random collection of peptide epitopes. The hybridoma cells were created by fusion of spleenocytes isolated from a naive (non-immunized) mouse with myeloma cells. After a stable culture was generated, approximately 10-30,000 individual cell clones (monoclonals) were isolated and grown separately in 96-well plates. The culture supernatants from this collection were screened by ELISA with an anti-IgG antibody to identify cultures secreting significant amounts of antibody. Cultures with low antibody production were discontinued. Antibodies from this monoclonal collection were separated from culture supernatants using HiTrap® Protein G-columns using the Akta® Prime chromatography system following the manufacturer's protocol (AP Biotech).

Purified antibodies were used to screen for high affinity epitopes on phage-displayed peptide libraries (PhD7, PhD12 or C7C from New England Biolabs) as described below.

### a. Biopanning

The antibodies were diluted in 0.1 M NaHCO₃ to give a final concentration of 5 μg/ml. Wells of a 8 well strip were coated with 50 μl of antibody and left at 4° C. overnight. Four 8 well strips were coated per antibody for use in all 4 rounds of biopanning. The following day, a loopful of ER2738 E. Coli cells were inoculated in 20 ml 2×YT and grown on the shaker at 37° C. until the OD was between 0.5-0.8. Meanwhile, the coating antibodies were aspirated off and 200 μl of 3% non-fat milk (NFM) in 1×TBS-T was added and incubated at 37° C. for 1 hour. The wells were washed with 100 μl 1×TBS-T two times. The phage library was added at 1×10¹¹ particles per well (dilution was made in 3% NFM in 1×TBS-T to a final volume of 100 μl). This solution was the INPUT.

The wells were incubated at 37° C. for 1 hour followed by 5 washes with 1×TBS-T (1 minute per wash) for round 1. The bound phage were eluted by addition of 100 μl of 0.1 M glycine, pH 2.2. This eluate was transferred into an Eppendorf tube, followed by addition of 10 μl Tris, pH 8.0 to the same Eppendorf tube. The glycine and Tris steps were repeated once more and this solution was now the OUTPUT. The OUTPUT from the first round was now to be used as INPUT for the second/round.

The grown ER2738 cells were centrifuged at 3500 rpm for 15 min and the cells resuspended in 1/20 of the original volume (1 ml) using Min A salts. One hundred μl of the cells suspension was aliquoted into 15 ml Falcon tubes to which the OUTPUT (220 μl) was added and incubated at 37° C. for 30 min. The volume was increased to 1.0,ml with 2×YT (add 680 μl 2×YT) and incubated at 30° C. for 4 hours. The cells were spun at 8000 rpm for 15 min and the supernatants were transferred to Eppendorfs for use the next day as INPUT. These solutions were stored at 4° C.

Round 2 panning was a repeat of Round 1, however the wells were washed 10 times with 1×-TBS-T (1 min per wash).

Round 3 panning was a repeat of Round 1, however the wells were washed 20 times with 1×-TBS-T (1 min per wash).

Round 4 panning was a repeat of Round 1, however the wells were washed 20 times with 1 ×-TBS-T (1 min per wash).

### b. Titering of the INPUT and the OUTPUT

Appropriate dilutions were taken from the phage in culture tubes (e.g. 10⁸, 10¹⁰ and 100 μl for each dilution) and 300 μl of ER2738 E. coli cells were added to each aliquot. This suspension was kept at room temperature for 10 minutes. Three ml of Top Agar was added to each tube and poured on top of an LB Agar plate. The plate was incubated at 37° C. overnight and the number of plaques counted.

### c. Making Hybridomas

Hybridoma cells were prepared by methods well known to those of skill in the art (see, e.g., Harlow et al. (1988) Antibodies: A Laboratory Manual, Cold Spring Harbor Laboratory, Cold Spring Harbor). Hybridoma cells were created by the fusion of mouse spleenocytes and mouse myeloma cells. For the fusion, antibody-producing cells were isolated from the spleen of a non-immunized mouse, mixed with the myeloma cells and fused. Alternatively, the hybridoma cells were created from spleenocytes isolated from a mouse previously immunized chicken IgY.

A healthy, rapidly dividing culture of mouse myeloma cells were diluted into 20 ml of medium containing 20% fetal bovine serum (FBS) and 2×OPI. Growth medium is typically Dulbecco's modified Eagle's (DME) or RPMI 1640 medium. Ingredients of mediums are well known (see, e.g., Harlow et al. (1988) Antibodies: A Laboratory Manual, Cold Spring Harbor Laboratory, Cold Spring Harbor).

Antibody producing cells were prepared by aseptic removal of a spleen from a mouse, disruption of the spleen into cells and removal of the larger tissue by washing with 2×OPI medium. A typical mouse spleen contains approximately 5×10⁷ to 2×10⁸ lymphocytes. Equal numbers of spleen cells and myeloma cells were pelleted by centrifugation (400× g for 5 min) and the pellets were separately resuspended in 5 ml of medium without serum and then combined. Polyethylene glycol (PEG) is added to 0.84% from a 43% solution. The cells were gently resuspended in the PEG-containing medium and then repelleted by centrifugation at 400× g for 5 minutes, washed by resuspension in 5 ml of medium containing 20% FBS, repelleted and washed a second time in medium supplemented with 20% FBS, 1×OPI, and 1×AH (AH is a selection medium; 1×AH contains 5.8 μM azaserine and 0.1 mM hypoxanthine). Cells were incubated at 37° C. in a CO₂ incubator. Clones generally are visible by microscopy after 4 days.

### d. Isolating Hybridoma-Cells

Stable hybridomas were selected by growth for several days in poor medium. The medium then was replaced with fresh medium and single hybridomas were isolated by limited dilution cloning. Because hybridoma cells have a very low plating efficiency, single cell cloning was performed in the presence of feeder cells or conditioned medium. Freshly isolated spleen cells can be used as feeder cells as they do not grow in normal tissue culture conditions and are lost during expansion of the hybridoma cells. In this procedure, a spleen was aseptically removed from a mouse and disrupted. Released cells were washed repeatedly in medium containing 1 0% FBS. A spleen typically produces 1 00 ml of 10⁶ cells per ml. The feeder cells were plated in 96-well plates, 50 μl per well, and grown for 24 hours. Healthy hybridoma cells were diluted in medium containing 20% FBS, 2×OPI to a concentration of 20 cells per milliliter. Cells should be as free of clumps as possible. Fifty μl of the diluted hybridoma cells were added to the feeder cells to a final volume of 100 μl. Clones began to appear in 4 days.

Alternatively, single cells can be isolated by single-cell picking by individually pipetting single cells and then depositing in wells containing feeder cells. Single cells also can be obtained by growth in soft agar. Once healthy, stable cultures were achieved the cells are maintained by growth in DME (or RPMI 1640) medium supplemented with 10% FBS. Stable cells were stored in liquid nitrogen by slow freezing in medium containing a cryoprotectant such as dimethylsulfoxide (DMSO). The amount of antibody being produced by the cells was determined by measuring the amount of antibody in the culture supernatants by the ELISA method.

### 2. Recovery of Phage after Panning and Sequencing the Epitopes

### a. Identification of Positive Phage Clones by ELISA.

In a 96-deep well plate, 100 μl of E. coli 2738 cells grown previously to an OD of 0.5 were added. To each well, 96 individual plaques from the titer plates were added and the plates then were kept at 37° C. for 30 minutes. To each well was added 400 μl of 2×YT with tetracycline. The plates then were kept at 30° C. overnight with shaking. In the meantime, 96-well polystyrene plates (Maxisorp, NUNC) were coated with the appropriate antibody for detection and kept. overnight at 4° C.

The following day, the antibody was aspirated off, 100μl of 3% non-fat milk in 1×TBST was added to each well and the plate incubated at 37° C. for 1 hour. The plate then was washed with 2× with TBS-T. Ten μl of 10% milk in 5×TBS-T was added to each well followed by addition of 40 μl of sample from deep well plate to the corresponding well in the ELISA plate. The ELISA plate was incubated at 37° C. for 1 hour. The plate then was washed 4 times with TBS-T.

Then, 50μl of the anti-M13 antibody-HRP conjugate was added to each well at 1 in 5000 dilution prepared in 3% non-fat milk in 1×TBS-T and incubated at 37° C. for 1 hour. The plate was washed 4 times with TBS-T, followed by addition of 50 μl OPD in each well. After yellow color develops, the reaction was stopped by the addition of 13 μl 3 N HCl. The absorbance was read at 492 nm.

### b. Sample Preparation for Sequencing

Eight positive phage clones were picked and added to a 96-deep well plate that contained 100 μl of E. coli 2738 cells. The plate was incubated at 37° C. for 30 min followed by addition of 900 μl of 2×YT media and an additional incubation at 37° C. for 4 hour. This plate then was sent to MJ Research (Waltham, Calif.) for sequencing.

### B. Selective Infection

Selective infection technologies, such as phage display, are used to identify interacting protein-peptide pairs. These systems take advantage of the requirement for protein-protein interactions to mediate the infection process between a bacteria and an infecting virus (phage). The filamentous M13 phage normally infects E. coli by first binding to the F pilus of the bacteria. The virus binds to the pilus at a distinct region of the F pilin protein encoded by the traA gene. This binding is mediated by the minor coat protein (protein 3) on the tip of the phage. The phage binding site on the F pilin protein (a 13 amino acid sequence on the traA gene) can be engineered to create a large population of bacteria expressing a random mixture of phage binding sites.

The phage coat protein (protein 3) also can be engineered to display a library of diverse single chain antibody structures. Infection of the bacteria and internalization of the virus is therefore mediated by an appropriate antibody-peptide epitope interaction. By placing appropriate antibiotic resistance markers on the bacteria and virus DNA, individual colonies can be selected that contain both genes for the antibody and its corresponding peptide epitope. The recombinant antibody phage display library prepared from non-immunized mice and the bacterial strains containing a random peptide sequence in the phage binding site in the traA gene are commercially available (Biolnvent, Lund, Sweden). Creation of a recombinant antibody library is described below.

### c. Expression and Purification of Antibodies

Purification of antibodies from hybridoma supernatants was achieved by affinity binding. A number of affinity binding substrates are commercially available. The procedure described below is based on commercially available substrates (Protein A-Sepharose®) and follows the procedure described above.

Recombinant antibodies were expressed and purified as described (McCafferty et al. (1996) Antibody engineering: A practical Approach, Oxford University Press, Oxford). Briefly, the gene encoding the recombinant antibody was cloned into an expression plasmid containing an inducible promoter. The production of an active recombinant antibody was dependent on the formation of a number of intramolecular disulfide bonds. The environment of the bacterial cytoplasm is reducing, thus preventing disulfide bond formation. One solution to this problem was to genetically fuse a secretion signal peptide onto the antibody which directs its transport to the non-reducing environment of the periplasm (Hanes et al. (1997) Proc. Natl. Acad. Sci. U.S.A. 94:4937-4942).

Alternatively, the antibodies can be expressed as insoluble inclusion bodies and then refolded in vitro under conditions that promote the formation of the disulfide bonds.

### D. Exemplary Array and use Thereof for Capture of Proteins with Polypeptide Tags and Detection Thereof

To demonstrate the functioning of the methods herein, capture antibodies, specific, for example,.for various peptide epitopes, such as the human influenza virus hemagglutinin (HA) protein epitope, which has the amino acid sequence YPYDVPDYA, were used to tag, for example, scFvs. For example, an scFv with antigen specificity for human fibronectin (HFN) was tagged with an HA epitope, thus generating a molecule (HA-HFN), which was recognized by an antibody specific for the HA peptide and which has antigen specificity of HFN. After depositing various concentrations of the capture antibodies (from 800 μg/ml to 200 μg/ml), including anti-HA tag capture antibodies, onto a glass slide coated with a surface for capturing proteins, such as a nitrocellulose-coated slide (FAST™, Schleicher and Schuell), they were allowed to bind at ambient temperature and humidity of 50 to 60%. After binding, slides with deposited anti-HA capture antibodies were blocked with a protein-containing solution such as Blocker BSA™ (Pierce) diluted to 1× in phosphate-buffered saline (PBS) with Tween-20 (polyoxyethylenesorbitan monolaurate; Sigma) added to a final concentration of 0.05% (vol:vol) or with a 3% non-fat milk in the same buffer to eliminate background signal generated by non-specific protein binding to the membrane. For subsequent description contained herein PBS with 0.05% (vol:vol) Tween-20 is referred to as PBS-T. Blocking times can be varied from 60 min at ambient temperature to longer hours at ambient temperature or at 4° C., for example. Incubation temperatures for all subsequent steps can be varied from ambient temperature to about 37° C. In all instances, the precise conditions are determined empirically.

After blocking the membranes containing the deposited anti-HA capture antibodies, an incubation with peptide epitope-tagged scFvs can be performed. Purified scFvs (or bacterial culture supernatants, or various crude subcellular fractions obtained during purification of such scFvs from E. coil cultures harboring plasmid constructs that direct the expression of such scFvs upon induction, for example HA-HFN scFv, containing the HA peptide tag), can be diluted to various concentrations (for example, between 0.1 and 100 μg/ml) in BBSA-T. Membranes with deposited anti-peptide tag capture antibodies then were incubated with this HA-HFN scFv antigen solution. Membranes with deposited anti-HA capture antibodies and bound HA-HFN scFv antigen then were washed three times with PBST for suitable periods of time (e.g., 3-5 min per wash).

Membranes with deposited anti-HA capture antibodies and bound HA-HFN scFv then were incubated with, for purposes of demonstration, biotinylated human fibronectin (Bio-HFN), which is an antigen that will be recognized by the capture HA-HFN scFv. Bio-HFN was serially diluted (e.g., from 1 to 10 μg/ml) in BBSA-T. The resulting membranes were washed as before and then were incubated with Neutravidin●HRPO (Pierce) diluted 1 in 10000 in BBSA-T. The resulting slides were washed as before, rinsed with PBS and developed with a 1:1 mixture of freshly prepared Supersignal™ ELISA Femto Stable Peroxide Solution and Supersignal™ ELISA Femto Lumino Enhancer Solution (Pierce), and then imaged using an imaging system, such as, for example, a Kodak Image Station 440CF or IS1000 or other such imaging system. A small volume of the Supersignal solution was plated on the platen of the image station.

Slides then were placed array-side down into the center of the platen, thus placing the surface area of the antibody-containing portion of the membrane into the center of the imaging field of the camera lens. In this way, the small volume of developer, present on the platen, can then contact the entire surface area of the antibody-containing portion of the slide. The Image Station cover then was closed for antibody array image capture. Camera focus (zoom) varies depending on the size of the membrane being imaged. Exposure times can vary depending on the signal strength (brightness), emanating from the developed membrane. Camera f-stop settings are infinitely adjustable between 12 and 16.

Archiving and analysis of array images can be performed, for example, using the Kodak ID 3.5.2 software package. Intensity values for loci were measured using software. These data then were transformed, for example into Microsoft Excel, for statistical analyses.

### Example 2

### Construction of a scFv Master Library

### A. mRNA Isolation

Immunized mouse spleens with an ELISA titer within the range of 100,000. Spleens were quick frozen immediately upon removal by immersion in liquid nitrogen and stored at −80° C. after fast freeze. The mouse spleens then were weighed without thawing. Total RNA was isolated using Stratagene's RNA Isolation kit according to manufacture's protocol. For a naive library, the mRNA was isolated from total RNA using Stratagene's Poly(A) quick mRNA isolation kit according to manufacture's protocol. The concentration of mRNA was determined by making an appropriate dilution in RNAse-Free H₂O and measuring the optical density at 260 nm in a spectrophotometer. The quality of the RNA was tested by setting up one reaction of first strand cDNA synthesis and amplifying with a pair of primers for Fab or scFv light chain (see below).

### B. First Strand cDNA Synthesis

Library generation by PCR was performed in a laminar flow hood which was irradiated with UV light for more than 30 min prior to use. A RNA/primer mixture was prepared in sterile 0.2 ml PCR tubes on ice as follows:

| Component | Sample |
|---|---|
| 2 μg total RNA | x μl |
| Random hexamers (50 ng/μl) | 2 μl |
| 10 mM dNTP mix | 1 μl |
| DEPC-treated dH₂O | x μl |
| total volume | 10 μl |

The sample was incubated at 65° C. in a thermal cycler for 5 min and then chilled on ice for at least 1 minute. The following mixture was prepared on ice by adding each component in the order indicated below:

| Component | each reaction | 4 reactions |
|---|---|---|
| 10X RT buffer | 2 μl | 8 μl |
| 25 mM MgCl₂ | 4 μl | 16 μl |
| 0.1 M DTT | 2 μl | 8 μl |
| RNase OUT recombinant | 1 μl | 4 μl |
| RNase inhibitor | | |

Nine μl of reaction mix was added to each RNA/primer mixture, mixed gently and then spun briefly. The reaction was incubated at 25° C. in a thermal cycler for 2 minutes. One μl (50 units) of Superscript II RT was added to each tube, mixed gently and then spun quickly. The mixture was incubated for 10 minutes at 25° C., for 50 min at 42° C. and for 15 min at 70° C. The reaction then was chilled on ice. The reaction was spun briefly, 1 μl of RNase H was added to each tube and then incubated at 37° C. for 20 minutes. Samples then were used in the amplification section below or stored at −80° C.

### C. Amplification of First Strand cDNA

### PCR Reactions

Working dilutions of the mouse primers were prepared. Each primer was diluted to 100 pmol/μl (to be stored at −80° C. stock) and 10 pmol/μl (to be stored at −20° C. stock) with 10 mM Tris pH 8.0 (RNase free). Ten pmol/μl of primer mix were prepared of each variant at equal molar concentration as shown in Table 7 below:

TABLE 7

| | | | Volume of | |
|---|---|---|---|---|
| | | | variant at | Total volume |
| Primer Mix | SEQ ID NO. | Common Name | 10 pmol/μl | in mix |
| MK1-5 | 103 | MK1 | 10 μl | 100 μl |
| | 104 | MK2 | 20 μl | |
| | 105 | MK3 | 10 μl | |
| | 106 | MK4 | 20 μl | |
| | 107 | MK5 | 40 μl | |
| MK6-10 | 108 | MK6 | 20 μl | 120 μl |
| | 109 | MK7 | 40 μl | |
| | 110 | MK8 | 20 μl | |
| | 111 | MK9 | 30 μl | |
| | 112 | MK10 | 10 μl | |
| MK11-15 | 113 | MK11 | 10 μl | 120 μl |
| | 114 | MK12 | 20 μl | |
| | 115 | MK13 | 10 μl | |
| | 116 | MK14 | 40 μl | |
| | 117 | MK15 | 40 μl | |
| MK16-20 | 118 | MK16 | 40 μl | 110 μl |
| | 119 | MK17 | 10 μl | |
| | 120 | MK18 | 30 μl | |
| | 121 | MK19 | 20 μl | |
| | 122 | MK20 | 10 μl | |
| MK21-25 | 123 | MK21 | 20 μl | 100 μl |
| | 124 | MK22 | 20 μl | |
| | 125 | MK23 | 20 μl | |
| | 126 | MK24 | 20 μl | |
| | 127 | MK25 | 20 μl | |
| MKR1-4 | 128 | MKR1 | 40 μl | 160 μl |
| | 129 | MKR2 | 40 μl | |
| | 130 | MKR3 | 40 μl | |
| | 131 | MKR4 | 40 μl | |
| MH1-5 | 132 | MH1 | 40 μl | 180 μl |
| | 133 | MH2 | 40 μl | |
| | 134 | MH3 | 40 μl | |
| | 135 | MH4 | 20 μl | |
| | 136 | MH5 | 40 μl | |
| MH6-10 | 137 | MH6 | 20 μl | 180 μl |
| | 138 | MH7 | 60 μl | |
| | 139 | MH8 | 40 μl | |
| | 140 | MH9 | 40 μl | |
| | 141 | MH10 | 20 μl | |
| MH11-15 | 142 | MH11 | 10 μl | 190 μl |
| | 143 | MH12 | 40 μl | |
| | 144 | MH13 | 60 μl | |
| | 145 | MH14 | 40 μl | |
| | 146 | MH15 | 40 μl | |
| MH16-20 | 147 | MH16 | 20 μl | 130 μl |
| | 148 | MH17 | 20 μl | |
| | 149 | MH18 | 40 μl | |
| | 150 | MH19 | 40 μl | |
| | 151 | MH20 | 10 μl | |
| MH21-25 | 152 | MH21 | 80 μl | 200 μl |
| | 153 | MH22 | 60 μl | |
| | 154 | MH23 | 40 μl | |
| | 155 | MH24 | 10 μl | |
| | 156 | MH25 | 10 μl | |
| MHR1-4 | 157 | MHR1 | 40 μl | 160 μl |
| | 158 | MHR2 | 40 μl | |
| | 159 | MHR3 | 40 μl | |
| | 160 | MHR4 | 40 μl | |

The mixtures were stored at −20° C. PCR reaction mixtures were prepared on ice in 0.2 ml PCR tubes using Clontech's Advantage HF2 polymerase as follows:

For scFv-HC:

| | | | | template | | |
|---|---|---|---|---|---|---|
| 10X HF2 | 10X HF2 | F-primer | R-primer | (1st strand | Polymerase | |
| buffer | dNTP mix | (10 pmol/μl) | (10 pmol/μl) | cDNA) | Mix | dH₂O |
| 5 μl | 5 μl | 1 μl MH1-5 | 1 μl MHR1-4 | 2 μl | 1 μl | 35 μl |
| 5 μl | 5 μl | 1 μl MH6-10 | 1 μl MHR1-4 | 2 μl | 1 μl | 35 μl |
| 5 μl | 5 μl | 1 μl MH11-15 | 1 μl MHR1-4 | 2 μl | 1 μl | 35 μl |
| 5 μl | 5 μl | 1 μl MH16-20 | 1 μl MHR1-4 | 2 μl | 1 μl | 35 μl |
| 5 μl | 5 μl | 1 μl MH21-25 | 1 μl MHR1-4 | 2 μl | 1 μl | 35 μl |

For scFv-LC:

| | | | | template | | |
|---|---|---|---|---|---|---|
| 10X HF2 | 10X HF2 | F-primer | R-primer | (1st strand | Polymerase | |
| buffer | dNTP mix | (10 pmol/μl) | (10 pmol/μl) | cDNA) | Mix | dH₂O |
| 5 μl | 5 μl | 1 μl MK1-5 | 1 μl MKR1-4 | 2 μl | 1 μl | 35 μl |
| 5 μl | 5 μl | 1 μl MK6-10 | 1 μl MKR1-4 | 2 μl | 1 μl | 35 μl |
| 5 μl | 5 μl | 1 μl MK11-15 | 1 μl MKR1-4 | 2 μl | 1 μl | 35 μl |
| 5 μl | 5 μl | 1 μl MK16-20 | 1 μl MKR1-4 | 2 μl | 1 μl | 35 μl |
| 5 μl | 5 μl | 1 μl MK21-25 | 1 μl MKR1-4 | 2 μl | 1 μl | 35 μl |

The reactions were mixed gently then spun briefly. The tubes then were set in the thermal cycler preheated to 94° C. and the following cycle was started: 94° C. for 2 min, 94° C. for 1 min, 55° C. for 1 min, 72° C. for 1 min, 72° C. for 10 min for 30 cycles and then held at 4° C. The reactions then were spun briefly and proceed to gel purification steps

### 2. Gel Purification of PCR Products

A 1% low melting point agarose gel was prepared. Ten 10 μl of 6× loading buffer was added to each 50 μl PCR reaction. The entire sample was loaded onto 1% agarose gel. The gels were run at 100 volts until the dark blue dye runs ⅔ length of the gel. The gels then were photographed. Working quickly, the gels were visualized with UV light and the bands excised at the appropriate size

scFv-HC: ˜350 bp

scFv-LC: ˜325 bp

### 3. Frozen Phenol Purification of DNA from Low Melt Agarose

The appropriate bands were cut out and placed into eppendorf tubes (450 μl each tube) or in 15 ml conical tubes (4.5 ml each tube). The volume of agarose slice was estimated. 1/10^{th} volume 3 M NaOAc, pH 5.2 and 1/10^{th} volume 1 M Tris, pH 8.0, was added to the tube containing the excised slice. The slice then was melted at 65° C. in a heat block. Once the slice was completely melted, an equal volume of room temperature phenol was added. The solution was well-vortexed (30 seconds) until all chunks of agarose were dissolved. The solution then was frozen on dry ice until solid. To separate the phases, the solution was spun for 15 min at maximum speed at RT. The aqueous phase was transferred to a fresh tube without disturbing the interface. The separation and transfer steps were repeated once, followed by extraction by chloroform. The aqueous phase was transferred to a fresh tube and 1 μl of glycogen (20 mg/ml) was added. Two volumes of 100% EtOH were added. The solution then was incubated at −20° C. for 2 hours to overnight. Solution can optionally be incubated for 30 min at −80° C.). The DNA was pelleted at 4° C. for 15 min at maximum speed, then washed with 70% EtOH once. The pellet was resuspended in dH₂O or 10 mM Tris pH 8.0. The purified PCR product was quantified. The purified DNA then was stored at −20° C.

### D. Antibody Fragment Assembly

### 1. The scFv Linker

The scFv linker was generated using Clontech's Advantage HF2 polymerase kit as outlined by the manufacturer's instructions. Briefly, PCR mix was prepared in a 0.2 ml PCR tube on ice with the following:

5 μl 10×HF2 buffer

4 μl 10×HF2 dNTP mix

2 μl 10 pmol/μl of LinkF (SEQ ID No. 164)

2 μl 10 pmol/μl of PDK-125 LinkR (SEQ ID No. 165)

25 ng of pBADHA-HFN clone 10

1 μl polymerase mix

add dH₂O to total volume of 50 μl

The tubes were set in the thermal cycle block and the following cycle was started: 94° C. for 2 min; 94° C. for 1 min/55° C. for 1 min/72° C. for 1 min for 30 cycles then 72° C. for 10 min and holding at 4° C.

The prepared assembled scFv linker then was purified by gel electrophoresis. A 2% agarose gel was prepared. Ten μl of 6× loading buffer was added to each 50 μl PCR mix and loaded onto the gel. The gel was run at 100 volts until the dark blue dye ran ⅔ down the length of the gel. The scFv linker band (at. ˜50 bp) was excised from the gel.

The PCR product was purified from the excised gel slice using the MERmaid® kit (Qbiogene, Carlsbad Calif.) according to the manufacture's instruction. Optionally, the PCR product can be purified using “Frozen phenol” purification. The purified scFv linker was quantified using Picogreen® quantitation kit (Molecular Probes) according to the manufacturer's, protocol.

### 2. scFv Assembly

Two PCR mixtures were prepared in 0.2 ml PCR tubes on ice as follows:

4 μl 10×HF2 buffer

4 μl 10×HF2 dNTP mix

5 ng purified scFv-HC fragment

5 ng purified scFv-LC fragment

2 ng purified scFv-linker (from step above)

0.8 μl Advantage polymerase mix

bring to 40 μl with dH₂O

The tubes were placed in a thermal cycler block and the following cycle was started: 94° C. for 3 min; 94° C. for 30 seconds/55° C. for 30 seconds/72° C. for 1 min for 7 cycles; and hold at 4° C. The tubes then were spun briefly and placed on ice. A mixture of following components was prepared: 1 μl 10×HF2 buffer

1 μl 10×HF2 dNTP mix

2 μl primer SfiFor (SEQ ID No. 166)

2 μl primer NotRev (SEQ ID No. 167)

0.2 μl Advantage polymerase mix

bring to total of 10 μl with dH₂O

Ten μl of the mixture was added to each of the 40 μl PCR reactions. The solutions were mixed and then spun. The tubes then were placed in a thermal cycler block preheated to 94° C. and the following cycle was started: 94° C. for 2 min; 94° C. for 1 min/55° C. for 1 min/72° C. for 2 min for 30 cycles; 72° C. for 10 min; and held at 4° C.

The assembled scFv fragment was purified by gel electrophoresis. A 1% low melting agarose gel was prepared. Ten μl of 6× loading buffer was added to each 50 μl PCR mix and loaded onto the gel. The gel was run at 100 volts until the dark blue dye ran ⅔ down the length of the gel. Working quickly, the gel was visualized with UV, light and the scFv band at ˜700 bp was excised. The DNA was extracted from the gel slice using Frozen Phenol purification of DNA from low melt agarose. The amount of purified scFv fragment-was quantitated using the Picogreen® kit (Molecular Probes).

### E. Generate Fab and scFv Library in pBADHA or Equivalent

### 1. Generation of Sfil/Notl Digested pBADHA (or Equivalent)

Digestion reaction mix was prepared in a 1.5 ml eppendorf tubes as follows:

X μl pBADHA (˜20 μg)

20 μl 10× buffer #2 (NEB)

20 μl 10×BSA (100× stock)

10 μl Sfil (20 units/μl)

X μl dH₂O for a total of 200 μl

The solution was incubated at 50° C. for 4 hours. Following the incubation, the solution was spun briefly and he following components were added to each tube:

5 μl 10× buffer #3 (NEB)

5 μl 10×BSA (NEB, 100× stock)

8 μl 1M Tris pH 8.0

2 μl 5 M NaCl

10 μl Notl

20 μl dH₂O

The solution then was incubated at 37° C. for 4 hours.

For dephosphorylation, the following components were added to above digestion reaction:

5 μl 10× buffer #3

20 μl CIP alkaline phosphatase (1 unit/μl)

25 μl dH₂O

The solution then was incubated for 30 min at 37° C. The digested and dephosphorylated DNA was run on 1% agarose gel for purification. The Sfil/Notl fragment band was excised from the gel and the DNA was purified from the slice by extraction using Frozen Phenol purification of DNA from low melt agarose. The Picogreen® kit from Molecular Probes was used for quantitation of the purified pBADHA (Sfil/Notl/CIP) DNA.

The background of purified pBADHA (Sfil/Notl/CIP) DNA was determined. Briefly, the following ligation was prepared:

X μl 5 ng of pBAbHA (Sfil/Notl/CIP) DNA

0.5 μl T4 DNA ligase buffer

0.5 μl T4 DNA ligase (NEB; 400 units/μl)

add dH₂O to bring to total of 5 μl

The ligation reaction was incubated at 16° C. for ˜16 hours. The reaction then was chilled on ice for 5 min and spun briefly.

Electroporation cuvettes (VWR; 1 mm gap) and 0.5 ml eppendorf tubes were pre-chilled on ice. The frozen electrocompetent XL1-blue cells (with transformation efficiency at about 1×10⁸) were thawed on ice. Forty μl of cells were transferred to the 0.5 ml tube on ice and 1 μl of ligation (1 ng DNA) mix was added to the tube. In addition, 1 ng of pBADHA uncut was placed in a separate tube as a control. The mixtures were placed on ice for ˜1 min. The transformation mix were transferred to the prechilled electroporation cuvettes on ice and shaken to the bottom of the cuvette. The mixtures were electroporated once at 1.7 KV. Following the electroporation, 300 μl of 2×YT/glucose medium was added to the cuvettes. The solution was transferred to a 5 ml Falcon tube with a transfer pipette. The culture was incubated for 1 hour at 37° C. with shaking at 250 rmp. One μl, 10 μl and 30 μl of the transformed cells were plated onto 3 separate 2×YT/glucose/amp plates (100 mm) using sterile glass beads. Once dry, the plates were inverted and incubated at 37° C. overnight. The colony number on each plate was observed visually (pBADHA (Sfil/Notl/CIP) to ensure less than 10 colonies per plate. DNA should give the same or fewer colonies than uncut pBADHA.

### 2. Generation of Sfil/Notl Digested Fab or-ScFv Fragment

A digestion reaction mix was prepared in a 1.5 ml eppendorf tube as follows:

X μl Purified Fab or scFv DNA (˜1 μg)

5 μl 10× buffer #2 (NEB)

5 μl 10×BSA

2 μl Sfil (NEB; 20 units/μl)

add dH₂O to bring total volume of 50 μl

The digestion reaction was incubated at 50° C. for 2 hours. The reaction then was spun briefly and the following components were added to each tube:

5 μl 10× buffer #3 (NEB)

5 μl 10×BSA

2 μl 1M Tris pH 8.0

0.5 μl 5 M NaCl

4 μl Notl (NEB; 10 units/μl)

add 33.5 μl of dH₂O

The solution then was incubated at 37° C. for 2 hours. The digested DNA then was run on 1% agarose gel and the Fab (˜1.4 Kb) and scFv (˜700 bp) bands were excised. The DNA from the gel slices was purified by extraction using Frozen Phenol purification of DNA from low melt agarose. The purified Fab and scFv DNA was quantitated using the Picogreen® kit from Molecular Probes.

### 3. Ligation of scFv Fragment into Vector

The scFv DNA was ligated to pBADHA using the following ligation mix (keep the molar ratio of insert versus vector at 1-2:1)

X μl pBADHA (Sfil/Notl cut; 820 ng for scFv)

X μl Fab or ScFv (Sfil/Notl cut; 180 ng for ScFv)

5 μl T4 DNA ligase buffer

5 μl T4 DNA ligase (NEB; 400 units/μl)

add dH₂O to bring to total of 50 μl

The ligation reaction was incubated at 16° C. for ˜16 hours, then chilled on ice for 5 min and spun briefly. The ligation mixture was buffer exchanged using Princeton Separations' Centri-Spin 20 columns (Princeton Separations, Adelphia N.J.) according to manufacture's instruction. Briefly, the centri-spin 20 columns were hydrated with 650 μl ddH₂O at room temperature for at least 30 minutes. The ligation mix was heated to 66-68° C. for 10 min to inactivate the ligase and linearize any non-ligated molecules. The centri-spin 20 columns were placed in the 2 ml wash tube and spun at 750 × g for 2 minutes. The ligation mix (20-50 μl) was added on the top of the gel bed (be careful not to disturb the gel bed). The column was placed in the collection tube (1.5 ml tube) and spun at 750 × g for 2 min to collect the sample.

### 4. Transformation

The electroporation cuvettes (VWR; 1 mm gap) and 0.5 ml eppendorf tubes were prechilled on ice. The frozen electrocompetent cells were thawed on ice. Forty μl XL1 -Blue or TG1 cells were added to a 0.5 ml tube on ice, followed by addition of 1 μl of ligation mix to the tube. The tubes were placed on ice for ˜1 min.

The transformation mix then was transferred to the prechilled electroporation cuvettes on ice and shaken to the bottom of the cuvettes. The mixture was electroporated once at 1.7 KV (1.66 KV for DH12S from GIBCO). Immediately following electroporation, 300 μl of 2× YT/2% glucose medium was added to the cuvette. The transformation steps above were repeated 49 more times for total of 50 individual samples for each ligation. The contents of the 50 cuvettes (˜15 ml) was transferred to a 50 ml tube with transfer pipette (need two tubes). The culture was incubated for 1 hour at 37° C. with shaking at 250 rmp. Fifty lip for was set aside for titering (see below). Three hundred μl of the transformed cells were plated onto 50 separate 2×YT/2% glucose/Amp (0.1 mg/ml) plates (150 mm) using sterile glass beads. Once dry, the plates were inverted and incubated at 37° C. overnight. The cells were removed from the plates by flooding each plate with 5 ml 2×YT and scraping the cells into medium with a sterile spreader. Five ml of cells were reserved for phage rescue (see below). Frozen cell stock was prepared by adding glycerol to a final concentration of 15% and storing at −80° C. in 1 ml aliquots (10 aliquots is sufficient).

For cell titering, 1 μl, 10 μl and 30 μl of transformants from the above transformation were plated on 2×YT/2% glucose/Amp (0.1 mg/ml) plates (100 mm). The plates were incubated overnight at 37° C. Following the incubation, the colonies were visually counted and the colony forming units determined.

### 5. Rescue of the Library

One ml of the scraped cells were transferred to a 500 ml shake flask. The cells were diluted to OD600=0.2 with 2×YT/2% glucose. The culture was incubated for 1 hour at 37° C. with shaking at 250 rpm and measured the OD₆₀₀. M13KO7 (Stratagene, San Diego Calif.; Veira et al. (1987) Meth. Enz. 153:3) helper phage was added to the culture at a multiplicity of infection (moi) of 5:1 (10D600=8×10⁸ cells). The culture was incubated for 1 hour at 37° C. with shaking at 250 rpm, then spun at 1000× g for 20 min. Following the centrifugation, the supernatant was carefully remove and discarded. The pellet was gently resuspended in 500 ml of 2×YT/Amp/Kan medium in a 2 L shake flask. The culture was incubated overnight at 30° C.

Following the incubation, the cells were centrifuged at 8000 rmp for 30 min at 4° C. The resulting supernatant, which contained the recombinant phage, was transferred to 500 ml centrifuge bottles (2 bottles total). 4-(2-aminoethyl)benzenesulfonyl fluoride (AEBSF) was added to a final concentration of 0.2 μM.

### Example 3

### Creation and Production of scFv Libraries with Even Distribution of Polypeptide Tags

### A. Preparation of pBAD:Tag Expression Vectors

### 1. The pBAD: Tag Vector

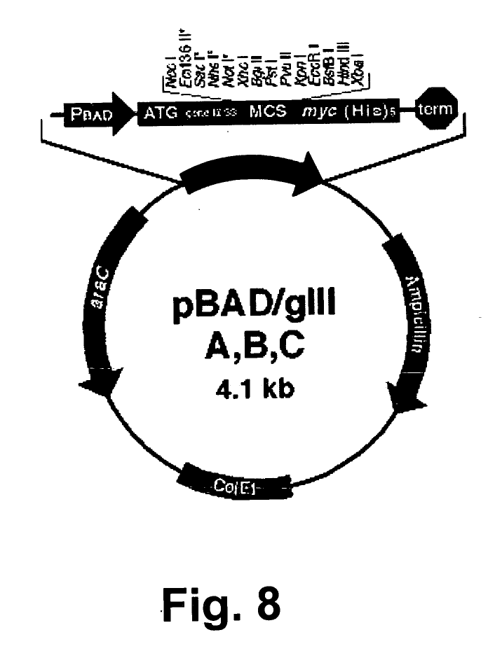

The A form of the pBAD/gill vector (FIG. 8; SEQ ID No. 163; Invitrogen) was modified for expression of scFvs by alteration of the multiple cloning sites to make it compatible with the Sfil and Notl sites used for most scFv construction protocols. The oligonucleotides SfilNotlFor and SfilNotlRev (SEQ ID Nos. 6 and 7) were hybridized and inserted into Ncol and HindIII digested pBAD/gIII DNA by ligation with T4 DNA ligase. The resultant vector (pBADmyc) permits insertion of scFvs in the same reading frame as the gene Ill leader sequence and the polypeptide tag, which has a sequence of EQKLISEEDL (SEQ ID No. 91).

For insertion of the scFv, the vector was incubated for 2 hours at 50° C. in a volume of 100 μl with 100 Units of Sfil (New England Biolabs) in 50 mM NaCl, 10 mM Tris-HCl, 10 mM MgCl₂, 1 mM dithiothreitol (DTT) pH 7.9 supplemented with 100 μg/ml bovine serum albumin (BSA). Following digestion with Sfil, the reaction was supplemented with additional H₂O, MgCl₂, Tris-HCl, NaCl, DTT, BSA, and Notl (New England Biolabs) such that the reaction volume is 150 μl containing 100 Units of Notl in 100 mM NaCl, 50 mM Tris-HCl, 10 mM MgCl₂, 1 mM DTT pH 7.9 and 100 μg/ml BSA. This reaction was incubated at 37° C. for 2 hours. Calf intestinal phosphatase (25 Units CIP, New England Biolabs) was added to the reaction and incubated at 37° C. for an additional 1 hour. Simultaneously, the scFv sub-library was digested with other features of the pBAD/gIII vector including an arabinose inducible promoter (araBAD) for tightly controlled expression, a ribosome binding sequence, an ATG initiation codon, the signal sequence from the M13 filamentous phage gene III protein for expression of the scFv in the periplasm of E. coli, a myc polypeptide tag for recognition by the 9E10 monoclonal antibody, a polyhistidine region for purification on metal chelating columns, the rrnB transcriptional terminator, as well as the araC and beta-lactamase open reading frames, and the ColE1 origin of replication. Additional vectors were created to contain the following polypeptide tags in place of the myc epitope:

| | | |
|---|---|---|
| Epitope | SEQ ID No. | Sequence |

| | | |
|---|---|---|
| T7 Tag | 96 | MASMTGGQQMG |
| HSV Tag | 97 | QPELAPEDPED |
| VSV-G | 101 | YTDIEMNRLGK |
| V5 | 95 | GKPIPNPLLGLDST |
| Glu—Glu | 94 | (C) EEEEYMPME |
| HA.11 | 92 | (C)YPYDVPDYA |
| E-tag | 100 | GAPVPYPDPLEPR |
| Flag | 93 | DYKDDDDK |
| Ab2 | 161 | LTPPMGPVIDQR |
| Ab4 | 162 | QPQSKGFEPPPP |

### 2. Screening for Antigen Reactivity

Cultures were screened for reactivity to antigen in a standard ELISA. Briefly, 96-well polystyrene plates were coated overnight with 10 μg/ml antigen (Sigma) in 0.1 M NaHCO₃, pH 8.6 at 4° C. Plates were rinsed twice with 50 mM Tris, 150 mM NaCl, 0.05% Tween-20, pH 7.4 (TBST), and then blocked with 3% non-fat dry milk in TBST (3% NFM-TBST) for 1 hour at 37° C. Plates were rinsed 4 times with TBST and 40 μl of unclarified culture was added to wells containing 10 μl 10% NFM in 5×PBS. Following incubation at 37° C. for 1 hour, plates were washed 4 times with TBST. The 9E10 monoclonal antibody (Covance) recognizing the myc polypeptide tag was diluted to 0.5 μg/ml in 3% NFM-TBST and incubated in wells for 1 hour at 37° C. Plates ware washed 4 times with TBST and incubated with horseradish peroxidase conjugated goat-anti-mouse IgG (Jackson Immunoresearch, 1:2500 in 3% NFM-TBST) for 1 hour at 37° C. After 4 additional washes with TBST, the wells were developed with o-phenylene diamine substrate (Sigma, 0.4 mg/ml in 0.05 Citrate phosphate buffer pH 5.0) and stopped with 3N HCl. Plates were read in a microplate reader at 492 nm. Cultures eliciting a reading above 0.5 OD units were scored positive and retested for lack of reactivity to a panel of additional antigens. Those clones that lacked reactivity to other antigens, and repeat reactivity to the specific antigen were grown up in culture. The DNA was prepared and the scFv was subcloned by standard methods into the pBADHA and pBADM2 vectors.

### B. Cloning of scFv Fragments into pBAD: Tag Vectors

### 1. Generation of Sfil/Notl Digested scFv Fragments and Digested pBAD:Tag Vector

Purified scFv DNA (1 μg×n where n is the number of tags) was digested with 4 μl SfiI (20 units/μl) in a total volume of 100 μl in 10 mM Tris-HCl, 10 mM MgCl₂, 50 mM NaCl, 1 mM DTT buffer (pH 7.9) for 2 hours at 50° C. The tube was spun briefly and the pH adjusted to 8.0. The DNA then was digested with 8 μl NotI (10 units/μl) in a total volume of 200 μl in a 50 mM Tris-HCl, 10 mM MgCl₂, 100 mM NaCl, 1 mM DTT buffer at 37° C. for 2 hours. The digested DNA was electrophoresed on a 1% agarose gel and the scFv band (˜700 bp) excised. The DNA was purified and quantified according to standard procedures well known to those with skill in the art.

Each of the pBAD: Tag Vectors (where each vector has a unique tag representing a single epitope) was separately digested with SfiI and NotI as described above. The digested DNA was electrophoresed on a 1% agarose gel and the linear vector band was excised. The DNA was, purified and quantified according to standard procedures well known to those with skill in the art.

### 2. Ligation of scFv Fragment into pBAD: Tag Vectors

Ligation mixtures were prepared such that the molar ratio of insert to vector was kept at 1-2:1. The digested scFv fragments were divided into a number of aliquots (equal to the number of pBAD: tag vectors) to which an aliquot of the SfiI/NotI digested pBAD: tag vector was added. The scFv was ligated into the vector by addition of T4 DNA ligase (400 units/μl) in 50 mM Tris-HCl (pH 7.5), 10 mM MgCl₂, 10 mM DTT, 1 mM ATP, 25 μg/ml bovine serum albumin buffer in a total volume of 50 μl. The ligation reaction was incubated at 16° C. for ˜16 hours, followed by chilling the reaction on ice for 5 min and a brief spin.

### 3. Transformation into E. coli and Growth of Recombinant Expression Vector

Freshly thawed frozen electro-competent Top 10 E. coli cells (40 μl; Invitrogen) were added to pre-chilled electroporation cuvettes (1 mm gap) along with 1 μl of each ligation reaction (the number of transformations will equal the number of ligations and hence the number of tags) and the cuvettes were placed on ice for ˜1 min. The cells were transformed by electroporation at 1.7 KV (1.66 KV for DH12S from GIBCO) and recovered by the immediate addition of 500 μl of SOC medium to the cuvette. The content of each cuvette was transferred to snap-cap culture tubes and the cells incubated for 45 minutes at 37° C. with shaking at 260 RPM. Frozen stocks of each of the transformed cells were prepared by adding-glycerol to a final concentration of 15% followed by storage at −80° C. in 0.1 ml aliquots.

### 4. Titering

An aliquot of each of the transformed cells was thawed and 5 μl aliquots were plated on LB/Amp (0.1 mg/ml) plates (100 mm). The plates were incubated overnight at 37° C. and the titer determined. The titer for each single tag library (single tag library is an aliquot of the scFv library cloned into each pBAD: tag vector) was the number of colony forming units (cfu) per ml of transformed cells.

### C. Distribution of Tagged scFv Libraries into Pools

### 1. Normalization of Titers

After the titers were determined as described above, a frozen aliquot of each-single tag library was thawed and 2×YT/2% glucose was added such that the titers are all normalized to be similar to the single tag library with the lowest titer.

### 2. Pooling the Tagged Libraries

The tagged libraries were pooled by either determining the diversity of scFvs to be displayed (e.g., 10⁹) or by determining the number of tags to be used for displaying the scFvs (e.g., 10²). The amount of aliquot of each normalized tagged library to be pooled was calculated using the formula: diversity to be displayed/number of tags (e.g., 10⁹/10²=10⁷). The calculated amount of each aliquot for each tag was added to a 15 ml tube and kept on ice.

### 3. Splitting the Mixed Library

The mixed library was split into aliquots such that 1000 scFvs were represented per tag within each aliquot (e.g., for 10² tags, each aliquot will have 1000 scFvs per tag which corresponds to a total of 10⁵ scFvs per aliquot). Each of these aliquots was called an array library.

### D. Expression of scFv Array Libraries

### 1. Starter Culture for scFv Protein Expression

Each array library was inoculated into 1 ml 2×YT supplemented with 50 μg/mL of carbenicillin. The culture was grown at 37° C. for 4 hours with shaking at 260 RPM. The culture then was added to 100 ml of 2×YT containing carbenicillin and grown at 37° C. for an additional 16 hours.

### 2. Preparation of Glycerol Stocks

Sterile glycerol was added to a final concentration of 15% to a 5 ml aliquot of the culture and stored at −80° C. in 0.5 ml aliquots.

### 3. Induction and Harvesting of E. coli Cells

Each of the starter cultures was diluted 4-fold by adding 300 mL 2×YT supplemented with 50 μg/mL of carbenicillin. To induce expression, arabinose was added to a final concentration of 0.1% and the cultures were grown at 30° C. with shaking at 260 RPM for 12 hours. Cells were harvested by centrifugation at 5000 g for 20 min at 4° C.

### E. Periplasmic Extraction of scFvs

Each pellet was resuspended in 12 mL of Periplasting Buffer (200 mM Tris-HCl, pH 7.5, 20% sucrose, 1 mM EDTA) followed by addition of 6 μl of lysozyme (to a final concentration of 30 units/μL) and incubation at room temperature for 5 min. The tubes then were placed on ice, with 36 mL of chilled, pure H₂O added to each tube followed by incubation on ice for 10 min. Periplasmic lysates were clarified by centrifugation at 10,000 g for 20 minutes. The supernatants then were transferred into clean tubes.

### F. Parallel Purification of scFv Array Libraries

### 1. Preparation and Equilibration of Affinity Columns

The following components were added to the periplasmic lysate described above such that the final concentration of each component was as indicated below:

500 mM NaCl

10 mM MgCl₂

20 mM Tris, pH 8.0

5 mM Imidazole

For each 50 ml of periplasmic lysate, 1 ml of Ni-NTA slurry was added. Pre-equilibration of the Ni-NTA was performed by adding the required amount of resin in a centrifuge tube, followed by centrifugation at 4000 g for 5 min. The supernatant was aspirated off and an equal volume of Lysis Buffer (50 mM NaH₂PO₄ (pH 8), 300 mM NaCl, and 10 mM imidazole) was added to resuspend the resin. The resin was centrifuged again at 4000 g for 5 min followed by aspiration of the supernatant. An equal volume of Lysis Buffer was used to resuspend the resin and the appropriate volume of slurry (corresponding to 1 mL Ni-NTA) was added to each lysate. Binding of scFv to the Ni-NTA was allowed to occur by incubation overnight at 4° C. on a rocker.

### 2. Manifold Chromatography

The columns were placed on the manifold (up to 20 columns can be accommodated per batch) with the stopcocks in the closed position before beginning. Syringes were placed on each column and the slurry poured into the syringes. Vacuum (˜0.1 bar) was applied and the stopcock-opened-to allow flow through the columns. Once the entire load volume has passed through the column, the stopcock was closed. (Once the load has passed through the column, it is important to shut the stopcock immediately to avoid drying the resin). Wash Buffer (50 mM NaH₂PO₄ (pH 8), 300 mM NaCl, 20 mM imidazole; 3 ml) was poured into the syringe and the vacuum applied as before. Once the entire Wash Buffer passed through the columns, the stopcocks were closed and the vacuum turned off. The manifold was opened and collection tubes were placed under each column. Elution Buffer (50 mM NaH₂PO₄ (pH 8), 300 mM NaCl, 250 mM imidazole, 50 mM EDTA; 1 ml) was applied to each column and a vacuum was applied. Once the entire aliquot of Elution Buffer passed through the column, the stopcocks were closed and the vacuum turned off. The tubes containing the elution material were capped and stored on ice until buffer exchange.

### 3. Buffer Exchange and Storage of scFv Array Libraries

Ten μL of 10% Tween-20 solution was added to each elution tube. The eluate then was added to a dialysis cassette, which was placed in 1 L of phosphate buffered saline, pH 7.4 (PBS). The buffer exchange was allowed to take place overnight with stirring at 4° C. Glycerol was added to each dialyzed sample to a final concentration of 20% and each sample was aliquoted and stored at −80° C.

### Example 4

### Preparation of Arrays and use thereof for Capturing Antibodies

### A. Sandwich Assay ELISA Kits

The components of Enzyme-linked immunosorbent assay (ELISA) CytoSets™ kits (BioSource), available for the detection of human cytokines, were used to generate “sandwich assays” for certain experiments. The “sandwich” as used in the below description was composed of a bound capture antibody, a purified cytokine antigen, a detector antibody, and streptavidine•HRPO. These kits allowed for the detection of the following human cytokines: human tumor necrosis factor alpha (Hu TNF-α; catalog #CHC1754, lot #001901) and human interleukin 6 (Hu IL-6; catalog #CHC1264, lot #002901).

### B. Anti-tag Capture Antibodies

For microarray analyses of scFv function and specificity, capture antibodies specific for hemagglutinin (HA. 11, specific for the influenza virus hemagglutinin epitope YPYDVPDYA; Covance catalog #MMS-101P, lot #139027002) and Myc (9E10, specific for the EQKLISEEDL amino acid region of the Myc oncoprotein; Covance catalog #MMS-1 50P, lot #139048002) were used. A negative control mouse IgG antibody (FLOPC-21; Sigma catalog #M3645) was also included in these assays.

### C. Capture Antibody Printing

### 1. Preparation of CytoSets™ Capture Antibodies for Printing with Either Modified Inkjet Printer or a Pin-Style Microarray Printer

Prior to printing CytoSets™ antibodies using a modified inkjet printer or a pin-style microarray printer (see below), capture antibodies from these kits were diluted in glycerol (Sigma catalog #G-6297, lot #20K0214) to 1-2 mg/ml, in a final glycerol concentration of 1% or 10%. Typically these mixtures were made in bulk and stored in microcentrifuge tubes at 4° C.

### 2. Preparation of Anti-Peptide Tag Capture Antibodies for Printing with a Pin-Style Microarray Printer

Capture antibodies specific for peptide tags present on certain scFvs were prepared by serial two-fold dilution. Capture antibody stocks (1 mg/ml) were diluted into a final concentration of 20% glycerol to yield typical final capture antibody concentrations of from 800 to 6 μg/ml: Capture antibody dilutions were prepared in bulk, stored in microcentrifuge tubes at 4° C. and loaded into 96-well microtiter plates (VWR catalog #62406-241) immediately prior to printing. Alternatively, capture antibody dilutions were made directly in a 96-well microtiter plate immediately prior to printing.

### 3. Capture Antibody Printing using a Modified Inkjet Printer

CytoSets™ capture antibodies were printed with an inkjet printer (Canon model BJC 8200 color inkjet) modified for this application. The six color ink cartridges were first removed from the print head. One-milliliter pipette tips then were cut to fit, in a sealed fashion, over the inkpad reservoir wells in the print head. Various concentrations of capture antibodies, in glycerol, then were pipetted into the pipette tips which were seated on the inkpad reservoirs (typically the pad for the black ink reservoir was used).

For generation of printed images using the modified printer, Microsoft PowerPoint was used to create various on-screen images in black-and-white. The images then were printed onto nitrocellulose paper (Schleicher and Schuell (S&S) Protran BA85, pore size 0.45 μm, VWR catalog #10402588, lot #CF0628-1) which was cut to fit and taped over the center of an 8.5×11 inch piece of printer paper. This two-paper set was hand fed into the printer immediately prior to printing. After printing of the image, the antibodies were dried at ambient temperature for 30 min. The nitrocellulose then was removed from the printer paper, and processed as described below (see Basic protocol for antibody and antigen incubations: FAST™ slides and nitrocellulose filters printed with CytoSets™ capture antibodies).

### 4. Capture Antibody Printing using a Pin-Style Microarray Printer

Capture antibody dilutions were printed onto nitrocellulose slides (Schleicher and Schuell FAST™ slides; VWR catalog #10484182, lot #EMDZ018) using a pin-printer-style microarrayer (MicroSys 5100; Cartesian Technologies; TeleChem Arraylt™ Chipmaker 2 microspotting pins, catalog #CMP2). Printing was performed using the manufacturer's printing software program (Cartesian Technologies' AxSys version 1,7, 0, 79) and a single pin (for some experiments), or four pins (for some experiments). Typical print program parameters were as follows: source well dwell time 3 sec; touch-off 16 times; microspots printed at 0.5 mm pitch; pins down speed to slide (start at 10 mm/sec, top at 20 mm/sec, acceleration at 1 000 mm/sec²); slide dwell time 5 millisec; wash cycle (2 moves+5 mm in rinse tank; vacuum dry 5 sec); vacuum dry 5 sec at end. Microarray patterns were pre-programmed (in-house) to suit a particular microarray configuration. In many cases, replicate arrays were printed onto a single slide, allowing subsequent analyses of multiple analyte parameters (as one example) to be performed on a single printed slide. This in turn maximized the amount of experimental data generated from such slides. Microtiter plates (96-well for most experiments, 384-well for some experiments) containing capture antibody dilutions were loaded into the microarray printer for printing onto the slides. Based on the reported print volume (post-touch-off, see above) of 1 nl/microspot for the Chipmaker 2 pins, the capture antibody concentrations contained in the printed microspots typically ranged from 800 to 6 pg/microspot.

Printing was performed at 50-55% relative humidity (RH) as recommended by the microarray printer manufacturer. RH was maintained at 50-55% via a portable humidifier built into the microarray printer. Average printing times ranged from 5-1 5 min; print times were dependent on the particular microarray that was printed. When printing was completed, slides were removed from the printer and dried at ambient temperature and RH for 30 min.

### D. Blocking Agent, PBS, and PBS-T

Following capture antibody printing, blocking of slides was performed with Blocker BSA™(10% or 10× stock; Pierce catalog #37525) diluted in phosphate-buffered saline (PBS) (BupH™ modified Dulbecco's PBS packs; Pierce catalog #28374). Tween-20 (polyoxyethylene-sorbitan monolaurate; Sigma catalog #P-7949) then was added to a final concentration of 0.05% (vol:vol). The resulting blocker is hereafter referred to as BBSA-T, while the resulting PBS with 0.05% (vol:vol) Tween-20 is referred to as PBS-T.

### E. Incubation Chamber Assemblies for FAST™ Slides

For isolation of individual microarrays of capture antibodies on a single FAST™ slide, slotted aluminum blocks were machined to match the dimensions of the FAST™ slides. Silicone isolator gaskets (Grace BioLabs; VWR catalog #s 10485011 and 10485012) were hand-cut to fit the dimensions of the slotted aluminum blocks. A “sandwich” consisting of a printed slide, gasket, and aluminum block then was assembled and held together with 0.75 inch binder clips. The minimum and maximum volumes for one such isolation chamber, isolating one antibody microarray, were 50 and 200 μl, respectively.

### F. Basic Protocol for Antibody and Antigen Incubations

### 1. FAST™ Slides and Nitrocellulose Filters Printed with CytoSets™ Capture Antibodies

After printing CytoSets™ capture antibodies onto FAST™ slides or nitrocellulose filters, these support media were allowed to dry as described. Slides and filters then were blocked with BBSA-T, for 30 min to 1 hr, at ambient temperature (filters) or 37° C. (slides). All incubations were done on an orbital table (ambient temperature incubations) or in a shaking incubator (37° C. incubations).

Purified, recombinant cytokine antigen (contained in each CytoSets™ kit) then was diluted to various concentrations (typically between 1-10 ng/ml) in BBSA-T. Slides or filters, containing CytoSets™ capture antibodies, then were incubated with this antigen solution at ambient temperature (filters) or 37° C. (slides). Slides and filters then were washed three times with PBS-T, 3-5 min per wash, at ambient temperature. These slides and filters, containing capture antibody with bound antigen, then were incubated with detector antibody (contained in each kit) diluted 1:2500 in BBSA-T for 1 hr, at ambient temperature (filters) or 37° C. (slides). Slides and filters then were washed with PBS-T as described above.

These slides and filters, containing capture antibody, bound antigen, and bound detector antibody, then were incubated with streptavidin•HRPO (contained in each kit) diluted 1:2500 in BBSA-T for. 1 hr, at ambient temperature (filters) or 37° C. (slides). Slides and filters then were washed with PBS-T as described above. The slides and filters then were developed and imaged as described below.

### 2. FAST™ Slides Printed with Anti-Peptide Tag Capture Antibodies

After printing anti-peptide tag capture antibodies onto FAST™ slides, the slides were allowed to dry as described. Slides then were blocked with BBSA-T, for 30 min to 1 hr, at 37° C. in a shaking incubator (37° C. incubations).

Purified scFvs, containing peptide tags, then were diluted to various concentrations (typically between 0.1 and 100 μg/ml) in BBSA-T. Slides containing anti-peptide tag capture antibodies then were incubated with this antigen solution for 1 hr at 37° C. Slides then were washed three times with PBS-T, 3-5 min per wash, at ambient temperature.

Slides containing anti-peptide tag capture antibodies and bound scFvs then were incubated with biotinylated human fibronectin or biotinylated human glycophorin (as antigens) diluted to various concentrations (typically 1-10 μg/ml) in BBSA-T, for 1 hr at 37° C. Slides then were washed with PBS-T as described above.

Slides containing anti-peptide tag capture antibodies, bound scFvs, and bound biotinylated antigens then were incubated with NeutravidinoHRPO diluted 1:1000 or 1:100,000 in BBSA-T, for 1 hr at 37° C. Slides then were washed with PBS-T as described above. These slides then were developed and imaged as described below.

### G. Developing and Imaging of FAST™ Slides and Nitrocellulose Filters Containing Antibody Microarrays

After washing in PBS-T, slides containing anti-peptide tag antibodies, bound scFvs, antigens, and Neutravidin∘HRPO, or nitrocellulose filters containing CytoSets™ antibodies, bound cytokine antigens, detector antibody, and streptavidin∘HRPO, were rinsed with PBS, then developed with Supersignal™ ELISA Femto Stable Peroxide Solution and Supersignal™ ELISA Femto Luminol Enhancer Solution (Pierce catalog #37075) following the manufacturer's recommendations.

FAST™ slides and filters were imaged using the Kodak Image Station 440CF. A 1:1 mixture of peroxide solution:luminol was prepared, and a small volume of this mixture was placed onto the platen of the image station. Slides then were placed individually (microarray-side down) into the center of the platen, thus placing the surface area of the nitrocellulose-containing portion of the slide (containing the microarrays) into the center of the imaging field of the camera lens. In this way the small volume of developer, present on the platen, contacted the entire surface area of the nitrocellulose-containing portion of the slide. Nitrocellulose filters were treated in the same manner, using somewhat larger developer volumes on the platen. The Image Station cover then was closed and microarray images were captured. Camera focus (zoom) was set to 75 mm (maximum; for FAST™ slides ) or 25 mm for filters. Exposure times ranged from 30 sec to 5 min. Camera f-stop settings ranged from 1.2 to 8 (Image Station f-stop settings are infinitely adjustable between 1.2 and 16).

### H. Archiving and Analysis of Microarray Images

Archiving and analysis of microarray images was performed using the Kodak 1D 3.5.2 software package. Regions of interest (ROIs) were drawn to frame groups of capture antibodies (printed at known locations on the microarrays), typically in groups of four (two-by-two) or 64 (eight-by-eight) microspots. Numerical ROI values, representing net, sum, minimum, maximum, and mean intensities, as well standard deviations and ROI pixel areas, were automatically calculated by the software. These data then were transformed into Microsoft Excel for statistical analyses.

### I. Results

### 1. Human Tumor Necrosis Factor α Array

Two microarray-type patterns of human tumor necrosis factor a (TNF-α) capture antibody (from CytoSets™ kit) were printed onto nitrocellulose with a modified inkjet printer using Microsoft PowerPoint. TNF-α capture antibody was diluted to 1.25 ng/ml in 1% glycerol for printing. After drying, the filter was blocked with BBSA-T. The microarrays then were probed with purified recombinant human TNF-α (5.65 ng/ml) as antigen. The filter then was washed with PBS-T. Detector antibody and streptavidin•HRPO then were used for detection of bound antigen. After washing in PBS-T, the microarrays were developed using chemiluminescence and imaged on a Kodak Image Station 440CF. High resolution images were generated with feature sizes below 50 μm.

A single microarray of human interleukin-6 (IL-6) capture antibody (from CytoSets™ kit) was printed onto a FAST™ slide with a pin-style microarray printer (4-pin print pattern) programmed to print the pattern. IL-6 capture antibody was diluted to 0.5 mg/ml in 10% glycerol. One nanoliter microspots of capture antibody were printed which contained 500 pg/microspot. After drying, the slide was blocked with BBSA-T. The microarray then was probed with purified recombinant human IL-6 (5 ng/ml) as antigen. Following incubation with the antigen, the slide was washed with PBS-T. Detector antibody and streptavidin∘HRPO then were used for detection of bound antigen. After washing in PBS-T, the microarrays were developed using chemiluminescence and imaged on a Kodak Image Station 440CF. The method produced bright images with array feature sizes corresponding to 300 μm loci. In additional experiments, dilution of capture antibody or antigen gave increased or reduced signals corresponding to a direct relationship between the amount of antigen bound and the signal produced.

### 2. Microarrays of Anti-Peptide Tags

Microarrays (8-by-8 microspots) of anti-peptide tag capture antibodies (HA.11, specific for the influenza virus hemagglutinin epitope, YPYDVPDYA; 9E10, specific for the EQKLISEEDL (SEQ ID No. 91) amino acid region of the Myc oncoprotein; and FLOPC-21, a negative control antibody of unknown specificity) were printed onto a FAST™ slide with a pin-style microarray printer (4-pin print pattern) programmed to print the pattern. The capture antibodies were diluted to 0.5 mg/ml in 20% glycerol. One nanoliter microspots were printed which contained serial two-fold dilutions of 500, 250, 125 and 62.5 pg/microspot. After drying, the filter was blocked with BBSA-T. The microarrays then were successively probed with aliquots of culture supernatant and periplasmic lysate harvested from an E. coli strain harboring the plasmid construct which directs the expression of the HA-HFN scFv upon arabinose induction. The slide then was washed with PBS-T. The microarrays then were probed with biotinylated human fibronectin (3.3 μg/ml). After washing with PBS-T, the microarrays were probed with excess Neutravidin•HRPO (1:1000). After washing in PBS-T, the microarrays were developed using chemiluminescence and imaged on a Kodak Image Station 440CF.

### 3. Microarrays of Human Interleukin-6

Microarrays of human interleukin-6 (IL-6) capture antibody (from CytoSets™ kit) were printed onto a FAST™ slide, and 4 different surfaces, with a pin-style microarray printer (4-pin print pattern) programmed to print the pattern. Human IL-6 capture antibody was diluted in 20% glycerol and printed to yield serial three-fold dilutions ranging from 300, 100, 33, 11, 3.6, 1, 0.3, and 0.1 pg/microspot. A negative control capture antibody, specific for human interferon-α (IFN-α) was also printed at 50 pg/microspot. After drying, the slide was blocked with BBSA-T. The microarrays then were probed with purified recombinant human IL-6 (5 ng/ml) as antigen followed by washing with PBS-T. Detector antibody and streptavidin•HRPO then were used for detection of bound antigen. After washing in PBS-T, the microarrays were developed using chemiluminescence and imaged on a Kodak Image Station 440CF. Signal was seen from loci containing 1 pg/locus and higher concentrations.

### Example 5

Quality Control of scFv Array Libraries

The three methods described below were used to monitor the quality of the scFv array libraries produces by the methods described in EXAMPLE 3. The basic protocol for each analytic method listed as well as other methods not exemplified here are known to those of skill in the art.

### A. Protein Assay

All scFv sub-libraries purified as in Example 4 above were diluted 1 to 2 with PBS and 40 μl aliquots were added to the top row of a 96-well polystyrene plate in duplicate. Each sample then was serially diluted 2-fold along each column of the 96-well plate. A BSA standard was added for calibration of the concentration range. Modified Lowry reagent was added to each of the wells and mixed briefly. After a 10 min incubation, Folin-Ciocalteau Phenol reagent was added and mixed per the manufacture's protocol (Pierce Endogen). The absorbance was measured at 750 nm after a 30 min incubation at room temperature.

### B. SDS-PAGE Analysis

Each purified scFv sub-library (15 μl) was mixed with 15 μl of 2× Laemmli Reducing Sample Buffer and heated at 100° C. for 10 minutes. Each sample then was loaded on a 12% SDS-PAGE gel and electrophoresed until the tracking dye was ˜1 cm from the bottom of the gel. The gel was stained to visualize proteins and a densitometric scan performed to measure the percentage homogeneity of each sample.

### C. MicroELISA Assay

An equal volume of 2× Print Buffer (2×PBS, 40% glycerol and 0.002% Tween-20) was added to each of the scFv sub-libraries to a final volume of 40 μl in a 96-well PCR plate. The solution was mixed and then spun briefly. The array libraries were printed on nitrocellulose-coated glass slides (FAST, Schleicher and Schuell, NH) using Telechem pins (CM-2) on a Cartesian printer (MicroSys 5100) such that 20 replicate arrays were printed on each slide. Printing was performed under 55 to 60% humidity and the plates air-dried for 1 hour followed by storage at 4° C.

After incubating each array with Blocking Buffer I (3% non-fat milk in PBS containing 0.1% Tween20 (PBS-T)) for 1 hour, the Blocking Buffer was aspirated off and each sub-array was incubated with an appropriate dilution of anti-tag antibody in Blocking Buffer II (1% BSA in PBS-T). Incubation was performed at room temperature for 1 hour. After aspiration, the wells were rinsed three times for 1 min each with PBS-T. This step was followed by incubation with an appropriate dilution of goat anti-mouse IgG-conjugated to horseradish peroxidase in Blocking Buffer II and three rinses with PBS-T. The array then was exposed to Luminol and the chemiluminescence detected using a CCD camera. The intensity of each locus was measured using software and the amount of individual tagged scFv in each pool determined.

### D. Assay for Quantification of Tag Distribution with Pools of scFv

Capture anti-tag antibodies were printed at 800, 200, and 50 μg/ml in ten replicate arrays onto n/10 FAST™ slides (where n=number of scFv pools to be analyzed). An extra slide was printed for use in obtaining the standard curve. Slides were incubated in Blocking solution (5% non-fat milk in PBS containing 0.1% Tween 20) for 1 hour at 37° C. Each pool of scFv was diluted to appropriate concentration (typically between 1 and 10 μg/ml) in Blocking Buffer and incubated with individual arrays for 1 hour at room temperature. A standard curve was generated with known amounts of scFV:huFN:tag (scFv recognizing human fibronectin conjugated to individual tags) by serial dilutions onto one slide so that samples can be quantified. Unbound scFv were removed by aspiration and slides were washed three times with Blocking solution. Rabbit anti-His₆ polyclonal antibody conjugated to HRP was incubated with all arrays at a 1:20,000 dilution from a 1 mg/ml stock solution for 30 minutes at room temperature. Slides were washed with PBS containing 0.1% Tween 20, prior to the addition of Luminol for detection on a Kodak IS1000 imaging station. The intensity of each locus was measured and the amount of individual tagged scFv in each pool determined by measuring against the standard curve.

### Example 6

### Determination of Anti-Idiotype

### A. MicroArray Printing

Stock solutions of the anti-IgM antibody (S1C5; anti-idiotype monoclonal antibody), the goat anti-mouse Fc antibody (this antibody recognizes the constant (Fc) regions of mouse antibodies) and anti-flag antibody were prepared at a concentration of 1 mg/ml or greater in PBS. For printing, the antibodies were brought to 800 μg/ml in 1× Print Buffer (1×PBS, 20% glycerol, 0.001% Tween-20) by adding ¼ volume of 4× Print Buffer (4×PBS, 80% glycerol, 0.004% Tween-20) to ¾ volume of a 1 mg/ml antibody solution in PBS. Two-fold serial dilutions were made of each antibody such that all antibodies were at 9 different concentrations in 1× Print Buffer (Table 8). Forty μl of antibody solution was transferred to a 96-well PCR plate.

Each of the antibodies were printed on FAST™ nitrocellulose-coated glass slides (Schleicher and Schuell) using a Telechem pin (CM-2) in a Cartesian printer (MicroSys 5100). Printing was performed at 55 to 60% relative humidity. The slides were subsequently incubated overnight at 4° C. for maximum adsorption to the nitrocellulose.

### B. Preparation of 38C13 Cell Extract

B cells (38C13) were grown in culture (Growth medium: RPMI 1640, 10% fetal calf serum, 55 μl 2-mercaptoethanol, penicillin and streptomycin) in 5% CO₂, 90% relative humidity and 37° C. to a density of 0.7×10⁶ cells/ml. A 2.5 ml aliquot (1.75×10⁶ cells total) was spun down at 1200 rpm for 5 minutes at 4° C. The pellet was washed one time with 4 ml of RPMI 1640 (Gibco), and spun down again at 1200 rpm for 5 minutes at 4° C. The pellet was resuspended at 4° C. in 175 μl of RPMI 1640 (Gibco), giving a concentration of 10⁶ cells per 100 μl. Resuspension was carried out by gently pipetting up and down 3-4 times.

Small (less than 1 ml) aliquots of tissue culture cells (38C13 and C6VL cells) prepared as described above were stored frozen in liquid nitrogen or at −80° C. in Freezing Medium (frequently 90% fetal calf serum/10% DMSO). The frozen cells were thawed quickly by rolling tube containing the aliquot between the palms. The cells were diluted immediately 10-fold with 4° C. PBS and centrifuged at 1200 rpm for 5 minutes at 4° C. Cells then were washed three times with 4° C. PBS at a density of 10⁶ cells/ml, based on the number of cells that were frozen for storage. The resuspended cells were used immediately for capture.

TABLE 8
Array Map (μg/ml)

| | 1 | 2 | 3 | 4 | 5 | 6 | 7 | 8 | 9 | 10 | 11 |
|---|---|---|---|---|---|---|---|---|---|---|---|
| A | NV-HRP 400 | — | S1C5 400 | S1C5 200 | S1C5 100 | S1C5 50 | S1C5 25 | S1C5 12.5 | S1C5 6.25 | S1C5 3.12 | — |
| B | NV-HRP 200 | — | S1C5 400 | S1C5 200 | S1C5 100 | S1C5 50 | S1C5 25 | S1C5 12.5 | S1C5 6.25 | S1C5 3.12 | — |
| C | NV-HRP 100 | — | g α-m Fc | g α-m Fc | g α-m Fc | g α-m Fc | g α-m Fc | g α-m Fc | g α-m Fc | g α-m Fc | — |
| | | | 121.9 | 60.95 | 30.475 | 15.238 | 7.619 | 3.809 | 1.905 | 0.952 | |
| D | — | — | g α-m Fc | g α-m Fc | g α-m Fc | g α-m Fc | g α-m Fc | g α-m Fc | g α-m Fc | g α-m Fc | — |
| | | | 121.9 | 60.95 | 30.475 | 15.238 | 7.619 | 3.809 | 1.905 | 0.952 | |
| E | — | — | g α-m Fc | g α-m Fc | g α-m Fc | g α-m Fc | g α-m Fc | g α-m Fc | g α-m Fc | g α-m Fc | — |
| | | | 121.9 | 60.95 | 30.475 | 15.238 | 7.619 | 3.809 | 1.905 | 0.952 | |
| F | NV-HRP 50 | — | g α-m Fc | g α-m Fc | g α-m Fc | g α-m Fc | g α-m Fc | g α-m Fc | g α-m Fc | g α-m Fc | NV-HRP 100 |
| | | | 121.9 | 60.95 | 30.475 | 15.238 | 7.619 | 3.809 | 1.905 | 0.952 | |
| G | NV-HRP 100 | — | anti-Flag | anti-Flag | anti-Flag | anti-flag | anti-Flag | anti-Flag | anti-Flag | anti-Flag | NV-HRP 200 |
| | | | 121.9 | 60.95 | 30.475 | 15.238 | 7.619 | 3.809 | 1.905 | 0.952 | |
| H | NV-HRP 200 | — | anti-Flag | anti-Flag | anti-Flag | anti-flag | anti-Flag | anti-Flag | anti-Flag | anti-Flag | NV-HRP 400 |
| | | | 121.9 | 60.95 | 30.475 | 15.238 | 7.619 | 3.809 | 1.905 | 0.952 | |

### C. Array Incubations

The printed slides were brought to room temperature and washed three times each for one minute with PBS. Following the wash step, the slides were blocked with 1 ml of Block Buffer (3% NMF/PBS/1% Triton X-100) on an orbital shaker in a humidified chamber for 1 hour at room temperature. The slides then were incubated with 38C13 cell extract and control 38C13 purified antibody as shown in Table 9 below. The extract was diluted 1:1 with Block Buffer for the highest concentration, then serially by factors of 10. Fifty μl of each sample was added to the wells and incubated with the array for 1 hour at room temperature on an orbital shaker.

TABLE 9

| Array Number | Sample |
|---|---|
| 1 | Block Buffer control |
| 2 | Extract (1:2000) |
| 3 | Extract (1:200) |
| 4 | Extract (1:20) |
| 5 | Extract (1:1) |
| 6 | 38C13 Ab 10 μg/ml |
| 7 | 38C13 Ab 1 μg/ml |
| 8 | 38C13 Ab 0.1 μg/ml |
| 9 | 38C13 Ab 0.01 μg/ml |
| 10 | Block Buffer Control |

Following the incubation, the wells then were washed three times with 200 μl of PBS/1% Triton X-100 for one minute on an orbital shaker. Fifty microliters of detection antibody (goat anti-mouse IgM HRP 1:5,000 in Block Buffer) then were added to each well and incubated for one hour at room temperature on an orbital shaker. The wells then were washed again three times with 200 μl of PBS/1% Triton X-100 for one minute on an orbital shaker. The slides then were removed from the chamber and rinsed with 500 μl PBS/1% Triton X-100. The arrays then were imaged on Kodak IS1000 in a petri dish, raised from the surface of the dish with two layers of plastic cover slips, with about 1 ml of luminol.

### D. Results

The purified IgM antibody (38C13) gave a strong signal on the S1C5 monoclonal antibody loci, down to a concentration of 25 μg/ml spotted protein and at an IgM concentration of 0.1 μg/ml, the lowest IgM concentration used. The 38C13 IgM in the 38C13 cell extracts were detected at a 1:2000 dilution of the extract, the lowest used, down to a concentration of 50 μg/ml printed S1C5. The 38C13 IgM did not bind to the anti-Flag monoclonal negative control, though non-specific binding of the Goat anti-Mouse IgM-HRP antibody can be seen (FIG. 10).

### Example 7

### Cell Capture MicroArrays

### A. MicroArray Printing

Stock solutions of the anti-M2 capture monoclonal antibody (M2), anti-Myc capture monoclonal antibody (Myc), anti-IgM (S1 C5; anti-idiotype monoclonal antibody) and anti-T cell receptor antibody (C6VL) were prepared at concentrations of 1 mg/ml or greater in PBS. For printing, the antibodies were brought to 800 μg/ml in 1× Print Buffer (1×PBS, 20% glycerol, 0.001% Tween-20) by adding ¼ volume of 4× Print Buffer (4×PBS, 80% glycerol, 0.004% Tween-20) to % volume of a 1 mg/ml antibody solution in PBS. Two-fold serial dilutions were made of each antibody such that all antibodies were at 9 different concentrations in 1× Print Buffer (Tables 10 and 11). Forty pl of antibody solution was transferred to a 96-well PCR plate.

Each of the antibodies were printed on FAST™ nitrocellulose-coated glass slides (Schleicher and Schuell) using a Telechem pin (CM4) in a Cartesian printer (MicroSys 5100). Printing was performed at 55 to 60% relative humidity. The slides were subsequently incubated overnight at 4° C. for maximum adsorption to the nitrocellulose.

TABLE 10
Array Map (μg/ml)

| | 1 | 2 | 3 | 4 | 5 | 6 | 7 | 8 | 9 | 10 | 11 |
|---|---|---|---|---|---|---|---|---|---|---|---|
| A | NV-HRP 200 | HA 200 | S1C5 200 | S1C5 200 | M2 200 | M2 200 | myc 200 | myc 200 | C6VL 200 | C6VL 200 | PB |
| B | NV-HRP 200 | HA 100 | S1C5 100 | S1C5 100 | M2 100 | M2 100 | myc 100 | myc 100 | C6VL 100 | C6VL 100 | PB |
| C | NV-HRP 100 | HA 50 | S1C5 50 | S1C5 50 | M2 50 | M2 50 | myc 50 | myc 50 | C6VL 50 | C6VL 50 | PB |
| D | NV-HRP 50 | HA 25 | S1C5 25 | S1C5 25 | M2 25 | M2 25 | myc 25 | myc 25 | C6VL 25 | C6VL 25 | PB |
| E | S1C5 200 | HA 12.5 | S1C5 12.5 | S1C5 12.5 | M2 12.5 | M2 12.5 | myc 12.5 | myc 12.5 | C6VL 12.5 | C6VL 12.5 | PB |
| F | NV-HRP 50 | HA 6.25 | S1C5 6.25 | S1C5 6.25 | M2 6.25 | M2 6.25 | myc 6.25 | myc 6.25 | C6VL 6.25 | C6VL 6.25 | NV-HRP 50 |
| G | NV-HRP 100 | HA 3.12 | S1C5 3.12 | S1C5 3.12 | M2 3.12 | M2 3.12 | myc 3.12 | myc 3.12 | C6VL 3.12 | C6VL 3.12 | NV-HRP 100 |
| H | NV-HRP 200 | HA 1.06 | S1C5 1.06 | S1C5 1.06 | M2 1.06 | M2 1.06 | myc 1.06 | myc 1.06 | C6VL 1.06 | C6VL 1.06 | NV-HRP 200 |

TABLE 11
Source Plate (μg/ml)

| | 1 | 2 | 3 | 4 | 5 | 6 | 7 | 8 | 9 | 10 | 11 |
|---|---|---|---|---|---|---|---|---|---|---|---|
| A | NV-HRP | C6VL 200 | M2 100 | S1C5 50 | PB | myc 25 | M2 12.5 | alpha5 6.25 | C6VL 6.25 | myc 3.25 | S1C5 1.06 |
| | 200 | | | | | | | | | | |
| B | alpha 5 | C6VL 200 | myc 100 | S1C5 50 | S1C5 200 | C6VL 25 | M2 12.5 | S1C5 6.25 | NV-HRP 50 | myc 3.25 | M2 1.06 |
| | 200 | | | | | | | | | | |
| C | S1C5 200 | PB | myc 100 | M2 50 | alpha5 25 | C6VL 25 | myc 12.5 | S1C5 6.25 | NV-HRP | C6VL 3.25 | M2 1.06 |
| | | | | | | | | | 100 | | |
| D | S1C5 200 | NV-HRP | C6VL 100 | S1C5 25 | S1C5 25 | PB | myc 12.5 | M2 6.25 | alpha5 3.12 | C6VL 3.25 | myc 1.06 |
| | | 100 | | | | | | | | | |
| E | M2 200 | alpha5 | C6VL 100 | S1C5 12.5 | S1C5 25 | NV-HRP 50 | C6VL 12.5 | M2 6.25 | S1C5 3.12 | NV-HRP | myc 1.06 |
| | | 100 | | | | | | | | 100 | |
| F | M2 200 | S1C5 100 | PB | S1C5 6.25 | M2 25 | alpha5 12.5 | C6VL 12.5 | myc 6.25 | S1C5 3.12 | NV-HRP | C6VL 1.06 |
| | | | | | | | | | | 200 | |
| G | myc 200 | S1C5 100 | NV-HRP | S1C5 3.12 | M2 25 | S1C5 12.5 | PB | myc 6.25 | M2 3.12 | alpha5 1.06 | C6VL 1.06 |
| | | | 50 | | | | | | | | |
| H | myc 200 | M2 100 | alpha5 50 | S1C5 1.06 | myc 25 | S1C5 12.5 | NV-HRP 50 | C6VL 6.25 | M2 3.12 | S1C5 1.06 | NV-HRP |
| | | | | | | | | | | | 200 |

### B. Preparation of Non-Adherent Cells for Capture

### 1. Tissue Culture Cells

B cells (38C13) and T cells (C6VL) were grown in culture (Growth medium: RPMI 1640, 10% fetal calf serum, 55 μl 2-mercaptoethanol, penicillin and streptomydin) in 5% CO₂, 90% relative humidity and 37° C. 38C13 B cells were grown to a density of 0.7×10⁶ cells/ml in growth medium. A 2.5 ml aliquot (1.75×10⁶ cells total) was spun down at 1200 rpm for 5 minutes at 4° C. The C6VL T cells were grown to a density of 0.35×10⁶ cells/ml in growth medium. A 5 ml aliquot (1.75×10⁶ cells total) was spun down at 1200 rpm for 5 minutes at 4° C. The two pellets then were washed one time with 4 ml each of RPMI 1640, and spun down again at 1200 rpm for 5 minutes at 4° C. The two pellets then were resuspended at 4° C. in 175 μl of RPMI 1640, giving a concentration of 10⁶ cells per 100 μl. Resuspension was carried out by gently pipetting up and down 3-4 times. The resuspended cells were used immediately for capture.

### 2. Frozen Cells

Small (less than 1 ml) aliquots of tissue culture cells (38C13 and C6VL cells) prepared as described above were stored frozen in liquid nitrogen or at −80° C. in Freezing Medium (frequently 90% fetal calf serum/10% DMSO). The frozen cells were thawed quickly by rolling tube containing the aliquot between the palms. The cells were diluted immediately 10-fold with 4° C. PBS and centrifuged at 1200 rpm for 5 minutes at 4° C. Cells then were washed with 10 volumes of Incubation Buffer, centrifuged as above, and resuspended in 4° C. Incubation Buffer at a density of 10⁶ cells/ml, based on the number of cells that were frozen for storage. The resuspended cells were used immediately for capture.

### C. Cell Capture Assay

### 1. Monoclonal Anti-Cell Surface Antigen Arrays

The printed slides were brought to room temperature and washed three times each for one minute with PBS. Following the wash step, the slides were blocked with 1 ml of PBS containing 0.5% Bovine Serum Albumin on an orbital shaker in a humidified chamber for 1 hour at room temperature.

Following the blocking, excess Block Buffer was removed by tilting the slide and absorbing liquid from the lower end with a Kimwipe. One hundred μl (containing 10⁶ cells total in Incubation Buffer) of C6VL cells (T cells) were added to one slide and 100 μl (containing 10⁶ cells total in Incubation Buffer) of 38C13 cells (B cells) were added to the second slide by pipetting cells down the middle of the slides in sequential drops. The slides then were incubated again for 20-30 minutes at room temperature on an orbital shaker. Following the incubation, the slides were viewed immediately in a microscope differential interference contrast (DIC) microscopy (Nikon E800 with Locus CCD Camera). Optionally, the slides were gently washed first in Incubation Buffer at room temperature then viewed as above. In all cases, the printed slide was situated in the microscope such that the printed side with the cells was facing up.

### 2. Monoclonal Anti-Tag/Tag-scFv Arrays

Printed slides were incubated for 1 hour in Block Buffer as described above. Following the incubation, a mask was placed on the slide to create wells to separate the arrays. Peptide tag-scFv fusion protein, previously purified from bacteria by His-tag metal affinity chromatography as described in EXAMPLE 4, and stored in PBS at about. 1 mg/ml, was diluted 10-fold or more into Incubation Buffer. The slides then were incubated for 1 hour at room temperature with the purified peptide tag-scFv (1 ml/slide or if slides are in the 10-well mask, 50 μl/well) on an orbital shaker in either a humidified chamber or with an adhesive seal over the mask. The slides were washed 3 times with 200 μl of Incubation Buffer, 1 minute each time on an orbital shaker and then incubated with cells at 10⁷ cells/ml in Incubation Buffer for 20-30 minutes. One hundred μl was used for an entire slide. If slides were masked, then 50 μl of a 2×10⁶ cells/ml solution were applied per well. Slides were viewed directly in a microscope, or, optionally, gently washed first in Incubation Buffer then viewed in a microscope. In a mask, slides were washed 3 times with 400 μl Wash Buffer (0.5% BSA with buffered salt solution containing either culture medium with 10 mM Hepes pH 7.4, lacking phenol red, or PBS) one minute each time, on an orbital shaker at room temperature. Excess Wash Buffer was removed after each wash by aspirating all but about 100 μl of Buffer.

### D. Chemical Fixation of Cells to Arrays

Following cell capture on the arrays, cells were fixed with a 4% Formaldehyde Solution. The 4% solution was prepared by diluting 37% formaldehyde (Histology Grade, Sigma) 10-fold into the buffered salt solution used for capture. Following capture, excess Wash Solution was removed from the slide by tilting it and absorbing the run-off with a Kimwipe. The slide then was placed horizontally in a humidified chamber and 1 ml of the 4% Formaldehyde Solution was added to the array surface in drops along the length of the slide. The slide then was incubated at room temperature for 10 minutes and washed 3 times for 5 minutes each with 50 ml each time of PBS in either Complin jars or 50 ml conical tubes. Cells were permeabilized with Permeabilization Solution (0.1%-TX-100, PBS and 0.02% sodium azide) for 5 minutes at room temperature. The slides then were stored at 4° C. in the Permeabilization Solution.

### E. Results

The source plate is the 96-well plate used for printing the monoclonal antibodies on the FAST slides. The controls for this experiment were anti-cell surface antigen monoclonal antibodies that did not bind to the cell surface due to the lack of expression of that particular antigen on the cell. For example, anti-C6VL monoclonal antibody, which recognizes the T-cell receptor on C6VL cells, was used as a negative control when incubating 38C13 cells with an array, and SlC5 monoclonal antibody (which recognizes IgM on the 38C13 cells, was used as a negative control when incubating with the C6VL cells. When incubating the cells with arrays that had been loaded with ScFv's, the HFN (which recognizes human fibronectin) was used as the negative control for the 38C13 cells. A specific ScFv that recognizes the C6VL cells is not currently available. The results were that cells bound only to monoclonal antibodies and/or ScFv's that were specific for antigens expressed on that cell's surface. After binding the anti-cell surface antigen monoclonal antibodies captured the appropriate cell type, these were used as positive controls. The concentrations used for negative controls were identical to those used for cell-specific monoclonal antibodies and ScFv's.

### 1. Array Capture of Previously Frozen Cells

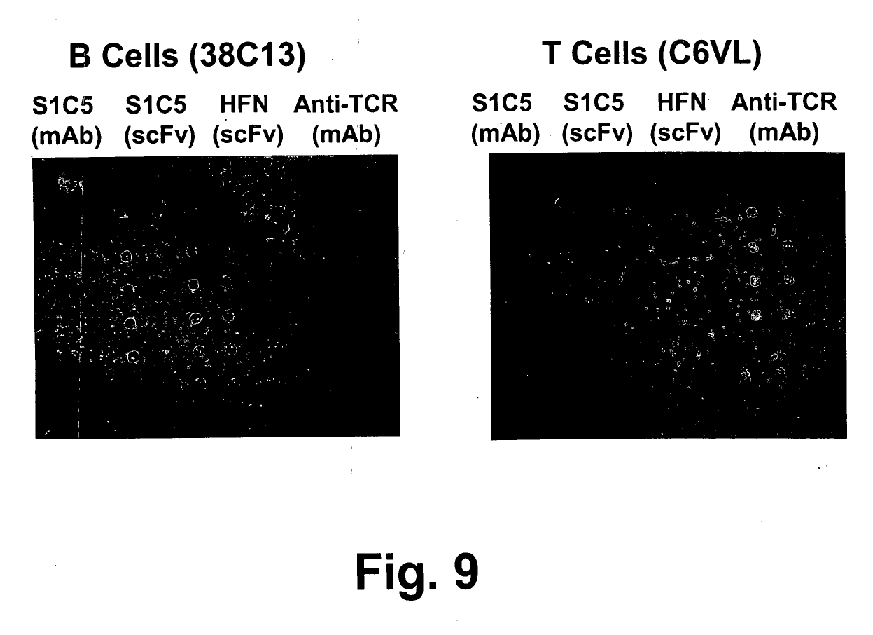

S1C5 mouse monoclonal antibody (stock concentration 3.6 mg/ml in PBS) was diluted to 400 μg/ml in 1× Print Buffer and then serially diluted 2-fold, 9 times for printing. Anti-tag monoclonal antibodies were diluted to 800 μg/ml from 1 mg/ml stocks as described above, and serially diluted 9 times for printing. With a mask, 10-fold serial dilutions of the S1C5 scFv containing the appropriate peptide tag, prepared and purified as described in EXAMPLE 4, were incubated with the arrays in PBS/0.5% BSA. Previously frozen 38C13 B lymphoma cells, which contained an IgM surface receptor recognized by the SlC5 antibody and scFv, were incubated with the array in PBS only. Cells captured on specific antibody or scFv containing loci were imaged with the Nikon E800 and Spot CCD camera. Cells were detected bound to loci printed from solutions down to 6.25 μg/ml of S1C5 antibody, and about 12.5 μg/ml anti-tag antibody printed and incubated with 0.1 μg/ml of scFv (the lowest concentration of scFv used in this experiment). No capture was apparent on negative control loci that contained identical concentrations of a different anti-tag monoclonal antibody incubated with identical concentrations of non-specific scFv containing the tag (FIG. 9).

### 2. Array Capture of Cells Growing in Culture

Arrays were prepared as for previously frozen cells, but the starting concentrations of S1C5 and anti-tag antibodies was 200 μg/ml. Two-fold serial dilutions were made 6 times for printing. In addition, the monoclonal antibody, anti-C6VL, which recognizes the T-cell receptor on the C6VL T-cell line, was added. In the mask, arrays were incubated with 10-fold serial dilutions of a 10 μg/ml solution of tag-S1C5 scFv, starting with 10 μg/ml. All incubations were carried out in RPMI 1640 Medium with 10 mM Hepes (pH 7.4), 0.5 or 0.25% BSA, and no phenol red. The slides then were incubated with either 38C13 B-cells, or C6VL T-cells and viewed immediately, with no washing. 38C13 cells were detected bound to loci printed from 3.12 μg/ml solutions of S1C5 antibody (the lowest concentration used in this experiment) and loci printed with 6.25 μg/ml solutions of anti-tag antibody and loaded with as little as 0.01 μg/ml solutions of specific scFv (FIG. 9). No binding was detected on negative control antibodies and scFvs (FIG. 9).

### 3. Chemical Fixation of Captured Cells

Slides were prepared as for the previous experiment, but were stored 1.5 weeks longer at 4° C. Incubations were carried out as above, except that only 38C13B cells were used, and wells in the mask were washed as described above. After the mask was removed, excess Wash Buffer was absorbed and Formaldehyde Solution was applied as described in-above. After washing and permeabilization, slides were viewed and images recorded using the Nikon E800 and Spot CCD Camera (FIG. 9).

### Example 8

### Cell Capture on Antibody Array with Immunofluorescent Detection

### A. MicroArray Printing

Stock solutions of the anti-M2 capture monoclonal antibody (M2), anti-Myc capture monoclonal antibody (Myc), anti-IgM (S1C5; anti-idiotype monoclonal antibody) and anti-T cell receptor antibody (C6VL) were prepared at a concentration of 1 mg/ml or greater in PBS. Neutravidin (Nv), which was conjugated to HRP, was used as a Luminol reaction negative control. For printing, the antibodies were brought to 800 μg/ml in 1× Print Buffer (1×PBS, 20% glycerol, 0.001% Tween-20) by adding ¼ volume of 4× Print Buffer (4×PBS, 80% glycerol, 0.004% Tween-20) to ¾ volume of a 1 mg/ml antibody solution in PBS. Two-fold serial dilutions were made of each antibody such that all antibodies were at 9 different concentrations in 1× Print Buffer (Table 12). Forty μl of antibody solution was transferred to a 96-well PCR plate.

Each of the antibodies were printed in ten arrays on four FAST™ nitrocellulose-coated glass slides (Schleicher and Schuell) using a Telechem pin (CM4) in a Cartesian printer (MicroSys 5100). Printing was performed at 55 to 60% relative humidity. The slides were subsequently incubated overnight at 4° C. for maximum adsorption to the nitrocellulose and then stored at 4° C. until use.

### B. Preparation of Non-adherent Cells for Capture

B cells (38C13) and T cells (C6VL) were grown, isolated and stored as described in EXAMPLE 7 above. The 38C13 B cells (8 ml; 1.9×10⁶ cells/ml) and C6VL T cells (8 ml; 1.1×10⁶ cells/ml) were removed from storage and placed on ice. Once thawed, the cells were spun down at 1000 g for 10 minutes at 4° C. The cells were gently resuspended in the same volume of Cell Incubation Medium from which the cells were initially pelleted (i.e., 8 ml). The resuspended cells then were spun down again at 1000 g for 10 minutes at 4° C. The cells then were resuspended again in 1 ml of Cell Incubation Medium using a 1 ml pipet tip and pipetman. The C6VL T cells were at a final concentration of 1×10⁷ cells/ml as determined by counting with a heamacytometer and an inverted microscope. The 38C13B cells were diluted to the same concentration by adding another 600 μl of Cell Incubation Medium. The cells were placed on ice until use.

TABLE 12
Array Map (μg/ml)

| | 1 | 2 | 3 | 4 | 5 | 6 | 7 | 8 | 9 | 10 | 11 |
|---|---|---|---|---|---|---|---|---|---|---|---|
| A | NV-HRP 200 | Nv 800 | S1C5 800 | S1C5 200 | S1C5 50 | S1C5 12.5 | S1C5 3.12 | S1C5 0.825 | S1C5 0.2 | S1C5 0.05 | PB |
| B | NV-HRP 100 | Nv 400 | C6VL 800 | C6VL 200 | C6VL 50 | C6VL 12.5 | C6VL 3.12 | C6VL 0.825 | C6VL 0.2 | C6VL 0.05 | PB |
| C | NV-HRP 50 | Nv 200 | M2 800 | M2 200 | M2 50 | M2 12.5 | M2 3.12 | M2 0.825 | M2 0.2 | M2 0.05 | PB |
| D | PB | Nv 100 | M2 800 | M2 200 | M2 50 | M2 12.5 | M2 3.12 | M2 0.825 | M2 0.2 | M2 0.05 | PB |
| E | PB | Nv 50 | HA 800 | HA 200 | HA 50 | HA 12.5 | HA 3.12 | HA 0.825 | HA 0.2 | HA 0.05 | PB |
| F | NV-HRP 50 | Nv 25 | HA 800 | HA 200 | HA 50 | HA 12.5 | HA 3.12 | HA 0.825 | HA 0.2 | HA 0.05 | NV-HRP 50 |
| G | NV-HRP 100 | Nv 12.5 | myc 800 | myc 200 | myc 50 | myc 12.5 | myc 3.12 | myc 0.825 | myc 0.2 | myc 0.05 | NV-HRP |
| | | | | | | | | | | | 100 |
| H | NV-HRP 200 | Nv 6.25 | myc 800 | myc 200 | myc 50 | myc 12.5 | myc 3.12 | myc 0.825 | myc 0.2 | myc 0.05 | NV-HRP |
| | | | | | | | | | | | 200 |

### C. Array Incubations

### 1. Incubation with Primary Antibody or scFv

The printed slides were brought to room temperature and washed three times each for one minute with PBS. Following the wash step, each slide was wet in 3 ml Block Buffer (PBS/0.5% BSA (Sigma)) then blocked with 200 μl Block Buffer for one hour at room temperature on an orbital shaker in a humidified chamber. The slides then were placed in a mask and incubated for 1 hour at room temperature with 100 μl of the primary antibody or scFv as indicated in Table 13 below. The primary antibodies were prepared as shown in Table 14 below. Following incubation, the wells were washed 3 times with 200 μl Cell Incubation Medium (RPMI 1640 (Gibco), 10 mM Hepes, pH 7.4, 0.5% BSA, no Phenol Red and sterile filtered) for 1 minute on an orbital shaker. After the third wash, 50 μl of fresh Cell Incubation Medium was added.

TABLE 13

| | | Tube # | | |
|---|---|---|---|---|
| Slide | | (from | Concentration | |
| No. | 1° Antibody | below) | of scFv | Cells Incubated |
| 33900 | M2-S1C5 scFv\ | 1 | 1.0 μg/ml each | 38C13 arrays 1-5 |
| | HA-HFN | | | C6VL arrays 6-10 |
| 33901 | M2-S1C5 scFv\ | 1 | 1.0 μg/ml each | 38C13 arrays 1-5 |
| | HA-HFN scFv | | | C6VL arrays 6-10 |
| 33902 | M2-HFN scFv\ | 2 | 1.0 μg/ml each | 38C13 arrays 1-5 |
| | HA-S1C5 scFv | | | C6VL arrays 6-10 |
| 33903 | M2-HFN scFv\ | 2 | 1.0 μg/ml each | 38C13 arrays 1-5 |
| | HA-S1C5 scFv | | | C6VL arrays 6-10 |

TABLE 14

| scFv | Stock Conc. | Stock vol. | | Final Conc. | | |
|---|---|---|---|---|---|---|
| (Expt. #) | (μg/ml) | (μl) | Block Buffer | (μg/ml) | Final Vol., μl | Tube # |
| M2-S1C5 | 500.0 | 2.0 | 996.6 | 1.0 | 1000 | 1 |
| (B25E16) | | | | | | |
| HA-HFN | 710.0 | 1.4 | — | 1.0 | — | 1 |
| (1.24.02) | | | | | | |
| M2-HFN | 1150.0 | 1.0 | 993.5 | 1.0 | 1200 | 2 |
| (B25E16) | | | | | | |
| HA-S1C5 | 0.022 | 5.5 | — | 1.0 | — | 2 |
| (B25E16) | | | | | | |

### 2. Incubation with Cells

The slides then were incubated with 38C13B cells and C6VL T cells as shown in Table 13 above. Fifty ∥l of cells were added per well and incubated for 30 minutes on an orbital shaker at room temperature. The wells then were washed 3 times by gently adding 300 μl of Cell Incubation Medium. Following the last wash, the Cell Incubation Medium was left in the wells. The mask was removed and the remaining wash solution was allowed to flow down the length of the slide. The excess wash medium was absorbed from the slides with a kimwipe at one edge. The slides then were placed in a humidified chamber with 1 ml of formaldehyde solution and allowed to incubate for 10 minutes at room temperature. The slides then were washed 3 times with 50 ml PBS each time. Following washing, the slides were placed in fresh PBS with 0.02% sodium azide and stored at 4° C.

### D. Immunofluorescence Staining

The slides were permeabilized by incubating 5 minute with 0.1% TX-100 in PBS followed by rinsing 3 times with 50 ml of PBS. The slides then were transferred to jig. Each well was blocked with Block Buffer (1% BSA/PBS) for 1 hour on orbital shaker at room temp.

The Fluorescence Labeling Solution was prepared as follows: Goat anti-Mouse IgM—Oregon Green (Molecular Probes) was diluted in Block Buffer to a final concentration of 5 μg/ml. Five μl per 200 μl of Fluorescence Labeling Solution of Rhodamine-Phalloidin (Molecular Probes) then was added from a stock (300 Units/ml).

The Block Buffer was aspirated from the wells followed by addition of 50 μl of Labeling Solution per well. The slides were incubated for 1 hour at room temperature on an orbital shaker. After the incubation, the slides were washed 3 times for 3 minutes each in 200 μl of Block Buffer on an orbital shaker at room temperature. One ml of ProLong® mounting medium was added to a vial containing the ProLong® antifade reagent (ProLong® Antifade Kit; Molecular Probes) in preparation of the antifade solution. The slide was removed from jig, drained and dried along edge with a Kimwipe. Several drops of mixed AntiFade were added along the length of the slide. After the addition, the slide was covered with a cover slip. The slide then was examined in a Nikon E800 fluorescence microscope and photographed with a Spot digital camera.

### E. Results

Arrays were printed with anti-tag antibodies (800, 200, and 50 μg/ml solution were printed) and loaded with anti-cell surface receptor scFv fused to the appropriate tag (1 μg/ml solution). The cells were fixed in a 4% formaldehyde solution, permeabilized with TX-100 and double-fluorescently labeled for both an intracellular protein, actin, as well as a cell surface receptor, membrane-bound IgM. Actin-was visualized with Rhodamine and the IgM with Oregon Green fluorescent dye. In the bottom panel, the cells were imaged by differential interference contrast microscopy.

### Example 9

### Preparation of Arrays on 96-Well Plates

Capture-antibody arrays can be printed into 96-well plate format and used in a similar manner to arrays printed onto FAST™ slides and nitrocellulose filters. This example demonstrates the use of the 96-well plate format to assay the Tag distribution in an scFv Tag library. Other assays, including functional assays, are performed in 96-well plate arrays in a similar manner/

### A. Capture Antibody Printing onto 96-Well Plates

Capture antibody dilutions were printed onto 96-well Maxisorp Immunoplates (NUNC; catalog #442404) using a pin-printer-style microarrayer (MicroSys 5100; Cartesian Technologies; TeleChem Arrayl™ Chipmaker 2 microspotting pins, catalog #CMP2). Printing was performed using the manufacturer's printing software program (Cartesian Technologies' AxSys version 1, 7, 0, 79) and a single pin. Microarray patterns were pre-programmed (in-house) to suit a particular microarray configuration, for example as a 5×5 pattern of 35 spots per well in each of 96 wells.

Microtiter plates (96-well) containing capture antibody dilutions (typically 400 μg/ml in 20% glycerol 1×PBS, 0.001% Tween-20 and MilliQ water) were loaded into the microarray printer for printing onto the plates. Based on the reported print volume (post-touch-off, see above) of 1 nl/microspot for the Chipmaker 2 pins, the capture antibody concentrations contained in the printed microspots typically ranged from 800 to 6 pg/microspot.

Source plate map

| Well # | Protein/Antibody |
|---|---|
| 1 | HRPO°Alexa |
| 2 | 4C10 |
| 3 | HA-11 |
| 4 | B34 |
| 5 | HSV |
| 6 | E-Tag |
| 7 | myc |
| 8 | M2 (Flag) |
| 9 | T7 |
| 10 | Glu-Glu |
| 11 | V5 |

| | | | | |
|---|---|---|---|---|
| HRPO°Alexa | 4C10 | 4C10 | HA-11 | HA-11 |
| HRPO°Alexa | VSV-G | VSV-G | HSV | HSV |
| Print Buffer | E-tag | E-tag | myc | myc |
| Print Buffer | M2 | M2 | T7 | T7 |
| HRPO°Alexa | Glu-Glu | Glu-Glu | V5 | V5 |

The printed 96 well plates were washed with three washes of TBST-T. Washed plates then were blocked by incubating with 100 μl 3% NFDM in 1×TBST for 1 hour at 37° C. The plates then were washed again with TBST-T.

### B. Basic Protocol for Capture Agent and Tag Library Incubations

### 1. Preparation of the ScFv Tag Library Standards with 10 Tags

Tag libraries were prepared using the tags corresponding to the antibodies in the source plate above (wells 2-11). The tag libraries were prepared and purified as in Example 3. A master mix of Tag Library standards was prepared based on the least concentrated of the 10 purified tag libraries such that the final concentration of each Tag library in the mix was 10 μg/ml in BBSA (Blocker BSA™; Pierce catalog #37525).

### 2. Addition of the Tag Library to the Capture Agent Array

For assay purposes, the master mix of Tag Libraries was first diluted 1:10 to give a starting concentration of 1 μg/ml for each tag library in BBSA. The master mix tag library was subsequently diluted through a series of 7 serial 2-fold dilutions into 3% NFDM in TBST.

The serial dilutions of the master mix Tag library were added to the wells of capture agents array plates. The tag library and the capture agents then were incubated together for 1 hour at 37° C. and then washed with TBST-T.

### 3. Detection of bound ScFvs to the Capture Agent Array

Polyclonal anti-6His antibody∘HRPO (Abcam) was diluted 1:10,000 in BBSA-T in a sufficient volume to distribute 50 μl of the solution to each well of the capture agent array plates. After addition of the solution to each well, plates were incubated for 1 hour at 37° C. and then washed with TBST-T.

Supersignal ELISA Femto Reagents (Pierce) were prepared by mixing the two developer components in equal volumes. Fifty microliters of developer was added to each well of the capture agent-tag library plates. Each plate then was imaged on a Kodak Image Station 440 using pre-set image parameters for half-plate imaging as specified by the manufacturer (Kodak, Rochester, N.Y.). Images were saved as JPEG files and archived for processing and then processed using a software analysis imaging program. The experimental data was plotted relative to standard curves to obtain the relative amounts of each tag in the Tag library.

### Example 10

### High-Throughput Preparation of ScFv Tag Libraries

### A. Preparation of Starter Blocks

Tag Libraries are prepared and titered as in Example 3. After calculating the required volumes needed for each tag library, glycerol stocks of each library are thawed on ice. The tag library volumes are mixed together in a single 50 ml Falcon tube on ice. This mixture is designated the array library starter culture.

2×YT media (VWR; ) with 100 μg of carbenicillin was added to bring the total volume to 0.1 ml×the number of library pools to be expressed. For example, typically ˜2000 pools were expressed and thus the array library starter culture volume was brought to 200 ml with the media addition. The array library starter culture in the media then was distributed to deep-well 96 well blocks at 100 μl/well. 2×YT media with 100 μg of carbenicillin was added to each well to bring the total well volume to 1 ml. The blocks then were incubated for 6 hours at 37° C. with shaking at 260 rpm. Blocks then were stored at 4° C. for up to 5 days.

One milliliter of culture from each of the wells of the starter blocks was added to a separate corresponding labeled Falcon tube containing 5 ml of 2×YT media with 100 μg of carbenicillin. The tubes were incubated for 15-17 hours at 37° C. with shaking at 260 rpm.

Glycerol stocks were prepared in 96-well cluster tubes by aliquoting 200 μl of 80% glycerol pre-warmed to 45° C. to each tube (one for each of the above cultures) and then adding 600 μl from the corresponding of the starter culture tube. The tubes were mixed and then stored at −80° C.

### B. Induction of the Array Library

Four liters of induction media (2×YT+100 μg of carbenicillin) was prepared and 24 ml of 20% arabinose was added. Twenty milliliters of media was added to each array library culture tube (above). Cultures then were incubated for 5 hours at 30° C. with shaking at 260 rpm.

### C. Lysis and Incubation with Ni-NTA Resin

Cultures were removed from the 30° C. incubator and centrifuged at 400 rpm (2250×g) for 15 minutes. Supernatants were decanted and then the tubes were inverted and drained for an additional 3 minutes. Periplasting solution was prepared by adding 50 μl of lysozyme (30 U/ml) to 100 ml of periplasting buffer (200 mM Tris-HCl, pH 7.5, 20% sucrose, 1 mM EDTA). Each cell pellet was resuspended in 500 μl of periplasting solution by gentle vortexing and pipetting, and then incubated at room temperature for 10 minutes. Individual periplasted cultures were transferred to wells of deep-well 96-well blocks and 500 μl of milliQ water added to each well with gentle mixing. Blocks were incubated on ice for 10 minutes followed by centrifugation at 4000 rpm for 30 minutes at 4° C.

From the centrifugation, 800 μl of supernatant was transferred from each well to corresponding new wells of deep-well 96-well blocks. The blocks were re-centrifuged at 4000 rpm for 30 minutes at 4° C. to clarify the suspensions and 600 μl was transferred from each well to corresponding new wells of 96-well tube blocks (VWR). To each tube, 266 μl of adjustment buffer was added ( adjustment buffer was made from 230 ml 5M NaCl, 9 ml 5M imidazole, 12 ml 1 M MgCl2, 58 ml 1 M NaH₂PO₄, 144 ml 80% glycerol,, 10 ml 10% Triton X-100 and 0.51 ml 1000× protease inhibitor AEBSF (VWR)), followed by 200 μl of NI-NTA Superflow slurry (QIAGEN). The blocks placed on their sides for maximum mixing and were incubated overnight at 4° C. with rocking.

### D. Washing and Elution from the Ni-NTA Resin

After the overnight incubation, the N-NTA slurry preps were transferred to 96-well Turbo Filter blocks (QIAGEN). Filter blocks were incubated 10 minutes on ice to allow the resin to settle out of solution. Each filter block then was positioned on top of a QiaVAC manifold (QIAGEN) with a deep-well 96-well block placed below into the vacuum chamber of the manifold. The vacuum was attached to the manifold following manufacturer's instructions and vacuum applied to drain the flow-through solution from the filter block. Two hundred microliters of wash buffer (50 mM NaH₂PO₄ pH 8.0, 1.5 M NaCl and 40 mM imidazole) was applied and washed through each well and then the wash steps repeated for a total of three washes.

After the third wash, the vacuum was applied to dry the resin. A new 96-well deep-well block was put into the vacuum chamber. Elution buffer (50 mM NaH₂PO₄ pH 8.0, 1.5 M NaCl and 500 mM imidazol) then was applied to the filter block, 150 μl per well and allowed to sit for 1 minute. Vacuum then was applied and then an additional 150 μl of elution buffer was applied and eluted in the same manner.

The eluted samples from the 96-well deep-well blocks were transferred to wells of DispoDialyzer blocks (Nest Group) which had been pre-wet with 1×PBS. The wells of the blocks were capped and the blocks -placed in 2 l of 1×PBS with stirring overnight. After dialysis, samples were transferred to wells of 96-well deep-well blocks. Sample volume was estimated and glycerol was added to each well to a final concentration of 20%. Aliquots from the wells were transferred to wells of additional 96-well plates for analysis (protein concentration, SDS-PAGE analysis, Tag distribution assay) and for use in functional assays. These plates were stored at 4° C. The blocks containing the remaining samples were stored at −80° C.

### E. Results

An aliquot from each well of a 96-well block was analyzed for protein concentration (see Example 5). Each well contained approximately 1000 scFvs×10 tags (10,000 scFv-tag molecules/culture). An average of 0.03 mg of protein (±10%) was recovered from each well, enough material for approximately 100 screening capture agent array assays. Tag distribution was also assessed from these samples. Since 10 tags were used for this library, each tag was expected to be represented ˜10% of the total. The analysis indicated an average of ˜10% for each tag with a variation between samples from ˜5% to ˜20%. Increasing the number of tags decreases the range of variation from the expected distribution.

### Example 11

Generation of Binding Partner-Capture Agent Pairs

### A. Generation of 6-mer Polypeptide Epitope Tags

A collection of 6 amino acid polypeptides (6-mers) were designed using the method described in Example A. The polypeptides were designed for screening suitability and use as binding partners paired with capture agents.

Peptides (6-mers) were synthesized with a C-terminal cysteine residue as: cysteine-(amino acid)₆-NH2. Diphtheria toxoid was activated using MCS to add maleimido groups to lysine side chains (Lee A C J, Powell J E, Tregear G W, Niall H D and Stevens V C (1985) Mol. Immunol. 17:749-756). A 1.5 molar excess of the activated carrier protein was incubated with the polypeptides. The ratio ensures the lack of free unconjugated polypeptides such that unconjugated polypeptides or carrier proteins are not separated from the conjugated sample.

The 6 mer polypeptides are also synthesized with biotin at the C-terminal end with a 4-mer linker polypeptide for use in screening assays: Biotin-SGSG-(amino acid)6-NH2.

### B. Immunization of Mice with DT-Peptide Conjugates

The DT-peptide conjugates were dissolved in PBS. To formulate the mixture of conjugates, 0.5 mg of each of 4 peptides is added into one tube and the volume made to 2 ml with sterile PBS. The conjugates are mixed well before dispensing so that any particulate is well suspended. Each group of 4 polypeptide conjugates is designated by a group name, for example, as Grp1, Grp2, Grp3, and so on.

Three mice were immunized with each group of polypeptide conjugates. Mice were immunized with 200 μg protein/mouse for initial immunization (day 0) and boosts of 100 pg protein/mouse at days 21, 35, 49 and 63. Tail bleeds were taken at day 42 and day 70 and analyzed by ELISA assays. Samples of serum were taken from tail bleeds of the mice before day 0 immunizations to serve as pre-immune control serum.

Mice were analyzed by ELISA as follows. Biotinylated polypeptides were dissolved in DMSO at final concentrations of 5 mg/ml. NUNC Maxisorp plates are coated with 5 μg/ml Neutravidin in PBS and incubated at 4° C. until use (up to 30 days). The NeutrAvidin is aspirated off and the plates incubated with biotinylated polypeptides at 5 μg/ml in PBS for 60 min at 37° C. as indicated in the table below.

| | Plate 1 | Plate 2 | Plate 3 | Plate 4 | Plate 5 | Plate 6 |
|---|---|---|---|---|---|---|
| A | Peptide | Peptide 9 | Peptide 17 | Peptide 25 | Peptide 33 | Peptide 41 |
| | 1 | | | | | |
| B | Peptide | Peptide 10 | Peptide 18 | Peptide 26 | Peptide 34 | Peptide 42 |
| | 2 | | | | | |
| C | Peptide | Peptide 11 | Peptide 19 | Peptide 27 | Peptide 35 | Peptide 43 |
| | 3 | | | | | |
| D | Peptide | Peptide 12 | Peptide 20 | Peptide 28 | Peptide 36 | Peptide 44 |
| | 4 | | | | | |
| E | Peptide | Peptide 13 | Peptide 21 | Peptide 29 | Peptide 37 | Peptide 45 |
| | 5 | | | | | |
| F | Peptide | Peptide 14 | Peptide 22 | Peptide 30 | Peptide 38 | Peptide 46 |
| | 6 | | | | | |
| G | Peptide | Peptide 15 | Peptide 23 | Peptide 31 | Peptide 39 | Peptide 47 |
| | 7 | | | | | |
| H | Peptide | Peptide 16 | Peptide 24 | Peptide 32 | Peptide 40 | Peptide 48 |
| | 8 | | | | | |

The plates were blocked with 1× Blocker BSA in PBS-T for 60 min at 37° C. One hundred microliters of each tail-bleed sample is added to Row A at a 1:100 dilution (2.5 μl of a 1:10 diluted tail-bleed and 22.5 μl Blocker BSA). To each plate, tail bleeds were added as follows (group refers to the groups of polypeptide-conjugates used for immunization, Mu1-Mu9 refer to the individual mice that were immunized with each group of peptides, described above).

| 1 | 2 | 3 | 4 | 5 | 6 | 7 | 8 | 9 |
|---|---|---|---|---|---|---|---|---|
| Tail | Tail | Tail | Tail | Tail | Tail | Tail | Tail | Tail |
| bleed | bleed | bleed | bleed | bleed | bleed | bleed | bleed | bleed |
| Grp1 | Grp1 | Grp1 | Grp2 | Grp2 | Grp2 | Grp3 | Grp3 | Grp3 |
| Mu1 | Mu2 | Mu3 | Mu4 | Mu5 | Mu6 | Mu7 | Mu8 | Mu9 |

The plates were incubated for 60 min at 37° C. and then washed 3× with 1×TBS-T. They then were incubated with 100 μl of a 1:2000 dilution of goat anti-mouse IgG-HRP conjugate for 60 min at 37° C., washed again 3 times with TBS-T and developed with OPD. The absorbance measured at 492 nm.

### C. Generation of a Library of Hybridoma Cells

An additional 1.2 mg of conjugate-peptide mixtures (0.3 mg of each) was prepared for injection into mice prior to fusion. The mice were boosted with injections of polypeptides for three days prior to fusion. Fusion of spleen cells with mouse myeloma cells was performed on Day 84 and the hybridoma cells were grown in selection medium for 4 weeks. The medium was removed 3 weeks after fusion and fresh medium was added. The medium was harvested on Week 4 after fusion and tested for presence of anti-peptide antibodies by ELISA as described above. The assay was performed only for determination of antibodies to the immunized polypeptides and not for cross-reactivity. The cells were harvested, aliquoted and stored (Fusion library) until the results from analysis of supernatants were obtained.

### D. Cloning of Hybridomas to Generate Monoclonal Antibodies

A vial of the fusion library was thawed and the cells grown in medium for 2 weeks. Cells then were sorted using a FACS into ten 96-well plates such that each well received a single cell. The cells were grown for 2 weeks and the supernatant from each clone analyzed for presence of anti-peptide antibody as for the fusion library supernatant.

Positive clones were identified and ranked in order of ELISA signal intensities. Twelve clones with the highest signal intensities were scaled-up and assayed for polypeptide-specific antibody after 2 weeks. The supernatants then were assayed for antibody titre determination and two clones showing the highest anti-peptide antibody titre were selected for scale-up and storage. The clones were grown to obtain 100 ml of medium and the cells then were frozen at −80° C.

### E. Purification and Isotyping of IgG from Hybridoma Lines

The selected clones were grown for 2 weeks and the medium was used for analysis of antibody class and for specificity of binding to polypeptides by performing the assay described above. IgG was isotyped using Isotype mouse isotyping kits (Roche). The antibody from the supernatant was purified using Protein G affinity chromatography and stored in liquid nitrogen.

### F. Results

Peptides used for the immunizations were as follows:

| | | | |
|---|---|---|---|
| SEQ ID NO: | Peptide | SEQ ID NO: | Peptide |

| | | | |
|---|---|---|---|
| 949 | EPNGYF | 324 | QGKEYF |
| 953 | EGYPNF | 381 | NSFEGP |
| 1085 | PEQGYN | 383 | NFKSGH |
| 1089 | PGYEQN | 387 | NSGFKH |
| 273 | QESGPD | 388 | NGFKYH |
| 288 | QPGYEH | 409 | NTSGHK |
| 366 | NQHGYD | 416 | NKGYHL |
| 378 | NGYFEP | 465 | FPSGNE |
| 956 | ESPNGF | 487 | FNPSGE |
| 958 | EPHSGK | 491 | FSGNPE |
| 962 | ESGPHK | 492 | FGNPYE |
| 963 | EGPHYK | 518 | FTLGYQ |
| 967 | EQGYPN | 522 | FGYTLQ |
| 976 | EQSGFH | 525 | FSTLGQ |
| 1092 | PSEQGN | 603 | HSGQEL |
| 1094 | PEFSGQ | 607 | HQTSGN |
| 187 | PSGEFQ | 622 | HNDGYT |
| 188 | PGEFYQ | 632 | HFGYTK |
| 192 | PEGYKD | 673 | HDSGTL |
| 209 | PNSGEF | 728 | TLGYNF |
| 298 | QGYNHE | 772 | KGQNYT |
| 301 | QSNHGE | 784 | KNGYDQ |
| 302 | QFEGYK | 810 | KGYHPD |
| 319 | QKESGF | 813 | KSHPGD |

Peptides were injected singly or in groups of 2-4 polypeptides/animal as described above. Antisera were analyzed as described. All of the injected polypeptides raised antisera that was high specificity and affinity.

Since modifications will be apparent to those of skill in this art, it is intended that this invention be limited only by the scope of the appended claims.

1. A method for evenly distributing tags among members of a starting library, comprising:
a) optionally adjusting the diversity of a starting library so that the diversity is within an order of magnitude of the number of molecules in the library;
b) dividing the starting library into “n” sublibraries designated 1 to n, wheren n is equal to or less than the number of unique tags, wherein each unique tag specifically binds to a different capture agent;
c) attaching a tag to a plurality of members of each sublibrary to produce “n” tagged sublibraries containing tagged members, wherein each member has the same tag, and the tag is unique to each sublibrary;
d) mixing some or all of the tagged sublibraries to produce a mixed library, wherein the number of tagged molecules added from each sublibrary is the same; and
e) splitting the mixed library into “q” array libraries, wherein q is from 1 up to a predetermined number of arrays.

2. A method for evenly distributing nucleic acid molecules that encode polypeptide tags among members of a starting library, comprising:
a) optionally, adjusting the diversity of a starting library so that the diversity is within an order of magnitude of the number of members in the library;
b) dividing the starting library into “n” sublibraries designated 1 to n, wherein n is equal to or less than the number of different nucleic acid molecules having nucleic acid molecules encoding different polypeptide tags;
c) attaching a nucleic acid molecule encoding a polypeptide tag to members of each sublibrary to produce “n” tagged sublibraries containing tagged members, wherein the encoded polypeptide tag is unique to each sublibrary;
d) mixing some or all of the tagged sublibraries to produce a mixed library, wherein the number of tagged nucleic acid molecules added from each sublibrary is the same;
e) splitting the mixed library into “q” array libraries, wherein q is from 1 to a predetermined number of arrays.

3. The method of claim 2, wherein the starting library is a nucleic acid library, and at step c) the polypeptide tag encoding portion of the tag is in reading frame with polypeptides encoded by the members of the sublibrary.

4. The method of claim 3, further comprising expressing the encoded polypeptides to produce tagged polypeptides in each array library.

5. The method of claim 1, further comprising:
contacting the array libraries with 1 up to q collections of addressed capture agents under conditions in which the tags bind to the capture agents to produce 1 to q capture systems, wherein the capture agents at each locus in the addressed collection specifically bind to the same tag.

6. The method of claim 1, further comprising contacting array libraries with addressed capture agents, wherein agents at each addressed locus bind to the same polypeptide tag, thereby sorting the tagged molecules according to their tag.

7. The method of claim 1, further comprising:
f) preparing up to “q” arrays from the array libraries.

8. The method of claim 3, wherein the tagged polypeptides in each array library are produced by translation of nucleic acid molecules encoding tagged polypeptides.

9. The method of claim 1, wherein, on the average, each tagged molecule is unique in each array library.

10. The method claim 1, wherein the diversity of the starting library is about equal to the number of molecules in the library.

11. The method of claim 1, wherein the diversity of the starting library is about within about half an order of magnitude of the number of molecules in the library.

12. The method of claim 1, wherein the diversity of the starting library is with about 0.05 or 0.01 order of magnitude of the number of molecules in the library.

13. The method of claim 1, wherein the diversity of each sublibrary of tagged molecules is the about same.

14. The method of claim 13, wherein the diversity of each sublibrary of tagged molecules is within about 0.5 order of magnitude of all other tagged sublibraries.

15. The method of claim 13, wherein the diversity of each sublibrary of tagged molecules is within about 0.1 order of magnitude of all other tagged sublibraries.

16. The method of claim 13, wherein the diversity of each sublibrary of tagged molecules is within about 0.05 order of magnitude of all other tagged sublibraries.

17. The method of claim 13, wherein the diversity of each sublibrary of tagged molecules is within about 0.01 order of magnitude of all other tagged sublibraries.

18. The method of claim 2, wherein the polypeptide tag encoding portion of the tag is in reading frame with a polypeptide encoded by the nucleic acid molecule in the library.

19. The method of claim 2, wherein the nucleic acid molecule encoding the polypeptide tag is linked via a sequence of nucleic acid molecules that encode an additional polypeptide linker to nucleic acid molecule members of the library.

20. The method of claim 1, wherein the diversity of the starting library is 10², 10³, 10⁴, 10⁵, 10⁶, 10⁷, 10⁸, 10⁹, 10¹⁰, 10¹¹, 10¹² or greater.

21. The method of any of claim 1, wherein the diversity of the starting library is adjusted.

22. The method of 21, wherein the diversity is adjusted to be about equal to the number of molecules in the library.

23. The method of claim 21, wherein the diversity is adjusted to be within about 0.5 order of magnitude of the number of molecules in the library.

24. The method of claim 21, wherein the diversity is adjusted to about within an about 0.1 of an order of magnitude of the number of molecules in the library.

25. The method of claim 1, wherein the starting library is a nucleic acid library.

26. The method of claim 1, wherein the starting library is a cDNA library.

27. The method of claim 1, wherein the starting library encodes antibodies or fragments thereof or is comprised of antibodies or fragments thereof, wherein the antibodies or fragments thereof specifically bind to antigens.

28. The method of claim 25, wherein the library encodes single-chain antibody fragments (scFvs).

29. The method of claim 5, wherein the capture system comprises tagged polypeptides bound to antibodies or fragments thereof.

30. The method of claim 29,, wherein the antibodies or fragments that bind to tagged polypeptides comprise two polypeptide chains.

31. The method of claim 2, wherein:
the starting library is a nucleic acid library; and
the step of attaching a nucleic acid molecule encoding a polypeptide tag to molecules of each sublibrary is effected by cloning members of the nucleic acid sublibraries into sets of plasmids that comprise nucleic acid encoding the polypeptide tags;
there are up to “n” sets of plasmids;
each set of plasmids comprises nucleic acid that encodes a single polypeptide tag and each set encodes a unique polypeptide tag;
the molecules of each sublibrary are cloned into one set of plasmids, whereby the molecules of each sublibrary are tagged with the same tag-encoding nucleic acid, and each sublibrary is tagged with a unique tag-encoding nucleic acid.

32. The method of claim 31, further comprising transforming host cells with the sets of plasmids to produce sets of host cells; and maintaining them under conditions whereby the number of plasmids does not increase.

33. The method of claim 32, further comprising titering an aliquot of the transformed host cells from a plurality of sets of host cells that comprise tagged sublibraries.

34. The method of claim 32, further comprising normalizing the titer of plasmids in each of the tagged sublibraries in the sets of host cells so that the titer of each sublibrary is within 1, 0.5, 0.1, 0.05, or 0.01 order(s) of magnitude of the other tagged sublibrary titres.

35. The method of claim 34, wherein normalizing is effected by mixing sets of host cells.

36. The method of claim 35, further comprising splitting the mixed cells into from 2 to “q” equal portions.

37. The method of claim 34, further comprising expressing and purifying the tagged polypeptides encoded in the plasmids to produce from 1 to q array libraries of tagged polypeptides.

38. The method of claim 37, further comprising contacting the array libraries, with a corresponding number of addressed capture agents to produce from 1 to q capture systems.

39. The method of claim 31, wherein the nucleic acid library encodes a library of antibodies.

40. The method of claim 39, wherein the antibodies are ScFvs.

41. A collection of tagged molecules produced by the method of claim 1, wherein:
the starting library is a nucleic acid library or a polypeptide library; and
the tagged molecules comprise tagged polypeptides.

42. A capture system, comprising:
tagged polypeptides of claim 41; and
an addressable collection of capture agents, wherein:
each locus in the collection contains capture agents that specifically bind to the same tag; and
the tagged molecules are specifically bound to capture agents.

43. A capture system, comprising:
an addressable collection of capture agents, wherein each locus in the collection contains capture agents that specifically bind to the same polypeptide tag, wherein the tags are evenly distributed among the tagged polypeptides;
a plurality of different polypeptide-tagged molecules bound to the capture agents, wherein the polypeptide-tagged molecules are sorted according to their specificity for the capture agents, wherein the tags are evenly distributed among the tagged molecules such that the diversity of tagged molecules at each locus in the collection is within one order of magnitude between and among loci.

44. A capture system, comprising:
an addressable collection of capture antibodies, wherein each locus in the collection contains antibodies that specifically bind to the same polypeptide tag;
a plurality of different polypeptide-tagged antibodies or fragments thereof bound to the capture antibodies;
wherein the polypeptide-tagged antibodies or fragments thereof are sorted according to their specificity for the capture antibodies; and
wherein the tags are evenly distributed among the tagged polypeptides such that the diversity of tagged molecules at each locus in the collection is within one order of magnitude.

45. The capture system of claim 42, wherein the diversity of tagged molecules at each locus in the collections is within 0.05 or 0.01 order of magnitude between and among loci.

46. The capture system of 42, wherein each locus in the capture system further comprises an additional agent or plurality thereof at one or more loci, wherein the additional agents are common to a plurality of loci, and bind to and/or interact with captured biological particles and/or captured molecules.

47. The capture system of claim 46, wherein a plurality of additional agents are added.

48. The capture system of claim 46, wherein the amounts of the additional agents vary from locus to locus.

49. The capture system of claim 46, wherein the additional agents are selected from the group consisting of antibodies known to bind to captured biological particles and molecules, adhesion molecules, drugs, receptors, enzymes and combinations thereof

50. The capture system of claim 46, where the additional agent serves to anchor molecules and/or biological particles, to act as a co-stimulatory molecule, to bind to surface receptors different from the first capture agents, to exert a biological effect, to further select the biological particles and/or captured molecules that bind to a locus.

51. The capture sytem of claim 46, wherein the additional agent is selected from the group consisting of trastuzumab and rituximab.

52. The capture system of claim 46, wherein the diversity of tagged molecules at each locus in the collection is within 0.5 order of magnitude or is within 0.1 order of magnitude.

53. The capture system of claim 42, wherein the polypeptide tagged molecules or polypeptides are polypeptide-tagged single-chain antibody fragments (scFvs).

54. A capture system, comprising:
a collection of tagged molecules produced by the method of claim 1; and
an addressable collection of capture agents, wherein:
each locus in the collection contains capture agents that specifically bind to the same tag;
the tagged molecules are specifically bound to capture agents; and
the diversity of tagged polypeptides or tagged molecules is 10³, 10⁴, 10⁵, 10⁶, 10⁷, 10⁸, 10⁹, 10¹⁰, 10¹¹, 10¹² or more.

55. A collection of tagged molecules, wherein:
the tags are evenly distributed among the tagged molecules such that the number of molecules having each tag is within 1.0, 0.5, 0.1, 0.05, or 0.01 order of magnitude; and
the collection has a diversity of at least 10³.

56. The collection of claim 55 that has a diversity of at least 10⁴.

57. The collection of claim 55 that has a diversity of at least 10⁵.

58. The collection of claim 55 that has a diversity of at least 10⁶.

59. The collection of claim 55 that has a diversity of at least 10⁷.

60. The collection of claim 55 that has a diversity of at least 10⁸.

61. The collection of claim 55 that has a diversity of at least 10⁹.

62. The collection of claim 55 that has a diversity of at least 10¹⁰.

63. The collection of claim 55, wherein the collection is a nucleic acid library.

64. The collection of claim 55, wherein the collection is a nucleic acid library tagged with oligonucleotides that encode polypeptide tags.

65. The collection of claim 55, wherein the collection is tagged with polypeptide tags.

66. The collection of claim 55, wherein the collection comprises polypeptides tagged with polypeptide tags.

67. The collection of claim 64 that is an addressable collection, wherein the diversity of different tagged molecules at each locus in the array is within one order of magnitude.

68. A capture system, comprising capture agents; and
a collection of claim 55 bound thereto.

69. A method for capturing molecules, comprising:
contacting a capture system with molecules under conditions whereby molecules bind to the capture system, wherein:
the capture system comprises a plurality of addressed loci;
the capture system comprises an addressed collection of polypeptide-tagged molecules bound to addressed capture agents at each locus;
the capture agents at each locus bind to the same polypeptide tag;
the polypeptide tag to which the capture agent binds is different among the loci;
each locus in capture system contains a plurality of different molecules each with the same tag bound to the capture agents; and
the polypeptide tags are evenly distributed among the tagged molecules such that the diversity of tagged molecules at each locus in the capture system is within one order of magnitude.

70. The method of claim 69, wherein the diversity of tagged molecules among the loci is within 0.5 order of magnitude.

71. The method of claim 69, wherein the diversity of tagged molecules among the loci is within 0.1 order of magnitude.

72. The method of claim 69, wherein the diversity of tagged molecules among the loci is within 0.05 or 0.01 order of magnitude.

73. The method of claim 69, wherein the tagged molecules are polypeptides.

74. The method of claim 69, wherein the tagged molecules comprise tagged nucleic acid molecules.

75. The method of claim 69, wherein the tagged molecules comprise tagged antibodies or fragments thereof.

76. The method of claim 75, wherein the polypeptide tagged antibodies or fragments are polypeptide-tagged single-chain antibodies (scFvs).

77. The method of claim 69, wherein the tagged molecules comprise a library of molecules.

78. The method of claim 77, wherein the library is an antibody library or a library of nucleic acid molecules encoding an antibody library.

79. The method of claim 77, wherein the library is an scFv library or a nucleic acid library encoding the scFvs.

80. The method of claim 69, wherein the capture agents comprise polypeptides or nucleic acids or analogs thereof.

81. The method of claim 69, wherein the capture agents comprise receptors, ligands, drugs, enzymes, or enzymes that are modified to have reduced catalytic activity.

82. The method of claim 69, wherein the capture agents comprise antibodies or fragments thereof.

83. The method of claim 69, wherein the capture system comprises a positionally addressable array.

84. The method of claim 83, wherein the capture agents are immobilized at discrete loci on a solid support.

85. The method of claim 84, wherein the solid support is selected from the group consisting of silicon, celluloses, metal, polymeric surfaces, and radiation grafted supports.

86. The method of claim 84, wherein the support comprises a well or a pit or plurality thereof in a surface of the solid support.

87. The method of claim 69, wherein the capture agents are addressably tagged by linking them to electronic, chemical, optically or color-coded labels.

88. The method of claim 87, wherein the labels comprise particulate supports.

89. The method of claim 88, wherein the particulate support is selected from the group consisting of silicon, celluloses, metal, polymeric surfaces and radiation grafted supports.

90. The method of claim 88, wherein the particulate support is selected from the group consisting of gold, nitrocellulose, polyvinylidene fluoride (PVDF), radiation grafted polytetrafluoroethylene, polystyrene, glass and activated glass.

91. The method of claim 69, wherein the tagged molecules have a diversity of at least about 10², 10³, 10⁴, 10⁵, 10⁶, 10⁷, 10⁸, 10⁹, 10¹⁰, 10¹¹ or 10¹².

92. The method of claim 69, wherein each locus in the capture system further comprises an additional agent or plurality thereof at one or more loci, wherein the additional agents are common to a plurality of loci, and bind to and/or interact with the captured biological particles and/or captured molecules.

93. The method of claim 92, wherein a plurality of additional agents are added.

94. The method of claim 92, wherein the amounts of the additional agents vary from locus to locus.

95. The method of claim 92, wherein the additional agents are selected from the group consisting of antibodies known to bind to the captured biological particles and/or captured molecules, adhesion molecules, drugs, receptors, enzymes and combinations thereof.

96. The method of claim 92, wherein the additional agent is selected from the group consisting of trastuzumab and ritimab.

97. The method of claim 69, wherein the molecules comprise biological particles; and wherein the biological particles are cells selected from the group consisting of immune cells, neurons, cancer cells, bacterial cells and infected cells.

98. The method of claim 69, wherein the molecules are biological particles selected from the group consiting of subcellular compartments, organelles, viral particles and pathogens.

99. The method of claim 69, wherein the cells are dendritic cells, T cells, or B cells.

100. The method of claim 69, wherein the capture agents are cell surface receptors, T cell receptors, MHC peptides, MHC peptide complexes, B cell receptors, ICAMs, Toll-like receptors, PPAR ligands, ion channels, chemokine receptors, nicotinic acetylcholine receptors, dopamine receptors, muscarinic receptors, small molecule receptors, ICAMs, TNF receptors, interleukin receptors, BCAMS, or interferons.

101. The method of claim, 69, further comprising:
assessing the effects of capture on a captured molecule or plurality thereof.

102. The method of claim 101, wherein the effect is selected from the group consisting of a change in structure, a change in activity, a physical change, and a chemical change.

103. The method of claim 101, wherein an effect is detected by visualizing the captured molecules.

104. The method of claim 101, wherein an effect is detected by staining or labeling captured molecules.

105. The method of claim 69, further comprising:
detecting or identifying captured molecules.

106. The method of claim 105, wherein identification is effected by staining or visualizing captured molecules.

107. The method of claim 69, wherein the molecules are labeled prior to capture.

108. The method of claim 69, further comprising: identifying tagged molecules that capture the molecules.

109. The method of claim 69, further comprising: identifying tagged molecules that capture labeled molecules.

110. The method of claim 106, wherein the stain specifically reacts with a one or a plurality of the captured molecules.

111. The method of claim 106, wherein a plurality of stains are applied.

112. The method of claim 111, wherein one stain reacts with a feature common to all molecules of a particular type, and at least one other stain reacts with a subset thereof.

113. The method of claim 106, wherein a stain is selected from the group consisting of fluorescent dyes, luminescent labels, enzyme labels, and immunostains.

114. The method of claim 106, wherein a stain is are selected from the group consisting of green fluorescent protein, red fluorescent protein, blue fluorescent protein, an immunostain and semiconductor crystals.

115. The method of claim 69, wherein contacting is performed in the presence and absence of a test compound, and the results are compared to identify test compounds that alter binding of molecules to the capture system.

116. The method of claim 69, further comprising:
adding a test compound or exposing the capture system to a condition before, during or after contacting the capture system with the molecules; and after contacting assessing the effects of the test compound on the captured molecules.

117. A method for identifying modulators of interactions between capture systems and molecules, comprising:
a) performing the method of claim 69;
b) adding a test compound or exposing the capture system to a condition before, during or after contacting the capture system with molecules or before, during or after contacting the capture agents with the tagged molecules; and
c) identifying a change in an interaction of the molecules with the capture system or tagged molecules with the capture agents to identify a test compound that modulates the interaction between the molecules and the capture system or between tagged molecules and capture agents.

118. The method of claim 117, wherein the change is assessed by detecting a change in binding pattern or a physical or chemical change in the bound molecules or a conformational change in the bound molecules and/or tagged molecules.

119. A method of sorting molecules or reducing the diversity thereof, comprising:
a) contacting a collection of tagged molecules with an array of addressed capture agents, wherein:
the agents at each addressed locus specifically bind the same tag, which differs from the tag to which agents at other loci bind;
the tags are evenly distributing among the tagged molecules; and
on the average, each tagged molecule is unique in each array library;
b) identifying from among the tagged molecules those having a predetermined activity or property;
c) based upon the tag(s) of the identified molecules, identifying the molecules linked to the tag, thereby sorting the molecules based upon the tag.

120. A method of reducing the diversity of a collection of molecules, comprising:
a) contacting a collection of tagged molecules with an array of addressed capture agents, wherein:
the agents at each addressed locus specifically bind the same tag, which differs from the tag to which agents at other loci bind;
the tags are evenly distributing among the tagged molecules; and
on the average, each tagged molecule is unique in each array library;
b) identifying from among the tagged molecules those having a predetermined activity or property;
c) based upon the tag(s) of the identified molecules, identifying the molecules linked to the tag;
d) selecting the molecules linked to the tag, thereby reducing the diversity of the collection of molecules.

121. The method of claim 2, further comprising:
contacting the array libraries with 1 up to q collections of addressed capture agents under conditions in which the tags bind to the capture agents to produce 1 to q capture systems, wherein the capture agents at each locus in the addressed collection specifically bind to the same tag.

122. The method of claim 2, further comprising contacting array libraries with addressed capture agents, wherein agents at each addressed locus bind to the same polypeptide tag, thereby sorting the tagged molecules according to their tag.

123. The method of claim 1, further comprising:
f) producing a capture system from each array library by contacting members of the array library with addressable collections of capture agents.

124. The method of claim 2, further comprising:
f) producing a capture system from each array library by contacting members of the array library with addressable collections of capture agents.

125. The method of claim 2, further comprising:
f) preparing up to “q” arrays from the array libraries.

126. The method of claim 2, wherein, on the average, each tagged molecule is unique in each array library.

127. The method of claim 2, wherein the diversity of each sublibrary of tagged molecules is the about same.

128. The method of claim 2, wherein the diversity of the starting library is 10², 10³, 10⁴, 10⁵, 10⁶, 10⁷, 10⁸, 10⁹, 10¹⁰, 10¹¹, 10¹² or greater.

129. The method of any of claim 2, wherein the diversity of the starting library is adjusted.

130. The method of 129, wherein the diversity is adjusted to be about equal to the number of molecules in the library.

131. The method of claim 2, wherein the starting library is a cDNA library.

132. The method of claim 2, wherein the starting nucleic acid library encodes single-chain antibody fragments (scFvs).

133. The collection of claim 65 that is an addressable collection, wherein the diversity of different tagged molecules at each locus in the array is within one order of magnitude.

134. The collection of claim 66 that is an addressable collection, wherein the diversity of different tagged molecules at each locus in the array is within one order of magnitude.
